(19) United States
(12) Patent Application Publication
(10) Pub. No.: US 2007/0275962 A1
(43) Pub. Date: Nov. 29, 2007

(54) Heterobicyclic Compounds as Pharmaceutically Active Agents

(75) Inventors: Anil Koul, Berchem (BE); Bert Klebl, Obesschweinbad (DE); Gerhard Muller, Utting (DE); Andrea Missio, Sovizzo (IT); Wilfried Schwab, Neuried (DE); Doris Hafenbradl, Pullach (DE); Lars Neumann, Munich (DE); Marc-Nicola Sommer, Munich (DE); Stefan Muller, Munich (DE); Edmund Hoppe, Krailling (DE); Achim Freisleben, Munich (DE); Alexander Backes, Munich (DE); Christian Hartung, Munich (DE); Beatrice Felber, Eching (DE); Birgit Zech, Munich (DE); Ola Engkvist, Bjarnum (SE); Gyorgy Keri, Budapest (HU); Laszlo Orfi, Budapest (HU); Peter Banhegyi, Budapest (HU); Zoltan Greff, Budapest (HU); Zoltan Horvath, Budapest (HU); Zoltan Varga, Kolesd (HU); Peter Marko, Budapest (HU); Janos Pato, Budapest (HU); Istvan Szabadkai, Budapest (HU); Zsolt Szekelyhid, Budapest (HU); Frigyes Waczek, Budapest (HU)

Correspondence Address: BROOKS KUSHMAN P.C., 1000 TOWN CENTER, TWENTY-SECOND FLOOR, SOUTHFIELD, MI, 48075, US

(73) Assignee: GPC BIOTECH AG, Munich (DE)

(21) Appl. No.: 10/597,120

(22) PCT Filed: Sep. 10, 2004
(86) PCT No.: PCT/EP04/10161

§ 371 Date: Mar. 6, 2007

Related U.S. Application Data
(60) Provisional application No. 60/502,606, filed on Sep. 15, 2003.
(60) Provisional application No. 60/551,341, filed on Mar. 10, 2004.
(60) Provisional application No. 60/577,043, filed on Jun. 7, 2004.

(30) Foreign Application Priority Data
May 28, 2004 (EP) 04012814.2
Sep. 10, 2003 (EP) 03020616.3
Mar. 2, 2004 (EP) 04004891.0

(51) Int. Cl. C07D 495/04; A61P 31/04; C07D 209/42; C07D 333/66; C07D 333/68; C07D 495/10; C07D 409/12; C07D 413/14; C07D 491/04; C07D 493/04

(52) U.S. Cl. 514/233.8; 435/184; 514/408; 514/432; 514/443; 544/146; 546/114; 546/281.1; 548/516; 549/23; 549/50; 549/57

(57) ABSTRACT

Described are heterobicyclic compounds such as 4,5,6,7-tetrahydrobenzo[b]thiophene-3-carboxylic acid amides, 4,7-dihydro-5H-thieno[2,3-c]thiopyran 3-carboxylic acid amides, 4,7-dihydro-5H-thieno[2,3-c]pyran-3-carboxylic acid amides, or benzo[b]thiophene-3-carboxylic acid amides and pharmaceutically acceptable salts thereof, the use of these derivatives for the prophylaxis and/or treatment of various diseases such as infectious diseases, including mycobacteriainduced infections and opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke, as well as compositions containing at least one heterobicyclic compound and/or pharmaceutically acceptable salts thereof. Furthermore, reaction procedures for the synthesis of the heterobicyclic compound are disclosed.

The present invention relates to heterobicyclic compounds and pharmaceutically acceptable salts thereof, the use of these compounds for the prophylaxis and/or treatment of various diseases such as infectious diseases, including mycobacteria-induced infections and opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke, as well as compositions containing at least one heterobicyclic compound and/or pharmaceutically acceptable salts thereof. Furthermore, reaction procedures for the synthesis of said heterobicyclic compounds are disclosed.

Object of the present invention is to provide pharmaceutically active compounds for prophylaxis and treatment of various diseases such as infections, inflammations, immunological diseases, cardiovascular diseases, cell proliferative diseases, transplant rejections, or neurodegenerative diseases, methods for the synthesis of said compounds and pharmaceutical compositions containing at least one pharmaceutically active compound.

This object is solved by the heterobicyclic compound according to claim 1 and/or pharmaceutically acceptable salts of said compounds, the use of at least one of those compounds and/or the pharmaceutically acceptable salts thereof as pharmaceutically active agents according to independent claim 8, the use of the compounds as an inhibitor for a protein kinase according to independent claim 9, the use of the compounds for prophylaxis and/or treatment of various diseases according to independent claim 19, the pharmaceutical composition according to independent claim 38, and a method of amidation of a carboxylic acid ester according to independent claim 39. Further advantageous features, aspects and details of the invention are evident from the dependent claims, the description, the examples and the drawings.

The present invention relates to compounds of the general formula (I)

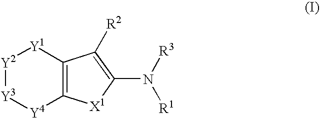

wherein

X¹ is selected from S, O, NH, NR⁴′;

Y¹—Y²—Y³—Y⁴ represent the following residues:

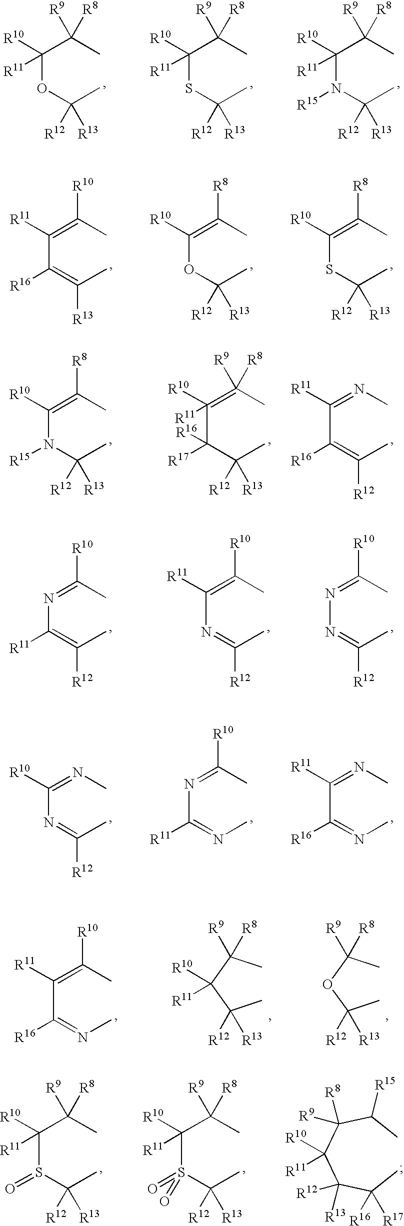

R¹, R⁴, R⁴′, R⁴″, R⁴′″, R⁴″″, R⁸, R⁹ and R¹⁵ are independently of each other selected from —H, substituted or unsubstituted C₁-C₈-alkyl, substituted or unsubstituted C₃-C₁₀-cycloalkyl, substituted or unsubstituted C₁-C₆-heterocyclyl, substituted or unsubstituted C₂-C₆-alkenyl, substituted or unsubstituted C₂-C₆-alkinyl, -Ph, —CH₂Ph, substituted phenyl, substituted benzyl, substituted or unsubstituted C₁-C₈-acyl;

R² represents the residue —CO—NH—R⁴, —CS—NH—R⁴, —SO₂—NH—R⁴, —C(═NH)—NH—R⁴, —COOR⁴, —SO₃—R⁴;

R³ is selected from —H, —CO—R⁵, —CS—R⁵, —C(═NR⁴″)—R⁵, —SO₂R⁵, —CO—R⁵′, —CS—R⁵′, —C(═NR⁴″)—R⁵′, —SO₂R⁵′, —CO—NR⁴″—R⁵, —CS—NR⁴″—R⁵, —CO—CO—NR⁴″—R⁵, —CO—CO—O—R⁵, —CO—OR⁵, —CS—OR⁵, —C(═NH)NR⁴″—R⁵;

R⁵ is selected from substituted or unsubstituted C₃-C₁₀-cycloalkyl, substituted or unsubstituted C₁-C₈-alkyl, substituted or unsubstituted aryl, substituted or unsubstituted heteroaryl, substituted or unsubstituted C₁-C₆-heterocyclyl, substituted or unsubstituted C₂-C₆-alkenyl, substituted or unsubstituted alkylaryl, substituted or unsubstituted C₂-C₆-alkinyl, adamantyl, —H, —R¹⁴, —R¹⁸, —(CH₂)ₙ—C₁-C₆-heterocyclyl, —(CH₂)ₙ—C₃-C₁₀-cycloalkyl,

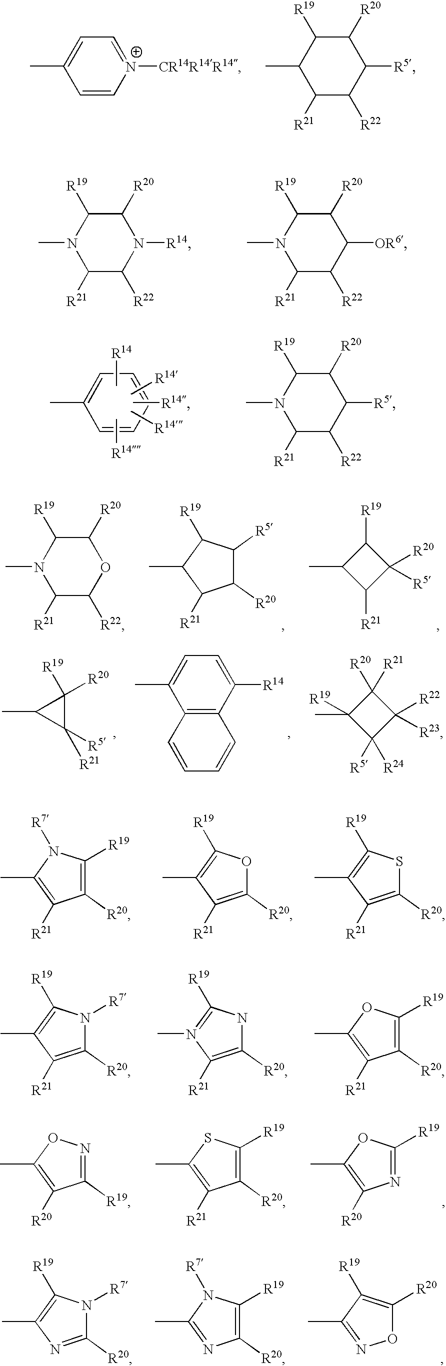

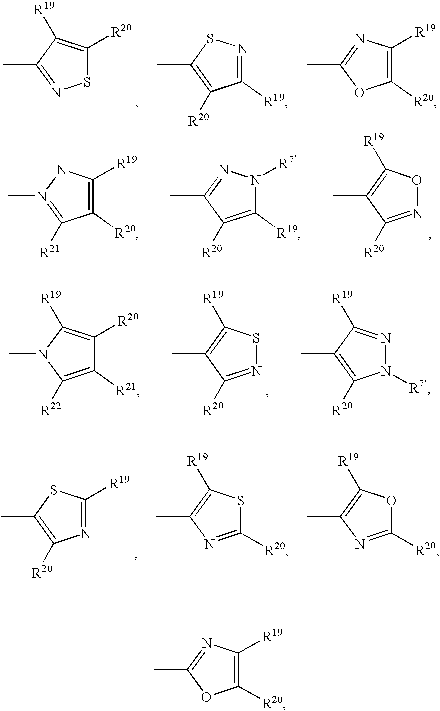

R⁶, R⁶′, R⁷ and R⁷′ are independently of each other selected from —H, substituted or unsubstituted C₃-C₁₀-cycloalkyl, substituted or unsubstituted C₁-C₈-alkyl, substituted or unsubstituted aryl, -Ph, —CH₂Ph, substituted phenyl, substituted benzyl, substituted or unsubstituted heteroaryl, substituted or unsubstituted C₁-C₆-heterocyclyl, substituted or unsubstituted C₂-C₆-alkenyl, substituted or unsubstituted C₂-C₆-alkinyl, adamantyl;

—R⁵′, R¹⁰, R¹¹, R¹², R¹³, R¹⁶ and R¹⁷ are independently of each other selected from —H, —OH, —NH₂, —R¹⁴, —R¹⁴′, —R¹⁴″, —R¹⁴′″, —R¹⁴″″, —R¹⁸, —R¹⁸′, —R¹⁸″, —R¹⁸′″, —R¹⁸″″, -Ph, —CH₂Ph, substituted phenyl, substituted benzyl, substituted or unsubstituted C₁-C₈-alkyl, substituted or unsubstituted C₃-C₁₀-cycloalkyl, substituted or unsubstituted C₁-C₆-heterocyclyl, substituted or unsubstituted C₂-C₆-alkenyl, substituted or unsubstituted C₂-C₆-alkinyl, adamantyl, substituted or unsubstituted aryl, substituted or unsubstituted heteroaryl, substituted or unsubstituted C₁-C₆-alkoxyl, substituted or unsubstituted —O—C₃-C₁₀-cycloalkyl, substituted or unsubstituted —O—C₁-C₆-heterocyclyl, substituted or unsubstituted —O-aryl, substituted or unsubstituted —O-heteroaryl, —OOC—O—R¹⁴, —O—CO—R¹⁴′, —COO—R¹⁴′, —O—CO—NR⁷R¹⁴, —NR¹⁴″″—CO—NR⁷R¹⁴, —NR¹⁴″″—CO—R¹⁴, —NR¹⁴″″—CS—NR⁷R¹⁴, —NR¹⁴″″—CS—OR¹⁴, —NR¹⁴″″—C(═NR¹⁴′″)—NR⁷R¹⁴, —O—CS—NR⁷R¹⁴, —CO—NR⁷R¹⁴, —NR⁷R¹⁴, —CH₂—SR⁴″″, —O—R¹⁴′, —OCR¹⁴′R¹⁴″R¹⁴′″, —OCH₂—R¹⁴′, —O—(CH₂)ₘ—R¹⁴′, —OCH₂—CR¹⁴′R¹⁴″R¹⁴′″, —O—(CH₂)ₘ—CR¹⁴′R¹⁴″R¹⁴′″, —O—CR¹⁹R²⁰—CR¹⁴′R¹⁴″R¹⁴′″, —OCH₂—OR¹⁴′, —O—(CH₂)ₚ—OR¹⁴′, —OCH₂—OCR¹⁴′R¹⁴″R¹⁴′″, —O—(CH₂)ₚ—OCR¹⁴′R¹⁴″R¹⁴′″, —O—CR¹⁹R²⁰—OCR¹⁴′R¹⁴″R¹⁴′″, —OCH₂—NR¹⁴″R¹⁴′″, —O—(CH₂)ₚ—NR¹⁴″R¹⁴′″, —O—CR¹⁹R²⁰—NR¹⁴″R¹⁴′″, —OCH₂—CO—R¹⁴′, —O—(CH₂)ₘ—CO—R¹⁴′, —OCH₂—CO—CR¹⁴′R¹⁴″R¹⁴′″, —O—(CH₂)ₘ—CO—CR¹⁴′R¹⁴″R¹⁴′″, —O—CR¹⁹R²⁰—CO—CR¹⁴′R¹⁴″R¹⁴′″, —OCH₂—O—CO—R¹⁴′, —O—(CH₂)ₚ—O—CO—R¹⁴′, —OCH₂—O—CO—CR¹⁴′R¹⁴″R¹⁴′″, —O—(CH₂)ₚ—O—CO—CR¹⁴′R¹⁴″R¹⁴′″, —O—CR¹⁹R²⁰—O—CO—CR¹⁴′R¹⁴″R¹⁴′″, —OCH₂—O—CO—OR¹⁴′, —O—(CH₂)ₚ—O—CO—OR¹⁴′, —OCH₂—O—CO—OCR¹⁴′R¹⁴″R¹⁴′″, —O—(CH₂)ₚ—O—CO—OCR¹⁴′R¹⁴″R¹⁴′″, —O—CR¹⁹R²⁰—O—CO—OCR¹⁴′R¹⁴″R¹⁴′″, —OCH₂—NR¹⁴′—CO—R¹⁴″, —O—(CH₂)ₚ—NR¹⁴′—CO—R¹⁴″, —OCH₂—NR¹⁴′—CO—CR¹⁴″R¹⁴′″R¹⁴″″, —O—(CH₂)ₚ—NR¹⁴′—CO—CR¹⁴″R¹⁴′″R¹⁴″″, —O—CR¹⁹R²⁰—NR¹⁴′—CO—CR¹⁴″R¹⁴′″R¹⁴″″, —OCH₂—NR¹⁴′—CO—OR¹⁴″, —O—(CH₂)ₚ—NR¹⁴′—CO—OR¹⁴″, —OCH₂—NR¹⁴′—CO—OCR¹⁴″R¹⁴′″R¹⁴″″, —O—(CH₂)ₚ—NR¹⁴′—CO—OCR¹⁴″R¹⁴′″R¹⁴″″, —O—CR¹⁹R²⁰—NR¹⁴′—CO—OCR¹⁴″R¹⁴′″R¹⁴″″, —OCH₂—CO—OR¹⁴′, —OCH₂—CO—OCR¹⁴′R¹⁴″R¹⁴′″, —O—(CH₂)ₘ—CO—OR¹⁴′, —O—(CH₂)ₘ—CO—OCR¹⁴′R¹⁴″R¹⁴′″, —O—CR¹⁹R²⁰—CO—OCR¹⁴′R¹⁴″R¹⁴′″, —OCH₂—CO—NR¹⁴″R¹⁴′″, —O—(CH₂)ₘ—CO—NR¹⁴″R¹⁴′″, —OCH₂—O—CO—NR¹⁴″R¹⁴′″, —O—(CH₂)ₚ—O—CO—NR¹⁴″R¹⁴′″, —O—CR¹⁹R²⁰—O—CO—NR¹⁴″R¹⁴′″, —OCH₂—NR¹⁴″—CO—NR¹⁴′″R¹⁴″″, —O—(CH₂)ₚ—NR¹⁴″—CO—NR¹⁴′″R¹⁴″″, —O—CR¹⁹R²⁰—NR¹⁴″—CO—NR¹⁴′″R¹⁴″″, —OCH₂—NR¹⁴″—C(═NR¹⁴′)—NR¹⁴′″R^{″″, —O—(CH}₂)ₚ—NR¹⁴″—C(═NR¹⁴′)—NR¹⁴′″R¹⁴″″, —CR¹⁴′R¹⁴″R¹⁴′″, —CH₂—R¹⁴′, —(CH₂)ₘ—R¹⁴′, —CH₂—CR¹⁴′R¹⁴″R¹⁴′″, —(CH₂)ₘ—CR¹⁴′R¹⁴″R¹⁴′″, —CR¹⁹R²⁰—CR¹⁴′R¹⁴″R¹⁴′″, —CH₂—OR¹⁴′, —(CH₂)ₘ—OR¹⁴′, —CH₂—OCR¹⁴′R¹⁴″R¹⁴′″, —(CH₂)ₘ—OCR¹⁴′R¹⁴″R¹⁴′″, —CR¹⁹R²⁰—OCR¹⁴′R¹⁴″R¹⁴′″, —CH₂—NR¹⁴″R¹⁴′″, —(CH₂)ₘ—NR¹⁴″R¹⁴′″, —CR¹⁹R²⁰—NR¹⁴″R¹⁴′″, —CH₂—CO—R¹⁴′,—(CH₂)ₘ—CO—R¹⁴′, —CH₂—CO—CR¹⁴′R¹⁴″R¹⁴′″, —(CH₂)ₘ—CO—CR¹⁴′R¹⁴″R¹⁴′″, —CR¹⁹R²⁰—CO—CR¹⁴′R¹⁴″R¹⁴′″, —CH₂—O—CO—R¹⁴′, —(CH₂)ₘ—O—CO—R¹⁴′, —CH₂—O—CO—CR¹⁴′R¹⁴″R¹⁴′″, —(CH₂)ₘ—O—CO—CR¹⁴′R¹⁴″R¹⁴′″, —CR¹⁹R²⁰—O—CO—CR¹⁴′R¹⁴″R¹⁴′″, —CH₂—O—CO—OR¹⁴′, —(CH₂)ₘ—O—CO—OR¹⁴′, —CH₂O—CO—OCR¹⁴′R¹⁴″R¹⁴′″, —(CH₂)ₘ—O—CO—OCR¹⁴′R¹⁴″R¹⁴′″, —CR¹⁹R²⁰—O—CO—OCR¹⁴′R¹⁴″R¹⁴′″, —CH₂—NR¹⁴′—CO—R¹⁴″, —(CH₂)ₘ—NR¹⁴′—CO—R¹⁴″, —CH₂—NR¹⁴′—CO—CR¹⁴″R¹⁴′″R¹⁴″″, —(CH₂)ₘ—NR¹⁴′—CO—CR¹⁴″R¹⁴′″R¹⁴″″, —CR¹⁹R²⁰—NR¹⁴′—CO—CR¹⁴″R¹⁴′″R¹⁴″″, —CH₂—NR¹⁴′—CO—OR¹⁴″, —(CH₂)ₘ—NR¹⁴′—CO—OR¹⁴″, —CH₂—NR¹⁴′—CO—OCR¹⁴″R¹⁴′″R¹⁴″″, —(CH₂)ₘ—NR¹⁴′—CO—OCR¹⁴″R¹⁴′″R¹⁴″″, —CR¹⁹R²⁰—NR¹⁴′—CO—OCR¹⁴″R¹⁴′″R¹⁴″″, —CH₂—CO—OR¹⁴′, —CH₂—CO—OCR¹⁴′R¹⁴″R¹⁴′″,—(CH₂)ₘ—CO—OR¹⁴′, —(CH₂)ₘ—CO—OCR¹⁴′R¹⁴″R¹⁴′″, —CR¹⁹R²⁰—CO—OCR¹⁴′R¹⁴″R¹⁴′″, —CH₂—CO—NR¹⁴″R¹⁴′″, —(CH₂)ₘ—CO—NR¹⁴″R¹⁴′″, —CR¹⁹R²⁰—CO—NR¹⁴″R¹⁴′″, —CH₂—O—CO—NR¹⁴″R¹⁴′″, —(CH₂)ₘ—O—CO—NR¹⁴″R¹⁴′″, —CR¹⁹R²⁰—O—CO—NR¹⁴″R¹⁴′″, —CH₂—NR¹⁴″—CO—NR¹⁴′″R¹⁴″″, —(CH₂)ₘ—NR¹⁴″—CO—NR¹⁴′″R¹⁴″″, —CR¹⁹R²⁰—NR¹⁴″—CO—NR¹⁴′″R¹⁴″″, —CH₂—NR¹⁴″—C(═NR¹⁴′)—NR¹⁴′″R¹⁴″″,—(CH₂)ₘ—NR¹⁴″—C(═NR¹⁴′)—NR¹⁴″″, —CR¹⁹R²⁰—NR¹⁴″—C(═NR¹⁴′)—NR¹⁴′″R¹⁴″″, —NR¹⁴—CR¹⁴′R¹⁴″R¹⁴′″, —NR¹⁴—CH₂—R¹⁴′, —NR¹⁴—(CH₂)ₘ—R¹⁴′, —NR¹⁴—CH₂—CR¹⁴′R¹⁴″R¹⁴′″, —NR¹⁴—(CH₂)ₘ—CR¹⁴′R¹⁴″R¹⁴′″, —NR¹⁴—CR¹⁹R²⁰—CR¹⁴′R¹⁴″R¹⁴′″, —NR¹⁴—CH₂—OR¹⁴′, —NR¹⁴—(CH₂)ₚ—OR¹⁴′, —NR¹⁴—CH₂—OCR¹⁴′R¹⁴″R¹⁴′″, —NR¹⁴—(CH₂)ₚ—OCR¹⁴′OR¹⁴″R¹⁴′″, —NR¹⁴—CR¹⁹R²⁰—OCR¹⁴′R¹⁴″R¹⁴′″, —NR¹⁴—CH₂—NR¹⁴″R¹⁴′″, —NR¹⁴—(CH₂)ₚ—NR¹⁴″R¹⁴′″, —NR¹⁴—CR¹⁹R²⁰—NR¹⁴″R¹⁴′″, —NR¹⁴—CH₂—CO—R¹⁴′, —NR¹⁴—(CH₂)ₘ—CO—R¹⁴′, —NR¹⁴—CH₂—CO—CR¹⁴′R¹⁴″R¹⁴′″, —NR¹⁴—(CH₂)ₘ—CO—CR¹⁴′R¹⁴″R¹⁴′″, —NR¹⁴—CR¹⁹R²⁰—CO—CR¹⁴′R¹⁴″R¹⁴′″, —NR¹⁴—CH₂—O—CO—R¹⁴′, —NR¹⁴—(CH₂)ₚ—O—CO—R¹⁴′, —NR¹⁴—CH₂—O—CR¹⁴′R¹⁴″R¹⁴′″, —NR¹⁴—(CH₂)ₚ—O—CO—CR¹⁴′R¹⁴″R¹⁴′″, —NR¹⁴—CR¹⁹R²⁰—O—CO—CR¹⁴′R¹⁴″R¹⁴′″, —NR¹⁴—CH₂—O—CO—OR¹⁴′, —NR¹⁴—(CH₂)ₚ—O—CO—OR¹⁴′, —NR¹⁴—CH₂—O—CO—OCR¹⁴′R¹⁴″R¹⁴′″, —NR¹⁴—(CH₂)ₚ—O—CO—OCR¹⁴′R¹⁴″R¹⁴′″, —NR¹⁴—CR¹⁹R²⁰—O—CO—OCR¹⁴′R¹⁴″R¹⁴′″, —NR¹⁴—CH₂—NR¹⁴′—CO—R¹⁴″,—NR¹⁴—(CH₂)ₚ—NR¹⁴′—CO—R¹⁴″, —NR¹⁴—CH₂—NR¹⁴′—CO—CR¹⁴″R¹⁴′″R¹⁴″″, —NR¹⁴—(CH₂)ₚ—NR¹⁴′—CO—CR¹⁴″R¹⁴′″R¹⁴″″, —NR¹⁴—CR¹⁹R²⁰—NR¹⁴′—CO—CR¹⁴″R¹⁴′″R¹⁴″″, —NR¹⁴—CH₂—NR¹⁴′—CO—OR¹⁴″, —NR¹⁴—(CH₂)ₚ—NR¹⁴′—CO—OR¹⁴″, —NR¹⁴—CH₂—NR¹⁴′—CO—OCR¹⁴″R¹⁴′″R¹⁴″″, —NR¹⁴—(CH₂)ₚ—NR¹⁴′—CO—OCR¹⁴″R¹⁴′″R¹⁴″″, —NR¹⁴—CR¹⁹R²⁰—NR¹⁴′—CO—OCR¹⁴″R¹⁴′″R¹⁴″″,—NR¹⁴—CH₂—CO—OR¹⁴′, —NR¹⁴—CH₂—CO—OCR¹⁴′R¹⁴″R¹⁴′″, —NR¹⁴—(CH₂)ₘ—CO—OR¹⁴′, —NR¹⁴—(CH₂)ₘ—CO—OCR¹⁴′R¹⁴″R¹⁴′″, —NR¹⁴—CR¹⁹R²⁰—CO—OCR¹⁴′R¹⁴″R¹⁴′″, —NR¹⁴—CH₂—CO—NR¹⁴″R¹⁴′″, —NR¹⁴—(CH₂)ₘ—CO—NR¹⁴″R¹⁴′″, —NR¹⁴—CR¹⁹R²⁰—CO—NR¹⁴″R¹⁴′″, —NR¹⁴—CH₂—O—CO—NR¹⁴″R¹⁴′″, —NR¹⁴—(CH₂)ₚ—O—CO—NR¹⁴″R¹⁴′″, —NR¹⁴—CR¹⁹R²⁰—O—CO—NR¹⁴″R¹⁴′″, —NR¹⁴—CH₂—NR¹⁴″—CO—NR¹⁴′″R¹⁴″″, —NR¹⁴—(CH₂)ₚ—NR¹⁴″—CO—NR¹⁴′″R¹⁴″″, —NR¹⁴—CR¹⁹R²⁰—NR¹⁴″—CO—NR¹⁴′″R¹⁴″″, —NR¹⁴—CH₂—NR¹⁴″—C(═NR¹⁴′)—NR¹⁴′″R¹⁴″″, —NR¹⁴—(CH₂)ₚ—NR¹⁴″—C(═NR¹⁴′)—NR¹⁴′″R¹⁴″″, —NR¹⁴—CR¹⁹R²⁰—NR¹⁴″—C(═NR¹⁴′)—NR¹⁴′″R¹⁴″″, —NR⁶R⁷, —(CH₂)ₙ—NR⁶R¹⁴, —(CH₂)ₙ—OR¹⁴, —(CH₂)ₙ—R¹⁴, —(CH₂)ₙ—SR¹⁴, —(CH₂)ₙ—SO—R¹⁴, —(CH₂)ₙ—SO₂—R¹⁴, —(CH₂)ₙ—SO₃—R¹⁴, —(CH₂)ₙ—CO—R¹⁴, —(CH₂)ₙ—COO—R¹⁴, —(CH₂)ₙ—O—CO—R¹⁴, —(CH₂)ₙ—NH—CO—R¹⁴, —(CH₂)ₙ—CO—NR⁶R¹⁴, —(CH₂)ₙ—O—CO—OR¹⁴, —(CH₂)ₙ—NH—CO—OR¹⁴, —(CH₂)ₙ—O—CO—NR⁶R¹⁴, —(CH₂)ₙ—NH—CO—NR⁶R¹⁴, —(CH₂)ₙ—NH—CS—NR⁶R¹⁴, —(CH₂)ₙ—NH—C(═NH)—NR⁶R¹⁴, —(CH₂)ₙ-aryl, —(CH₂)ₙ-heteroaryl, —CR¹⁹R²⁰—NR⁶R¹⁴, —CR¹⁹R²⁰—OR¹⁴, —CR¹⁹R²⁰R¹⁴, —CR¹⁹R²⁰—SR¹⁴, —CR¹⁹R²⁰—SO—R¹⁴, —CR¹⁹R²⁰—SO₂—R¹⁴, —CR¹⁹R²⁰—SO₃—R¹⁴, —CR¹⁹R²⁰—CO—R¹⁴, —CR¹⁹R²⁰—COO—R¹⁴, —CR¹⁹R²⁰—O—CO—R¹⁴, —CR¹⁹R²⁰—NH—CO—R¹⁴, —CR¹⁹R²⁰—CO—NR⁶R¹⁴, —CR¹⁹R²⁰—O—CO—OR¹⁴, —CR¹⁹R²⁰—NH—CO—OR¹⁴, —CR¹⁹R²⁰—O—CO—NR⁶R¹⁴, —CR¹⁹R²⁰—NH—CO—NR⁶R¹⁴, —CR¹⁹R²⁰—NH—CS—NR⁶R¹⁴, —CR¹⁹R²⁰—NH—C(═NH)—NR⁶R¹⁴, —CR¹⁹R²⁰-aryl, —CR¹⁹R²⁰-heteroaryl, —CH═CH—R¹⁸, —CH═CH—R¹⁴, —CR¹⁸′═CR¹⁸″R¹⁸′″, —CR¹⁴′═CR¹⁴″R¹⁴′″;

R¹¹ and R¹⁶ can form together with Y² and Y³ a five-membered or six-membered saturated substituted or unsubstituted carbocyclic ring;

R⁸ and R⁹ represent together ═O, ═N—OH, ═N—OR⁴″″, ═N—R⁴″″, ═N—NH₂, ═N—NHR⁴″″, ═N—NR⁴′″R⁴″″, 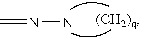
thus forming a carbonyl group, or an oxime, or a hydrazone, together with the carbon atom Y¹, or R⁸ and R⁹ form together a carbocyclic or heterocyclic ring;

R¹⁰ and R¹¹ if bound to the same carbon atom Y² represent together ═O, ═N—OH, ═N—OR⁴″″, ═N—R⁴″″, ═N—NH₂, ═N—NHR⁴″″, ═N—NR⁴′″R⁴″″, 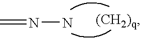
thus forming a carbonyl group, or an oxime, or a hydrazone, together with the carbon atom Y², or R¹⁰ and R¹¹ form together a carbocyclic or heterocyclic ring;

R¹² and R¹³ represent together ═O, ═N—OH, ═N—OR⁴″″, ═N—R⁴″″, ═N—NH₂, ═N—NHR⁴″″, ═N—NR⁴′″R⁴″″, 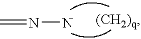
thus forming a carbonyl group, or an oxime, or a hydrazone, together with the carbon atom Y⁴, or R¹² and R¹³ form together a carbocyclic or heterocyclic ring;

R¹⁶ and R¹⁷ represent together ═O, ═N—OH, ═N—OR⁴″″, ═N—R⁴″″, ═N—NH₂, ═N—NHR⁴″″, ═N—NR⁴′″R⁴″″, 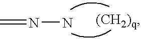
thus forming a carbonyl group, or an oxime, or a hydrazone, together with the carbon atom Y³, or R¹⁶ and R¹⁷ form together a carbocyclic or heterocyclic ring;

R¹⁴, R¹⁴′, R¹⁴″, R¹⁴′″, and R¹⁴″″ are independently of each other selected from —H, substituted or unsubstituted C₃-C₁₀-cycloalkyl, substituted or unsubstituted C₁-C₈-alkyl, substituted or unsubstituted aryl, substituted or unsubstituted heteroaryl, substituted or unsubstituted C₁-C₆-heterocyclyl, substituted or unsubstituted C₂-C₆-alkenyl, substituted or unsubstituted C₂-C₆-alkinyl, adamantyl, substituted or unsubstituted alkylaryl, —R¹⁸, —R¹⁸′, —R¹⁸″, —R¹⁸′″, —R¹⁸″″, —R¹⁹, —R²⁰, —R²¹, —R²², —R²³, —(CH₂)ₙ—O—CO—R¹⁸″″, —(CH₂)ₙ—NH—CO—R¹⁸″″, —(CH₂)ₙ—NH—CO—R¹⁸″″, —(CH₂)ₙ—R¹⁸′, —(CH₂)ₙ—R¹⁸″, —(CH₂)ₙ—R¹⁸′″, —(CH₂)ₙ—SO—R¹⁸′″, —(CH₂)ₙ—SO₂—R¹⁸′″, —(CH₂)ₙ—CO—R¹⁸″″, —(CH₂)ₙ—NH—CO—R¹⁸″″, —(CH₂)ₙ—CO—NH—NH₂, —(CH₂)ₙ—SR¹⁸″, —C≡C—R¹⁸″″, —CR¹⁸′═CR¹⁸″R¹⁸″″,

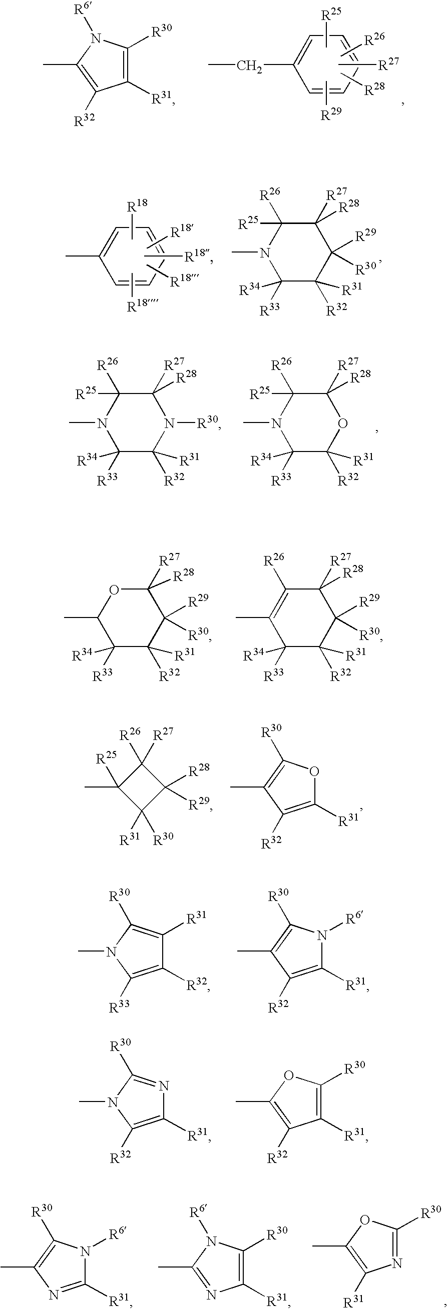

R¹⁸, R¹⁸′, R¹⁸″, R¹⁸′″, R¹⁸″″, R¹⁹-R³³ are independently of each other selected from —H, —OH, —OCH₃, —OC₂H₅, —OC₃H₇, —O-Cyclo-C₃H₅, —OCH(CH₃)₂, —OC(CH₃)₃, —OPh, —OCH₂-Ph, —OCPh₃, —OR⁶, —OR⁷, —SH, —SCH₃, —SC₂H₅, —SC₃H₇, —S-cyclo-C₃H₅, —SCH(CH₃)₂, —SC(CH₃)₃, —SR⁶, —SR⁷, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —COOR⁶, —COOR⁷, —OOC—CH₃, —OOC—C₂H₅, —OOC—C₃H₇, —OOC-cyclo-C₃H₅, —OOC—CH(CH₃)₂, —OOC—C(CH₃)₃, —OOC—R⁶, —OOC—R⁷, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —CONR⁶R⁷, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —NR⁶R⁷, —SOCH₃, —SOC₂H₅, —SOC₃H₇, —SO-cyclo-C₃H₅, —SOCH(CH₃)₂, —SOC(CH₃)₃, —SO—R⁶, —SO—R⁷, —SO₂CH₃, —SO₂C₂H₅, —SO₂C₃H₇, —SO₂-cyclo-C₃H₅, —SO₂CH(CH₃)₂, —-SO₂C(CH₃)₃, —SO₂R⁶, —SO₂R⁷, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —SO₃—R⁶, —SO₃—R⁷, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —O—COOC—R⁶, —O—COOC—R⁷, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CO—N(R⁶)₂, —NH—CO—N(R⁷)₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH-CS—N[CH(CH₃)₂]₂, —NH—CS—N[C(CH₃)₃]₂, —NH—CS—N(R⁶)₂, —NH—CS—N(R⁷)₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N[CH(CH₃)₂]₂, —NH—C(═NH)—N[C(CH₃)₃]₂, —NH—C(═NH)—N(R⁶)₂, —NH—C(═NH)—N(R⁷)₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—N(R⁶)₂, —O—CO—N(R⁷)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —O—CO—OR⁶, —O—CO—OR⁷, —CH₂F, —CHF₂, —CF₃, —CH₂Cl, —CHCl₂, —CCl₃, —CH₂Br, —CHBr₂, —CBr₃, —CH₂I, —CHI₂, —Cl₃, —CH₂—CH₂F, —CH₂—CHF₂, —CH₂—CF₃, —CH₂—CH₂Cl, —CH₂—CHCl₂, —CH₂—CCl₃, —CH₂—CH₂Br, —CH₂—CHBr₂, —CH₂—CBr₃, —CH₂—CH₂I, —CH₂—CHI₂, —CH₂—Cl₃, —CH₂OH, —CH₂SH, —CH₂NH₂, —CH₂N(CH₃)₂, —CH₂N(C₂H₅)₂, —C₂H₄—OH, —C₂H₄—SH, —C₂H₄—NH₂, —C₂H₄—N(CH₃)₂, —C₂H₄—N(C₂H₅)₂, —CH₂OCH₃, —CH₂SCH₃, —CH₂OC₂H₅, —CH₂SC₂H₅, —C₂H₄—OCH₃, —C₂H₄—SCH₃, —C₂H₄—OC₂H₅, —C₂H₄—SC₂H₅, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH═CHF, —CH═CF₂, —CF═CF₂, —CH═CHCl, —CH═CCl₂, —CCl═CCl₂, —CH═CHBr, —CH═CBr₂, —CBr═CBr₂, —CH₂—CH═CH₂, —C(CH₃)═CH₂, —CH═CH—CH₃, —CH═CH—CH₂—OH, —CH═CH—COOH, —CH═CH—COOCH₃, —CH═CH—COOC₂H₅, —CH═CH-Ph, —C(CH₃)═CH—CH₃, —C₂H₄—CH═CH₂, —CH═C(CH₃)₂, —C≡CH, —C≡C—CH₃, —CH₂—C≡CH, —CPh₃; 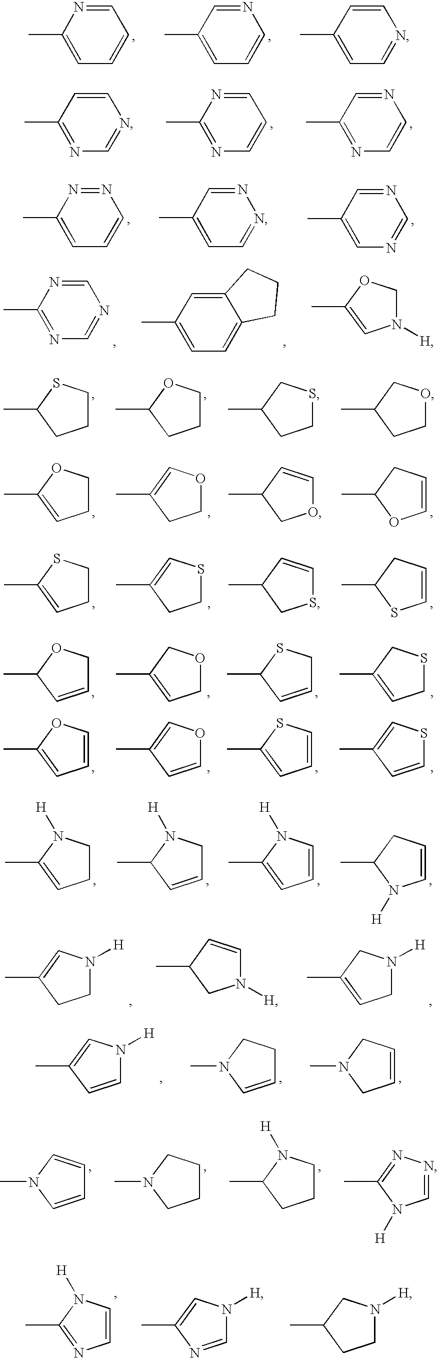 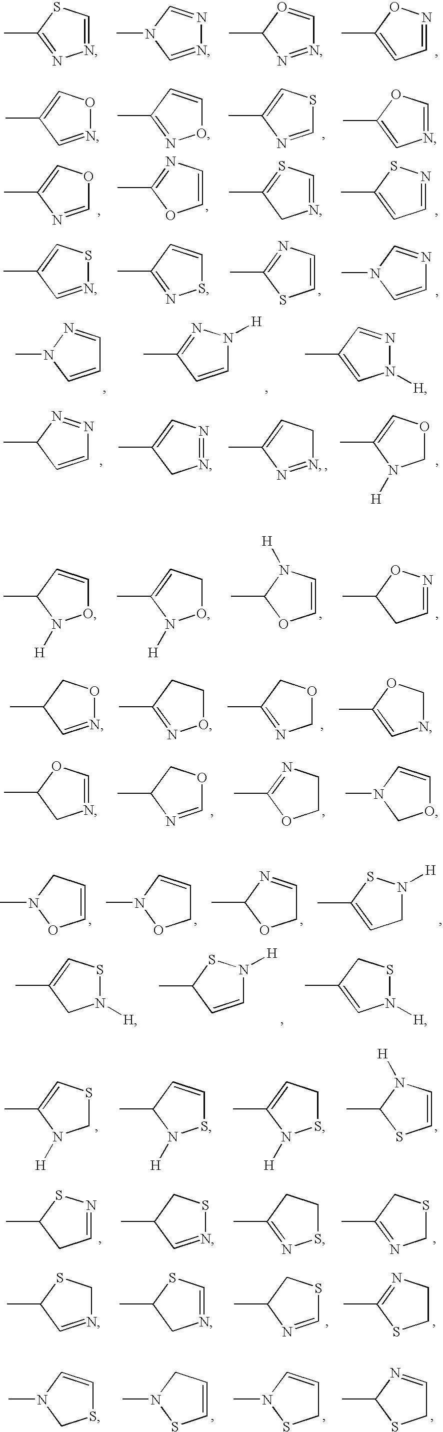 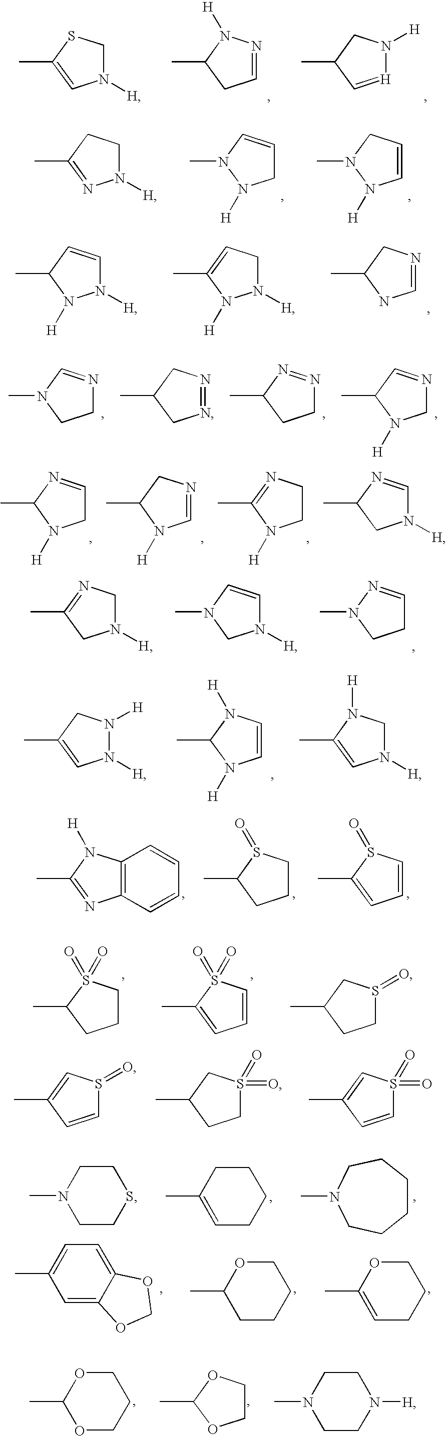 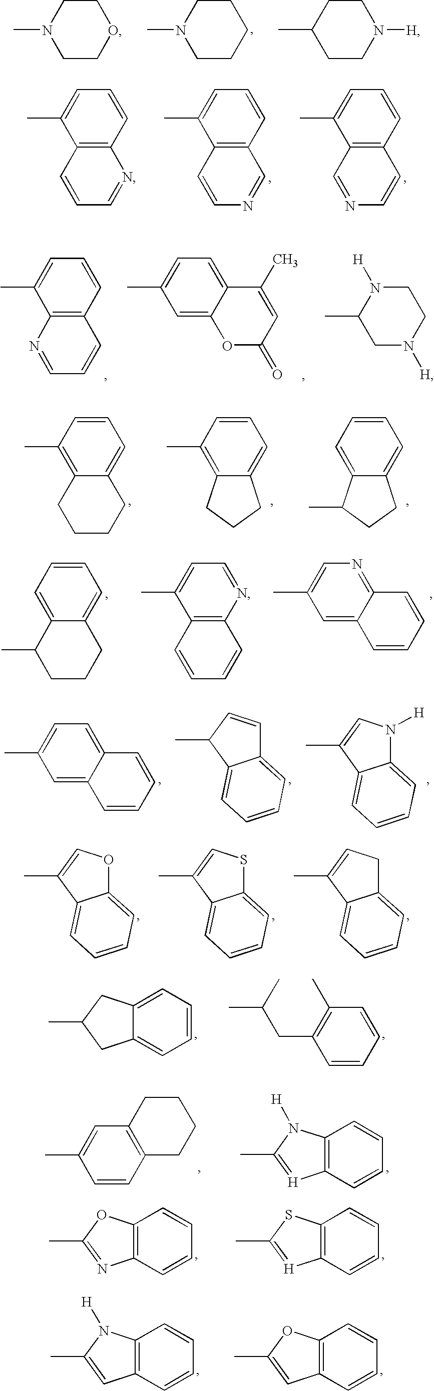 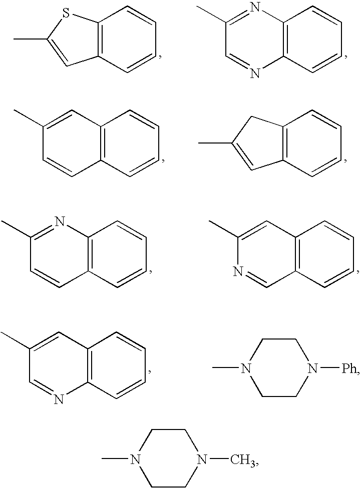
substituted or unsubstituted C₁-C₈-alkyl, substituted or unsubstituted C₂-C₆-alkenyl, substituted or unsubstituted C₂-C₆-alkinyl, substituted or unsubstituted C₃-C₁₀-cycloalkyl, substituted or unsubstituted aryl, -Ph, —CH₂Ph, substituted phenyl, substituted benzyl, substituted or unsubstituted heteroaryl, substituted or unsubstituted C₁-C₆-heterocyclyl;

m and n are independently of each other integers from 0-6;

p is an integer from 1-6;

q is an integer from 2-6;

and stereoisomeric and regioisomeric forms and pharmaceutically acceptable salts of these compounds;

under the proviso that if Y¹—Y²—Y³—Y⁴ represent

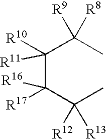

R² does not represent —COOR⁴. In this case, R² represents preferably —CO—NH₂.

The compounds No. 1-287 mentioned in the PCT application WO 03/084947 A1 on pages 9-25 are herewith excluded from the scope of the present invention by disclaimer.

The following compounds are also excluded by disclaimer:

2-(Toluene-4-sulfonylamino)-4,5,6,7-tetrahydro-benzo[b]thiophene-3-carboxylic acid amide;

2-[(5-Bromo-2-hydroxy-benzylidene)-amino]-4,5,6,7-tetrahydro-benzo[b]thiophene-3-carboxylic acid amide;

2-[(2-Chloro-benzylidene)-amino]-4,5,6,7-tetrahydro-benzo[b]thiophene-3-carboxylic acid amide;

Furan-2-carboxylic acid[3-(4-methoxy-phenylcarbamoyl)4,5,6,7-tetrahydro-benzo[b]thiophen-2-yl]-amide;

2-[3-(4-Methoxy-phenyl)-3-(2,2,2-trifluoro-acetylamino)-propionyl-amino]-4,5,6,7-tetrahydro-benzo[b]thiophene-3-carboxylic acid amide;

2-(7-Ethyl-4-oxo-3-phenyl-3 ,4,5,6,7,8-hexahydro-benzo)[4,5]thieno[2,3-d]pyrimidin-2-ylsulfanyl)-N-(2-isopropoxy-phenyl)-acetamide;

N,N-Diethyl-2-(7-ethyl-4-oxo-3-phenyl-3,4,5,6,7,8-hexahydro-benzo)[4,5]thieno[2,3-d]pyrimidin-2-ylsulfanyl)-acetamide;

2-(2,2,3,3-Tetrafluro-propionylamino)-4,5,6,7-tetrahydro-benzo[b]thiophene-3-carboxylic acid amide;

2-[3-(2,2,2-Trichloro-1-propionylamino-ethyl)-thioureido]-4,5,6,7-tetrahydro-benzo[b]thiophene-3-carboxylic acid amide;

2-(2,4-Dichlorophenoxy)-N-(4-oxo-2-propyl-5,6,7,8-tetrahydro-4H-benzo[4,5]thieno[2,3-d]pyrimidin-3-yl)-acetamide;

6-Methyl-2-[(thiophene-2-carbonyl)-amino]-4,5,6,7-tetrahydro-benzo[b]thiophene-3-carboxylic acid amide;

2-[3-(3,4-Dichloro-phenyl-ureido]-4,5,6,7-tetrahydro-benzo[b]thiophene-3-carboxylic acid amide;

2-(2-Piperidin-1-yl-acetylamino)-4,5,6,7-tetrahydro-benzo[b]thiophene-3-carboxylic acid amide;

2-[2-(4-Methyl-piperazin-1-yl)-acetylamino]-4,5,6,7-tetrahydro-benzo[b]thiophene-3-carboxylic acid amide;

2-(3-Furan-2-yl-acryloylamino)-4,5,6,7-tetrahydro-benzo[b]thiophene-3-carboxylic acid amide;

2-[(2-Ethoxy-benzylidene)-amino]-4,5,6,7-tetrahydro-benzo[b]thiophene-3-carboxylic acid amide;

6-Methyl-2-(3-phenyl-propionylamino)-4,5,6,7-tetrahydro-benzo[b]thiophene-3-carboxylic acid amide;

Tetrahydro-furan-2-carboxylic acid(3-carbamoyl-6-methyl-4,5,6,7-tetrahydro-benzo[b]thiophen-2-yl)-amide;

2-(4-Fluoro-benzenesulfonylamino)-4,5,6,7-tetrahydro-benzo[b]thiophene-3-carboxylic acid amide;

6-tert-Butyl-2-[2-(5-methyl-3-trifluormethyl-pyrazol-1-yl)-acetyl-amino]-4,5,6,7-tetrahydro-benzo[b]thiophene-3-carboxylic acid amide;

6-tert-Butyl-2-[2-(3,5-dimethyl-4-nitro-pyrazol-1-yl)-acetylamino]-4,5,6,7-tetrahydro-benzo[b]thiophene-3-carboxylic acid amide;

2-Amino-6-tert-butyl-4,5,6,7-tetrahydro-benzo[b]thiophene-3-carboxylic acid amide;

2-[2-(3,5-Dimethyl-4-nitro-pyrazol-1-yl)-acetylamino]-4,5,6,7-tetrahydro-benzo[b]thiophene-3-carboxylic acid amide;

2-(3-Carboxy-acryloylamino)-6-(1,1-dimethyl-propyl)-4,5,6,7-tetrahydro-benzo[b]thiophene-3-carboxylic acid amide;

6-(1,1-Dimethyl-propyl)-2-[(5-methyl-furan-2-carbonyl)-amino]-4,5,6,7-tetrahydro-benzo[b]thiophene-3-carboxylic acid amide;

2-(Cyclopropanecarbonyl-amino)-4,5,6,7-tetrahydro-benzo[b]thiophene-3-carboxylic acid amide;

2-[2-(4-Nitrophenyl)-acetylamino]-4,5,6,7-tetrahydro-benzo[b]thiophene-3-carboxylic acid amide;

6-Methyl-2-[2-(3-nitro-[1,2,4]triazol-1-yl)-acetylamino]-4,5,6,7-tetrahydro-benzo[b]thiophene-3-carboxylic acid amide;

Furan-2-carboxylic acid[3-(2-hydroxy-ethylcarbamoyl)-4,5,6,7-tetrahydro-benzo[b]thiophen-2-yl]-amide;

2-Acetylamino-4,5,6,7-tetrahydrobenzo[b]thiophene-3-carboxylic acid amide;

2-(2,2,-Dimethyl-propionylamino)-4,5,6,7-tetrahydrobenzo[b]thiophene-3-carboxylic acid amide;

2-Isobutyrylamino-4,5,6,7-tetrahydrobenzo[b]thiophene-3-carboxylic acid amide;

2-(2-Methyl-acryloylamino)-4,5,6,7-tetrahydrobenzo[b]thiophene-3-carboxylic acid amide;

2-[(Thiophene-2-carbonyl)-amino]-4,5,6,7-tetrahydrobenzo[b]thiophene-3-carboxylic acid amide;

Furan-2-carboxylic acid(3-carbamoyl-4,5,6,7-tetrahydro-benzo[b]thiophen-2-yl)-amide;

2-(Cyclobutanecarbonyl-amino)-4,5,6,7-tetrahydrobenzo[b]thiophene-3-carboxylic acid amide;

2-(2-Methyl-butyrylamino)-4,5,6,7-tetrahydrobenzo[b]thiophene-3-carboxylic acid amide;

2-(Cyclopropanecarbonyl-amino)-4-methyl-4,5,6,7-tetrahydrobenzo[b]thiophene-3-carboxylic acid amide;

6-tert-Butyl 2-(cyclopropanecarbonyl-amino)-4,5,6,7-tetrahydrobenzo[b]thiophene-3-carboxylic acid amide;

2-(Cyclopropanecarbonyl-amino)-6-methyl-4,5,6,7-tetrahydrobenzo[b]thiophene-3-carboxylic acid amide;

2-(Cyclopropylmethyl-amino)-4,5,6,7-tetrahydrobenzo[b]thiophene-3-carboxylic acid amide;

2-(Cyclohexanecarbonyl-amino)4,5,6,7-tetrahydrobenzo[b]thiophene-3-carboxylic acid amide;

2-Acetylamino-6-methyl-4,5,6,7-tetrahydrobenzo[b]thiophene-3-carboxylic acid amide;

2-Amino-4,7-dihydro-5H-thieno[2,3-c]-thiopyran-3-carboxylic acid amide;

2-(Cyclopropanecarbonyl-amino)-4,7-dihydro-5H-thieno[2,3-c]-thiopyran-3-carboxylic acid amide;

2-(Cyclopropanecarbonyl-amino)-6λ⁴-oxo-4,5,6,7-tetrahydro-thieno[2,3-c]thiopyran-3-carboxylic acid amide.

Not preferred are compounds wherein R¹ and R³ are both hydrogen.

The afore-mentioned disclaimer is only directed to the substance claim 1, but not to any one of the use claims 9-37.

Preferred are compounds wherein X¹ is S.

Furthermore, compounds are preferred wherein R⁸ and R⁹ represent hydrogen.

Also preferred are compounds wherein R¹ represents hydrogen. Preferred are compounds wherein the residue Y¹-Y²-Y³-Y⁴ bears at least one further substituent selected from R⁸-R¹⁷ which is different from hydrogen, i.d. Y¹ or Y² or Y³ or Y⁴ bears a further substituent R⁸-R¹⁷ which is different from hydrogen.

Preferably, —R⁵′, R¹⁰, R¹¹, R¹², R¹³, R¹⁶ and R¹⁷ represent independently of each other —F, —Cl, —Br, —I, —H, —OH, —OCH₃, —OC₂H₅, —COCH₃, and especially preferred —F, —Cl, —Br, —I.

Preferred are compounds wherein R² represents —CO—NH—R⁴, and more preferred wherein R² represents —CO—NH₂.

Still preferred are compounds wherein R¹⁰ and R¹¹ represent a smaller group, such as —CH₂F, —CHF₂, —CF₃, —CH₂OH, —CH₂NH₂, —CH₃, —C₂H₅, —C₃H₇, -cyclo-C₃H₅, —CH(CH₃)₂, —CH═CH₂, —OH, —OCH₃, —OC₂H₅, —OC₃H₇, —F, —Cl, —H, —COCH₃, —COOH, —COOCH₃, —CONH₂, —NH₂, —N(CH₃)₂, —SOCH₃, —SO₂CH₃, —SO₃H, —OCF₃.

Preferred are the following substructures (IIa)-(IIf), wherein R², R³, R⁸, R⁹, R¹⁰, R¹¹, R¹², R¹³, R¹⁶, and R¹⁷ have the meanings as defined in claim 1 or any part of the description:

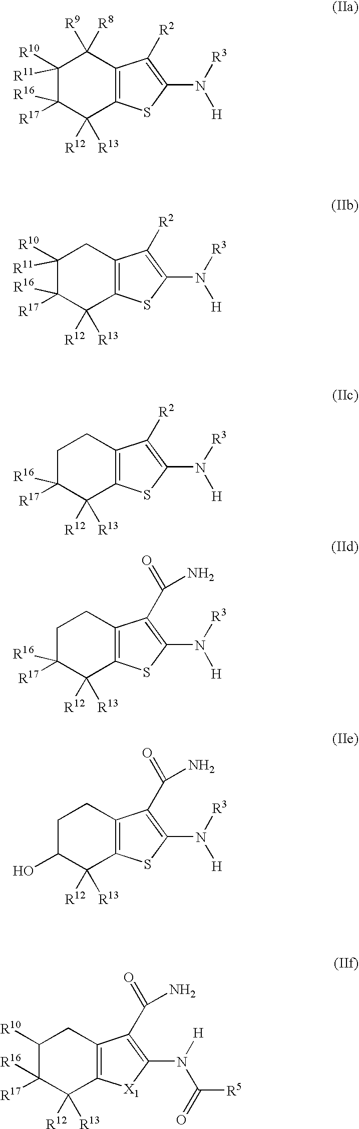

Further preferred are compounds of general formula (IIa) wherein R² represents —CONH₂ and/or wherein R³ represents —CO—R⁵ or —CO—R⁵′. Preferred are compounds of formula (IIa), wherein at least one substituent R⁸-R¹³ or R¹⁶ or R¹⁷ is not hydrogen.

Not preferred are compounds of formula (IIa) wherein R⁸-R¹⁷ are hydrogen and R² represents —CONH₂, —CONH—CH₃, —CONH—C₂H₅, and R³ represents —CONH—R*, and —R* represents —CH₃, —C₂H₅, —C₃H₇, —CH(CH₃)₂, —C₄H₉, cyclohexyl, -Ph, chlorophenyl.

Disclaimed are the following compounds:

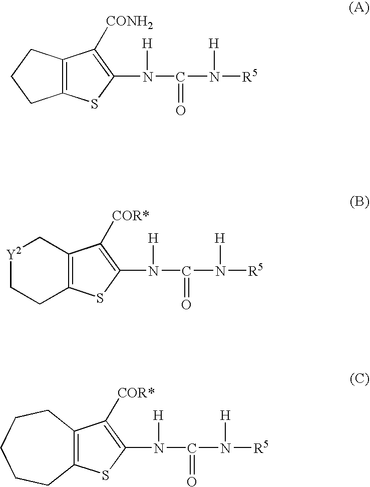

wherein R⁵ in formula (A) represents methyl, 1-propyl, n-butyl, cyclohexyl, phenyl, or 4-chlorophenyl;

in formula (B) R⁵ represents methyl, ethyl, 1-propyl, and R* represents —NH—C₂H₅, —NH₂, —OCH₃, —OC₂H₅, and Y² represents —CH₂—, —O—, —S—, or —NH—;

and in formula (C) R* represents —NH₂, —OCH₃, and R⁵ represents methyl or ethyl.

The following compounds are also disclaimed:

N-[3-carbamoyl-4,5,6,7-tetrahydrobenzo[b]thien-2-yl]-acetamide;

N-[3-carbamoyl-4,5,6,7-tetrahydrobenzo[b]thien-2-yl]-propionamide;

N-[3-carbamoyl-4,5,6,7-tetrahydrobenzo[b]thien-2-yl]-2-butynoic amide;

N-[3-carbamoyl-4,5,6,7-tetrahydrobenzo[b]thien-2-yl]-cyanoacetamide;

N-[3-carbamoyl-4,5,6,7-tetrahydrobenzo[b]thien-2-yl]-cyclopropanecarboxamide;

N-[3-carbamoyl-4,5,6,7-tetrahydrobenzo[b]thien-2-yl]-isobutyramide;

N-[3-carbamoyl-4,5,6,7-tetrahydrobenzo[b]thien-2-yl]-3,3-dimethylacrylic amide;

N-[3-carbamoyl-4,5,6,7-tetrahydrobenzo[b]thien-2-yl]-2-ketobutyramide;

N-[3-carbamoyl-4,5,6,7-tetrahydrobenzo[b]thien-2-yl]-N,N-dimethylglycinamide;

N-[3-carbamoyl-4,5,6,7-tetrahydrobenzo[b]thien-2-yl]-3-chloropropionamide;

N-[3-carbamoyl-4,5,6,7-tetrahydrobenzo[b]thien-2-yl]-imidazol-4-carboxamide;

N-[3-carbamoyl-4,5,6,7-tetrahydrobenzo[b]thien-2-yl]-pyrrole-2-carboxamide;

N-[3-carbamoyl-4,5,6,7-tetrahydrobenzo[b]thien-2-yl]-cyclopentanecarboxamide;

N-[3-carbamoyl-4,5,6,7-tetrahydrobenzo[b]thien-2-yl]-1-cyanocyclopropane-carboxamide;

N-[3-carbamoyl-4,5,6,7-tetrahydrobenzo[b]thien-2-yl]-N-acetylglycinamide;

N-[3-carbamoyl-4,5,6,7-tetrahydrobenzo[b]thien-2-yl]-pyrrole-3-carboxamide;

N-[3-carbamoyl-4,5,6,7-tetrahydrobenzo[b]thien-2-yl]-benzamide;

N-[3-carbamoyl-4,5,6,7-tetrahydrobenzo[b]thien-2-yl]-4-pyrazolecarboxamide;

N-[3-carbamoyl-4,5,6,7-tetrahydrobenzo[b]thien-2-yl]-picolinic amide;

N-[3-carbamoyl-4,5,6,7-tetrahydrobenzo[b]thien-2-yl]-nicotinic amide;

N-[3-carbamoyl-4,5,6,7-tetrahydrobenzo[b]thien-2-yl]-isonicotinic amide;

N-[3-carbamoyl-4,5,6,7-tetrahydrobenzo[b]thien-2-yl]-2-pyrazinecarboxamide;

N-[3-carbamoyl-4,5,6,7-tetrahydrobenzo[b]thien-2-yl]-1-methylpyrrole-2-carboxamide;

N-[3-carbamoyl-4,5,6,7-tetrahydrobenzo[b]thien-2-yl]-3-methyl-2-furoic amide;

N-[3-carbamoyl-4,5,6,7-tetrahydrobenzo[b]thien-2-yl]-5-methylisoxazole-4-carboxamide;

N-[3-carbamoyl-4,5,6,7-tetrahydrobenzo[b]thien-2-yl]-3-methylisoxazole-4-carboxamide;

N-[3-carbamoyl-4,5,6,7-tetrahydrobenzo[b]thien-2-yl]-thiophene-2-carboxamide;

N-[3-carbamoyl-4,5,6,7-tetrahydrobenzo[b]thien-2-yl]-thiophene-3-carboxamide;

N-[3-carbamoyl-4,5,6,7-tetrahydrobenzo[b]thien-2-yl]-dl-pyroglutamic amide;

N-[3-carbamoyl-4,5,6,7-tetrahydrobenzo[b]thien-2-yl]-1-(aminocarbonyl)-1-cyclopropanecarboxamide;

N-[3-carbamoyl-4,5,6,7-tetrahydrobenzo[b]thien-2-yl]-o-toluic amide;

N-[3-carbamoyl-4,5,6,7-tetrahydrobenzo[b]thien-2-yl]-5-methylisoxazole-3-carboxamide;

N-[3-carbamoyl-4,5,6,7-tetrahydrobenzo[b]thien-2-yl]-m-toluic amide;

N-[3-carbamoyl-4,5,6,7-tetrahydrobenzo[b]thien-2-yl]-3-aminopyrazole-4-carboxamide;

N-[3-carbamoyl-4,5,6,7-tetrahydrobenzo[b]thien-2-yl]-p-toluic amide;

N-[3-carbamoyl-4,5,6,7-tetrahydrobenzo[b]thien-2-yl]-salicylic amide;

N-[3-carbamoyl-4,5,6,7-tetrahydrobenzo[b]thien-2-yl]-3-hydroxybenzamide;

N-[3-carbamoyl-6-methyl-4,5,6,7-tetrahydrothieno[2,3-c]pyridin-2-yl]-3,4,5-trimethoxybenzamide;

N-[3-carbamoyl-6-methyl-4,5,6,7-tetrahydrothieno[2,3-c]pyridin-2-yl]-2,4,6-trimethoxybenzamide;

N-[3-carbamoyl-6-methyl-4,5,6,7-tetrahydrothieno[2,3-c]pyridin-2-yl]-3-chlorobenzo[b]thiophene-2-carboxamide;

N-[3-carbamoyl-6-methyl-4,5,6,7-tetrahydrothieno[2,3-c]pyridin-2-yl]-3-(phenylsulfonyl)propionamide;

N-[3-carbamoyl-6-methyl-4,5,6,7-tetrahydrothieno[2,3-c]pyridin-2-yl]-4-toluenesulfonylacetamide;

N-[3-carbamoyl-6-methyl-4,5,6,7-tetrahydrothieno[2,3-c]pyridin-2-yl]-4-methylsulfonylphenylacetamide;

N-[3-carbamoyl-6-methyl-4,5,6,7-tetrahydrothieno[2,3-c]pyridin-2-yl]-5-fluoroindole-3-acetamide;

N-[3-carbamoyl-6-methyl-4,5,6,7-tetrahydrothieno[2,3-c]pyridin-2-yl]-3-phthalimido-propionamide;

N-[3-carbamoyl-6-methyl-4,5,6,7-tetrahydrothieno[2,3-c]pyridin-2-yl]-5-methoxy-2-methyl-3-indoleacetamide;

N-[3-carbamoyl-6-methyl-4,5,6,7-tetrahydrothieno[2,3-c]pyridin-2-yl]-5-methoxy-1-indanone-3-acetamide;

N-[3-carbamoyl-6-methyl-4,5,6,7-tetrahydrothieno[2,3-c]pyridin-2-yl]-5-(4-chlorophenyl)-2-furoic amide;

N-[3-carbamoyl-6-methyl-4,5,6,7-tetrahydrothieno[2,3-c]pyridin-2-yl]-6-chlorokynurenic amide;

N-[3-carbamoyl-6-methyl-4,5,6,7-tetrahydrothieno[2,3-c]pyridin-2-yl]-N′-(4-chlorophenyl)maleamic amide;

N-[3-carbamoyl-6-methyl-4,5,6,7-tetrahydrothieno[2,3-c]pyridin-2-yl]-N′-p-tosylglycinamide;

N-[3-carbamoyl-6-methyl-4,5,6,7-tetrahydrothieno[2,3-c]pyridin-2-yl]-5-chloroindole-2-carboxamide;

N-[3-carbamoyl-6-methyl-4,5,6,7-tetrahydrothieno[2,3-c]pyridin-2-yl]-N′-(1-naphthyl)maleamic amide;

N-[3-carbamoyl-6-methyl-4,5,6,7-tetrahydrothieno[2,3-c]pyridin-2-yl]-3-iodobenzamide;

N-[3-carbamoyl-6-methyl-4,5,6,7-tetrahydrothieno[2,3-c]pyridin-2-yl]-4-iodobenzamide;

N-[3-carbamoyl-6-methyl-4,5,6,7-tetrahydrothieno[2,3-c]pyridin-2-yl]-N-m-tolylphthalamic amide;

N-[3-carbamoyl-6-methyl-4,5,6,7-tetrahydrothieno[2,3-c]pyridin-2-yl]-N′-acetyl-dl-histidine;

N-[3-carbamoyl-6-methyl-4,5,6,7-tetrahydrothieno[2,3-c]pyridin-2-yl]-3-acetamino-6-bromobenzamide;

N-[3-carbamoyl-6-methyl-4,5,6,7-tetrahydrothieno[2,3-c]pyridin-2-yl]-2-acetamido-5-bromobenzamide;

N-[3-carbamoyl-6-methyl-4,5,6,7-tetrahydrothieno[2,3-c]pyridin-2-yl]-2-iodophenylacetamide;

N-[3-carbamoyl-6-methyl-4,5,6,7-tetrahydrothieno[2,3-c]pyridin-2-yl]-4-iodophenylacetamide;

N-[3-carbamoyl-6-methyl-4,5,6,7-tetrahydrothieno[2,3-c]pyridin-2-yl]-8-(3-carboxamidopropyl)-1,3-dimethylxanthine;

N-[3-carbamoyl-6-methyl-4,5,6,7-tetrahydrothieno[2,3-c]pyridin-2-yl]-7-bromokynurenic amide;

N-[3-carbamoyl-6-methyl-4,5,6,7-tetrahydrothieno[2,3-c]pyridin-2-yl]-N′-benzoyl-dl-phenylalaninamide;

N-[3-carbamoyl-6-methyl-4,5,6,7-tetrahydrothieno[2,3-c]pyridin-2-yl]-indole-3-butyramide;

N-[3-carbamoyl-6-methyl-4,5,6,7-tetrahydrothieno[2,3-c]pyridin-2-yl]-4-chloroindole-3-acetamide;

N-[3-carbamoyl-6-methyl-4,5,6,7-tetrahydrothieno[2,3-c]pyridin-2-yl]-dl-desthiobiotin;

N-[3-carbamoyl-6-methyl-4,5,6,7-tetrahydrothieno[2,3-c]pyridin-2-yl]-4,6-dichloroindole-2-carboxamide;

N-[3-carbamoyl-6-methyl-4,5,6,7-tetrahydrothieno[2,3-c]pyridin-2-yl]-N′-benzoyl-histidinamid.

Also preferred are the following substructures (IIIa)-(IIIf), wherein R², R³, R⁹, R¹⁰, R¹¹, R¹², R¹³, and X¹ have the meanings as defined in claim 1 or any part of the description:

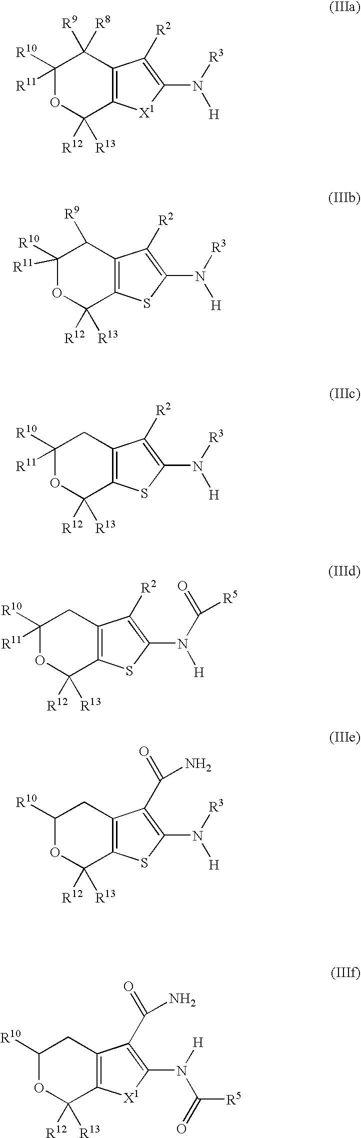

Especially the compounds of general formula (IIIa) are preferred

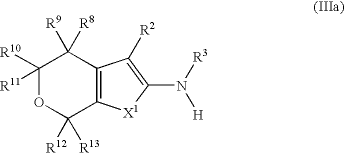

wherein

X¹ is selected from S, O, NR⁴′,

R⁴′ is selected from H, substituted or unsubstituted C₁-C₆-alkyl,

R² is selected from —CO—NH—R⁴, —CS—NH—R⁴, —SO₂—NH—R⁴;

wherein R⁴ is selected from —H, HO-substituted, H₂N-substituted or HS-substituted C₁-C₆-alkyl,

R³ is selected from H, —C(═O)R⁵, —C(═S)R⁵, —C(═NH)R⁵ and —SO₂R⁵,

wherein

R⁵ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, adamantyl,

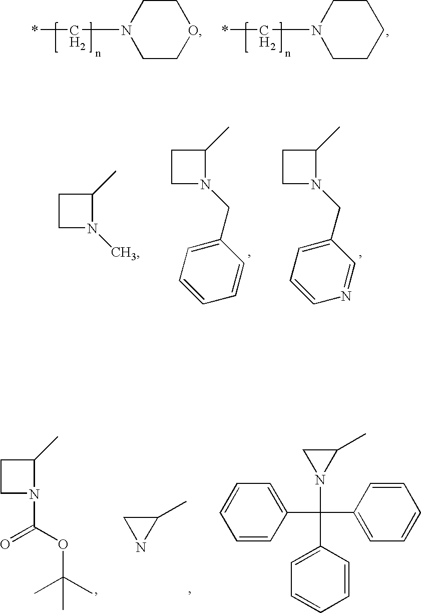

or —(CH₂)ₙ—NR⁶R⁷,

wherein R⁶ and R⁷ are independently selected from substituted or unsubstituted C₁-C₄-alkyl or C₂-C₄-alkenyl and wherein n=1 to 6,

or NR⁶R⁷,

wherein

R⁶ is selected from H, C₁-C₆-alkyl, and

R⁷ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, or adamantyl,

R⁸ is H and R⁹ is selected from H, substituted or unsubstituted C₁-C₆-alkyl

R¹⁰ is selected from H, substituted or unsubstituted C₁-C₆-alkyl, C₁-C₆-alkoxy, or OH

R¹¹ is selected from H and substituted or unsubstituted C₁-C₆-alkyl

R₁₂ is selected from H and substituted or unsubstituted C₁-C₆-alkyl, C₁-C₆-alkoxy, or OH, and

R¹³ is selected from H or substituted or unsubstituted C₁-C₆-alkyl,

and stereoisomeric and regioisomeric forms and pharmaceutically acceptable salts of these compounds.

In a preferred embodiment of general formula (IIIa) X¹ is S.

In a further preferred embodiment of general formula (IIIa) X¹ is NR⁴′, and NR⁴′ is selected from H, substituted or unsubstituted C₁-C₆-alkyl, and preferably is methyl, ethyl, n-propyl, iso-propyl, n-butyl, sec.-butyl, iso-butyl, tert.-butyl, or benzyl.

In a further preferred embodiment of general formula (IIIa) X¹ is O.

In a further preferred embodiment of general formula (IIIa) R² is —CO—NH—R⁴ and R⁴ is selected from H, HO-substituted, H₂N-substituted or HS-substituted C₁-C₄-alkyl, and preferably is H.

In a further preferred embodiment of general formula (IIIa) R² is —CS—NH—R⁴ and R⁴ is selected from H, HO-substituted, H₂N-substituted or HS-substituted C₁-C₄-alkyl, and preferably is H.

In a further preferred embodiment of general formula (IIIa) R² is —SO₂—NH—R⁴ and R⁴ is selected from H, HO-substituted, H₂N-substituted or HS-substituted C₁-C₄-alkyl, and preferably is H.

In yet another preferred embodiment of general formula (IIIa) R⁴ is selected from the group consisting of —H, —CH₂—CH₂—OH, —CH₂—CH₂—NH₂, —CH₂—CH₂—SH, —CH₂—CH(OH)—CH₃, —CH₂—CH(SH)CH₃, or —CH₂—CH(NH₂)—CH₃.

In a further preferred embodiment of general formula (IIIa) R³ is —CO—R⁵, —CS—R⁵, —CO—R⁵′, or —CS—R⁵′, and more preferably —CO—R⁵ or —CO—R⁵′ and most preferably —CO—R⁵.

In a further preferred embodiment of general formula (IIIa) R³ is —SO₂—R⁵ or —SO₂—R⁵′ and more preferably —SO₂—R⁵.

In yet another preferred embodiment of general formula (IIIa) R⁵ or R⁵′ is selected from the group consisting of substituted or unsubstituted methyl, ethyl, propyl, butyl, cyclopropyl, cyclobutyl, cyclopentyl, cyclohexyl, C₁-C₆-cycloalkyles substituted by at least one methyl or carboxyl group, phenyl, furanyl, thienyl, pyrrolyl, pyridyl, pyrrolidinyl, piperidinyl, tetrahydrofuranyl, ethenyl, prop-1-enyl, prop-2-enyl, but-1-enyl, but-2-enyl, but-3-enyl, prop-1-inyl, prop-2-inyl, but-1-inyl, but-2-inyl, but-3-inyl, adamantyl, or NR⁶R⁷, wherein R⁶ is H and R⁷ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, or adamantyl.

In yet another preferred embodiment of general formula (IIIa) R⁵ or R⁵′ is selected from the group consisting of cyclopropyl, cyclobutyl, cyclopentyl, cyclohexyl, phenyl-substituted cyclopropyl, cyclobutyl, cyclopentyl, or cyclohexyl, methyl-substituted cyclopropyl, cyclobutyl, cyclopentyl, or cyclohexyl, carboxyl substituted cyclopropyl, cyclobutyl, cyclopentyl, or cyclohexyl, furanyl, methyl, ethyl, n-propyl, iso-propyl, n-butyl, iso-butyl, tert.-butyl, prop-1-enyl, but-1-enyl, adamantyl, 3,4-difluorophenyl or NR⁶R⁷, wherein R⁶ is H and R⁷ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, or adamantyl, and R⁷ preferably is phenyl or 3,4-difluorophenyl.

In another preferred embodiment of general formula (IIIa) R⁷ is selected from substituted or unsubstituted C₃-C₆-cycloalkyl, C₁-C₆-alkyl, heteroaryl, heterocycloalkyl, C₂-C₄-alkenyl, C₂-C₄-alkinyl, or adamantyl. In a further embodiment of general formula (IIIa), the compound 5,5-dimethyl-2-(3-phenyl-ureido)-4,7-dihydro-5H-thieno[2,3-c]pyran-3-carboxylic acid amide is excluded from the compounds according to the present invention.

In yet another embodiment of general formula (IIIa) R⁷ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, or adamantyl, and R¹⁰ is selected from H, substituted or unsubstituted C₁-C₆-alkoxy, or OH.

In yet another preferred embodiment of general formula (IIIa) R⁸ is H and R⁹ is selected from H, or substituted or unsubstituted C₁-C₆-alkyl.

In a further preferred embodiment of general formula (IIIa) R¹⁰, R¹¹, R¹², and R¹³ are independently selected from H and substituted or unsubstituted C₁-C₆-alkyl, and preferably from H or methyl, ethyl, n-propyl, iso-propyl, n-butyl, iso-butyl or tert.-butyl.

In yet another preferred embodiment of general formula (IIIa) R¹⁰ and R¹¹ are methyl and R¹² and R¹³ are H, or R¹⁰, R¹¹, R¹², and R¹³ are H, or R¹⁰, R¹¹, R¹², and R¹³ are methyl, or R¹⁰ and R¹¹ are H and R¹² and R¹³ are methyl.

In yet another preferred embodiment of general formula (IIIa) R¹⁰ is selected from substituted or unsubstituted C₁-C₆-alkoxy or OH and R¹¹ is selected from H or substituted or unsubstituted C₁-C₆-alkyl.

In yet another preferred embodiment of general formula (IIIa) R¹² is selected from substituted or unsubstituted C₁-C₆-alkoxy or OH and R¹³ is selected from H or substituted or unsubstituted C₁-C₆-alkyl.

In a further preferred embodiment of general formula (IIIa) R⁴′ and/or R¹ is selected from the group consisting of methyl, ethyl, n-propyl, iso-propyl, n-butyl, iso-butyl, tert.-butyl or benzyl.

In a further preferred embodiment of general formula (IIIa) R⁶ and R⁷ are independently selected from methyl, ethyl and propyl or allyl, and preferably are methyl.

Preferred are the following substructures (IVa)-(IVf) of general formula (I), wherein R², R³, R⁸, R⁹, R¹⁰, R¹¹, R¹², R¹³, R¹⁵, and X¹ have the meanings as defined in claim 1 or any part of the description:

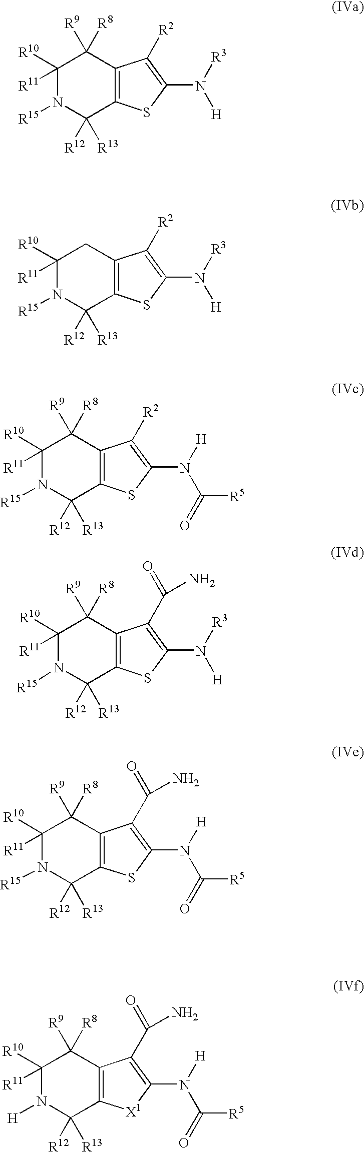

Furthermore, preferred are the following substructures (Va)-(Vd), wherein R², R³, R¹⁰, R¹¹, R¹², and X¹ have the meanings as defined in claim 1 or any part of the description:

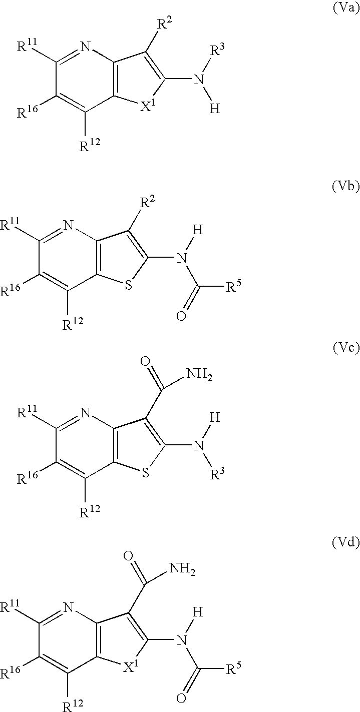

Still preferred are the following substructures (VIa)-(VId), wherein R², R³, R¹⁰, R¹¹, R¹², R¹³, and X¹ have the meanings as defined in claim 1 or any part of the description:

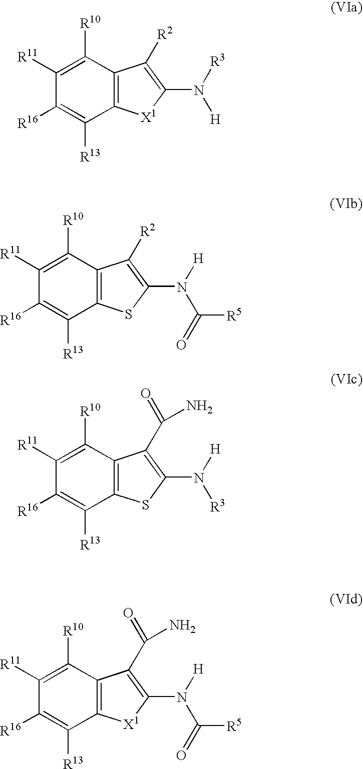

Excluded from the scope of the present invention via disclaimer are the following compounds:

2-Amino-6-(4-fluoro-phenyl)-benzo[b]thiophene-3-carboxylic acid amide;

2-Ureido-benzo[b]thiophene-3-carboxylic acid amide.

Not preferred are compounds of the general formula (VIc), wherein R³ represents —CO—NH₂. Also not preferred are compounds of the general formula (VIc), wherein R³ represents —CO—NH₂ and R¹⁰, R¹¹, R¹³ are hydrogen and R¹⁶ represents —H, —CN, —CF₃, halogen, aryl, heteroaryl, alkyl, O-alkyl, or S-alkyl.

Preferred are still the following substructures (VIIa)-(VIId), wherein R¹, R², R³, R⁸, R¹⁰, R¹⁷, and X¹ have the meanings as defined in claim 1 or any part of the description:

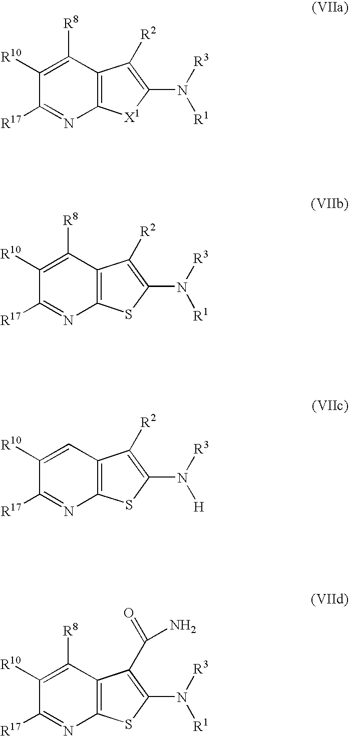

Still preferred are the following substructures (VIIIa)-(VIIId), wherein R¹, R², R³, R¹⁰, R¹⁷, and X¹ have the meanings as defined in claim 1 or any part of the description:

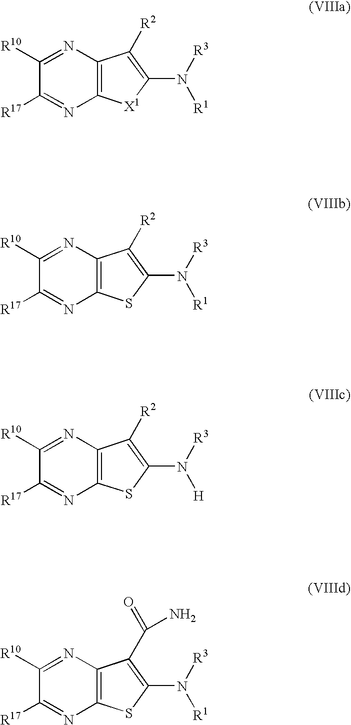

Preferred are still the following substructures (IXa)-(IXd), wherein R¹, R², R³, R¹⁰, R¹², and X¹ have the meanings as defined in claim 1 or any part of the description:

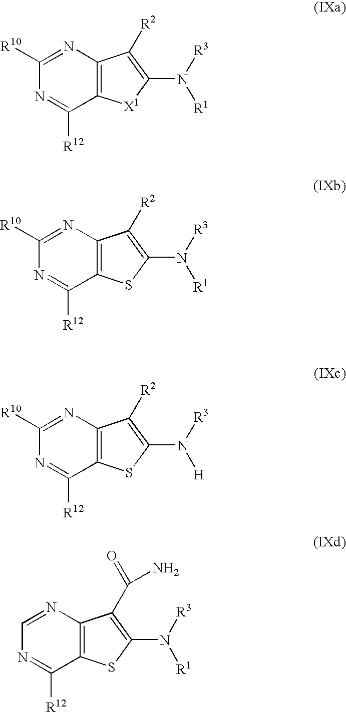

Preferred are still the following substructures (Xa)-(Xd), wherein R¹, R², R³, R⁸, R⁹, R¹⁰, R¹¹, R¹², R¹³, and X¹ have the meanings as defined in claim 1 or any part of the description:

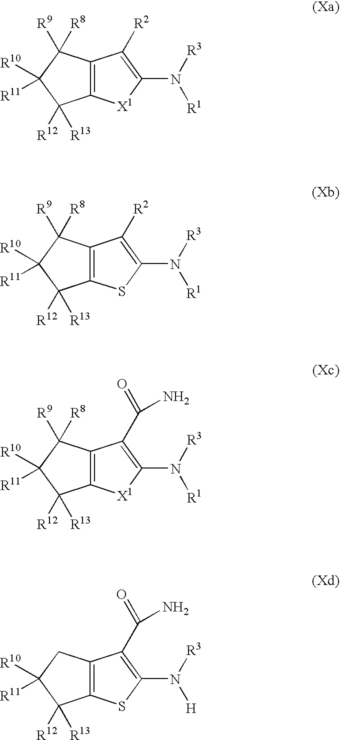

Preferred are still the following substructures (XIa)-(XId), wherein R¹, R², R³, R⁸, R⁹, R¹⁰, R¹¹, R¹², R¹³, R¹⁵, R¹⁶, R¹⁷, and X¹ have the meanings as defined in claim 1 or any part of the description:

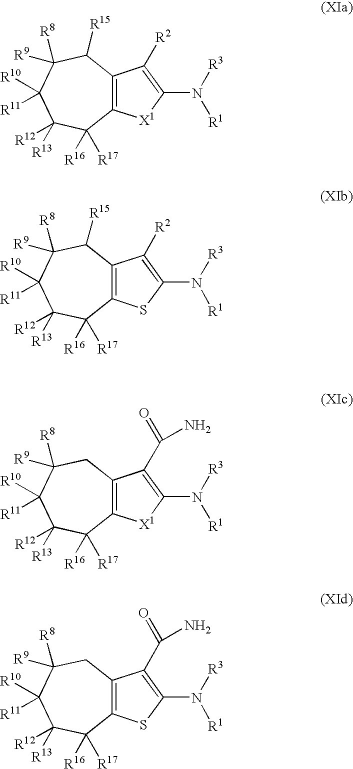

Preferred are especially all compounds of any of the above-mentioned general formula (I)-(XI), wherein at least one of the substituents R³-R¹³, R¹⁵-R¹⁷ are different from hydrogen, more preferably wherein at least one substituent R¹², R¹³, R¹⁶, or R¹⁷ is different from hydrogen. Still further preferred are compounds wherein R¹², R¹³ and/or R¹⁶ are residues containing at least one oxygen and/or nitrogen atom such as the residues: —OOC—O—R¹⁴′, —O—CO—R¹⁴′, —COO—R¹⁴′, —O—CO—NR⁷R¹⁴, —CO—NR⁷R¹⁴, —NR⁷R¹⁴, —O—R¹⁴′, —OCR¹⁴′R¹⁴″R¹⁴′″, —OCH₂—R¹⁴′, —O—(CH₂)ₘ—R¹⁴′, —OCH₂—CR¹⁴′R¹⁴″R¹⁴′″, —O—(CH₂)ₘ—CR¹⁴′R¹⁴″R¹⁴′″, —O—(CH₂)ₚ—OCR¹⁴′R¹⁴″R¹⁴′″, —O—(CH₂)ₚ—NR¹⁴″R¹⁴′″, —O—(CH₂)ₘ—CO—R¹⁴′, —O—(CH₂)ₚ—O—CO—R¹⁴′, —O—(CH₂)ₚ—O—CO—OR¹⁴′, —O—(CH₂)ₚ—NR¹⁴′—CO—R¹⁴″, —O—(CH₂)ₚ—NR¹⁴′—CO—OR¹⁴″, —O—(CH₂)ₘ—CO—OCR¹⁴′R¹⁴″R¹⁴′″, —O—CR¹⁹R²⁰—CO—OCR¹⁴′R¹⁴″R¹⁴′″, —O—(CH₂)ₘ—CO—NR¹⁴″R¹⁴′″, —O—(CH₂)ₚ—O—CO—NR¹⁴″R¹⁴′″, —O—CR¹⁹R²⁰—O—CO—NR¹⁴″R¹⁴′″, —(CH₂)ₘ—OR¹⁴′, —(CH₂)ₘ—NR¹⁴″R¹⁴′″, —(CH₂)ₘ—O—CO—R¹⁴′, —(CH₂)ₘ—NR¹⁴′—CO—R¹⁴″, —(CH₂)ₘ—NR¹⁴′—CO—OR¹⁴″, —(CH₂)ₘ—CO—OR¹⁴′, —(CH₂)ₘ—CO—NR¹⁴″R¹⁴′″, —(CH₂)ₘ—O—CO—NR¹⁴″R¹⁴′″.

As used herein, the term “unsubstituted C₁-C₈-alkyl” refers to —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₇H₁₅, —C₈H₁₇, —C₄H₈—CH(CH₃)₂, —C₅H₁₀—CH(CH₃)₂. Consequently, C₁-C₇-alkyl will refer the the residues disclosed before having 1 to 7 carbon atoms and C₁-C₃-alkyl will refer to the residues mentioned before having 1 to 3 carbon atoms.

Preferred are —CH₃, —C₂H₅, —C₃H₇, —CH(CH₃)₂, —C₄H₉, —CH₂—CH(CH₃)₂, —CH(CH₃)—C₂H₅, —C(CH₃)₃, and —C₅H₁₁. Especially preferred are —CH₃, —C₂H₅, —C₃H₇, and —CH(CH₃)₂.

The term “unsubstituted C₂-C₆-alkenyl” refers to —CH═CH₂, —CH₂—CH═CH₂, —C(CH₃)═CH₂, —CH═CH—CH₃, —C₂H₄—CH═CH₂, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃)—CH═CH—CH₃, —CH═CH—CH(CH₃)₂, —CH═C(CH₃)—C₂H₅, —C(CH₃)═CH—C₂H₅, —C(CH₃)═C(CH₃)₂, —C(CH₃)₂—CH═CH₂, —CH(CH₃)—C(CH₃)═CH₂, —C(CH₃)═CH—CH═CH₂, —CH═C(CH₃)—CH═CH₂, —CH═CH—C(CH₃)═CH₂, —C₄H₈—CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)—CH═CH₂, —CH₂—CH(CH₃)—CH₂—CH═CH₂, —CH(CH₃)—C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)—CH₂—CH═CH—CH₃, —CH₂—CH═CH—CH(CH₃)₂, —CH₂—CH═C(CH₃)—C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃)—C₂H₅, —CH═C(CH₃)—C₃H₇, —C(CH₃)═CH—C₃H₇, —CH₂—CH(CH₃)—C(CH₃)═CH₂, —CH(CH₃)—CH₂—C(CH₃)═CH₂, —CH(CH₃)—CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —CH═CH—C(CH₃)₃, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₃H₇)═CH₂, —CH₂—C(C₂H₅)═CH—CH₃, —CH(C₂H₅)—CH═CH—CH₃, —C(C₄H₉)═CH₂, —C(C₃H₇)═CH—CH₃, —C(C₂H₅)═CH—C₂H₅, —C(C₂H₅)═C(CH₃)₂, —C[C(CH₃)₃]═CH₂, —C[CH(CH₃)(C₂H₅)]═CH₂, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH₂—CH═CH—CH═CH—CH₃, —CH═CH—CH₂—CH═CH—CH₃, —CH═CH—CH═CH—C₂H₅, —CH₂—CH═CH—C(CH₃)═CH₂, —CH₂—CH═C(CH₃)—CH═CH₂, —CH₂—C(CH₃)═CH—CH═CH₂, —CH(CH₃)—CH═CH—CH═CH₂, —CH═CH—CH₂—C(CH₃)═CH₂, —CH═CH—CH(CH₃)—CH═CH₂, —CH═C(CH₃)—CH₂—CH═CH₂, —C(CH₃)═CH—CH₂CH═CH₂, —CH═CH—CH═C(CH₃)₂, —CH═CH—C(CH₃)═CH—CH₃, —CH═C(CH₃)—CH═CH—CH₃, —C(CH₃)═CH—CH═CH—CH₃, —CH═C(CH₃)—C(CH₃)═CH₂, —C(CH₃)═CH—C(CH₃)═CH₂, —C(CH₃)═C(CH₃)—CH═CH₂, and —CH═CH—CH═CH—CH═CH₂.

Preferred are —CH═CH₂, —CH₂—CH═CH₂, —C(CH₃)═CH₂, —CH═CH—CH₃, —C₂H₄—CH═CH₂, —CH₂—CH═CH—CH₃. Especially preferred are —CH═CH₂, —CH₂—CH═CH₂, and —CH═CH—CH₃.

The term “unsubstituted C₂-C₆-alkynyl” refers to —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C—C₃H₇, —C≡C—C₄H₉, —C₂H₄—CH(CH₃)—C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C≡C—CH(CH₃)—C₂H₅, —C≡C—CH₂—CH(CH₃)₂, —C≡C—C(CH₃)₃, —CH(C₂H₅)—C≡C—CH₃, —C(CH₃)₂—C≡C—CH₃, —CH(C₂H₅)—CH₂—C≡CH, —CH₂—CH(C₂H₅)—C≡CH, —C(CH₃)₂—CH₂—C≡CH, —CH₂—C(CH₃)₂—C≡CH, —CH(CH₃)—CH(CH₃)—C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)—C≡C—C≡CH, —CH(C≡CH)—CH₂—C≡CH, —C(C≡CH)₂—CH₃, —CH₂—CH(C≡CH)₂, —CH(C≡CH)—C≡C—CH₃, —C≡C—CH≡CH₂, —CH≡CH—C≡CH, —CH₂—C≡C—CH═CH₂, —CH₂—CH═CH—C≡CH, —C≡C—CH═CH—CH₃, —CH═CH—C≡C—CH₃, —C≡C—CH₂—CH═CH₂, —CH═CH—CH₂—C≡CH, —C≡C—CH₂—C≡CH, —C(CH₃)═CH—C≡CH, —CH═C(CH₃)—C≡CH, —C≡C—C(CH₃)═CH₂, and —C≡C—C≡C—C≡CH.

Preferred are —C≡CH, —C≡C—CH₃.

As used herein, the terms “substituted C₁-C₈-alkyl”, “substituted C₂-C₆-alkenyl”, and “substituted C₂-C₆-alkynyl” refer to the above-mentioned “unsubstituted C₁-C₈-alkyl”, “unsubstituted C₂-C₆-alkenyl”, and “unsubstituted C₂-C₆-alkynyl” residues which may be substituted with one, two, three, four, five, six, or seven substituents independently selected from the group referred to as R¹⁹-R³³. Preferred are the following substituents: —OH, —OCH₃, —OC₂H₅, —OC₃H₇, —O-cyclo-C₃H₅, —OCH(CH₃)₂, —OC(CH₃)₃, —OPh, —OCH₂-Ph, —OCPh₃, —OR⁶, —OR⁷, —SH, —SCH₃, —SC₂H₅, —NO₂, —F, —Cl, —Br, —I, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —OOC—CH₃, —OOC—C₂H₅, —OOC—C₃H₇, —CONH₂, —CON(CH₃)₂, —CON(C₂H₅)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —SOCH₃, —SOC₂H₅, —SO₂CH₃, —SO₂C₂H₅, —SO₃H, —SO₃CH₃, —SO₃C₂H₅, —OCF₃, —O—COOCH₃, —O—COOC₂H₅, —O—COOC₃H₇, —O—CO—NH₂, —O—CO—N(CH₃)₂, —O—CO—N(C₂H₅)₂, —CH₂F, —CF₃, —CH₂Cl, —CCl₃, —CH₂Br, —CH₂I, —CH₂OH, —CH₂SH, —CH₂NH₂, —CH₂N(CH₃)₂, —CH₂N(C₂H₅)₂, —C₂H₄—OH, —C₂H₄—SH, —C₂H₄—NH₂, —C₂H₄—N(CH₃)₂, —C₂H₄—N(C₂H₅)₂, —CH₂OCH₃, —CH₂SCH₃, —CH₂OC₂H₅, —C₂H₄—OCH₃, —C₂H₄—OC₂H₅, —CH₃, —C₂H₅, —C₃H₇, -cyclo-C₃₅, —CH(CH₃)₂, —C(CH₃)₃, —C₄H₉, —CH₂—CH(CH₃)₂, —CH(CH₃)—s C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH═CCl₂, —CH₂—CH═CH₂, —C(CH₃)═CH₂, —CH═CH—CH₃, —CH═CH—CH₂—OH, —CH═CH—COOH, —CH═CH—COOCH₃, —CH═CH—COOC₂H₅, —C(CH₃)═CH—CH₃, —C₂H₄—CH═CH₂, —CH═C(CH₃)₂, —C≡CH, —C≡C—CH₃, —CH₂—C≡CH.

As used herein, the term “unsubstituted C₃-C₁₀-cycloalkyl” refers to

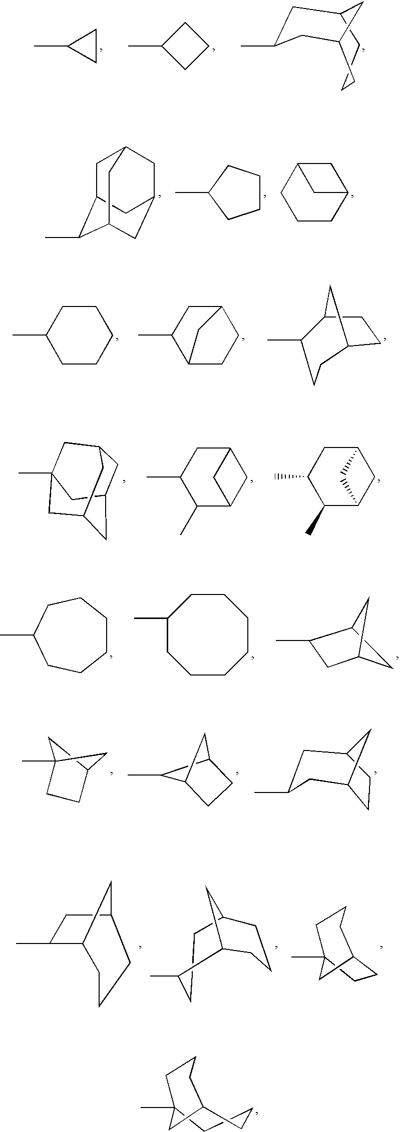

The term “substituted C₃-C₁₀-cycloalkyl” refers to the above-mentioned carbocyclic residues which are substituted with one, two, three, four, five, six, or seven substituents independently selected from the group referred to as R¹⁹-R³³. Preferred substituents are listed above and are the same as summarized for the “substituted C₁-C₈-alkyl”, “substituted C₂-C₆-alkenyl”, and “substituted C₂-C₆-alkynyl” residues.

As used herein, the term “—O—C₃-C₁₀-cycloalkyl” refers to a substituted or unsubstituted C₃-C₁₀-cycloalkyl residue bound via an oxygen to the bicyclic scaffold. For example, “—O—C₃-cycloalkyl” refers to —O-cyclo-C₃H₅.

Accordingly, the term “—O—C₁-C₆-heterocyclyl” refers to a substituted or unsubstituted heterocyclic ring containing at least one carbon atom. Said heterocyclic ring is bound via said at least one carbon atom to the bicyclic scaffold through an oxygen linker. Examples for “—O—C₄-heterocyclyl” are:

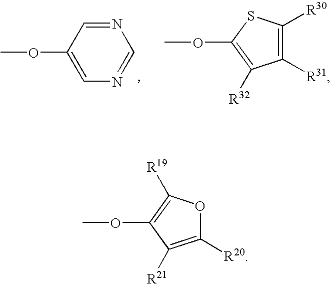

As used herein, the term “substituted phenyl” refers to a phenyl ring substituted with one, two, three, four, or five substituents independently selected from the group referred to as R¹⁹-R³³.

As used herein, the term “substituted benzyl” refers to the residue —CH₂-Ph wherein Ph represents a substituted phenyl as defined above.

As used herein, the term “unsubstituted aryl” refers to phenyl, indenyl, indanyl, naphthyl, 1,2-dihydro-naphthyl, 2,3-dihydronaphthyl, 1,2,3,4-tetrahydronaphthyl (tetralinyl), fluorenyl, anthryl(anthracenyl), 9,10-dihydroanthryl, 1,2,3,4-tetrahydro-anthryl, 1,2,3,4,5,6,7,8-octahydro-anthryl, azulenyl, diphenylmethyl, benzyl, triphenylmethyl(trityl), styryl, naphthoquinonyl, acenaphthyl, anthraquinonyl, phenanthryl(phenanthrenyl).

As used herein, the term “substituted aryl” refers to any one of the residues “unsubstituted aryl” substituted with one, two, three, four, five, six, or seven substituents independently selected from the group referred to as R¹⁹-R³³.

The term “alkylaryl” refers to a substituted or unsubstituted aryl moieties linked to the rest of the molecule via a carbon chain. In its easiest form, “alkylaryl” refers to benzyl. Other examples are styryl, phenylethyl, phenylpropyl.

As used herein, the term “substituted C₁-C₆-alkoxy” or “substituted C₁-C₆-alkyloxy” refers to substituted or unsubstituted C₁-C₆-alkyl, wherein an additional oxygen is present at a non-terminal position. Examples for C₁-C₆-alkoxy are: —O—CH₃, —CH₂—O—CH₃, —C₂H₄—O—CH(CH₃)₂, —CH₂—O—C₂H₅, —CH₂—O—CH₂—C(CH₃)₃.

As used herein, the term “unsubstituted C₁-C₈-acyl” refers to the residues —CO—C₁-C₇-alkyl, wherein C₁-C₇-alkyl has the meanings as defined above. Accordingly, the term “substituted C₁-C₈-acyl” refers to the residues referred to as “unsubstituted C₁-C₈-acyl” which were substituted with one, two, three, four, five, six, or seven substituents independently selected from the group referred to as R¹⁹-R³³.

As used herein, the term “unsubstituted C₁-C₆-heterocyclyl” or “unsubstituted C₁-C₆-heterocyclyl” refers to carbocycles having at least one heteroatom in the ring such as oxygen, nitrogen, or sulfur. Such heterocycles may be saturated or partially unsaturated but not aromatic. Examples for heterocyclic residues are 1,3-dioxolane, benzo[1,3]dioxolyl, pyrazolinyl, pyranyl, thiomorpholinyl, pyrazolidinyl, piperidyl, piperazinyl, 1,4-dioxanyl, imidazolinyl, pyrrolinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranl. The term “C₄-heterocyclyl” refers to heterocyclic residues with 4 carbon atoms and at least one heteroatom such as tetrahydrofuran, optionally substituted or unsubstituted. Examples for “C₁-heterocyclyl” are diaziridine and oxaziridine.

As used herein, the term “substituted C₁-C₆-heterocyclyl” or “substituted C₁-C₆-heterocyclyl” refers to the afore-mentioned heterocycles having one, two, three, four, five, six, or seven substituents independently selected from the group referred to as R¹⁹-R³³.

As used herein, the term “unsubstituted heteroaryl” refers to heteroaromatic groups which have from 4 to 9 ring atoms, from 1 to 4 of which are selected from O, N and/or S. Preferred groups have 1 or 2 heteroatoms in a 5- or 6-membered aromatic ring. Mono and bicyclic ring systems are included. Typical heteroaryl groups include pyridyl, furyl, thienyl, pyrrolyl, oxazolyl, thiazolyl, imidazolyl, isoxazolyl, isothiazolyl, oxadiazolyl, pyridazinyl, pyrimidyl, pyrazinyl, 1,3,5-triazinyl, 1,2,3-triazolyl, 1,3,4-thiadiazolyl, indolizinyl, indolyl, isoindolyl, benzo[b]furyl, benzo[b]thienyl, indazolyl, benzimidazolyl, benzthiazolyl, purinyl, quinolizinyl, quinolyl, isoquinolyl, quinazolinyl, quinoxalinyl, 1,8-naphthyridinyl, tetrahydroquinolyl, benzooxazolyl, chrom-2-onyl, indazolyl, and the like.

As used herein, the term “substituted heteroaryl” refers to heteroaromatic groups as disclosed before having one, two, three, four, or five substituents independently selected from the group referred to as R¹⁹-R³³.

As used in the present invention, the term substituted or unsubstituted C₁-C₆-alkyl or C₁-C₄-alkyl or C₁-C₃-alkyl is preferably meant to include linear or branched alkyl groups in which optionally one, two or three of the hydrogen atoms bonded to the carbon chain are substituted by a halogen atom such as F, Cl, Br, or I, preferably F or Cl, a —OH or —SH group, a —NH₂ group, methoxy or ethoxy group, or phenyl group. These terms therefore especially comprise, depending on the number of carbon atoms in each respective term, methyl, ethyl, n-propyl, iso-propyl, n-butyl, sec-butyl, iso-butyl, tert.-butyl, —C₅H₁₁, —CH₂—C(CH₃)₃, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, optionally substituted in the above described manner, especially to give phenyl substituted alkyles such as benzyl.

Similarly, the term substituted or unsubstituted C₃-C₆-cycloalkyl is preferably meant to include cyclolalkanes in which optionally one, two or three of the hydrogen atoms bonded to the carbon atoms of the cycle are substituted by a halogen atom such as F, Cl , Br, or I, preferably F or Cl, a —OH or —SH group, a —NH₂, methoxy or ethoxy or methyl, ethyl or phenyl group. This term therefore preferably includes cyclopropyl, cyclobutyl, cyclopentyl, cyclohexyl as well as methyl substituted cyclopropyl, cyclobutyl, cyclopentyl, cyclohexyl, ethyl substituted cyclopropyl, cyclobutyl, cyclopentyl, cyclohexyl or phenyl substituted cyclopropyl, cyclobutyl, cyclopentyl, cyclohexyl, optionally substituted in the above described manner.

Similarly, the term unsubstituted or substituted C₂-C₄-alkenyl is preferably meant to include branched or linear alkenyles in which optionally one, two, three or four of the hydrogen atoms bonded to the carbon atoms of the alkyl are substituted by a halogen atom such as F, Cl , Br, or I, preferably F or Cl. These terms therefore are meant to preferably comprise ethenyl, cis-prop-1-enyl, trans-prop-1-enyl, cis-prop-2-enyl, trans-prop-2-enyl, but-1-enyl, cis-but-2-enyl, trans-but-2-enyl, but-3-enyl, optionally substituted in the above described manner.

Similarly, the term unsubstituted or substituted C₂-C₄-alkinyl is preferably meant to include branced or linear alkinyles in which optionally one, two, three or four of the hydrogen atoms bonded to the carbon atoms of the alkyl are substituted by a halogen atom such as F, Cl , Br, or I, preferably F or Cl. These terms therefore are meant to preferably comprise prop-1-inyl, prop-2-inyl, but-1-inyl, but-2-inyl, and but-3-inyl, optionally substituted in the described above manner.

The term substituted or unsubstituted aryl is preferably meant to include aromatic compounds, in which one, two or three of the hydrogen atoms bonded to the aromatic ring are substituted by an halogen, such as F, Cl, Br or I, preferably F and Cl, or substituted by —NO₂, —OH, —SH, —NH₂, —CN, methyl or methoxy. This term is therefore meant to preferably comprise phenyl, 2,3-halogen substituted phenyl, 3,4-halogen substituted phenyl.

The term substituted or unsubstituted heteroaryl is preferably meant to include aromatic groups in which the aromatic ring comprises at least one heteroatom selected from the group N, O, or S, and in which one, two or three of the hydrogen atoms bonded to the aromatic ring are optionally substituted by an halogen, such as F, Cl, Br or I, preferably F and Cl, or substituted by —NO₂, —OH, —SH, methyl or methoxy. This term therefore includes preferably furanyl, pyrrolyl, thienyl, and pyridinyl which optionally can be substituted in the above described manner.

The term substituted or unsubstituted heterocycloalkyl is preferably meant to include cycloalkyles in which at least one of the carbon atoms of the ring, preferably 1 or 2 atoms, have been substituted by a heteroatom selected from the group consisting of N, O, and S which optionally and in which one, two or three of the hydrogen atoms bonded to the ring are substituted by an halogen, such as F, Cl, Br or I, preferably F and Cl, or substituted by methyl or methoxy. This term therefore includes preferably pyrrolidinyl, piperidinyl and tetrahydrofuranyl, which optionally can be substituted in the above described manner.

In yet another preferred embodiment of the invention compound according to formula (I) is selected from the group comprising:
(Compound A1) 2-(3-Cyclohexyl-ureido)-6-hydroxy-benzo[b]thiophene-3-carboxylic acid amide,
(Compound A2) 2-(Cyclopropanecarbonyl-amino)-6-methoxy-benzo[b]thiophene-3-carboxylic acid amide,
(Compound A3) Acetic acid 3-carbamoyl-2-(cyclopropanecarbonyl-amino)-benzo[b]thiophen-6-yl ester,
(Compound A4) 2-[(2-Methyl-cyclopropanecarbonyl)-amino]-benzo[b]thiophene-3-carboxylic acid amide,
(Compound A5) 6-Hydroxy-2-[(2-methyl-cyclopropanecarbonyl)-amino]-benzo[b]thiophene-3-carboxylic acid amide,
(Compound A6) 2-(cyclopropanecarbonyl-amino)-6-methyl-benzo[b]thiophene-3-carboxylic acid amide,
(Compound A7) 2-(Cyclopropanecarbonyl-amino)-6-methylcarbamoylmethoxy-benzo[b]thiophene-3-carboxylic acid amide,
(Compound A8) 2-(Cyclopropanecarbonyl-amino)-6-[(2-hydroxy-ethylcarbamoyl)-methoxy]-benzo[b]thiophene-3-carboxylic acid amide,
(Compound A9) 2-(Cyclopropanecarbonyl-amino)-6-ethoxy-benzo[b]thiophene-3-carboxylic acid amide,
(Compound A10) 7-Chloro-2-(cyclopropanecarbonyl-amino)-6-(2-piperidin-1-yl-ethoxy)-benzo[b]thiophene-3-carboxylic acid amide,
(Compound A11) 6-Hydroxy-2-[3-(4-methoxy-phenyl)-ureido]-benzo[b]thiophene-3-carboxylic acid amide,
(Compound A12) 6-Hydroxy-2-(3-phenyl-ureido)-benzo[b]thiophene-3-carboxylic acid amide,
(Compound A13) N-(3-Carbamoyl-6-hydroxy-benzo[b]thiophen-2-yl)-oxalamide,
(Compound A14) 5-Bromo-2-(cyclopropanecarbonyl-amino)-benzo[b]thiophene-3-carboxylic acid amide,
(Compound A15) 6-Bromo-2-(cyclopropanecarbonyl-amino)-7-hydroxy-benzo[b]thiophene-3-carboxylic acid amide,
(Compound A16) 2-(Cyclopropanecarbonyl-amino)-benzo[b]thiophene-3-carboxylic acid amide,
(Compound A17) 2-(Cyclopropanecarbonyl-amino)-6-hydroxy-benzo[b]thiophene-3,5-dicarboxylic acid 3-amide-5-diethylamid,
(Compound A18) 2-(Cyclopropanecarbonyl-amino)-6-hydroxy-benzo[b]thiophene-3,7-dicarboxylic acid 3-amide-7-diethylamid,
(Compound A19) 6-(3-Amino-propoxy)-2-(cyclopropanecarbonyl-amino)-benzo[b]thiophene-3-carboxylic acid amide,
(Compound A20) 2-(Cyclopropanecarbonyl-amino)-6-(2-hydroxy-ethoxy)-benzo[b]thiophene-3-carboxylic acid amide,
(Compound A21) 2-(Cyclopropanecarbonyl-amino)-6-[2-(tetrahydro-pyran-2-yloxy)-ethoxy]-benzo[b]thiophene-3-carboxylic acid amide,
(Compound A22) (3-Carbamoyl-6-hydroxy-benzo[b]thiophen-2-yl)-carbamic acid benzyl ester,
(Compound A23) 6-Ethoxy-2-[3-(4-methoxy-phenyl)-ureido]-benzo[b]thiophene-3-carboxylic acid amide,
(Compound A24) 7-Chloro-2-[3-(4-fluoro-phenyl)-ureido]-6-hydroxy-benzo[b]thiophene-3-carboxylic acid amide,
(Compound A25) Diethyl-carbamicacid-3-carbamoyl-2-(cyclopropane-carbonyl-amino)-benzo[b]thiophen-6-yl ester,
(Compound A26) 6-Hydroxy-2-[3-(4-trifluoromethyl-phenyl)-ureido]-benzo[b]hiophene-3-carboxylic acid amid,
(Compound A27) 2-[3-(4-Bromo-phenyl)-ureido]-6-methyl-benzo[b]thiophene-3-carboxylic acid amide,
(Compound A28) 3-Carbamoyl-2-(cyclopropanecarbonyl-amino)-benzo[b]thiophen-6-carboxylic acid ethyl ester,
(Compound A29) [3-Carbamoyl-2-(cyclopropanecarbonyl-amino)-benzo[b]thiophen-6-yloxy]-acetic acid methyl ester
(Compound A30) 2-(Cyclopropanecarbonyl-amino)-6-hydroxy-benzo[b]thiophene-3-carboxylic acid amide,
(Compound A31) 2-[3-(4-Fluoro-phenyl)-ureido]-6-hydroxy-benzo[b]thiophene-3-carboxylic acid amid,
(Compound A32) 7-Bromo-2-(cyclopropanecarbonyl-amino)-6-hydroxy-benzo[b]thiophene-3-carboxylic acid amide,
(Compound A33) 6-Hydroxy-2-[3-(4-iodo-phenyl)-ureido]-benzo[b]thiophene-3-carboxylic acid amide,
(Compound A34) 2-(Cyclopropanecarbonyl-amino)-6-(2-piperidin-1-yl-ethoxy)-benzo[b]thiophene-3-carboxylic acid amide,
(Compound A35) 7-Chloro-2-(cyclopropanecarbonyl-amino)-6-hydroxy-benzo[b]thiophene-3-carboxylic acid amide,
(Compound A36) 2-(Cyclopropanecarbonyl-amino)-benzo[b]thiophene-3-carboxylic acid methylamide,
(Compound A37) 7-Chloro-6-hydroxy-2-[(2-methyl-cyclopropanecarbonyl)-amino]-benzo[b]thiophene-3-carboxylic acid amide,
(Compound A38) 5,7-Dibromo-2-(cyclopropanecarbonyl-amino)-6-hydroxy-benzo[b]thiophene-3-carboxylic acid amide,
(Compound A39) 2-[3-(4-Bromo-phenyl)-ureido]-6-hydroxy-benzo[b]thiophene-3-carboxylic acid amide,
(Compound A40) 2-Cyclopropyl-7-methyl-3H-benzo[4,5]thieno[2,3-d]pyrimidin-4-one,
(Compound A41) 2-[3-(4-Ethoxy-phenyl)-ureido]-6-hydroxy-benzo[b]thiophene-3-carboxylic acid amide,
(Compound A42) 2-[3-(3-Chloro4-methyl-phenyl)-ureido]-6-hydroxy-benzo[b]thiophene-3-carboxylic acid amide,
(Compound A43) 5,7-dichloro-2-(cyclopropanecarbonyl-amino)-6-hydroxy-benzo[b]thiophene-3-carboxylic acid amide,
(Compound A44) 5,7-Dichloro-2-(cyclopropanecarbonyl-amino)-6-methoxy-benzo[b]thiophene-3-carboxylic acid amide,
(Compound A45) 5,7-Dichloro-2-(cyclopropanecarbonyl-amino)-6-ethoxy-benzo[b]thiophene-3-carboxylic acid amide,
(Compound A46) 5,7-Dichloro-2-(cyclopropanecarbonyl-amino)-6-isopropoxy-benzo[b]thiophene-3-carboxylic acid amide,
(Compound A47) 2-[3-(3-Chloro-2-methyl-phenyl)-ureido]-6-hydroxy-benzo[b]thiophene-3-carboxylic acid amide,
(Compound A48) 6-Hydroxy-2-[3-(2-methylsulfanyl-phenyl)-ureido]-benzo[b]thiophene-3-carboxylic acid amide,
(Compound A49) 5,7-Dichloro-2-(cyclopropanecarbonyl-amino)-6-(2-hydroxyethoxy)-benzo[b]thiophene-3-carboxylic acid amide,
(Compound A50) 6-(2-Amino-ethoxy)-5,7-dichloro-2(cyclopropanecarbonyl-amino)-benzo[b]thiophene-3-carboxylic acid amide hydrochloride,
(Compound A51) 2-(Cyclopropanecarbonyl-amino)-6-(2-morpholin-4-yl-2-oxo-ethoxy)-benzo[b]thiophene-3-carboxamide,
(Compound A52) 2-(Cyclopropanecarbonyl-amino)-6-[(3-hydroxy-propyl-carbamoyl)-methoxy]-benzo[b]thiophene-3-carboxamide,
(Compound A53) 6-{[Bis-(2-hydroxy-ethyl)-carbamoyl]-methoxy}-2-(cyclopropanecarbonyl-amino)-benzo[b]thiophene-3-carboxamide,
(Compound A54) 2-(Cyclopropanecarbonyl-amino)-6-[2-(4-methyl-piperazin-1-yl)-2-oxo-ethoxy]-benzo[b]thiophene-3-carboxamide,
(Compound A55) 2-(Cyclopropanecarbonyl-amino)-6-[(3,4-dimethoxy-benzylcarbamoyl)-(methoxy]-benzo[b]thiophene-3-carboxamide,
(Compound A56) 2-(Cyclopropanecarbonyl-amino)-6-[(1-phenyl-ethylcarbamoyl)-methoxy]-benzo[b]thiophene-3-carboxamide,
(Compound A57) 6-Hydroxy-2-(3-pyridin-3-yl-ureido)-benzo[b]thiophene-3-carboxylic acid amide,
(Compound A58) 6-Hydroxy-2-[3-(6-methoxy-pyridin-3-yl)-ureido]-benzo[b]thiophene-3-carboxylic acid amide,
(Compound A59) 6-Hydroxy-2-(3-pyridin-4-yl-ureido)-benzo[b]thiophene-3-carboxylic acid amide,
(Compound A60) 2-[3-(4-Cyano-3-isopropyl-isoxazol-5-yl)-ureido]-6-hydroxy-benzo[b]thiophene-3-carboxylic acid amide,
(Compound A61) 2-[3-(4-Cyano-3-cyclopropyl-isoxazol-5-yl)-ureido]-6-hydroxy-benzo[b]thiophene-3-carboxylic acid amide,
(Compound A62) 2-[3-(2,3-Dichloro-phenyl)-ureido]-6-hydroxy-benzo[b]thiophene-3-carboxylic acid amide,
(Compound A63) 6-Hydroxy-2-[3-(2-trifluoromethoxy-phenyl)-ureido]-benzo[b]thiophene-3-carboxylic acid amide,
(Compound A64) 6-Hydroxy-2-[3-(4-nitro-phenyl)-ureido]-benzo[b]thiophene-3-carboxylic acid amide,
(Compound A65) 6-Hydroxy-2-[3-(3,4,5-trimethoxy-phenyl)-ureido]-benzo[b]thiophene-3-carboxylic acid amide,
(Compound A66) 6-Hydroxy-2-(3-p-tolyl-ureido)-benzo[b]thiophene-3-carboxylic acid amide,
(Compound A67) 6-Hydroxy-2-[3-(2-thiophen-2-yl-ethyl)-ureido]-benzo[b]thiophene-3-carboxylic acid amide,
(Compound A68) 6-Hydroxy-2-[3-(4-methyl-cyclohexyl)-ureido]-benzo[b]thiophene-3-carboxylic acid amide,
(Compound A69) 2-(3-Cyclopentyl-ureido)-6-hydroxy-benzo[b]thiophene-3-carboxylic acid amide,
(Compound A70) 2-[3-(2-Cyclohex-1-enyl-ethyl)-ureido]-6-hydroxy-benzo[b]thiophene-3-carboxylic acid amide,
(Compound A71) 6-Hydroxy-2-{3-[2-(4-methoxy-phenyl)-ethyl]-ureido}-benzo[b]thiophene-3-carboxylic acid amide,
(Compound A72) 6-Hydroxy-2-[3-(2-hydroxy-ethyl)-ureido]-benzo[b]thiophene-3-carboxylic acid amide,
(Compound A73) 6-Hydroxy-2-[3-(3-hydroxy-propyl)-ureido]-benzo[b]thiophene-3-carboxylic acid amide,
(Compound A74) 6-Hydroxy-2-(3-piperidin-1-yl-ureido)-benzo[b]thiophene-3-carboxylic acid amide,
(Compound A75) 2-(3-Bicyclo[2.2.1]hept-2-yl-ureido)-6-hydroxy-benzo[b]thiophene-3-carboxylic acid amide,
(Compound A76) 2-Amino-6-ethoxy-benzo[b]thiophene-3-carboxylic acid amide,
(Compound A77) 6-Ethoxy-2-[3-(4-fluoro-phenyl)-ureido]-benzo[b]thiophene-3-carboxylic acid amide,
(Compound A78) 2-[3-(4-Bromo-phenyl)-ureido]-6-ethoxy-benzo[b]thiophene-3-carboxylic acid amide,
(Compound A79) 6-Ethoxy-2-(3-p-tolyl-ureido)-benzo[b]thiophene-3-carboxylic acid amide,
(Compound A80) 2-[3-(2,4-Difluoro-phenyl)-ureido]-6-ethoxy-benzo[b]thiophene-3-carboxylic acid amide,
(Compound A81) 6-Ethoxy-2-[3-(3,4,5-trimethoxy-phenyl)-ureido]-benzo[b]thiophene-3-carboxylic acid amide,
(Compound A82) 2-[3-(2-Difluoromethoxy-phenyl)-ureido]-6-hydroxy-benzo[b]thiophene-3-carboxylic acid amide,
(Compound A83) 6-Hydroxy-2-[3-(4-trifluoromethylsulfanyl-phenyl)-ureido]-benzo[b]thiophene-3-carboxylic acid amide,
(Compound A84) 7-Chloro-2-(4-fluoro-3-chlorobenzoyl-amino)-6-hydroxy-benzo[b]thiophene-3-carboxylic acid amide,
(Compound A85) 7-Chloro-2-(3,5-dichlorobenzoyl-amino)-6-hydroxy-benzo[b]thiophene-3-carboxylic acid amide,
(Compound A86) 7-Chloro-6-hydroxy-2-[3-(2-methylsulfanyl-phenyl)-ureido]-benzo[b]thiophene-3-carboxylic acid amide,
(Compound A87) 7-Chloro-2-(4-cyanobenzoyl-amino)-6-hydroxy-benzo[b]thiophene-3-carboxylic acid amide,
(Compound A88) 7-Chloro-2-(2,3-dichlorobenzoyl-amino)-6-hydroxy-benzo[b]thiophene-3-carboxylic acid amide,
(Compound A89) 7-Chloro-2-(4-thiomethylbenzoyl-amino)-6-hydroxy-benzo[b]thiophene-3-carboxylic acid amide,
(Compound A90) 7-Chloro-2-(3,4-dimethylbenzoyl-amino)-6-hydroxy-benzo[b]thiophene-3-carboxylic acid amide,
(Compound A91) 7-Chloro-2-(4-isopropylbenzoyl-amino)-6-hydroxy-benzo[b]thiophene-3-carboxylic acid amide,
(Compound A92) 7-Chloro-2-(3,5-bis trifluoromethylbenzoyl-amino)-6-hydroxy-benzo[b]thiophene-3-carboxylic acid amide,
(Compound A93) 7-Chloro-2-(4-chlorobenzoyl-amino)-6-hydroxy-benzo[b]thiophene-3-carboxylic acid amide,
(Compound A94) 7-Chloro-2-(4-trifluoromethylbenzoyl-amino)-6-hydroxy-benzo[b]thiophene-3-carboxylic acid amide,
(Compound A95) 7-Chloro-2-(2,4-difluorobenzoyl-amino)-6-hydroxy-benzo[b]thiophene-3-carboxylic acid amide,
(Compound A96) 7-Chloro-2-(3-fluorobenzoyl-amino)-6-hydroxy-benzo[b]thiophene-3-carboxylic acid amide,
(Compound A97) 7-Chloro-2-(3,4,5-trimethoxybenzoyl-amino)-6-hydroxy-benzo[b]thiophene-3-carboxylic acid amide,
(Compound A98) 2-(3-benzo[1,3]dioxol-5yl-ureido)-6-hydroxy-benzo[b]thiophene-3-carboxylic acid amide,
(Compound A99) N-(3-carbamoyl-6-hydroxy-benzo[b]thiophen-2-yl)-oxalamic acid,
(Compound A100) 6-Ethoxy-2-[3-(4-trifluoromethyl-phenyl)-ureido]-benzo[b]thiophene-3-carboxylic acid amide,
(Compound A101) 6-Ethoxy-2-[3-(4-nitro-phenyl)-ureido]-benzo[b]thiophene-3-carboxylic acid amide,
(Compound A102) 6-Ethoxy-2-(3-phenyl-ureido)-benzo[b]thiophene-3-carboxylic acid amide,
(Compound A103) 6-Ethoxy-2-[3-(4-ethoxy-phenyl)-ureido]-benzo[b]thiophene-3-carboxylic acid amide,
(Compound A104) 6-Ethoxy-2-[3-(4-methylsulfanyl-phenyl)-ureido]-benzo[b]thiophene-3-carboxylic acid amide,
(Compound A105) 2-(Cyclopropanecarbonyl-amino)-5-[3-(4-methylsulfanyl-phenyl)-ureido]-benzo[b]thiophene-3-carboxylic acid amide,
(Compound A106) 5-[3-(4-Bromo-phenyl )-ureido]-2-(cyclopropanecarbonyl-amino)-benzo[b]thiophene-3-carboxylic acid amide,
(Compound A107) 2-(Cyclopropanecarbonyl-amino)-5-[3-(4-iodo-phenyl)-ureido]-benzo[b]thiophene-3-carboxylic acid amide,
(Compound A108) 2-(Cyclopropanecarbonyl-amino)-5-(3-phenyl-ureido)-benzo[b]thiophene-3-carboxylic acid amide,
(Compound A109) 7-Bromo-2-[3-(4-bromo-phenyl)-ureido]-6-hydroxy-benzo[b]thiophene-3-carboxylic acid amide,
(Compound A110) 7-Bromo-6-hydroxy-2-[3-(4-trifluoromethoxy-phenyl)-ureido]-benzo[b]thiophene-3-carboxylic acid amide,
(Compound A111) 7-Bromo-6-hydroxy-2-[3-(4-methylsulfanyl-phenyl)-ureido]-benzo[b]thiophene-3-carboxylic acid amide,
(Compound A112) 7-Chloro-2-[3-(4-difluoromethoxy-phenyl)-ureido]-6-hydroxy-benzo[b]thiophene-3-carboxylic acid amide,
(Compound A113) 2-(Cyclopropanecarbonyl-amino)-5-nitro-benzo[b]thiophene-3-carboxylic acid amide,
(Compound A114) 5-Amino-2-(cyclopropanecarbonyl-amino)-benzo[b]thiophene-3-carboxylic acid amide,
(Compound A115) 6-Hydroxy-2-[3-(4-trifluoromethoxy-phenyl)-ureido]-benzo[b]thiophene-3-carboxylic acid amide,
(Compound A116) 6-Hydroxy-2-[3-(4-methylsulfanyl-phenyl)-ureido]-benzo[b]thiophene-3-carboxylic acid amide,
(Compound A117) 2-(3,3-Dimethyl-ureido)-6-hydroxy-benzo[b]thiophene-3-carboxylic acid amide,
(Compound A118) 2-(3-Cyclopropyl-ureido)-6-hydroxy-benzo[b]thiophene-3-carboxylic acid amide,
(Compound A119) 6-Hydroxy-2-[3-(3-methylsulfanyl-phenyl)-ureido]-benzo[b]thiophene-3-carboxylic acid amide,
(Compound A120) (3-Carbamoyl-5,7-dichloro-6-hydroxy-benzo[b]thiophen-2-yl)-carbamic acid ethyl ester,
(Compound A121) 2-[3-(3-Bromo-phenyl)-ureido]-6-hydroxy-benzo[b]thiophene-3-carboxylic acid amide,
(Compound A122) 6-Hydroxy-2-[3-(3-trifluoromethyl-phenyl)-ureido]-benzo[b]thiophene-3-carboxylic acid amide,
(Compound B1) 2-(Cyclopropanecarbonyl-amino)4,7-dihydro-5H-thieno[2,3-c]pyran-3-carboxylic acid amide,
(Compound B2) 2-Isobutyrylamino-4,7-dihydro-5H-thieno[2,3-c]pyran-3-carboxylic acid amide,
(Compound B3) 2-[(2-Phenyl-cyclopropanecarbonyl)-amino]-4,7-dihydro-5H-thieno[2,3-c]pyran-3-carboxylic acid amide,
(Compound B4) 2-[(2-Methyl-cyclopropanecarbonyl)-amino]-4,7-dihydro-5H-thieno[2,3-c]pyran-3-carboxylic acid amide,
(Compound B5) 2-[(Furan-2-carbonyl)-amino]-4,7-dihydro-5H-thieno[2,3-c]pyran-3-carboxylic acid amide,
(Compound B6) 2-[(Adamantane-1-carbonyl)-amino]-4,7-dihydro-5H-thieno[2,3-c]pyran-3-carboxylic acid amide,
(Compound B7) 2-(Cyclohexanecarbonyl-amino)4,7-dihydro-5H-thieno[2,3-c]pyran-3-carboxylic acid amide,
(Compound B8) 2-(3-Cyclohexyl-ureido)-4,7-dihydro-5H-thieno[2,3-c]pyran-3-carboxylic acid amide,
(Compound B9) 2-(3-Phenyl-ureido)-4,7-dihydro-5H-thieno[2,3-c]pyran-3-carboxylic acid amide,
(Compound B10) 2-(Cyclopentanecarbonyl-amino)-4,7-dihydro-5H-thieno[2,3-c]pyran-3-carboxylic acid amide,
(Compound B11) 2-[3-(4-Acetyl-phenyl)-ureido]-4,7-dihydro-5H-thieno[2,3-c]pyran-3-carboxylic acid amide,
(Compound B12) 2-[3-(4-Methyl-phenyl)-ureido]-4,7-dihydro-5H-thieno[2,3-c]pyran-3-carboxylic acid amide,
(Compound B13) 2-[3-(4-Fluoro-phenyl)-ureido]-4,7-dihydro-5H-thieno[2,3-c]pyran-3-carboxylic acid amide,
(Compound B14) 2-(2-Methyl-butyrylamino)-4 ,7-dihydro-5H-thieno[2,3-c]pyran-3-carboxylic acid amide,
(Compound B15) 2-(Cyclobutanecarbonyl-amino)-4,7-dihydro-5H-thieno[2,3-c]pyran-3-carboxylic acid amide,
(Compound B16) 2-But-2-enoylamino-4,7-dihydro-5H-thieno[2,3-c]pyran-3-carboxylic acid amide,
(Compound B17) 2-(3-Methyl-but-2-enoylamino)-4,7-dihydro-5H-thieno[2,3-c]pyran-3-carboxylic acid amide,
(Compound B18) 2-(2,2-Dimethyl-propionylamino)-4,7-dihydro-5H-thieno[2,3-c]pyran-3-carboxylic acid amide,
(Compound B19) 2-(3,4-Difluoro-benzoylamino)-4,7-dihydro-5H-thieno[2,3-c]pyran-3-carboxylic acid amide,
(Compound B20) 5,5-Dimethyl-2-(3-phenyl-ureido)4,7-dihydro-5H-thieno[2,3-c]pyran-3-carboxylic acid amide,
(Compound B21) 2-[3-(5-Phenyl-2-p-tolyl-2H-pyrazol-3-yl)-ureido]-4,7-dihydro-5H-thieno[2,3-c]pyran-3-carboxylic acid amide,
(Compound B22) 2-[3-(5-tert-Butyl-2-p-tolyl-2H-pyrazol-3-yl)-ureido]-4,7-dihydro-5H-thieno[2,3-c]pyran-3-carboxylic acid amide,
(Compound B23) 2-[2-(2-Bromo-phenylsulfanyl)-acetylamino]-4,7-dihydro-5H-thieno[2,3-c]pyran-3-carboxylic acid amide,
(Compound B24) 2-(Cyclopropanecarbonyl-amino)-7-dimethylamino-4,7-dihydro-5H-thieno[2,3-c]pyran-3-carboxylic acid amide,
(Compound B25) 2-(Cyclopropanecarbonyl-amino)-7-[4-(4-methyl-piperazin-1-yl)-phenylamino]-4,7-dihydro-5H-thieno[2,3-c]pyran-3-carboxylic acid amide,
(Compound B26) 2-(Cyclopropanecarbonyl-amino)-7-(4-methyl-piperazin-1-yl)-4,7-dihydro-5H-thieno[2,3-c]pyran-3-carboxylic acid amide,
(Compound B27) 2-(Cyclopropanecarbonyl-amino)-7-(2-dimethylamino-ethylamino)-4,7-dihydro-5H-thieno[2,3-c]pyran-3-carboxylic acid amide,
(Compound B28) 2-(3-Biphenyl-2-yl-ureido)-4,7-dihydro-5H-thieno[2,3-c]pyran-3-carboxylic acid amide,
(Compound B29) 2-[3-(2-Ethoxy-phenyl)-ureido]-4,7-dihydro-5H-thieno[2,3-c]pyran-3-carboxylic acid amide,
(Compound B30) 2-(3,3-Diethyl-ureido)-4,7-dihydro-5H-thieno[2,3-c]pyran-3-carboxylic acid amide,
(Compound B31) 4-(3-Carbamoyl4,7-dihydro-5H-thieno[2,3-c]pyran-2-ylcarbamoyl)-piperazine-1-carboxylic acid ethyl ester,
(Compound B32) 4-Phenyl-piperazine-1-carboxylic acid(3-carbamoyl4,7-dihydro-5H-thieno[2,3-c]pyran-2-yl)-amide,
(Compound B33) 2-{3-[3-(4-Chloro-phenyl)4-cyano-isoxazol-5-yl]-ureido}-4,7-dihydro-5H-thieno[2,3-c]pyran-3-carboxylic acid amide,
(Compound B34) 2-[3-(3-Imidazol-1-yl-propyl)-ureido]-4,7-dihydro-5H-thieno[2,3-c]pyran-3-carboxylic acid amide,
(Compound B35) 2-(3,3-Diallyl-ureido]-4,7-dihydro-5H-thieno[2,3-c]pyran-3-carboxylic acid amide,
(Compound B36) (3-Carbamoyl-4,7-dihydro-5H-thieno[2,3-c]pyran-2-yl)-carbamic acid 2-methoxy-phenyl ester,
(Compound B37) 2-[3-(4-Bromo-phenyl)-thioureido]-4,7-dihydro-5H-thieno[2,3-c]pyran-3-carboxylic acid amide,
(Compound B38) 2-[3-(4-Methylsulfanyl-phenyl)-thioureido]-4,7-dihydro-5H-thieno[2,3-c]pyran-3-carboxylic acid amide,
(Compound B39) 2-[3-(Hydrazinocarbonyl-phenyl-methyl)-ureido]-4,7-dihydro-5H-thieno[2,3-c]pyran-3-carboxylic acid amide,
(Compound B40) 2-[(2-Benzyloxymethyl-cyclopropanecarbonyl)-amino]-4,7-dihydro-5H-thieno[2,3-c]pyran-3-carboxylic acid amide,
(Compound B41) N-(3-Carbamoyl4,7-dihydro-5H-thieno[2,3-c]pyran-2-yl)-oxalamide,
(Compound B42) 2-{[3-(2,2-Dichloro-vinyl)-2,2-dimethyl-cyclopropanecarbonyl]-amino}-4,7-dihydro-5H-thieno[2,3-c]pyran-3-carboxylic acid amide,
(Compound B43) 2-[3-(4-Bromo-phenyl)-ureido]-4,7-dihydro-5H-thieno[2,3-c]pyran-3-carboxylic acid amide,
(Compound B44) 4-Hydroxy-piperidine-1-carboxylic-acid (3-carbamoyl-4,7-dihydro-5H-thieno[2,3-c]pyran-2-yl)-amide
(Compound B45) 2-[3-(5-Methyl-furan-2-ylmethyl)-ureido]-4,7-dihydro-5H-thieno[2,3-c]pyran-3-carboxylic acid amide,
(Compound B46) 2-[3,3-Bis-(2-hydroxy-ethyl)-ureido]-4,7-dihydro-5H-thieno[2,3-c]pyran-3-carboxylic acid amide,
(Compound B47) 2-[3-(4-Methyl-cyclohexyl)-ureido]-4,7-dihydro-5H-thieno[2,3-c]pyran-3-carboxylic acid amide,
(Compound B48) 2-(3-Cyclopentyl-ureido)-4,7-dihydro-5H-thieno[2,3-c]pyran-3-carboxylic acid amide,
(Compound B49) 2-(Cyclopropanecarbonyl-amino)-7-morpholyn-4-yl-4,7-dihydro-5H-thieno[2,3-c]pyran-3-carboxylic acid amide,
(Compound B50) 2-{3-[2-(4-Methoxy-phenyl)-ethyl]-ureido}-4,7-dihydro-5H-thieno[2,3-c]pyran-3-carboxylic acid amide,
(Compound B51) 2-[3-(2-tert-Butyl-5-phenyl-2H-pyrazol-3-yl)-ureido]-4,7-dihydro-5H-thieno[2,3-c]pyran-3-carboxylic acid amide,
(Compound B52) Morpholine-4-carboxylic-acid(3-carbamoyl-4,7-dihydro-5H-thieno[2,3-c]pyran-2-yl)-amide,
(Compound B53) 4-Methyl-piperazine-1-carboxylic-acid (3-carbamoyl-4,7-dihydro-5H-thieno[2,3-c]pyran-2-yl)-amide,
(Compound B54) Piperidine-1-carboxylic-acid (3-carbamoyl-4,7-dihydro-5H-thieno[2,3-c]pyran-2-yl)-amide,
(Compound B55) 2-[3-(2-Methyl-cyclohexyl)-ureido]-4,7-dihydro-5H-thieno[2,3-c]pyran-3-carboxylic acid amide,
(Compound B56) 2-[3-Bicyclo[2.2.1]hept-2-yl)-ureido]-4,7-dihydro-5H-thieno[2,3-c]pyran-3-carboxylic acid amide,
(Compound B57) 2-(3,3-Diisopropyl-ureido)-4,7-dihydro-5H-thieno[2,3-c]pyran-3-carboxylic acid amide,
(Compound B58) Thiomorpholine-4-carboxylic-acid(3-carbamoyl-4,7-dihydro-5H-thieno[2,3-c]pyran-2-yl)-amide,
(Compound B59) 2-[3-(5-Fluoro-2-methyl-phenyl)-ureido]-4,7-dihydro-5H-thieno[2,3-c]pyran-3-carboxylic acid amide,
(Compound B60) 2-[3-(2-Hydroxy-butyl)-ureido]-4,7-dihydro-5H-thieno[2,3-c]pyran-3-carboxylic acid amide,
(Compound B61) 2-(Cyclopropanecarbonyl-amino)-7-(3-dimethylamino-propylamino)-4,7-dihydro-5H-thieno[2,3-c]pyran-3-carboxylic acid amide,
(Compound B62) 2-[3-(2-Cyclohex-1-enyl-ethyl)-ureido]-4,7-dihydro-5H-thieno[2,3-pyran-3-carboxylic acid amide,
(Compound B63) 2-[3-(2,6,6-Trimethyl-bicyclo[3.1.1]hept-3-yl)-ureido]-4,7-dihydro-5H-thieno[2,3-c]pyran-3-carboxylic acid amide,
(Compound B64) 2-(2-Ethylamino-acetylamino)-4,7-dihydro-5H-thieno[2,3-c]pyran-3-carboxylic acid amide,
(Compound B65) 2-(Cyclopropanecarbonyl-amino)-7-(2-pyridin-2-yl-ethylamino)-4,7-dihydro-5H-thieno[2,3-c]pyran-3-carboxylic acid amide,
(Compound B66) 2-(Cyclopropanecarbonyl-amino)-7-(3-morpholin-4-yl-propylamino)-4,7-dihydro-5H-thieno[2,3-c]pyran-3-carboxylic acid amide,
(Compound B67) 2-[3-(4-Methylsulfanyl-phenyl)-ureido]-4,7-dihydro-5H-thieno[2,3-c]pyran-3-carboxylic acid amide,
(Compound B68) 2-[3-(3-Diethylamino-propyl)-ureido]-4,7-dihydro-5H-thieno[2,3-c]pyran-3-carboxylic acid amide,
(Compound B69) 2-[3-(2-Diethylamino-ethyl)-ureido]-4,7-dihydro-5H-thieno[2,3-c]pyran-3-carboxylic acid amide,
(Compound B70) 2-[3-(2-Dimethylamino-ethyl)-ureido]-4,7-dihydro-5H-thieno[2,3-c]pyran-3-carboxylic acid amide,
(Compound B71) 2-(Cyclopropanecarbonyl-amino)-7,7-dimethyl-4,7-dihydro-5H-thieno[2,3-c]pyran-3-carboxylic acid amide,
(Compound B72) 2-[3-(1-Benzyl-pyridin-4-yl)-ureido]-4,7-dihydro-5H-thieno[2,3-c]pyran-3-carboxylic acid amide,
(Compound B73) 2-(3-Pyridin-4-yl-ureido]-4,7-dihydro-5H-thieno[2,3-c]pyran-3-carboxylic acid amide,
(Compound B74) 2-[3-(4-Chloro-3-trifluoromethyl-phenyl)-ureido]-4,7-dihydro-5H-thieno[2,3-c]pyran-3-carboxylic acid amide,
(Compound B75) 2-[3-(2-Thiophen-2-yl-ethyl)-ureido]-4,7-dihydro-5H-thieno[2,3-c]pyran-3-carboxylic acid amide,
(Compound B76) 2-(3-Thiophen-2-yl-ureido)-4,7-dihydro-5H-thieno[2,3-c]pyran-3-carboxylic acid amide,
(Compound B77) 2-(2-Cyclopropyl-acetylamino)-4,7-dihydro-5H-thieno[2,3-c]pyran-3-carboxylic acid amide,
(Compound B78) 2-(3-Pyridin-3-yl-ureido)-4,7-dihydro-5H-thieno[2,3-c]pyran-3-carboxylic acid amide,
(Compound B79) 2-(3-Benzo[1,3]dioxol-5-yl-ureido)-4,7-dihydro-5H-thieno[2,3-c]pyran-3-carboxylic acid amide,
(Compound B80) 2-(3-Furan-2-ylmethyl-ureido)-4,7-dihydro-5H-thieno[2,3-c]pyran-3-carboxylic acid amide,
(Compound B81) 2-[3-(2,5-Di-tert-butyl-2H-pyrazol-3-yl)-ureido]-4,7-dihydro-5H-thieno[2,3-c]pyran-3-carboxylic acid amide,
(Compound B82) 2-[3-(4-Benzyloxy-phenyl)-ureido]-4,7-dihydro-5H-thieno[2,3-c]pyran-3-carboxylic acid amide,
(Compound B83) 2-(2-Methyl-acryloylamino)-4,7-dihydro-5H-thieno[2,3-c]pyran-3-carboxylic acid amide,
(Compound B84) 2-(3-Isopropyl-ureido)-4,7-dihydro-5H-thieno[2,3-c]pyran-3-carboxylic acid amide,
(Compound B85) 2-[3-(2,6-Diisopropyl-phenyl)-ureido]-4,7-dihydro-5H-thieno[2,3-c]pyran-3-carboxylic acid amide,
(Compound B86) 2-[3-(3-Chloro-phenyl)-ureido]-4,7-dihydro-5H-thieno[2,3-c]pyran-3-carboxylic acid amide,
(Compound B87) 2-(3-Benzyl-ureido)-4,7-dihydro-5H-thieno[2,3-c]pyran-3-carboxylic acid amide,
(Compound B88) 2-[3-(4-Isopropyl-phenyl)-ureido]-4,7-dihydro-5H-thieno[2,3-c]pyran-3-carboxylic acid amide,
(Compound B89) 2-[3-(3-Acetyl-phenyl)-ureido]-4,7-dihydro-5H-thieno[2,3-c]pyran-3-carboxylic acid amide,
(Compound B90) 2-[3-(3-Ethyl-phenyl)-ureido]-4,7-dihydro-5H-thieno[2,3-c]pyran-3-carboxylic acid amide,
(Compound B91) 2-[3-(1-Naphthalen-1-yl-ethyl)-ureido]-4,7-dihydro-5H-thieno[2,3-c]pyran-3-carboxylic acid amide,
(Compound B92) 2-(2-Azepan-1-yl-acetylamino)-4,7-dihydro-5H-thieno[2,3-c]pyran-3-carboxylic acid amide,
(Compound B93) 2-[3-(3,4-Dimethyl-phenyl)-ureido]-4,7-dihydro-5H-thieno[2,3-c]pyran-3-carboxylic acid amide,
(Compound B94) 2-(2-Ethyl-butyrylamino)-4,7-dihydro-5H-thieno[2,3-c]pyran-3-carboxylic acid amide,
(Compound B95) 2-[(1-Methyl-cyclopropanecarbonyl)-amino]-4,7-dihydro-5H-thieno[2,3-c]pyran-3-carboxylic acid amide,
(Compound B96) 2-[(3-Methyl-cyclohexanecarbonyl)-amino]-4,7-dihydro-5H-thieno[2,3-c]pyran-3-carboxylic acid amide,
(Compound B97) 2-[3-(5-Chloro-2-methoxy-phenyl)-ureido]-4,7-dihydro-5H-thieno[2,3-c]pyran-3-carboxylic acid amide,
(Compound B98) 2-(3-Methyl-butyrylamino)-4,7-dihydro-5H-thieno[2,3-c]pyran-3-carboxylic acid amide,
(Compound B99) 2-(Cyclopropanecarbonyl)-amino)-7-isopropoxy4,7-dihydro-5H-thieno[2,3-c]pyran-3-carboxylic acid amide,
(Compound B100) 2-[3-(3-Chloro-2-methyl-phenyl)-ureido]-4,7-dihydro-5H-thieno[2,3-c]pyran-3-carboxylic acid amide,
(Compound B101) 2-[3-(2-Chloro-3-methyl-phenyl)-ureido]-4,7-dihydro-5H-thieno[2,3-c]pyran-3-carboxylic acid amide,
(Compound B102) 2-(4-Bromo-benzoylamino)-4,7-dihydro-5H-thieno[2,3-c]pyran-3-carboxylic acid amide,
(Compound B103) 2-[3-(1-Methyl-cyclopropyl)-thioureido]-4,7-dihydro-5H-thieno[2,3-c]pyran-3-carboxylic acid amide,
(Compound B104) N-(3-Carbamoyl4,7-dihydro-5H-thieno[2,3-c]pyran-2-yl)-succinamic acid,
(Compound B105) 3-(3-Carbamoyl4,7-dihydro-5H-thieno[2,3-c]pyran-2-ylcarbamoyl)-acrylic acid,
(Compound B106) 2-[3-(3-Carbamoyl-4,7-dihydro-5H-thieno[2,3-c]pyran-2-yl)-ureido]-3-methyl-butyric acid methyl ester,
(Compound B107) 2-[(Tetrahydro-furan-3-carbonyl)-amino]-4,7-dihydro-5H-thieno[2,3-c]pyran-3-carboxylic acid amide,
(Compound B108) 2-(2-Bicyclo[2.2.1 ]hept-2-yl-acetylamino)-4,7-dihydro-5H-thieno[2,3-c]pyran-3-carboxylic acid amide,
(Compound B109) 2-(2-Cyclopentyl-acetylamino)-4,7-dihydro-5H-thieno[2,3-c]pyran-3-carboxylic acid amide,
(Compound B110) 2-[2-(3-Acetyl-2,2-dimethyl-cyclobutyl)-acetylamino]-4,7-dihydro-5H-thieno[2,3-c]pyran-3-carboxylic acid amide,
(Compound B111) 2-(2-Cyclopropylamino-acetylamino)4,7-dihydro-5H-thieno[2,3-c]pyran-3-carboxylic acid amide,
(Compound B112) 2-[(2,2,3,3-Tetramethyl-cyclopropanecarbonyl)-amino]-4,7-dihydro-5H-thieno[2,3-c]pyran-3-carboxylic acid amide,
(Compound B113) N-(3-Carbamoyl-4,7-dihydro-5H-thieno[2,3-c]pyran-2-yl)-oxalamic acid methyl ester,
(Compound B114) 2-[3-(3-Carbamoyl-4,7-dihydro-5H-thieno[2,3-c]pyran-2-yl)-ureido]-3-methyl-butyric acid allyl ester,
(Compound B115) 2-[2-(1,3-Dimethyl-butylamino)-acetylamino]-4,7-dihydro-5H-thieno[2,3-c]pyran-3-carboxylic acid amide,
(Compound B116) 2-[3-(3-Trifluoromethyl-phenyl)-ureido]-4,7-dihydro-5H-thieno[2,3-c]pyran-3-carboxylic acid amide,
(Compound B117) 2-[3-(2,3-Dichloro-phenyl)-ureido]-4,7-dihydro-5H-thieno[2,3-c]pyran-3-carboxylic acid amide,
(Compound B118) 2-[3-(2-Methoxy-benzyl)-ureido]-4,7-dihydro-5H-thieno[2,3-c]pyran-3-carboxylic acid amide,
(Compound B119) 2-[3-(4-Nitro-phenyl)-ureido]-4,7-dihydro-5H-thieno[2,3-c]pyran-3-carboxylic acid amide,
(Compound B120) 2-[3-(2,4-Difluoro-phenyl)-ureido]-4,7-dihydro-5H-thieno[2,3-c]pyran-3-carboxylic acid amide,
(Compound B121) 2-[3-(3,4,5-Trimethoxy-phenyl)-ureido]-4,7-dihydro-5H-thieno[2,3-c]pyran-3-carboxylic acid amide,
(Compound B122) 2-[3-(3,5-Dichloro-phenyl)-ureido]-4,7-dihydro-5H-thieno[2,3-c]pyran-3-carboxylic acid amide,
(Compound B123) 2-[3-(2-Nitro-phenyl)-ureido]-4,7-dihydro-5H-thieno[2,3-c]pyran-3-carboxylic acid amide,
(Compound B124) 2-[3-(3-Methoxy-phenyl)-ureido]-4,7-dihydro-5H-thieno[2,3-c]pyran-3-carboxylic acid amide,
(Compound B125) 2-[3-(2-Methyl-5-nitro-phenyl)-ureido]-4,7-dihydro-5H-thieno[2,3-c]pyran-3-carboxylic acid amide,
(Compound B126) 2-(3-Adamantan-1-yl-ureido)-4,7-dihydro-5H-thieno[2,3-c]pyran-3-carboxylic acid amide,
(Compound B127) 2-[3-(3-Nitro-phenyl)-ureido]-4,7-dihydro-5H-thieno[2,3-c]pyran-3-carboxylic acid amide,
(Compound B128) 2-[3-(2-Methyl-3-nitro-phenyl)-ureido]-4,7-dihydro-5H-thieno[2,3-c]pyran-3-carboxylic acid amide,
(Compound B129) 2-[3-(3-Bromo-phenyl)-ureido]-4,7-dihydro-5H-thieno[2,3-c]pyran-3-carboxylic acid amide,
(Compound B130) 2-(3-m-Tolyl-ureido)-4,7-dihydro-5H-thieno[2,3-c]pyran-3-carboxylic acid amide,
(Compound B131) 2-[3-(3-Methyl-butyl)-ureido]-4,7-dihydro-5H-thieno[2,3-c]pyran-3-carboxylic acid amide,
(Compound B132) 2-[3-(4-Methyl-3-nitro-phenyl)-ureido]-4,7-dihydro-5H-thieno[2,3-c]pyran-3-carboxylic acid amide,
(Compound B133) 2-[3-(4-Methoxy-phenyl)-ureido]-4,7-dihydro-5H-thieno[2,3-c]pyran-3-carboxylic acid amide,
(Compound B134) 2-[3-(3-Chloro-4-fluoro-phenyl)-ureido]-4,7-dihydro-5H-thieno[2,3-c]pyran-3-carboxylic acid amide,
(Compound B135) 2-(3-Phenyl-acryloylamino)4,7-dihydro-5H-thieno[2,3-c]pyran-3-carboxylic acid amide,
(Compound B136) 2-[3-(3-Amino-phenylsulfanyl)-acetylamino]-4,7-dihydro-5H-thieno[2,3-c]pyran-3-carboxylic acid amide,
(Compound B137) 2-(2-Methyl-but-2-enoylamino)4,7-dihydro-5H-thieno[2,3-c]pyran-3-carboxylic acid amide,
(Compound B138) 2-(4,4,4-Trifluoro-3-methyl-but-2-enoylamino)-4,7-dihydro-5H-thieno[2,3-c]pyran-3-carboxylic acid amide,
(Compound B139) 2-(3-Isopropyl-thioureido)4,7-dihydro-5H-thieno[2,3-c]pyran-3-carboxylic acid amide,
(Compound B140) 2-(3,3-Dimethyl-butyrylamino)-4,7-dihydro-5H-thieno[2,3-c]pyran-3-carboxylic acid amide,
(Compound B141) 2-[3-(3-Morpholyn-4-yl-propyl)-ureido]-4,7-dihydro-5H-thieno[2,3-c]pyran-3-carboxylic acid amide,
(Compound B142) 2-[3-(3,4-Dichloro-phenyl)-ureido]-4,7-dihydro-5H-thieno[2,3-c]pyran-3-carboxylic acid amide,
(Compound B143) 2-[3-(4-Phenoxy-phenyl)-ureido]-4,7-dihydro-5H-thieno[2,3-c]pyran-3-carboxylic acid amide,
(Compound B144) 2-[3-(4-Trifluoromethyl-phenyl)-ureido]-4,7-dihydro-5H-thieno[2,3-c]pyran-3-carboxylic acid amide,
(Compound B145) 2-[3-(2-Methyl-benzyl)-ureido]-4,7-dihydro-5H-thieno[2,3-c]pyran-3-carboxylic acid amide,
(Compound B146) 2-(3-Biphenyl-4-yl-ureido)-4,7-dihydro-5H-thieno[2,3-c]pyran-3-carboxylic acid amide,
(Compound B147) 2-[3-(4-Methoxy-benzyl)-ureido]-4,7-dihydro-5H-thieno[2,3-c]pyran-3-carboxylic acid amide,
(Compound B148) 2-[3-(4-Methyl-benzyl)-ureido]-4,7-dihydro-5H-thieno[2,3-c]pyran-3-carboxylic acid amide,
(Compound B149) 2-[3-(3-Methyl-benzyl)-ureido]-4,7-dihydro-5H-thieno[2,3-c]pyran-3-carboxylic acid amide,
(Compound B150) 2-(3-o-Tolyl-ureido)4,7-dihydro-5H-thieno[2,3-c]pyran-3-carboxylic acid amide,
(Compound B151) 5-Methyl-isoxazole-4-carboxylic acid (3-carbamoyl-4,7-dihydro-5H-thieno[2,3-c]pyran-2-yl)amide,
(Compound B152) 2-[2-(2-Thiophen-2-yl-ethylamino)-acetylamino]-4,7-dihydro-5H-thieno[2,3-c]pyran-3-carboxylic acid amide,
(Compound B153) 2-[2-(3,5-Dimethoxy-phenylamino)-acetylamino]-4,7-dihydro-5H-thieno[2,3-c]pyran-3-carboxylic acid amide,
(Compound B154) 2-[3-(3,5-Bis-trifluoromethyl-phenyl)-ureido]-4,7-dihydro-5H-thieno[2,3-c]pyran-3-carboxylic acid amide,
(Compound B155) 2-[3-(2-Chloro-5-trifluoromethyl-phenyl)-ureido]-4,7-dihydro-5H-thieno[2,3-c]pyran-3-carboxylic acid amide,
(Compound B156) 2-(Cyclopropanecarbonyl-amino)-5,5-dimethyl-4,7-dihydro-5H-thieno[2,3-c]pyran-3-carboxylic acid amide,
(Compound B157) 4-[3-(3-Carbamoyl-4,7-dihydro-5H-thieno[2,3-c]pyran-2-yl)-ureido]-1-methyl-pyridinium iodide,
(Compound B158) 2-[3-(6-Methyl-pyridin-2-yl)-ureido]-4,7-dihydro-5H-thieno[2,3-c]pyran-3-carboxylic acid amide,
(Compound B159) 2-[3-(6-Methoxy-pyridin-3-yl)-ureido]-4,7-dihydro-5H-thieno[2,3-c]pyran-3-carboxylic acid amide,
(Compound B160) 2-[3-(2-Chloro-pyridin-3-yl)-ureido]-4,7-dihydro-5H-thieno[2,3-c]pyran-3-carboxylic acid amide,
(Compound B161) 2-(3-Pyridin-3-yl-thioureido)-4,7-dihydro-5H-thieno[2,3-c]pyran-3-carboxylic acid amide,
(Compound B162) 2-[3-(2-Methyl-quinolin-4-yl)-ureido]-4,7-dihydro-5H-thieno[2,3-c]pyran-3-carboxylic acid amide,
(Compound B163) 2-[3-(2,6-Dimethoxy-pyridin-3-yl)-ureido]-4,7-dihydro-5H-thieno[2,3-c]pyran-3-carboxylic acid amide,
(Compound B164) 2-[3-(6-Chloro-pyridin-3-yl)-ureido]-4,7-dihydro-5H-thieno[2,3-c]pyran-3-carboxylic acid amide,
(Compound B165) 2-(Cyclopropanecarbonyl-amino)-7-ethoxy4,7-dihydro-5H-thieno[2,3-c]pyran-3-carboxylic acid amide,
(Compound B166) 2-[3-(3-Fluoro-phenyl)-ureido]-4,7-dihydro-5H-thieno[2,3-c]pyran-3-carboxylic acid amide,
(Compound B167) 7-(3-Chloro-4-fluoro-phenylamino)-2-(cyclopropanecarbonyl-amino)-4,7-dihydro-5H-thieno[2,3-c]pyran-3-carboxylic acid amide,
(Compound B168) 2-(Cyclopropanecarbonyl-amino)-7-(2-morpholin-4-yl-ethylamino)4,7-dihydro-5H-thieno[2,3-c]pyran-3-carboxylic acid amide,
(Compound B169) 2-{3-[4-(2-Morpholin-4-yl-ethoxy)-naphthalen-1-yl]-ureido}-4,7-dihydro-5H-thieno[2,3-c]pyran-3-carboxylic acid amide,
(Compound B170) 2-[2-(2-Chloro-phenylsulfanyl)-acetylamino]-4,7-dihydro-5H-thieno[2,3-c]pyran-3-carboxylic acid amide,
(Compound B171) 2-(3-Methyl-pentanoylamino)-4,7-dihydro-5H-thieno[2,3-c]pyran-3-carboxylic acid amide,
(Compound B172) 2-[(2-Methoxymethyl-cyclopropanecarbonyl)-amino]-4,7-dihydro-5H-thieno[2,3-c]pyran-3-carboxylic acid amide,
(Compound B173) 2-(2-Methyl-pentanoylamino)-4,7-dihydro-5H-thieno[2,3-c]pyran-3-carboxylic acid amide,
(Compound B174) 2-[(2-Allyloxymethyl-cyclopropanecarbonyl)-amino]-4,7-dihydro-5H-thieno[2,3-c]pyran-3-carboxylic acid amide,
(Compound B175) 2-[(2-Octyl-cyclopropanecarbonyl)-amino]-4,7-dihydro-5H-thieno[2,3-c]pyran-3-carboxylic acid amide,
(Compound B176) 2-Ureido4,7-dihydro-5H-thieno[2,3-c]pyran-3-carboxylic acid amide,
(Compound B177) 2-(3-Methyl-pent-4-enoylamino)4,7-dihydro-5H-thieno[2,3-c]pyran-3-carboxylic acid amide,
(Compound B178) 2-(3-Phenyl-propionylamino)4,7-dihydro-5H-thieno[2,3-c]pyran-3-carboxylic acid amide,
(Compound B179) 2-[3-(4-Ethoxy-phenyl )-ureido]-4,7-dihydro-5H-thieno[2,3-c]pyran-3-carboxylic acid amide,
(Compound B180) 2-[3-(5-Chloro-2,4-dimethoxy-phenyl)-ureido]-4,7-dihydro-5H-thieno[2,3-c]pyran-3-carboxylic acid amide,
(Compound B181) 4-[3-(3-Carbamoyl-4,7-dihydro-5H-thieno[2,3-c]pyran-2-yl)-ureido]-piperidine-1-carboxylic-acid ethyl ester,
(Compound B182) 2-(3,3-Dimethyl-ureido)-4,7-dihydro-5H-thieno[2,3-c]pyran-3-carboxylic acid amide,
(Compound B183) 2-[(2-Fluoro-cyclopropanecarbonyl)-amino]-4,7-dihydro-5H-thieno[2,3-c]pyran-3-carboxylic acid amide,
(Compound B184) 2-[2-(Naphthalen-2-ylsulfanyl)-acetylamino]-4,7-dihydro-5H-thieno[2,3-c]pyran-3-carboxylic acid amide,
(Compound B185) 2-[2-(2,4-Dimethyl-phenylsulfanyl)-acetylamino]-4,7-dihydro-5H-thieno[2,3-c]pyran-3-carboxylic acid amide,
(Compound B186) 2-[2-(Quinolin-8-ylsulfanyl)-acetylamino]-4,7-dihydro-5H-thieno[2,3-c]pyran-3-carboxylic acid amide,
(Compound B187) 2-[2-(2,6-Dichloro-phenylsulfanyl)-acetylamino]-4,7-dihydro-5H-thieno[2,3-c]pyran-3-carboxylic acid amide,
(Compound B188) 2-[2-(4-Methyl-2-oxo-2H-chromen-7-ylsulfanyl)-acetylamino]-4,7-dihydro-5H-thieno[2,3-c]pyran-3-carboxylic acid amide,
(Compound B189) 2-[2-(2-Nitro-4-trifluoromethyl-phenylsulfanyl)-acetylamino]-4,7-dihydro-5H-thieno[2,3-c]pyran-3-carboxylic acid amide,
(Compound B190) 2-(3,3-Dibutyl-ureido)-4,7-dihydro-5H-thieno[2,3-c]pyran-3-carboxylic acid amide,
(Compound B191) 2-[3-(2-Hydroxy-ethyl)-ureido]-4,7-dihydro-5H-thieno[2,3-c]pyran-3-carboxylic acid amide,
(Compound B192) 2-[3-(3-Hydroxy-propyl)-ureido]-4,7-dihydro-5H-thieno[2,3-c]pyran-3-carboxylic acid amide,
(Compound B193) 2-{[2-(4-Methyl-piperazin-1-ylmethyl)-cyclopropanecarbonyl]-amino}-4,7-dihydro-5H-thieno[2,3-c]pyran-3-carboxylic acid amide,
(Compound B194) 2-[(2,2-Dichloro-3,3-dimethyl-cyclopropanecarbonyl)-amino]-4,7-dihydro-5H-thieno[2,3-c]pyran-3-carboxylic acid amide,
(Compound B195) 2-[3-(3-Carbamoyl-4,7-dihydro-5H-thieno[2,3-c]pyran-2-yl)-ureido]-3-(4-chloro-phenyl)-propionic-acid methyl ester,
(Compound B196) 2-[(2-Morpholin-4-ylmethyl-cyclopropanecarbonyl)-amino]-4,7-dihydro-5H-thieno[2,3-c]pyran-3-carboxylic acid amide,
(Compound B197) 2-[(2-Dimethylaminomethyl-cyclopropanecarbonyl)-amino]-4,7-dihydro-5H-thieno[2,3-c]pyran-3-carboxylic acid amide,
(Compound B198) 2-[(3-Carbamoyl-4,7-dihydro-5H-thieno[2,3-c]pyran-2-ylcarbamoyl)-methylsulfanyl]-benzoic-acid methyl ester,
(Compound B199) 2-[2-(2,5-Dimethyl-phenylsulfanyl)-acetylamino]-4,7-dihydro-5H-thieno[2,3-c]pyran-3-carboxylic acid amide,
(Compound B200) 2-[2-(2-Methoxy-phenylsulfanyl)-acetylamino]-4,7-dihydro-5H-thieno[2,3-c]pyran-3-carboxylic acid amide,
(Compound B201) 2-(3-Cyclopropyl-thioureido)-4,7-dihydro-5H-thieno[2,3-c]pyran-3-carboxylic acid amide,
(Compound B202) 2-[2-(Cyclopropylmethyl-amino)-acetylamino]-4,7-dihydro-5H-thieno[2,3-c]pyran-3-carboxylic acid amide,
(Compound B203) 2-(2-Chloro-acetylamino)-4,7-dihydro-5H-thieno[2,3-c]pyran-3-carboxylic acid amide,
(Compound B204) 2-(2-Isopropylamino-acetylamino)-4,7-dihydro-5H-thieno[2,3-c]pyran-3-carboxylic acid amide,
(Compound B205) 2-(2-m-Tolylsulfanyl-acetylamino)-4,7-dihydro-5H-thieno[2,3-c]pyran-3-carboxylic acid amide,
(Compound B206) 2-(2-o-Tolylsulfanyl-acetylamino)-4,7-dihydro-5H-thieno[2,3-c]pyran-3-carboxylic acid amide,
(Compound B207) 2-[2-(3,4-Dichloro-phenylsulfanyl)-acetylamino]-4,7-dihydro-5H-thieno[2,3-c]pyran-3-carboxylic acid amide,
(Compound B208) 2-[2-(3-Methoxy-phenylsulfanyl)-acetylamino]-4,7-dihydro-5H-thieno[2,3-c]pyran-3-carboxylic acid amide,
(Compound B209) 2-[2-(2,3,5,6-Tetrafluoro-phenylsulfanyl)-acetylamino]-4,7-dihydro-5H-thieno[2,3-c]pyran-3-carboxylic acid amide,
(Compound B210) 2-[2-(2,5-Dichloro-phenylsulfanyl)-acetylamino]-4,7-dihydro-5H-thieno[2,3-c]pyran-3-carboxylic acid amide,
(Compound B211) 2-[2-(3-Chloro-phenylsulfanyl)-acetylamino]-4,7-dihydro-5H-thieno[2,3-c]pyran-3-carboxylic acid amide,
(Compound B212) 2-[2-(4-Methoxy-phenylsulfanyl)-acetylamino]-4,7-dihydro-5H-thieno[2,3-c]pyran-3-carboxylic acid amide,
(Compound B213) 2-[2-(3-Bromo-phenylsulfanyl)-acetylamino]-4,7-dihydro-5H-thieno[2,3-c]pyran-3-carboxylic acid amide,
(Compound B214) 2-(2-Pyrrolidin-1-yl-acetylamino)4,7-dihydro-5H-thieno[2,3-c]pyran-3-carboxylic acid amide,
(Compound B215) 2-[2-(4-Fluoro-phenylsulfanyl)-acetylamino-]-4,7-dihydro-5H-thieno[2,3-c]pyran-3-carboxylic acid amide,
(Compound B216) 2-[2-(4-Methyl-piperazin-1-yl))-acetylamino]-4,7-dihydro-5H-thieno[2,3-c]pyran-3-carboxylic acid amide,
(Compound B217) 2-(2-Bromo-acetylamino)-4,7-dihydro-5H-thieno[2,3-c]pyran-3-carboxylic acid amide,
(Compound B218) 2-[2-(2-Morpholin-4-yl-ethylamino))-acetylamino]-4,7-dihydro-5H-thieno[2,3-c]pyran-3-carboxylic acid amide,
(Compound B219) 2-(2-Butylamino-acetylamino)-4,7-dihydro-5H-thieno[2,3-c]pyran-3-carboxylic acid amide,
(Compound B220) 2-(2-Cyclohexylamino-acetylamino)-4,7-dihydro-5H-thieno[2,3-c]pyran-3-carboxylic acid amide,
(Compound B221) 2-[(2,2-Dimethyl-cyclopropanecarbonyl)-amino]-4,7-dihydro-5H-thieno[2,3-c]pyran-3-carboxylic acid amide,
(Compound B222) 2-[2-(2-Methyl-butylamino)-acetylamino]-4,7-dihydro-5H-thieno[2,3-c]pyran-3-carboxylic acid amide,
(Compound B223) 2-[2-(1,2-Dimethyl-propylamino)-acetylamino]-4,7-dihydro-5H-thieno[2,3-c]pyran-3-carboxylic acid amide,
(Compound B224) 2-(4-Methyl-pentanoylamino)4,7-dihydro-5H-thieno[2,3-c]pyran-3-carboxylic acid amide,
(Compound B225) (3-Carbamoyl-4,7-dihydro-5H-thieno[2,3-c]pyran-2-yl)-carbamic acid ethyl ester,
(Compound B226) 2-(3-Trifluoromethyl-benzoylamino)-4,7-dihydro-5H-thieno[2,3-c]pyran-3-carboxylic acid amide,
(Compound B227) 2-[3-(5-Chloro-2-methyl-phenyl)-ureido]-4,7-dihydro-5H-thieno[2,3-c]pyran-3-carboxylic acid amide,
(Compound B228) 2-[(2-Methyl-cyclohexanecarbonyl)-amino]-4,7-dihydro-5H-thieno[2,3-c]pyran-3-carboxylic acid amide,
(Compound B229) 2-[(2-Hydroxymethyl-cyclopropanecarbonyl)-amino]-4,7-dihydro-5H-thieno[2,3-c]pyran-3-carboxylic acid amide,
(Compound B230) 2-[3-(2,4-Dimethoxy-phenyl)-ureido]-4,7-dihydro-5H-thieno[2,3-c]pyran-3-carboxylic acid amide,
(Compound B231) 2-[3-(2-Hydroxy-1-hydroxymethyl-ethyl)-ureido]-4,7-dihydro-5H-thieno[2,3-c]pyran-3-carboxylic acid amide,
(Compound B232) 2-[3-(4-Diethylamino-1-methyl-butyl)-ureido]-4,7-dihydro-5H-thieno[2,3-c]pyran-3-carboxylic acid amide,
(Compound B233) 2-[3-(2-Pyridin-2-yl-ethyl)-ureido]-4,7-dihydro-5H-thieno[2,3-c]pyran-3-carboxylic acid amide,
(Compound B234) 2-[3-(1-Cyclohexyl-ethyl)-ureido]-4,7-dihydro-5H-thieno[2,3-c]pyran-3-carboxylic acid amide,
(Compound B235) 2-(Cyclopropanecarbonyl-amino)-7-(2-methoxy-ethoxy)-4,7-dihydro-5H-thieno[2,3-c]pyran-3-carboxylic acid amide,
(Compound B236) 2-[3-(1,2,3,4-Tetrahydro-naphthalen-1-yl)-ureido]-4,7-dihydro-5H-thieno[2,3-c]pyran-3-carboxylic acid amide,
(Compound B237) 2-[3-(5,6-Dichloro-pyridin-3-yl)-ureido]-4,7-dihydro-5H-thieno[2,3-c]pyran-3-carboxylic acid amide,
(Compound B238) 2-(Cyclopropanecarbonyl-amino)-7-(2-piperidin-1-yl-ethylamino)-4,7-dihydro-5H-thieno[2,3-c]pyran-3-carboxylic acid amide,
(Compound B239) 2-(Cyclopropanecarbonyl-amino)-7-[(pyridin-2-ylmethyl)-amino]-4,7-dihydro-5H-thieno[2,3-c]pyran-3-carboxylic acid amide,
(Compound B240) 2-(Cyclopropanecarbonyl-amino)-7-(2-ethoxy-ethoxy)-4,7-dihydro-5H-thieno[2,3-c]pyran-3-carboxylic acid amide,
(Compound B241) 2-(Cyclopropanecarbonyl-amino)-7-(2-diethylamino-ethoxy)-4,7-dihydro-5H-thieno[2,3-c]pyran-3-carboxylic acid amide,
(Compound B242) 2-(Cyclopropanecarbonyl-amino)-7-(2-chloro-ethoxy)-4,7-dihydro-5H-thieno[2,3-c]pyran-3-carboxylic acid amide,
(Compound B243) 2-[3-(1,2-Dimethyl-propyl)-ureido]-4,7-dihydro-5H-thieno[2,3-c]pyran-3-carboxylic acid amide,
(Compound B244) 2-(Hydrazino-carbonyl-amino)-4,7-dihydro-5H-thieno[2,3-c]pyran-3-carboxylic acid amide,
(Compound B245) 2-[3-(4-Chloro-2-trifluoromethyl-phenyl)-ureido]-4,7-dihydro-5H-thieno[2,3-c]pyran-3-carboxylic acid amide,
(Compound B246) 2-(Cyclopropanecarbonyl-amino)-7-(4-fluoro-benzylamino)-4,7-dihydro-5H-thieno[2,3-c]pyran-3-carboxylic acid amide,
(Compound B247) 2-[3-(2-Piperazin-2-yl-ethyl)-ureido]-4,7-dihydro-5H-thieno[2,3-c]pyran-3-carboxylic acid amide,
(Compound B248) 2-(Cyclopropanecarbonyl-amino)-4,7-dihydro-5H-thieno[2,3-c]pyran-3-carboxylic acid(4-chloro-phenyl)-amide,
(Compound B249) 2-[3-(2-Methylsulfanyl-phenyl)-ureido]-4,7-dihydro-5H-thieno[2,3-c]pyran-3-carboxylic acid amide,
(Compound B250) 2-[3-(2-Trifluoromethoxy-phenyl)-ureido]-4,7-dihydro-5H-thieno[2,3-c]pyran-3-carboxylic acid amide,
(Compound B251) 2-[3-(2-Difluoromethoxy-phenyl)-ureido]-4,7-dihydro-5H-thieno[2,3-c]pyran-3-carboxylic acid amide,
(Compound B252) 2-{3-[4-Cyano-3-(4-methoxy-phenyl)-isoxazol-5-yl]-ureido}-4,7-dihydro-5H-thieno[2,3-c]pyran-3-carboxylic acid amide,
(Compound B253) 2-[3-(4-Cyano-3-cyclopropyl-isoxazol-5-yl)-ureido]-4,7-dihydro-5H-thieno[2,3-c]pyran-3-carboxylic acid amide,
(Compound B254) 2-(3-Pyrimidin-2-yl-ureido)4,7-dihydro-5H-thieno[2,3-c]pyran-3-carboxylic acid amide,
(Compound B255) 2-[3-(4-Trifluoromethylsulfanyl-phenyl)-ureido]-4,7-dihydro-5H-thieno[2,3-c]pyran-3-carboxylic acid amide,
(Compound B256) 2-[3-(4-Cyano-3-phenyl-isoxazol-5-yl)-ureido]-4,7-dihydro-5H-thieno[2,3-c]pyran-3-carboxylic acid amide,
(Compound B257) 2-(3-Piperidin-1-yl-ureido)-4,7-dihydro-5H-thieno[2,3-c]pyran-3-carboxylic acid amide,
(Compound B258) 2-[3-(4-Methyl-piperazin-1-yl)-ureido]-4,7-dihydro-5H-thieno[2,3-c]pyran-3-carboxylic acid amide,
(Compound B259) 2-[3-(4-tert-Butyl-cyclohexyl)-ureido]-4,7-dihydro-5H-thieno[2,3-c]pyran-3-carboxylic acid amide,
(Compound B260) 2-[3-(1S,2S,3S,5R-2,6,6-Trimethyl-bicyclo[3.1.1]hept-3-yl)-ureido]4,7-dihydro-5H-thieno[2,3-c]pyran-3-carboxylic acid amide,
(Compound B261) 2-[3-(2,3-Dihydro-benzo[1,4]dioxin-6-yl)-ureido]-4,7-dihydro-5H-thieno[2,3-c]pyran-3-carboxylic acid amide,
(Compound B262) 2-[3-(2-Hydroxy-phenyl)-ureido]-4,7-dihydro-5H-thieno[2,3-c]pyran-3-carboxylic acid amide,
(Compound B263) N-(3-Carbamoyl-4,7-dihydro-5H-thieno[2,3]pyran-2-yl)-N′-(2-hydroxyethyl)-oxalamide,
(Compound B264) 4-[3-(3-Carbamoyl-4,7-dihydro-5H-thieno[2,3-c]pyran-2-yl)-ureido]-benzoic acid ethyl ester,
(Compound B265) 2-[3-(3-Methylsulfanyl-phenyl)-ureido]-4,7-dihydro-5H-thieno[2,3-c]pyran-3-carboxylic acid amide,
(Compound B266) 2-[3-(3-Methylsulfanyl-phenyl)-ureido]-4,7-dihydro-5H-thieno[2,3-c]pyran-3-carboxylic acid amide,
(Compound B267) 2-[3-(2-Ethyl-phenyl)-ureido]-4,7-dihydro-5H-thieno[2,3-c]pyran-3-carboxylic acid amide,
(Compound B268) 2-[3-(2-Chloro-phenyl)-ureido]-4,7-dihydro-5H-thieno[2,3-c]pyran-3-carboxylic acid amide,
(Compound B269) 2-[3-(2-Chloro-6-methyl-phenyl)-ureido]-4,7-dihydro-5H-thieno[2,3-c]pyran-3-carboxylic acid amide,
(Compound B270) 2-[3-(4-Bromo-2-fluoro-phenyl)-ureido]-4,7-dihydro-5H-thieno[2,3-c]pyran-3-carboxylic acid amide,
(Compound B271) 2-[3-(2-Fluoro-3-trifluoromethyl-phenyl)-ureido]-4,7-dihydro-5H-thieno[2,3-c]pyran-3-carboxylic acid amide,
(Compound B272) 2-[3-(2,3,4-Trifluoro-phenyl)-ureido]-4,7-dihydro-5H-thieno[2,3-c]pyran-3-carboxylic acid amide,
(Compound B273) 2-[3-(2,6-Dibromo4-fluoro-phenyl)-ureido]-4,7-dihydro-5H-thieno[2,3-c]pyran-3-carboxylic acid amide,
(Compound B274) 2-[3-(2,3-Dimethyl-phenyl)-ureido]-4,7-dihydro-5H-thieno[2,3-c]pyran-3-carboxylic acid amide,
(Compound B275) 2-[3-(3-Fluoro-4-methyl-phenyl)-ureido]-4,7-dihydro-5H-thieno[2,3-c]pyran-3-carboxylic acid amide,
(Compound B276) 2-[3-(3,4-Difluoro-phenyl)-ureido]-4,7-dihydro-5H-thieno[2,3-c]pyran-3-carboxylic acid amide,
(Compound B277) 2-[3-(4-Chloro-2-methyl-phenyl)-ureido]-4,7-dihydro-5H-thieno[2,3-c]pyran-3-carboxylic acid amide,
(Compound B278) 2-[3-(4-Ethyl-phenyl)-ureido]-4,7-dihydro-5H-thieno[2,3-c]pyran-3-carboxylic acid amide,
(Compound B279) 2-[3-(3-Iodo-phenyl)-ureido]-4,7-dihydro-5H-thieno[2,3-c]pyran-3-carboxylic acid amide,
(Compound B280) 2-[3-(2,5-Dimethyl-phenyl)-ureido]-4,7-dihydro-5H-thieno[2,3-c]pyran-3-carboxylic acid amide,
(Compound B281) 2-[3-(4-Butoxy-phenyl)-ureido]-4,7-dihydro-5H-thieno[2,3-c]pyran-3-carboxylic acid amide,
(Compound B282) 2-[3-(2,4-Dibromo-phenyl)-ureido]-4,7-dihydro-5H-thieno[2,3-c]pyran-3-carboxylic acid amide,
(Compound B283) 2-[3-(4-Butyl-phenyl)-ureido]-4,7-dihydro-5H-thieno[2,3-c]pyran-3-carboxylic acid amide,
(Compound B284) 2-[3-(4-Dimethylamino-phenyl)-ureido]-4,7-dihydro-5H-thieno[2,3-c]pyran-3-carboxylic acid amide,
(Compound B285) 2-[3-(4-Heptyloxy-phenyl)-ureido]-4,7-dihydro-5H-thieno[2,3-c]pyran-3-carboxylic acid amide,
(Compound B286) 2-[3-(2-Propyl-phenyl)-ureido]-4,7-dihydro-5H-thieno[2,3-c]pyran-3-carboxylic acid amide;
(Compound C1) 2-(Cyclopropanecarbonyl-amino)-4,7-dihydro-5H-thieno[2,3-c]thiopyran-3-carboxylic acid amide,
(Compound C2) 2-(Cyclopropanecarbonyl-amino)-6-oxo-4,5,6,7-tetrahydro-6λ⁴-thieno[2,3-c]thiopyran-3-carboxylic acid amide,
(Compound C3) 3-Carbamoyl-2-(cyclopropanecarbonyl-amino)4,7-dihydro-5H-thieno[2,3-c]pyridine-6-carboxylic acid ethyl ester,
(Compound C4) 6-Acetyl-2-(cyclopropanecarbonyl-amino)-4,5,6,7-tetrahydro-thieno[2,3-c]pyridine-3-carboxylic acid amide,
(Compound C5) 2-[3-(4-Methoxy-phenyl)-ureido]-4,7-dihydro-5H-thieno[2,3-c]thiopyran-3-carboxylic acid amide,
(Compound C6) 2-{3-[4-(2-Morpholin-4-yl-ethoxy)-naphthalen-1-yl]-ureido}-4,7-dihydro-5H-thieno[2,3-c]thiopyran-3-carboxylic acid amide,
(Compound C7) 2-[3-(3-Carbamoyl-4,7-dihydro-5H-thieno[2,3-c]thiopyran-2-yl)-ureido]-3-methyl-butyric acid methyl ester,
(Compound C8) 2-[3-(4-Benzyloxy-phenyl)-ureido]-4,7-dihydro-5H-thieno[2,3-c]thiopyran-3-carboxylic acid amide,
(Compound C9) 2-[3-(3-Morpholin-4-yl-propyl)-ureido]-4,7-dihydro-5H-thieno[2,3-c]thiopyran-3-carboxylic acid amide,
(Compound C10) 2-(3-Pyridin-3-yl-ureido)-4,7-dihydro-5H-thieno[2,3-c]thiopyran-3-carboxylic acid amide,
(Compound C11) 2-[3-(3-Chloro-4-fluoro-phenyl)-ureido]-4,7-dihydro-5H-thieno[2,3-c]thiopyran-3-carboxylic acid amide,
(Compound C12) 2-[3-(2-Methoxy-benzyl)-ureido)-4,7-dihydro-5H-thieno[2,3-c]thiopyran-3-carboxylic acid amide,
(Compound C13) 2-[3-(4-Nitro-phenyl)-ureido]-4,7-dihydro-5H-thieno[2,3-c]thiopyran-3-carboxylic acid amide,
(Compound C14) 2-[3-(4-Isopropyl-phenyl)-ureido]-4,7-dihydro-5H-thieno[2,3-c]thiopyran-3-carboxylic acid amide,
(Compound C15) 2-[3-(2,4-Difluoro-phenyl)-ureido]-4,7-dihydro-5H-thieno[2,3-c]thiopyran-3-carboxylic acid amide,
(Compound C16) 2-[3-(3,4,5-Trimethoxy-phenyl)-ureido]-4,7-dihydro-5H-thieno[2,3-c]thiopyran-3-carboxylic acid amide,
(Compound C17) 2-[3-(3,5-Dichloro-phenyl)-ureido]-4,7-dihydro-5H-thieno[2,3-c]thiopyran-3-carboxylic acid amide,
(Compound C18) 2-[3-(2-Nitro-phenyl)-ureido]-4,7-dihydro-5H-thieno[2,3-c]thiopyran-3-carboxylic acid amide,
(Compound C19) 2-[3-(3-Methoxy-phenyl)-ureido]-4,7-dihydro-5H-thieno[2,3-c]thiopyran-3-carboxylic acid amide,
(Compound C20) 2-[3-(2-Methyl-3-nitro-phenyl)-ureido]-4,7-dihydro-5H-thieno[2,3-c]thiopyran-3-carboxylic acid amide,
(Compound C21) 2-[3-(4-Chloro-3-nitro-phenyl)-ureido]-4,7-dihydro-5H-thieno[2,3-c]thiopyran-3-carboxylic acid amide,
(Compound C22) 2-[3-(2-Methoxy-5-nitro-phenyl)-ureido]-4,7-dihydro-5H-thieno[2,3-c]thiopyran-3-carboxylic acid amide,
(Compound C23) 2-[3-(3-Bromo-phenyl)-ureido]-4,7-dihydro-5H-thieno[2,3-c]thiopyran-3-carboxylic acid amide,
(Compound C24) 2-[3-(3,5-Dimethoxy-phenyl)-ureido]-4,7-dihydro-5H-thieno[2,3-c]thiopyran-3-carboxylic acid amide,
(Compound C25) 2-[3-(4-Chloro-3-trifluoromethyl-phenyl)-ureido]-4,7-dihydro-5H-thieno[2,3-c]thiopyran-3-carboxylic acid amide,
(Compound C26) 2-[3-(3,5-Bis-trifluoromethyl-phenyl)-ureido]-4,7-dihydro-5H-thieno[2,3-c]thiopyran-3-carboxylic acid amide,
(Compound C27) 2-(3-m-Tolyl-ureido)-4,7-dihydro-5H-thieno[2,3-c]thiopyran-3-carboxylic acid amide,
(Compound C28) 2-(3-p-Tolyl-ureido)-4,7-dihydro-5H-thieno[2,3-c]thiopyran-3-carboxylic acid amide,
(Compound C29) 2-[3-(3-Methyl-benzyl)-ureido]-4,7-dihydro-5H-thieno[2,3-c]thiopyran-3-carboxylic acid amide,
(Compound C30) 2-[3-(2-Chloro-5-trifluoromethyl-phenyl)-ureido]-4,7-dihydro-5H-thieno[2,3-c]thiopyran-3-carboxylic acid amide,
(Compound C31) 2-(3-o-Tolyl-ureido)-4,7-dihydro-5H-thieno[2,3-c]thiopyran-3-carboxylic acid amide,
(Compound C32) 2-[3-(4-Methoxy-benzyl)-ureido]-4,7-dihydro-5H-thieno[2,3-c]thiopyran-3-carboxylic acid amide,
(Compound C33) 2-[3-(2-Methyl-benzyl)-ureido]-4,7-dihydro-5H-thieno[2,3-c]thiopyran-3-carboxylic acid amide,
(Compound C34) 2-[3-(2,3-Dichloro-phenyl)-ureido]-4,7-dihydro-5H-thieno[2,3-c]thiopyran-3-carboxylic acid amide,
(Compound C35) 2-(3-Biphenyl-4-yl-ureido)-4,7-dihydro-5H-thieno[2,3-c]thiopyran-3-carboxylic acid amide,
(Compound C36) 2-[3-(3-Trifluoromethyl-phenyl)-ureido]-4,7-dihydro-5H-thieno[2,3-c]thiopyran-3-carboxylic acid amide,
(Compound C37) 2-[3-(4-Bromo-phenyl)-ureido]-4,7-dihydro-5H-thieno[2,3-c]thiopyran-3-carboxylic acid amide,
(Compound C38) 2-(3-Cyclohexyl-ureido)-4,7-dihydro-5H-thieno[2,3-c]thiopyran-3-carboxylic acid amide,
(Compound C39) 2-(2-Methyl-acryloylamino)-4,7-dihydro-5H-thieno[2,3-c]thiopyran-3-carboxylic acid amide,
(Compound C40) 2-[3-(3-Chloro-phenyl)-ureido]-4,7-dihydro-5H-thieno[2,3-c]thiopyran-3-carboxylic acid amide,
(Compound C41) 2-(3-Benzyl-ureido)-4,7-dihydro-5H-thieno[2,3-c]thiopyran-3-carboxylic acid amide,
(Compound C42) 2-[3-(3-Methyl-butyl)-ureido]-4,7-dihydro-5H-thieno[2,3-c]thiopyran-3-carboxylic acid amide,
(Compound C43) 2-[3-(4-Methyl-benzyl)-ureido]-4,7-dihydro-5H-thieno[2,3-c]thiopyran-3-carboxylic acid amide,
(Compound C44) 2-(3-Isopropyl-ureido)-4,7-dihydro-5H-thieno[2,3-c]thiopyran-3-carboxylic acid amide,
(Compound C45) 2-[3-(4-Fluoro-phenyl)-ureido]-4,7-dihydro-5H-thieno[2,3-c]thiopyran-3-carboxylic acid amide,
(Compound C46) 2-[3-(2-Methyl-5-nitro-phenyl)-ureido]-4,7-dihydro-5H-thieno[2,3-c]thiopyran-3-carboxylic acid amide,
(Compound C47) 2-[3-(4-Acetyl-phenyl)-ureido]-4,7-dihydro-5H-thieno[2,3-c]thiopyran-3-carboxylic acid amide,
(Compound C48) 2-[3-(4-Methyl-2-nitro-phenyl)-ureido]-4,7-dihydro-5H-thieno[2,3-c]thiopyran-3-carboxylic acid amide,
(Compound C49) 2-[3-(4-Phenoxy-phenyl)-ureido]-4,7-dihydro-5H-thieno[2,3-c]thiopyran-3-carboxylic acid amide,
(Compound C50) 2-[3-(3,4-Dichloro-phenyl)-ureido]-4,7-dihydro-5H-thieno[2,3-c]thiopyran-3-carboxylic acid amide,
(Compound C51) 2-(Cyclopropanecarbonyl-amino)-6-methyl-4,5,6,7-tetrahydro-thieno[2,3-c]pyridine-3-carboxylic acid amide,
(Compound C52) 2-[(2,2,3,3-Tetramethyl-cyclopropanecarbonyl)-amino]-4,7-dihydro-5H-thieno[2,3-c]thiopyran-3-carboxylic acid amide,
(Compound C53) 2-[3-(4-Trifluoromethyl-phenyl)-ureido]-4,7-dihydro-5H-thieno[2,3-c]thiopyran-3-carboxylic acid amide;
(Compound D1) 2-(Cyclopropanecarbonyl-amino)-4,5,6,7-tetrahydro-benzo[b]thiophene-3-carboxylic acid amide,
(Compound D2) Furan-2-carboxylic acid2-[3-(2-hydroxy-ethylcarbamoyl)-4,5,6,7-tetrahydro-benzo[b]thiophen-2-yl]-amide,
(Compound D3) 2-Acetylamino-4,5,6,7-tetrahydro-benzo[b]thiophene-3-carboxylic acid amide,
(Compound D4) 2-(2,2-Dimethyl-propionylamino)-4,5,6,7-tetrahydro-benzo[b]thiophene-3-carboxylic acid amide,
(Compound D5) 2-Isobutyrylamino)-4,5,6,7-tetrahydro-benzo[b]thiophene-3-carboxylic acid amide,
(Compound D6) 2-(2-Methyl-acryloylamino)-4,5,6,7-tetrahydro-benzo[b]thiophene-3-carboxylic acid amide,
(Compound D7) 2-[(Thiophene-2-carbonyl)-amino]-4,5,6,7-tetrahydro-benzo[b]thiophene-3-carboxylic acid amide,
(Compound D8) Furan-2-carboxylic acid (3-carbamoyl-4,5,6,7-tetrahydro-benzo[b]thiophen-2-yl]-amide,
(Compound D9) 2-(Cyclobutanecarbonylamino)-4,5,6,7-tetrahydro-benzo[b]thiophene-3-carboxylic acid amide,
(Compound D10) 2-(2-Methyl-butyrylamino)-4,5,6,7-tetrahydro-benzo[b]thiophene-3-carboxylic acid amide,
(Compound D11) 2-(Cyclopropanecarbonyl-amino)-4-methyl-4,5,6,7-tetrahydro-benzo[b]thiophene-3-carboxylic acid amide,
(Compound D12) 6-tert-Butyl-2-(cyclopropanecarbonyl-amino)-4,5,6,7-tetrahydro-benzo[b]thiophene-3-carboxylic acid amide,
(Compound D13) 2-(Cyclopropanecarbonyl-amino)-6-methyl-4,5,6,7-tetrahydro-benzo[b]thiophene-3-carboxylic acid amide,
(Compound D14) 2-(Cyclohexanecarbonyl-amino)-4,5,6,7-tetrahydro-benzo[b]thiophene-3-carboxylic acid amide
(Compound D15) 2-Acetylamino-6-methyl-4,5,6,7-tetrahydro-benzo[b]thiophene-3-carboxylic acid amide,
(Compound D16) N-(3-Carbamoyl-4,5,6,7-tetrahydro-benzo[b]thiophen-2-yl)-succinamic acid methyl ester,
(Compound D17) 6-Methyl-2-propyonylamino-4,5,6,7-tetrahydro-benzo[b]thiophene-3-carboxylic acid amide,
(Compound D18) 2-isobutyrylamino-6-methyl-4,5,6,7-tetrahydro-benzo[b]thiophene-3-carboxylic acid amide,
(Compound D19) 2-Butyrylamino-4,5,6,7-tetrahydro-benzo[b]thiophene-3-carboxylic acid amide,
(Compound D20) 3-(3-Carbamoyl-4,5,6,7-tetrahydro-benzo[b]thiophen-2-ylcarbamoyl)-acrylic acid,
(Compound D21) N-(3-Carbamoyl-4,5,6,7-tetrahydro-benzo[b]thiophen-2-yl)-succinamic acid,
(Compound D22) 2-Benzoylamino-5,6-dihydro-4H-cyclopenta[b]thiophene-3-carboxylic acid amide,
(Compound D23) Furan-2-carboxylic acid (3-carbamoyl-5,6-dihydro-4H-cyclopenta[b]thiophen-2-yl]-amide,
(Compound D24) 2-(2-Cyano-acetylamino)-4,5,6,7-tetrahydro-benzo[b]thiophene-3-carboxylic acid amide,
(Compound D25) 2-(2-Phenoxy-acetylamino)-4,5,6,7-tetrahydro-benzo[b]thiophene-3-carboxylic acid amide,
(Compound D26) 2-(3-Phenyl-ureido)-4,5,6,7-tetrahydro-benzo[b]thiophene-3-carboxylic acid amide,
(Compound D27) 2-(2-Piperidin-1-yl-acetylamino)-4,5,6,7-tetrahydro-benzo[b]thiophene-3-carboxylic acid amide,
(Compound D28) 2-(2-Diallylamino-acetylamino)-4,5,6,7-tetrahydro-benzo[b]thiophene-3-carboxylic acid amide,
(Compound D29) 2-(2-Morpholin-4-yl-acetylamino)-4,5,6,7-tetrahydro-benzo[b]thiophene-3-carboxylic acid amide,
(Compound D30) 4-Ethyl-piperazine-1-carboxylic acid (3-carbamoyl-4,5,6,7-tetrahydro-benzo[b]thiophen-2-yl]-amide,
(Compound D31) 2-(2-Diethylamino-acetylamino)-4,5,6,7-tetrahydro-benzo[b]thiophene-3-carboxylic acid amide,
(Compound D32) 2-(3-Allyl-thioureido)-4,5,6,7-tetrahydro-benzo[b]thiophene-3-carboxylic acid amide,
(Compound D33) 2-Pentanoylamino-4,5,6,7-tetrahydro-benzo[b]thiophene-3-carboxylic acid amide,
(Compound D34) 6-Methyl-2-(3-thiophen-2-yl-acryloylamino)-4,5,6,7-tetrahydro-benzo[b]thiophene-3-carboxylic acid amide,
(Compound D35) 2-[(Adamantane-1-carbonyl)-amino]-4,5,6,7-tetrahydro-benzo[b]thiophene-3-carboxylic acid amide,
(Compound D36) 5,6-Dihydro-[1,4]-dioxine-2-carboxylic acid (3-carbamoyl-4,5,6,7-tetrahydro-benzo[b]thiophen-2-yl]-amide,
(Compound D37) 2-(Cyclopropanecarbonyl-amino)-5-methyl-4,5,6,7-tetrahydro-benzo[b]thiophene-3-carboxylic acid amide,
(Compound D38) 2-Isobutyrylamino-5-methyl-4,5,6,7-tetrahydro-benzo[b]thiophene-3-carboxylic acid amide,
(Compound D39) 2-Phenylacetylamino-4,5,6,7-tetrahydro-benzo[b]thiophene-3-carboxylic acid amide,
(Compound D40) (3-Carbamoyl-4,5,6,7-tetrahydro-benzo[b]thiophen-2-yl)-carbamic acid butyl ester,
(Compound D41) 6-Methyl-2-(3-methyl-but-2-enoylamino)-4,5,6,7-tetrahydro-benzo[b]thiophene-3-carboxylic acid amide,
(Compound D42) (3-Carbamoyl-4,5,6,7-tetrahydro-benzo[b]thiophen-2-yl)-carbamic acid ethyl ester,
(Compound D43) 2-(2-Cyano-3-furan-2yl-acryloylamino)-4,5,6,7-tetrahydro-benzo[b]thiophene-3-carboxylic acid amide,
(Compound D44) 2-(3-Methyl-but-2-enoylamino)-4,5,6,7-tetrahydro-benzo[b]thiophene-3-carboxylic acid amide,
(Compound D45) 2-(3-Phenyl-acryloylamino)-4,5,6,7-tetrahydro-benzo[b]thiophene-3-carboxylic acid amide,
(Compound D46) 2-(2-Methoxy-acetylamino)-4,5,6,7-tetrahydro-benzo[b]thiophene-3-carboxylic acid amide,
(Compound D47) 2-[2-(1,1-Dioxo-tetrahydro-1λ⁶-thiophen-3-yl)-acetylamino]-4,5,6,7-tetrahydro-benzo[b]thiophene-3-carboxylic acid amide,
(Compound D48) 2-[2-(4-Methoxyphenyl)-acetylamino]-4,5,6,7-tetrahydro-benzo[b]thiophene-3-carboxylic acid amide,
(Compound D49) (3-Carbamoyl-6-methyl-4,5,6,7-tetrahydro-benzo[b]thiophen-2-yl)-carbamic acid phenyl ester,
(Compound D50) 2-(4-Phenoxy-butyrylamino)-4,5,6,7-tetrahydro-benzo[b]thiophene-3-carboxylic acid amide,
(Compound D51) (3-Carbamoyl-4,5,6,7-tetrahydro-benzo[b]thiophen-2-yl)-carbamic acid propyl ester
(Compound D52) Tetrahydro-furan-2-carboxylic-acid(3-Carbamoyl-4,5,6,7-tetrahydro-benzo[b]thiophen-2-yl)-amide,
(Compound D53) 2-(4-Phenyl-butyrylamino]-4,5,6,7-tetrahydro-benzo[b]thiophene-3-carboxylic acid amide,
(Compound D54) 2-(3-Pyrrolidin-1-yl-propionylamino)-4,5,6,7-tetrahydro-benzo[b]thiophene-3-carboxylic acid amide,
(Compound D55) 2-(2-Ethyl-hexanoylamino)-4,5,6,7-tetrahydro-benzo[b]thiophene-3-carboxylic acid amide,
(Compound D56) 2-(3-Cyclopentyl-propionylamino)-4,5,6,7-tetrahydro-benzo[b]thiophene-3-carboxylic acid amide,
(Compound D57) 2-(3-Cyclohexyl-propionylamino)-4,5,6,7-tetrahydro-benzo[b]thiophene-3-carboxylic acid amide,
(Compound D58) 2-(3-Methyl-butyrylamino)-4,5,6,7-tetrahydro-benzo[b]thiophene-3-carboxylic acid amide,
(Compound D59) 2-(2,2,2-Trifluoro-acetylamino)-4,5,6,7-tetrahydro-benzo[b]thiophene-3-carboxylic acid amide,
(Compound D60) 2-(3-Furan-2-yl-acryloylamino)-4,5,6,7-tetrahydro-benzo[b]thiophene-3-carboxylic acid amide,
(Compound D61) 2-[(2-Phenyl-cyclopropanecarbonyl)-amino]-4,5,6,7-tetrahydro-benzo[b]thiophene-3-carboxylic acid amide,
(Compound D62) 2-(Cyclopentanecarbonyl-amino)-6-4,5,6,7-tetrahydro-benzo[b]thiophene-3-carboxylic acid amide,
(Compound D63) 2-[(Bicyclo[2.2.1]heptane-2-carbonyl)-amino]-4,5,6,7-tetrahydro-benzo[b]thiophene-3-carboxylic acid amide,
(Compound D64) 2-(3-Adamantan-1-yl-ureido)-4,5,6,7-tetrahydro-benzo[b]thiophene-3-carboxylic acid amide,
(Compound D65) 2-[3-(4-Methoxy-benzyl)-ureido]-4,5,6,7-tetrahydro-benzo[b]thiophene-3-carboxylic acid amide,
(Compound D66) 2-(Cyclopropanecarbonyl-amino)-6-fluoro-4,5,6,7-tetrahydro-benzo[b]thiophene-3-carboxylic acid amide,
(Compound D67) 6,6-Dibromo-2-(cyclopropanecarbonyl-amino)-7-oxo-4,5,6,7-tetrahydro-benzo[b]thiophene-3-carboxylic acid amide,
(Compound D68) 2-[3-(4-Methyl-benzyl)-ureido]-4,5,6,7-tetrahydro-benzo[b]thiophene-3-carboxylic acid amide,
(Compound D69) 2-[3-(3-Methyl-benzyl)-ureido]-4,5,6,7-tetrahydro-benzo[b]thiophene-3-carboxylic acid amide,
(Compound D70) 2-(3-p-Tolyl-ureido)-4,5,6,7-tetrahydro-benzo[b]thiophene-3-carboxylic acid amide,
(Compound D71) 2-[3-(4-Methyl-2-nitro-phenyl)-ureido]-4,5,6,7-tetrahydro-benzo[b]thiophene-3-carboxylic acid amide,
(Compound D72) E-7-Benzyloxyimino-2-(cyclopropanecarbonyl-amino)-4,5,6,7-tetrahydro-benzo[b]thiophene-3-carboxylic acid amide,
(Compound D73) Z-7-Benzyloxyamino-2-(cyclopropanecarbonyl-amino)-4,5,6,7-tetrahydro-benzo[b]thiophene-3-carboxylic acid amide,
(Compound D74) 2-[3-(3-Methyl-butyl)-ureido]-4,5,6,7-tetrahydro-benzo[b]thiophene-3-carboxylic acid amide,
(Compound D75) 2-[3-(4-Acetyl-phenyl)-ureido]-4,5,6,7-tetrahydro-benzo[b]thiophene-3-carboxylic acid amide,
(Compound D76) 2-[3-(2-Methyl-5-nitro-phenyl)-ureido]-4,5,6,7-tetrahydro-benzo[b]thiophene-3-carboxylic acid amide,
(Compound D77) 2-(3-m-Tolyl-ureido)-4,5,6,7-tetrahydro-benzo[b]thiophene-3-carboxylic acid amide,
(Compound D78) 3-(3-Carbamoyl-4,5,6,7-tetrahydro-benzo[b]thiophen-2-ylcarbamoyl)-acrylic acid,
(Compound D79) 2-(Cyclopropanecarbonyl-amino)-4,5,6,7-tetrahydro-benzo[b]thiophene-3,6-dicarboxylic acid 3-amide 6-[(2-hydroxy-ethyl)-amide],
(Compound D80) 2-[3-(2-Methyl-benzyl)-ureido]-4,5,6,7-tetrahydro-benzo[b]thiophene-3-carboxylic acid amide,
(Compound D81) 2-[3-(3-Morpholin-4-yl-propyl)-ureido]-4,5,6,7-tetrahydro-benzo[b]thiophene-3-carboxylic acid amide,
(Compound D82) 3-Carbamoyl-2-(cyclopropanecarbonyl-amino)-4,5,6,7-tetrahydro-benzo[b]thiophene-6-carboxylic acid,
(Compound D83) 2-[3-(4-Phenoxy-phenyl)-ureido]4,5,6,7-tetrahydro-benzo[b]thiophene-3-carboxylic acid amide,
(Compound D84) E-2-(Cyclopropanecarbonyl-amino)-7-hydroxyimino-4,5,6,7-tetrahydro-benzo[b]thiophene-3-carboxylic acid amide,
(Compound D85) E-2-(Cyclopropanecarbonyl-amino)-7-methoxyimino-4,5,6,7-tetrahydro-benzo[b]thiophene-3-carboxylic acid amide,
(Compound D86) Cyclopropanecarboxylic acid (3-sulfamoyl-4,5,6,7-tetrahydro-benzo[b]thiophen-2-yl)-amide,
(Compound D87) 2-[3-(4-Trifluoromethyl-phenyl)-ureido]-4,5,6,7-tetrahydro-benzo[b]thiophene-3-carboxylic acid amide,
(Compound D88) Z-2-(Cyclopropanecarbonyl-amino)-7-methoxyimino-4,5,6,7-tetrahydro-benzo[b]thiophene-3-carboxylic acid amide,
(Compound D89) 2-(cyclopropanecarbonyl-amino)-6-oxo-4,5,6,7-tetrahydro-benzo[b]thiophene-3-carboxylic acid amide,
(Compound D90) 2-(cyclopropanecarbonyl-amino)-6-hydroxy-4,5,6,7-tetrahydro-benzo[b]thiophene-3-carboxylic acid amide,
(Compound D91) 2-Phenylmethanesulfonylamino-4,5,6,7-tetrahydro-benzo[b]thiophene-3-carboxylic acid amide,
(Compound D92) 1-Benzyl-2-(cyclopropanecarbonyl-amino)-4,5,6,7-tetrahydro-1H-indole-3-carboxylic acid amide,
(Compound D93) 2-(Cyclopropanecarbonyl-amino)-7-hydroxyimino-6-methyl-4,5,6,7-tetrahydro-benzo[b]thiophene-3-carboxylic acid amide,
(Compound D94) 2-[(2-Methyl-cyclopropanecarbonyl)-amino]-4,5,6,7-tetrahydro-benzo[b]thiophene-3-carboxylic acid amide,
(Compound D95) 2-(Cyclopropanecarbonyl-amino)-5,5,7,7-tetramethyl-4,5,6,7-tetrahydro-benzo[b]thiophene-3-carboxylic acid amide,
(Compound D96) 2-(Cyclopropanecarbonyl-amino)-4-methyl-4,6-dihydro-thieno[2,3-c]furan-3-carboxylic acid amide,
(Compound D97) 2-[(Cyclopropylcarbonyl)amino]-4,7-dihydro-5H-spiro[1-benzothiophene-6,2′-[1,3]dioxolane]-3-carboxamide,
(Compound D98) 2-(Cyclopropanecarbonyl-amino)-4,4a,5,6,7,8,8a,9-octahydro-naphtho[2,3-b]thiophene-3-carboxylic acid amide,
(Compound D99) Cyclopropanecarboxylic-acid(3-thiocarbamoyl-4,5,6,7-tetrahydro-benzo[b]thiophen-2-yl)-amide,
(Compound D100) 2-(Cyclopropanecarbothioyl-amino)-4,5,6,7-tetrahydro-benzo[b]thiophene-3-carbothioic acid amide,
(Compound D101) 2-(Cyclopropanecarbonyl-amino)-6-(1-hydroxy-1-methyl-ethyl)-4,5,6,7-tetrahydro-benzo[b]thiophene-3-carboxylic acid amide,
(Compound D102) 6-Ethyl-2-[(2-methyl-cyclopropanecarbonyl)-amino]-4,5,6,7-tetrahydro-benzo[b]thiophene-3-carboxylic acid amide,
(Compound D103) 2-[3-(3,4-Dichloro-phenyl)-ureido]-4,5,6,7-tetrahydro-benzo[b]thiophene-3-carboxylic acid amide,
(Compound D104) Z-2-(Cyclopropanecarbonyl-amino)-7-hydroxyimino-4,5,6,7-tetrahydro-benzo[b]thiophene-3-carboxylic acid amide,
(Compound D105) 2-[3-(4-Iodo-phenyl)-ureido]-6-oxo-4,5,6,7-tetrahydro-benzo[b]thiophene-3-carboxylic acid amide,
(Compound D106) 2-[3-(4-Methoxy-phenyl)-ureido]-6-oxo-4,5,6,7-tetrahydro-benzo[b]thiophene-3-carboxylic acid amide,
(Compound D107) 2-[3-(2,3-Dichloro-phenyl)-ureido]-4,5,6,7-tetrahydro-benzo[b]thiophene-3-carboxylic acid amide,
(Compound D108) 2-(Cyclopropanecarbonyl-amino)-6-hydroxymethyl-4,5,6,7-tetrahydro-benzo[b]thiophene-3-carboxylic acid amide,
(Compound D109) 2-[3-(4-Bromo-phenyl)-ureido]-6-methyl-4,5,6,7-tetrahydro-benzo[b]thiophene-3-carboxylic acid amide,
(Compound D110) 6-Methyl-2-[3-(4-trifluoromethoxy-phenyl)-ureido]-4,5,6,7-tetrahydro-benzo[b]thiophene-3-carboxylic acid amide,
(Compound D111) 2-[3-(4-trifluoromethoxy-phenyl)-ureido]-4,5,6,7-tetrahydro-benzo[b]thiophene-3-carboxylic acid amide,
(Compound D112) 2-(3-Biphenyl-4-yl-ureido)-4,5,6,7-tetrahydro-benzo[b]thiophene-3-carboxylic acid amide,
(Compound D113) 2-[3-(4-Fluoro-phenyl)-ureido]-4,5,6,7-tetrahydro-benzo[b]thiophene-3-carboxylic acid amide,
(Compound D114) 2-[3-(4-Benzyloxy-phenyl)-ureido]-4,5,6,7-tetrahydro-benzo[b]thiophene-3-carboxylic acid amide,
(Compound D115) 2-(Cyclopropanecarbonyl-amino)-6-hydrazono-4,5,6,7-tetrahydro-benzo[b]thiophene-3-carboxylic acid amide,
(Compound D116) 2-[({[4-(Trifluoromethyl)phenyl]amino}carbonyl)amino]-4,7-dihydro-5H-spiro[1-benzothiophene-6,2′-[1,3]dioxolane]-3-carboxamide,
(Compound D117) 2-({[(4-Acetylphenyl)amino]carbonyl}amino)-4,7-dihydro-5H-spiro[1-benzothiophene-6,2′-[1,3]dioxolane]-3-carboxamide,
(Compound D118) 2-(3-Cyclohexyl-ureido]-(6-oxo-ethylenylketal)-4,5,6,7-tetrahydro-benzo[b]thiophene-3-carboxylic acid amide,
(Compound D119) 6-Oxo-2-[3-(4-trifluoromethyl-phenyl)-ureido]-4,5,6,7-tetrahydro-benzo[b]thiophene-3-carboxylic acid amide,
(Compound D120) 2-[3-(4-Acetyl-phenyl)-ureido]-6-oxo-4,5,6,7-tetrahydro-benzo[b]thiophene-3-carboxylic acid amide,
(Compound D121) 2-[3-(4-Fluoro-phenyl)-ureido]-6-oxo-4,5,6,7-tetrahydro-benzo[b]thiophene-3-carboxylic acid amide,
(Compound D122) 2-(3-Cyclohexyl-ureido]-6-oxo-4,5,6,7-tetrahydro-benzo[b]thiophene-3-carboxylic acid amide,
(Compound D123) N-(3-Carbamoyl-6-oxo-4,5,6,7-tetrahydro-benzo[b]thiophen-2-yl)-oxalamic acid methyl ester,
(Compound D124) 2-[3-(4-Bromo-phenyl)-ureido]-4,5,6,7-tetrahydro-benzo[b]thiophene-3-carboxylic acid amide,
(Compound D125) 2-{[(1,3-Benzodioxol-5-ylamino)carbonyl]amino}-4,7-dihydro-5H-spiro[1-benzothiophene-6,2′-[1,3]dioxolane]-3-carboxamide,
(Compound D126) 2-{[(2-Methylcyclopropyl)carbonyl]amino}-4,7-dihydro-5H-spiro[1-benzothiophene-6,2′-[1,3]dioxolane]-3-carboxamide,
(Compound D127) 2-[(2-Methyl-cyclopropanecarbonyl)-amino]-(6-oxo-ethylenylketal)-4,5,6,7-tetrahydro-benzo[b]thiophene-3-carboxylic acid amide,
(Compound D128) 2-(Cyclopropanecarbonyl-amino)-6,6-difluoro-4,5,6,7-tetrahydro-benzo[b]thiophene-3-carboxylic acid amide,
(Compound D129) 2-[3-(3-Carbamoyl-4,5,6,7-tetrahydro-benzo[b]thiophen-2-yl)-ureido]-3-methyl-butyric acid methyl ester,
(Compound D130) N-(3-Carbamoyl-4,5,6,7-tetrahydro-benzo[b]thiophen-2-yl)-succinamic acid,
(Compound D131) 2-(3-o-Tolyl-ureido)-4,5,6,7-tetrahydro-benzo[b]thiophene-3-carboxylic acid amide,
(Compound D132) 3-Carbamoyl-2-[3-(4-methoxy-phenyl)-ureido]-4,5,6,7-tetrahydro-benzo[b]thiophene-6-carboxylic acid ethyl ester,
(Compound D133) Cyclopropanecarboxylic acid (3-carbamimidoyl-4,5,6,7-tetrahydro-benzo[b]thiophen-2-yl)-amide,
(Compound D134) 2-(Cyclopropanecarbonyl-amino)-6-hydroxyimino-4,5,6,7-tetrahydro-benzo[b]thiophene-3-carboxylic acid amide,
(Compound D135) 2-(3-Benzyl-ureido)-4,5,6,7-tetrahydro-benzo[b]thiophene-3-carboxylic acid amide,
(Compound D136) 2-[3-(2-Chloro-5-trifluoromethyl-phenyl)-ureido]4,5,6,7-tetrahydro-benzo[b]thiophene-3-carboxylic acid amide,
(Compound D137) 2-[3-(3,5-Bis-trifluoromethyl-phenyl)-ureido]-4,5,6,7-tetrahydro-benzo[b]thiophene-3-carboxylic acid amide,
(Compound D138) 2-[3-(4-Chloro-3-trifluoromethyl-phenyl)-ureido]-4,5,6,7-tetrahydro-benzo[b]thiophene-3-carboxylic acid amide,
(Compound D139) 2-[3-(3,5-Dimethoxy-phenyl)-ureido]-4,5,6,7-tetrahydro-benzo[b]thiophene-3-carboxylic acid amide,
(Compound D140) 2-[3-(4-Iodo-phenyl)-ureido]-6-hydroxy-4,5,6,7-tetrahydro-benzo[b]thiophene-3-carboxylic acid amide,
(Compound D141) 6-Hydroxy-2-[3-(4-methoxy-phenyl)-ureido]-4,5,6,7-tetrahydro-benzo[b]thiophene-3-carboxylic acid amide,
(Compound D142) 2-[3-(4-Fluoro-phenyl)-ureido]-6-hydroxy-4,5,6,7-tetrahydro-benzo[b]thiophene-3-carboxylic acid amide,
(Compound D143) 6-Hydroxy-2-(3-phenyl-ureido)-4,5,6,7-tetrahydro-benzo[b]thiophene-3-carboxylic acid amide,
(Compound D144) 2-(Cyclopropanecarbonyl-amino)-6-methoxy-4,5,6,7-tetrahydro-benzo[b]thiophene-3-carboxylic acid amide,
(Compound D145) 2-[3-(3-Bromo-phenyl)-ureido]-4,5,6,7-tetrahydro-benzo[b]thiophene-3-carboxylic acid amide,
(Compound D146) 3-Carbamoyl-2-(cyclopropanecarbonyl-amino)-4,5,6,7-tetrahydro-benzo[b]thiophene-6-carboxylic acid ethyl ester,
(Compound D147) 2-(3-Cyclopropyl-thioureido)-4,5,6,7-tetrahydro-benzo[b]thiophene-3-carboxylic acid amide,
(Compound D148) 2-[3-(3-Chloro-phenyl)-ureido]-4,5,6,7-tetrahydro-benzo[b]thiophene-3-carboxylic acid amide,
(Compound D149) 2-[3-(3-Trifluoromethyl-phenyl)-ureido]-4,5,6,7-tetrahydro-benzo[b]thiophene-3-carboxylic acid amide,
(Compound D150) 2-(3-Pyridin-3-yl-thioureido)-4,5,6,7-tetrahydro-benzo[b]thiophene-3-carboxylic acid amide,
(Compound D151) 5-Methyl-isoxazole-4-carboxylic acid (3-Carbamoyl-4,5,6,7-tetrahydro-benzo[b]thiophen-2-yl)-amide,
(Compound D152) 2-({[(2-Furylmethyl)amino]carbonyl}amino)-4,7-dihydro-5H-spiro[1-benzothiophene-6,2′-[1,3]dioxolane]-3-carboxamide,
(Compound D153) 2-[3-(2-Methoxy-5-nitro-phenyl)-ureido]-4,5,6,7-tetrahydro-benzo[b]thiophene-3-carboxylic acid amide,
(Compound D154) 2-(3-Isopropyl-ureido)-6-methyl-4,5,6,7-tetrahydro-benzo[b]thiophene-3-carboxylic acid amide,
(Compound D155) 2-(3-Cyclopropyl-ureido)-6-methyl-4,5,6,7-tetrahydro-benzo[b]thiophene-3-carboxylic acid amide,
(Compound D156) 6-Methyl-2-(2-methyl-butyrylamino)-4,5,6,7-tetrahydro-benzo[b]thiophene-3-carboxylic acid amide,
(Compound D157) 6-Hydroxy-2-[3-(4-methylsulfanyl-phenyl)-ureido]-4,5,6,7-tetrahydro-benzo[b]thiophene-3-carboxylic acid amide, (Compound D158) 2-(Cyclobutanecarbonyl-amino)-6-ethyl-4,5,6,7-tetrahydro-benzo[b]thiophene-3-carboxylic acid amide,
(Compound D159) 2-(Cyclopropanecarbonyl-amino)-6-propyl-4,5,6,7-tetrahydro-benzo[b]thiophene-3-carboxylic acid amide,
(Compound D160) 2-(Cyclopropanecarbonyl-amino)-6-methyl-4-oxo-4,5,6,7-tetrahydro-benzo[b]thiophene-3-carboxylic acid amide,
(Compound D161) 2-(Cyclopropanecarbonyl-amino)-6-methyl-7-oxo-4,5,6,7-tetrahydro-benzo[b]thiophene-3-carboxylic acid amide,
(Compound D162) 2-[3-(2-Methyl-3-nitro-phenyl)-ureido]-4,5,6,7-tetrahydro-benzo[b]thiophene-3-carboxylic acid amide,
(Compound D163) 2-{3-[4-(2-Morpholin-4-yl-ethoxy)-naphthalen-1-yl]-ureido]-4,5,6,7-tetrahydro-benzo[b]thiophene-3-carboxylic acid amide,
(Compound D164) 2-[3-(3-Chloro-4-fluoro-phenyl)-ureido]-4,5,6,7-tetrahydro-benzo[b]thiophene-3-carboxylic acid amide,
(Compound D165) 2-[3-(4-Isopropyl-phenyl)-ureido]-4,5,6,7-tetrahydro-benzo[b]thiophene-3-carboxylic acid amide,
(Compound D166) 2-[3-(3-Carbamoyl-4,5,6,7-tetrahydro-benzo[b]thiophen-2-yl)-ureido]-3-(4-chloro-phenyl)-propionic acid methyl ester,
(Compound D167) 2-[3-(3-Nitro-phenyl)-ureido]-4,5,6,7-tetrahydro-benzo[b]thiophene-3-carboxylic acid amide,
(Compound D168) 2-(3-Cyclopropyl-ureido)-6-hydroxy-4,5,6,7-tetrahydro-benzo[b]thiophene-3-carboxylic acid amide,
(Compound D169) 6-Hydroxy-2-[3-(4-trifluoromethoxy-phenyl)-ureido]-4,5,6,7-tetrahydro-benzo[b]thiophene-3-carboxylic acid amide,
(Compound D170) 2-[3-(4-Bromo-phenyl)-ureido]-6-hydroxy-4,5,6,7-tetrahydro-benzo[b]thiophene-3-carboxylic acid amide,
(Compound D171) 2-[3-(4-Methoxy-phenyl)-ureido]-4,5,6,7-tetrahydro-benzo[b]thiophene-3-carboxylic acid amide,
(Compound D172) 2-(Cyclopropanecarbonyl-amino)-7-oxo-4,5,6,7-tetrahydro-benzo[b]thiophene-3-carboxylic acid amide,
(Compound D173) 6-Hydroxy-2-[3-(4-trifluoromethyl-phenyl)-ureido]-4,5,6,7-tetrahydro-benzo[b]thiophene-3-carboxylic acid amide,
(Compound D174) 2-(3-Cyclohexyl-ureido)-6-hydroxy-4,5,6,7-tetrahydro-benzo[b]thiophene-3-carboxylic acid amide,
(Compound D175) 2-({[(4-Iodophenyl)amino]carbonyl}amino)-4,7-dihydro-5H-spiro[1-benzothiophene-6,2′-[1,3]dioxolane]-3-carboxamide,
(Compound D176) 2-({[(4-Methoxyphenyl)amino]carbonyl}amino)-4,7-dihydro-5H-spiro[1-benzothiophene-6,2′-[1,3]dioxolane]-3-carboxamide,
(Compound D177) Benzyl-3-(aminocarbonyl)-4,7-dihydro-5H-spiro[1-benzothiophene-6,2′-[1,3]dioxolan]-2-ylcarbamate,
(Compound D178) 2-({[(4-Fluorophenyl)amino]carbonyl}amino)-4,7-dihydro-5H-spiro[1-benzothiophene-6,2′-[1,3]dioxolane]-3-carboxamide,
(Compound D179) 2-[3-(3-Methoxy-phenyl)-ureido]-4,5,6,7-tetrahydro-benzo[b]thiophene-3-carboxylic acid amide,
(Compound D180) 2-[3-(2-Nitro-phenyl)-ureido]-4,5,6,7-tetrahydro-benzo[b]thiophene-3-carboxylic acid amide,
(Compound D181) 2-[3-(3,5-Dichloro-phenyl)-ureido]-4,5,6,7-tetrahydro-benzo[b]thiophene-3-carboxylic acid amide,
(Compound D182) 2-[3-(3,4,5-Trimethoxy-phenyl)-ureido]-4,5,6,7-tetrahydro-benzo[b]thiophene-3-carboxylic acid amide,
(Compound D183) 2-[3-(2,4-Difluoro-phenyl)-ureido]-4,5,6,7-tetrahydro-benzo[b]thiophene-3-carboxylic acid amide,
(Compound D184) 2-[3-(4-Nitro-phenyl)-ureido]-4,5,6,7-tetrahydro-benzo[b]thiophene-3-carboxylic acid amide,
(Compound D185) 2-(Cyclopropanecarbonyl-amino)-4,5,6,7-tetrahydro-benzo[b]thiophene-3-carboxylic acid methylamide,
(Compound D186) 2-(Cyclopentanecarbonyl-amino)-6-methyl-4,5,6,7-tetrahydro-benzo[b]thiophene-3-carboxylic acid amide,
(Compound D187) 2-(Cyclobutanecarbonyl-amino)-6-methyl-4,5,6,7-tetrahydro-benzo[b]thiophene-3-carboxylic acid amide,
(Compound D188) 2-(3-Isopropyl-ureido)-4,5,6,7-tetrahydro-benzo[b]thiophene-3-carboxylic acid amide,
(Compound D189) 6-Ethyl-2-[2-(4-nitro-phenyl)-acetylamino]-4,5,6,7-tetrahydro-benzo[b]thiophene-3-carboxylic acid amide,
(Compound D190) 6-Methyl-2-[2-(4-nitro-phenyl)-acetylamino]-4,5,6,7-tetrahydro-benzo[b]thiophene-3-carboxylic acid amide,
(Compound D191) 2-(Cyclopropanecarbonyl-amino)-4,5,6,7-tetrahydro-benzo[b]thiophene-3-carboxylic acid propylamide,
(Compound D192) 2-[3-(2-Methoxy-benzyl)-ureido]-4,5,6,7-tetrahydro-benzo[b]thiophene-3-carboxylic acid amide,
(Compound D193) 2-(Cyclopentanecarbonyl-amino)-6-phenyl-4,5,6,7-tetrahydro-benzo[b]thiophene-3-carboxylic acid amide,
(Compound D194) 2-(Cyclopentanecarbonyl-amino)-6-ethyl-4,5,6,7-tetrahydro-benzo[b]thiophene-3-carboxylic acid amide,
(Compound D195) (5-Amino-pentyl)-carbamic acid 3-carbamoyl-2-(cyclopropanecarbonyl-amino)-4,5,6,7-tetrahydro-benzo[b]thiophen-6-yl ester,
(Compound D196) 2-(Cyclopropanecarbonyl-amino)-5,6-dihydro-4H-cyclopenta[b]thiophene-3-carboxylic acid amide,
(Compound D197) 6-Hydroxy-2-(2-hydroxy-acetylamino)-4,5,6,7-tetrahydro-benzo[b]thiophene-3-carboxylic acid amide,
(Compound D198) {5-[3-Carbamoyl-2-(cyclopropanecarbonyl-amino)-4,5,6,7-tetrahydro-benzo[b]thiophen-6-yloxycarbonylamino]-pentyl}-carbamic acid tert-butyl ester,
(Compound D199) 2-[(Anilinocarbonyl)amino]-4,7-dihydro-5H-spiro[1-benzothiophene-6,2′-[1,3]dioxolane]-3-carboxamide,
(Compound D200) 2-({[(4-Ethoxyphenyl)amino]carbonyl}amino)-4,7-dihydro-5H-spiro[1-benzothiophene-6,2′-[1,3]dioxolane]-3-carboxamide,
(Compound D201) 2-(Cyclopropanecarbonyl-amino)-6-hydroxy-6-methyl-4,5,6,7-tetrahydro-benzo[b]thiophene-3-carboxylic acid amide,
(Compound D202) 6-Hydroxy-2-[(2-methyl-cyclopropanecarbonyl)-amino]-4,5,6,7-tetrahydro-benzo[b]thiophene-3-carboxylic acid amide,
(Compound D203) 6-Hydroxyimino-2-[3-(4-methoxy-phenyl)-ureido]-4,5,6,7-tetrahydro-benzo[b]thiophene-3-carboxylic acid amide,
(Compound D204) 2-[(2-Methyl-cyclopropanecarbonyl)-amino]-6-oxo-4,5,6,7-tetrahydro-benzo[b]thiophene-3-carboxylic acid amide,
(Compound D205) (3-Chloro-4-fluoro-phenyl)-carbamic acid 3-carbamoyl-2-(cyclopropanecarbonyl-amino)-4,5,6,7-tetrahydro-benzo[b]thiophen-6-yl ester,
(Compound D206) 6-Methyl-2-[(2-methyl-cyclopropanecarbonyl)-amino]-4,5,6,7-tetrahydro-benzo[b]thiophene-3-carboxylic acid amide,
(Compound D207) 2-[3-(4-Ethoxy-phenyl)-ureido]-6-hydroxy-4,5,6,7-tetrahydro-benzo[b]thiophene-3-carboxylic acid amide,
(Compound D208) 2-[3-(3-Carbamoyl-4,5,6,7-tetrahydro-benzo[b]thiophen-2-yl)-ureido]-4-methylsulfanyl-butyric acid methyl ester,
(Compound D209) 6-Hydroxy-2-{3-[4-(1-hydroxyethyl)-phenyl]-ureido}-4,5,6,7-tetrahydro-benzo[b]thiophene-3-carboxylic acid amide,
(Compound D210) 2-(Cyclopropanecarbonyl-amino)-4,5,6,7,8,9-hexahydro-cycloocta[b]thiophene-3-carboxylic acid amide,
(Compound D211) 6-Hydroxy-2-(3-isopropyl-ureido)-4,5,6,7-tetrahydro-benzo[b]thiophene-3-carboxylic acid amide,
(Compound D212) 2-(3-Benzo[1,3]dioxol-5-yl-ureido)-6-hydroxy-4,5,6,7-tetrahydro-benzo[b]thiophene-3-carboxylic acid amide,
(Compound D213) Phenyl-3-(aminocarbonyl)-4,7-dihydro-5H-spiro[1-benzothiophene-6,2′-[1,3]dioxolan]-2-ylcarbamate,
(Compound D214) 2-(Cyclopropanecarbonylamino)-6-methoxyimino-4,5,6,7-tetrahydro-benzo[b]thiophene-3-carboxylic acid amide,
(Compound D215) 6-Benzyloxyimino-2-(cyclopropanecarbonylamino)-4,5,6,7-tetrahydro-benzo[b]thiophene-3-carboxylic acid amide,
(Compound D216) 2-(Cyclopropanecarbonyl-amino)-4,5,6,7-tetrahydro-benzo[b]thiophene-3-sulfonic acid,
(Compound D217) 2-(3-exo-Bicyclo[2.2.1]hept-2-yl-methyl-ureido]-(6-oxo-ethylenylketal)-4,5,6,7-tetrahydro-benzo[b]thiophene-3-carboxylic acid amide,
(Compound D218) 2-[3-(4-tert-Butyl-cyclohexyl)-ureido]-(6-oxo-ethylenylketal)-4,5,6,7-tetrahydro-benzo[b]thiophene-3-carboxylic acid amide,
(Compound D219) 4-[3-(3-Carbamoyl-6-oxo-ethylenylketal-4,5,6,7-tetrahydro-benzo[b]thiophen-2-yl)-ureido]-piperidine-1-carboxylic acid ethyl ester,
(Compound D220) 2-[3-(1-Benzyl-piperidin-4-yl)-ureido]-6-oxo-ethylenylketal-4,5,6,7-tetrahydro-benzo[b]thiophene-3-carboxylic acid amide,
(Compound D221) 2-[3-(2-Hydroxy-ethyl)-ureido]-6-oxo-ethylenylketal-4,5,6,7-tetrahydro-benzo[b]thiophene-3-carboxylic acid amide,
(Compound D222) 2-[3-(2,3-Dihydroxy-propyl)-ureido]-6-oxo-ethylenylketal-4,5,6,7-tetrahydro-benzo[b]thiophene-3-carboxylic acid amide,
(Compound D223) 2-[3-(R(−)-1-Cyclohexyl-ethyl)-ureido]-6-oxo-ethylenylketal-4,5,6,7-tetrahydro-benzo[b]thiophene-3-carboxylic acid amide,
(Compound D224) 6-Oxo-ethylenylketal-2-[3-(2-thiophen-2-yl-ethyl)-ureido]-4,5,6,7-tetrahydro-benzo[b]thiophene-3-carboxylic acid amide,
(Compound D225) 6-Oxo-ethylenylketal-2-[3-(2-pyridin-2-yl-ethyl)-ureido]-4,5,6,7-tetrahydro-benzo[b]thiophene-3-carboxylic acid amide,
(Compound D226) 2-[3-(3-Morpholin-4-yl-propyl)-ureido]-6-oxo-ethylenylketal-4,5,6,7-tetrahydro-benzo[b]thiophene-3-carboxylic acid amide,
(Compound D227) 2-(3-Bicyclo[2.2.1]hept-2-yl-ureido)-6-oxo-ethylenylketal-4,5,6,7-tetrahydro-benzo[b]thiophene-3-carboxylic acid amide,
(Compound D228) 6-Oxo-ethylenylketal-2-[3-(1R,2R,3R,5S-2,6,6-trimethyl-bicyclo[3.1.1]hept-3-yl)-ureido]-4,5,6,7-tetrahydro-benzo[b]thiophene-3-carboxylic acid amide,
(Compound D229) 6-Oxo-ethylenylketal-2-[3-(1S,2S,3S,5R-2,6,6-trimethyl-bicyclo[3.1.1]hept-3-yl)-ureido]-4,5,6,7-tetrahydro-benzo[b]thiophene-3-carboxylic acid amide,
(Compound D230) 2-[3-(2-Cyclohex-1-enyl-ethyl)-ureido]-6-oxo-ethylenylketal-4,5,6,7-tetrahydro-benzo[b]thiophene-3-carboxylic acid amide,
(Compound D231) 2-[3-(3-Chloro-4-methyl-phenyl)-ureido]-(6-oxo-ethylenylketal)-4,5,6,7-tetrahydro-benzo[b]thiophene-3-carboxylic acid amide,
(Compound D232) 2-[3-(3-Chloro-2-methyl-phenyl)-ureido]-(6-oxo-ethylenylketal)-4,5,6,7-tetrahydro-benzo[b]thiophene-3-carboxylic acid amide,
(Compound D233) 2-(3-Benzo[1,3]dioxol-5-yl-ureido)-3-carbamoyl-4,5,6,7-tetrahydro-benzo[b]thiophene-6-carboxylic acid ethyl ester,
(Compound D234) 3-Carbamoyl-2-[3-(4-methylsulfanyl-phenyl)-ureido]-4,5,6,7-tetrahydro-benzo[b]thiophene-6-carboxylic acid ethyl ester,
(Compound D235) (3-Carbamoyl-6,6-ethylenedioxy-4,5,6,7-tetrahydro-benzo[b]thiophen-2-yl)-carbamic acid phenyl ester,
(Compound D236) 2-[3-(S(−)-1-Cyclohexyl-ethyl)-ureido]-6-oxo-ethylenylketal-4,5,6,7-tetrahydro-benzo[b]thiophene-3-carboxylic acid amide,
(Compound D237) 2-{(3-Chloro-4-fluorophenyl)-ureido]-4,7-dihydro-5H-spiro[1-benzothiophene-6,2′-[1 3]dioxolane]-3-carboxamide,
(Compound D238) 4-[({[7′-(aminocarbonyl)spiro[1,3-dioxolane-2,3′-[9]thiabicyclo[4.3.1]deca[6(10),7]dien]-8′-yl]amino}carbonyl)amino]-1-hydroxypyridinium,
(Compound D239) 2-{(3,4,5-Trimethoxyphenyl)-ureido]-4,7-dihydro-5H-spiro[1-benzothiophene-6,2′-[1,3]dioxolane]-3-carboxamide,
(Compound D240) (3-Carbamoyl-6-oxo-4,5,6,7-tetrahydrobenzo[b]thiophen-2-yl)-carbamic acid benzyl ester,
(Compound D241) 2-{[(3-Methylsulfanylphenyl)amino]carbonyl)amino}-4,7-dihydro-5H-spiro[1-benzothiophene-6,2′-[1,3]dioxolane]-3-carboxamide,
(Compound D242) 2-[({[4-Difluoromethoxyphenyl]amino}carbonyl)amino]-4,7-dihydro-5H-spiro[1-benzothiophene-6,2′-[1,3]dioxolane]-3-carboxamide,
(Compound D243) 2-[({[3-Trifluoromethylsulfanylphenyl]amino}carbonyl)amino]-4,7-dihydro-5H spiro[1-benzothiophene-6,2′-[1,3]dioxolane]-3-carboxamide,
(Compound D244) 6-Hydroxy-2-[3-(3-methylsulfanyl-phenyl)-ureido]-4,5,6,7-tetrahydro-benzo[b]thiophene-3-carboxylic acid amide,
(Compound D245) 2-[3-(4-Difluoromethoxy-phenyl)-ureido]-6-hydroxy-4,5,6,7-tetrahydro-benzo[b]thiophene-3-carboxylic acid amide,
(Compound D246) 2-[3-(2,4-difluoro-phenyl)-ureido]-(6-oxo-ethylenylketal)-4,5,6,7-tetrahydro-benzo[b]thiophene-3-carboxylic acid amide or 8′-({[(2,4-difluorophenyl)amino]carbonyl}amino)spiro[1,3-dioxolane-2,3′-[9]thiabicyclo[4.3.1]deca[6(10),7]diene]-7′-carboxamide,
(Compound D247) 2-[3-(2,4-Difluoro-phenyl)-ureido]-6-oxo-4,5,6,7-tetrahydro-benzo[b]thiophene-3-carboxylic acid amide,
(Compound D248) 2-[3-(3-Chloro-4-methyl-phenyl)-ureido]-6-hydroxy-4,5,6,7-tetrahydro-benzo[b]thiophene-3-carboxylic acid amide,
(Compound D249) 2-[3-(3-Chloro-2-methyl-phenyl)-ureido]-6-hydroxy-4,5,6,7-tetrahydro-benzo[b]thiophene-3-carboxylic acid amide,
(Compound D250) 2-[3-(2-Methylsulfanyl-phenyl)-ureido]-(6-oxo-ethylenylketal)-4,5,6,7-tetrahydro-benzo[b]thiophene-3-carboxylic acid amide,
(Compound D251) 2-[3-(2-Trifluoromethoxy-phenyl)-ureido]-(6-oxo-ethylenylketal)-4,5,6,7-tetrahydro-benzo[b]thiophene-3-carboxylic acid amide,
(Compound D252) 2-[3-(2-Difluoromethoxy-phenyl)-ureido]-(6-oxo-ethylenylketal)-4,5,6,7-tetrahydro-benzo[b]thiophene-3-carboxylic acid amide,
(Compound D253) 2-[3-(4-Trifluoromethylsulfanyl-phenyl)-ureido]-(6-oxo-ethylenylketal)-4,5,6,7-tetrahydro-benzo[b]thiophene-3-carboxylic acid amide,
(Compound D254) 6-Hydroxy-2-[3-(2-methylsulfanyl-phenyl)-ureido]-4,5,6,7-tetrahydro-benzo[b]thiophene-3-carboxylic acid amide,
(Compound D255) 6-Hydroxy-2-[3-(2-trifluoromethoxy-phenyl)-ureido]4,5,6,7-tetrahydro-benzo[b]thiophene-3-carboxylic acid amide,
(Compound D256) 2-[3-(2-Difluoromethoxy-phenyl)-ureido]-6-hydroxy-4,5,6,7-tetrahydro-benzo[b]thiophene-3-carboxylic acid amide,
(Compound D257) 6-Hydroxy-2-[3-(4-trifluoromethylsulfanyl-phenyl)-ureido]-4,5,6,7-tetrahydro-benzo[b]thiophene-3-carboxylic acid amide.

The present invention also comprises pharmaceutically active salts of these compounds, all stereoisomeric forms and regioisomeric forms of these compounds or prodrugs thereof.

Other aspects of the present invention relate to the heterobicyclic compounds disclosed herein, for instance 4,7-dihydro-5H-thieno[2,3-c]pyran derivatives or 4,5,6,7-tetrahydro-benzo[b]thiophene-3-carboxylic acid amides as outlined above in the general formula (I), for use as new pharmaceutically active agents, particularly for the prophylaxis and/or treatment of prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke virally or bacterially induced diseases or infections, especially infections induced by bacteria of the genus legionella, and especially legionnaires disease, or mycobateria-induced infections (including opportunistic infections) and diseases, especially mycobacteria induced meningitis, tuberculosis and leprosy, pharmaceutical compositions comprising these heterobicyclic compounds as active ingredients and methods for treating prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, virally and/or bacterially induced diseases, particularly mycobacteria-induced infections, in mammals, including humans, especially for the treatment of treatment of virally or bacterially induced diseases or infections, especially infections induced by bacteria of the genus legionella, and especially legionnaires disease, or mycobateria-induced infections (including opportunistic infections) and diseases, especially mycobacteria induced meningitis, tuberculosis and leprosy.

Surprisingly, it was found that the compounds according to general formula (I) as well as pharmaceutically acceptable salts of these compounds are effective against prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, virally and/or bacterially induced diseases, especially mycobacteria-induced infections and diseases at pharmaceutically acceptable concentrations while exhibiting enhanced metabolitic stability. It shall be stressed that the compounds which are excluded from claim 1 by disclaimer are herewith explicitly claimed for any pharmaceutical use thereof.

Additionally, the present invention relates to the use of the compounds of the present invention for the manufacturing of a pharmaceutical composition for the prophylaxis and/or treatment of prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke, virally and/or bacterially induced diseases, particularly those infections and diseases mentioned above.

The compounds of the present invention are effective against mycobacteria induced infections, particularly tuberculosis, but also e.g. leprosy and mycobacteria-induced meningitis. Mycobacteria which induce or cause these infectious diseases are members of the group comprising the tuberculous bacteria Mycobacterium tuberculosis, M. bovis, M. africanum and M. leprae as well as the non-tuberculous bacteria M. abscessus, M. avium, M. celatum, M. chelonae, M. fortuitum, M. genavense, M. gordonae, M. haemophilum, M. intracellulare, M. kansii, M. malmoense, M. marinum, M. scrofulaceum, M. simiae, M. szulgai, M. ulcerans and M. xenopi. Because of the outstanding clinical importance of tuberculosis, microbiologists have distinguished the so-called “Mycobacterium tuberculosis complex” consisting of Mycobacterium tuberculosis, M. bovis, and M. africanum from all other mycobacteria which form the group of the so-called “atypical mycobacteria” or “non-tuberculous mycobacteria (NTM)”.

The use of 4,5,6,7-tetrahydrobenzo[b]thiophene derivatives in the treatment of mycobacterial infections such as tuberculosis is described in the PCT patent application PCT/EP03/03697. The compounds described therein have been found to be effective in blocking the activity of mycobacterial protein serine/threonine kinases, particularly protein kinase G (PknG), which have been identified as an essential component involved in the persistence and enhanced survival of pathogenic mycobacterial within a macrophage cell line, and thereby provide a mode for the elimination of mycobacteria.

Mycobacterial protein serine/threonine kinases such as PknF, PknI, PknJ, PknL, and particularly protein kinase G (PknG), have been identified as an essential component involved in the persistence and enhanced survival of pathogenic mycobacteria within a macrophage cell line. Furthermore, it could be demonstrated that the activity of PknG is an essential factor for virulence of mycobacteria. In accordance with the present invention, compounds have been found which are blocking the activity of PknG in a submicromolar range thus showing that PknG, PknF, PknI, PknJ, and PknL are suitable targets for recognising diseases, monitoring diseases, and controlling therapy of diseases related to mycobacterial infections. These compounds (inhibitors) were able to induce efficient degradation of mycobacteria within host cells so that the present invention provides a novel mode for elimination of mycobacteria.

By means of an alkaline phosphatase secretion assay for PknG for different PhoA fusion constructs, it could be shown that PknG is a secretory protein that is secreted outside the mycobacterial cells. The secreted PknG can phosphorylate host cell proteins that might be critical in survival of mycobacterium in host cells.

Additionally, biologically active 4,7-dihydro-5-H-thieno[2,3-c]pyran and 4,7-dihydro-5-H-thieno[2,3-c]thiopyran derivatives are described in Biorg. Med. Chem. Letters 2002, 12, 1897-1900, in which compounds which inhibit TNF-alpha-production are described, in J. Med. Chem. 2002, 45, 4443-4459, in which compounds are described which act as protein-tyrosine phosphatase 1B (PTP1B) inhibitors, or in Japanese patent JP 2002308870, in which compounds are described, which act as Staphylococcus aureus inhibitors. Further derivatives are described in Armyanskii Khimicheskii Zhurnal 1987, 40(9), 581-7. These references do not disclose any PkNG inhibitory activity for these compounds.

In WO 01/98290 thiophene derivatives are described as active kinase inhibitors.

One important feature for pharmaceutical active agents in general is that these agents have a high degree of metabolitic stability. It was found that the compounds described in PCT/EP03/03697, while being pharmaceutically active as PkNG inhibitors, left room for further increase of metabolitic stability.

The present invention also provides a method for preventing or treating infections and diseases, especially virally or bacterially induced diseases or infections, more specially infections induced by bacteria of the genus legionella such as legionaires disease, mycobacteria-induced infections (including opportunistic infections) in mammals (including humans), which method comprises administering to the mammal an pharmaceutically effective amount of the compounds of the present invention to treat a infection or disease. Especially, the method is used for the treatment of tuberculosis, but also for other mycobacteria-induced infections like leprosy or mycobacteria-induced meningitis.

Further aspects of the present invention relate to the use of the compounds of general formula (I) for the preparation of a pharmaceutical composition useful for prophylaxis and/or treatment of infectious diseases including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, and stroke.

Infectious Diseases Including Opportunistic Infections

In yet another aspect of the present invention, the compounds according to the general formula (I) are for the preparation of a pharmaceutical composition for the prophylaxis and/or treatment of infectious diseases, including opportunistic diseases and opportunistic infections. The term infectious diseases comprises infections caused by viruses, bacteria, prions, fungi, and/or parasites.

Especially, virally induced infectious diseases, including opportunistic diseases are addressed. In a preferred embodiment of this aspect, the virally induced infectious diseases, including opportunistic diseases, are caused by retroviruses, human endogenous retroviruses (HERVs), hepadnaviruses, herpesviruses, flaviviridae, and/or adenoviruses. Preferably, the retroviruses are selected from lentiviruses or oncoretroviruses, wherein the lentivirus is preferably selected from the group comprising: HIV-1, HIV-2, feline immunodeficiency virus (FIV), bovine immunodeficiency virus (BIV), sivian immunodeficiency viruses (SIVs), chimeras of HIV and SIV (SHIV), caprine arthritis encephalitis virus (CAEV), visna/maedi virus (VMV) or equine infectious anemia virus (EIAV), preferably HIV-1 and HIV-2, and the oncoretrovirus is preferably selected from HTLV-I, HTLV-II or bovine leukemia virus (BLV), preferably HTLV-I and HTLV-II.

The hepadnavirus is preferably selected from HBV, ground squirrel hepatitis virus (GSHV) or woodchuck hepatitis virus (WHV), preferably HBV, the herpesvirus is selected from the group comprising: Herpes simplex virus I (HSV I), herpes simplex virus II (HSV II), Epstein-Barr virus (EBV), varicella zoster virus (VZV), human cytomegalovirus (HCMV) or human herpesvirus 8 (HHV-8), preferably HCMV, and the flaviviridae is selected from HCV, West nile or Yellow Fever.

It is to be understood, that all the viruses mentioned above, also comprise drug resistant virus strains.

Examples of infective diseases are AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever.

Bacterial Infections

As described above, the compounds according to the general formula (I) are also useful for the preparation of a pharmaceutical composition for prophylaxis and/or treatment of bacterially induced infectious diseases, including opportunistic diseases and opportunistic infections, wherein the bacterially induced infectious diseases, including opportunistic diseases, are selected from tuberculosis, leprosy or mycobacteria-induced meningitis. One advantage of the inventive compounds disclosed herein is there use against drug resistant bacterial strains.

Prion Diseases

Another aspect of the present invention is directed to the use of at least one compound of the general formula (I) and/or pharmaceutically acceptable salts thereof for prophylaxis and/or treatment of prion diseases.

Prions are infectious agents, which do not have a nucleic acid genome. It seems that a protein alone is the infectious agent. A prion has been defined as “small proteinaceous infectious particle, which resists inactivation, by procedures that modify nucleic acids”. The discovery that proteins alone can transmit an infectious disease has come as a considerable surprise to the scientific community. Prion diseases are often called “transmissible spongiform encephalopathies”, because of o the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders. Examples for prion diseases acquired by exogenous infection are the Bovine spongiform encephalitis (BSE) of cattle and the new variant of Creutzfeld-Jakob disease (vCJD) caused by BSE as well as scrapie of animals. Examples of human prion diseases include kuru, sporadic Creutzfeldt-Jakob disease (sCJD), familial CJD (fCJD), iatrogenic CJD (iCJD), Gerstmann-Sträussler-Scheinker (GSS) disease, fatal familial insomnia (FFI), and especially the new variant CJD (nvCJD or vCJD).

The name “prion” is used to describe the causative agents, which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence, that are folded into molecules with dramatically different tertiary structures. The normal cellular isoform of the prion protein (PrP^{C}) has a high a-helix content, a low b-sheet content, and is sensitive to protease digestion. The abnormal, disease-causing isoform (PrP^{Sc})has a lower a-helix content, a much higher b-sheet content, and is much more resistant to protease digestion.

As used herein the term “prion diseases” refers to transmissible spongiform encephalopathies. Examples for prion diseases comprise Scrapie (sheep, goat), TME (transmissible mink encephalopathy; mink), CWD (chronic wasting disease; muledeer, deer, elk), BSE (bovine spongiform encephalopathy; cows, cattles), CJD (Creutzfeld-Jacob Disease), vCJD, GSS (Gerstmann-Sträussler-Scheinker syndrome), FFI (Fatal familial Insomnia), Kuru, and Alpers Syndrome. Preferred are BSE, vCJD, and CJD.

Immunological Diseases

Another aspect of the present invention is directed to the use of at least one compound of the general formula (I) and/or pharmaceutically acceptable salts thereof for prophylaxis and/or treatment of immunological diseases, neuroimmunological diseases, and autoimmune diseases.

Immunological diseases are, for instance, asthma and diabetes, rheumatic and autoimmune diseases, AIDS, rejection of transplanted organs and tissues (cf. below), rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, and other manifestations of allergic disease, as well as uncommon problems such as primary immunodeficiencies, including antibody deficiency states, cell mediated immunodeficiencies (e.g., severe combined immunodeficiency, DiGeorge syndrome, Hyper-IgE syndrome, Wiskott-Aldrich syndrome, ataxia-telangiectasia), immune mediated cancers, and white cell defects.

In autoimmune diseases, such as systemic lupus erythematosus, rheumatoid arthritis (RA), multiple sclerosis (MS), immune-mediated or type 1 diabetes mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, and Hashimoto's disease, dermatomyositis, goodpastture syndrome, myasthenia gravis pseudoparalytica, ophtalmia sympatica, phakogene uveitis, chronical agressivce hepatitis, primary billiary cirrhosis, autoimunehemolytic anemy, Werlof disease, specific cells uncontrollably attack the body's own tissues and organs (autoimmunity), producing inflammatory reactions and other serious symptoms and diseases.

Hashimoto's thyroiditis is one of the most common autoimmune diseases. “Autoimmune disease” refers to a category of more than 80 chronic illnesses, each very different in nature, that can affect everything from the endocrine glands (like the thyroid) to organs like the kidneys, as well as to the digestive system.

There are many different autoimmune diseases, and they can each affect the body in different ways. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

Bipolar and Clinical Disorders

Another aspect of the present invention is directed to the use of at least one compound of the general formula (I) and/or pharmaceutically acceptable salts thereof for prophylaxis and/or treatment of bipolar and clinical disorders.

The term “bipolar and clinical disorders” shall refer to adjustment disorders, anxiety disorders, delirium, dementia, amnestic and other cognitive disorders, disorders usually first diagnosed in infancy, childhood, or adolescence, dissociative disorders, eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder, panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

Examples for anxiety disorders are: acute stress disorder, agoraphobia without history of panic disorder, anxiety disorder due to general medical condition, generalized anxiety disorder, obsessive-compulsive disorder, panic disorder with agoraphobia, panic disorder without agoraphobia, posttraumatic stress disorder, specific phobia, social phobia, substance-induced anxiety disorder.

Examples for delirium, dementia, amnestic and other cognitive disorders are: delirium due to a general medical condition, substance intoxication delirium, substance withdrawal delirium, delirium due to multiple etiologies, Alzheimer's, Creutzfeldt-Jakob disease, head trauma, Huntington's disease, HIV disease, Parkinson's disease, Pick's disease, substance-induced persisting, vascular, dementia due to other general medical conditions, dementia due to multiple etiologies, amnestic disorder due to a general medical condition, substance-induced persisting amnestic disorder.

Examples for disorders usually first diagnosed in infancy, childhood, or adolescence are: mental retardation, learning disorders, mathematics disorder, reading disorder, disorder of written expression, learning disorder, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder.

Examples for dissociative disorders are: dissociative amnesia, depersonalization disorder, dissociative fugue and dissociative identity disorder.

Examples for eating disorders are anorexia nervosa and bulimia nervosa.

Examples for mood disorders are: mood episodes, major depressive episode, hypomanic episode, manic episode, mixed episode, depressive disorders, dysthymic disorder, major depressive disorder, single episode, recurrent, bipolar disorders, bipolar I disorder, bipolar II disorder, cyclothymic disorder, mood disorder due to a general medical condition, substance-induced mood disorder.

Examples for schizophrenia and other psychotic disorders are: schizophreniform disorder, schizoaffective disorder, delusional disorder, brief psychotic disorder, shared psychotic disorder, psychotic disorder due to a general medical condition, delusions, hallucinations, substance-induced psychotic disorder.

Examples for sexual and gender identity disorders are: female sexual arousal disorder, orgasmic disorders, premature ejaculation, sexual pain disorders, dyspareunia, vaginismus, sexual dysfunction due to a general medical condition, female dyspareunia, female hypoactive sexual desire disorder, male erectile disorder, male hypoactive sexual desire disorder, male dyspareunia, other female sexual dysfunction, other male sexual dysfunction, substance-induced sexual dysfunction, sexual dysfunction, exhibitionism, fetishism, frotteurism, pedophilia, masochism, sadism, transvestic fetishism, voyeurism, paraphilia, gender identity disorder.

Examples for sleep disorders are: dyssomnias, breathing-related sleep disorder, circadian rhythm sleep disorder, hypersomnia, hypersomnia related to another mental disorder, insomnia, insomnia related to another mental disorder, narcolepsy, dyssomnia, parasomnias, nightmare disorder, sleep terror disorder, sleepwalking disorder, parasomnia.

Examples for somatoform disorders are: body dysmorphic disorder, conversion disorder, hypochondriasis, pain disorder, somatization disorder, undifferentiated somatoform disorder.

Examples for substance-related disorders are: alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic/disorder, withdrawal, withdrawal delirium, sexual dysfunction, sleep disorder.

Cardiovascular Diseases

The inventive compounds are also useful for prophylaxis and/or treatment of cardiovascular diseases such as adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Preferred are adult congenital heart disease, aneurysms, angina, angina pectoris, arrhythmias, cardiovascular disease prevention, cardiomyopathies, congestive heart failure, myocardial infarction, pulmonary hypertension, hypertrophic growth, restenosis, stenosis, thrombosis and arteriosclerosis.

Proliferative Disease

In yet another preferred embodiment, the cell proliferative disease is cancer, which is preferably selected from the group comprising:

The proliferation disorders and cancers are preferably selected from the group comprising adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

Preferred are the following cancer types: bladder, breast, central nervous system, colon, gastric, lung, kidney, melanoma, head and neck, ovarian, cervix, glioblastoma, pancreas, prostate, stomach, skin testis, leukemia, Hodgkin's lymphoma, liver and renal cancer.

Diabetes

In yet another preferred embodiment, said diabetes is selected from Type I diabetes or Type II diabetes.

Inflammation

In yet another preferred embodiment, said inflammation is mediated preferably by the cytokines TNF-α, IL-1β, GM-CSF, IL-6 and/or IL-8.

As described above, the compounds according to general formula (I) are pharmaceutically active agents for prophylaxis and/or treatment of inflammatory diseases. Thus, these compounds are used for the manufacture of a pharmaceutical formulation for prophylaxis and/or treatment of inflammations and inflammatory diseases in mammals, including humans.

Inflammatory diseases can emanate from infectious and non-infectious inflammatory conditions which may result from infection by an invading organism or from irritative, traumatic, metabolic, allergic, autoimmune, or idiopathic causes as shown in the following list.

| I. | Acute infections | |
|---|---|---|
| | A. Viral | B. Bacterial |

| II. | Noninfectious causes | |
|---|---|---|
| III. | Chronic (granulomatous) diseases | |
| | A. Bacterial | B. Spirochetal |
| | C. Mycotic (Fungal) | D. Idiopathic |

| IV. | Allergic, immune, and idiopathic disorders | |
|---|---|---|
| | A. Hypersensitivity reactions | |
| | B. Immune and idiopathic disorders | |
| V. | Miscellaneous inflammatory conditions | |
| | A. Parasitic infections | |
| | B. Inhalation causes: | Acute (thermal) injury |
| | | Pollution and inhalant allergy |
| | | Carcinogens |
| | C. Radiation injury: | Radionecrosis |

Thus, the compounds disclosed herein can be used for prophylaxis and/or treatment of inflammations caused by invading organisms such as viruses, bacteria, prions, and parasites as well as for prophylaxis and/or treatment of inflammations caused by irritative, traumatic, metabolic, allergic, autoimmune, or idiopathic reasons.

Consequently, the disclosed compounds are useful for prophylaxis and/or treatment of inflammatory diseases which are initiated or caused by viruses, parasites, and bacteria which are connected to or involved in inflammations.

The following bacteria are known to cause inflammatory diseases: mycoplasma pulmonis (causes e.g. chronic lung diseases (CLD), murine chronic respiratory disease), ureaplasma urealyticum (causes pneumonia in newborns), mycoplasma pneumoniae and chlamydia pneumoniae (cause chronic asthma), C. pneumoniae (causes atherosclerosis, pharyngitis to pneumonia with empyema, human coronary heart disease), Helicobacter pylori (human coronary heart disease, stomach ulcers).

The following viruses are known to cause inflammatory diseases: herpesviruses especially cytomegalovirus (causes human coronary heart disease).

The compounds disclosed herein are useful for prophylaxis and/or treatment of inflammatory diseases caused and/or induced and/or initiated and/or enhanced by the afore-mentioned bacteria or viruses.

Furthermore, the compounds of formula (I) are useful for prophylaxis and/or treatment of inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx.

Examples for inflammatory diseases of the central nervous system (CNS) are algal disorders, protothecosis, bacterial disorders, abscessation, bacterial meningitis, idiopathic inflammatory disorders, eosinophilic meningoencephalitis, feline polioencephalomyelitis, granulomatous meningoencephalomyelitis, meningitis, steroid responsive meningitis-arteritis, miscellaneous meningitis/meningoencephalitis, meningoencephalitis in greyhounds, necrotizing encephalitis, pug dog encephalitis, pyogranulomatous meningoencephalomyelitis, shaker dog disease, mycotic diseases of the CNS, parasitic encephalomyelitis, prion protein induced diseases, feline spongiform encephalopathy, protozoal encephalitis-encephalomyelitis, toxoplasmosis, neosporosis, sarcocystosis, encephalitozoonosis, trypanosomiasis, acanthamebiasis, babesiosis, leishmaniasis, rickettsial disorders, rocky mountain spotted fever, canine ehrlichiosis, salmon poisoning, viral disorders, aujeszky's disease, borna disease, canine herpes virus encephalomyelitis, canine distemper encephalomyelitis, canine distemper encephalomyelitis in immature animals, multifocal distemper encephalomyelitis in mature animals, old dog encephalitis, chronic relapsing encephalomyelitis, post-vaccinal canine distemper encephalitis, feline immunodeficiency virus, feline infectious peritonitis, feline leukemia virus, infectious canine hepatitis, La Crosse virus encephalitis, parvovirus encephalitis, rabies, post-vaccinal rabies, tick-borne encephalitis in dogs.

Examples for inflammatory rheumatic diseases are rheumatoid arthritis, scleroderma, lupus, polymyositis, dermatomyositis, psoriatic arthritis, ankylosing spondylitis, Reiters's syndrome, juvenile rheumatoid arthritis, bursitis, tendinitis (tendonitis), and fibromyositis.

Examples for inflammatory diseases of blood vessels are vasculitis, autoantibodies in vasculitis, microscopic polyangiitis, giant cell arteritis, Takayasu's arteritis, vasculitis of the central nervous system, thromboangiitis obliterans (Buerger's Disease), vasculitis secondary to bacterial, fungal, and parasitic infection, vasculitis and rheumatoid arthritis, vasculitis in systemic lupus erythematosus, vasculitis in the idiopathic inflammatory myopathies, relapsing polychondritis, systemic vasculitis in sarcoidosis, vasculitis and malignancy, and drug-induced vasculitis.

Examples for inflammatory diseases of the middle ear are acute suppurative otitis media, bullous myringitis, granular myringitis, and chronic suppurative otitis media, which can manifest as mucosal disease, cholesteatoma, or both.

Examples for inflammatory bowel diseases are ulcerative colitis, Crohn's disease.

Examples for inflammatory diseases of the skin are acute inflammatory dermatoses, urticaria (hives), spongiotic dermatitis, allergic contact dermatitis, irritant contact dermatitis, atopic dermatitis, erythemal multiforme (EM minor), Stevens-Johnson syndrome (SJS, EM major), toxic epidermal necrolysis (TEN), chronic inflammatory dermatoses, psoriasis, lichen planus, discoid lupus erythematosus, and acne vulgaris

Uveitis are inflammations located in and/or on the eye and may be associated with inflammation elsewhere in the body. In most circumstances, patients who have uveitis as part of a disease elsewhere in the body are aware of that illness. The majority of patients with uveitis do not have an apparent associated systemic illness. Causes of uveitis can be infectious causes, masquerade syndromes, suspected immune-mediated diseases, and/or syndromes confined primarily to the eye.

The following viruses are associated with inflammations: human immunodeficiency virus-I, herpes simplex virus, herpes zoster virus, and cytomegalovirus.

Bacterial or spirochetal caused, induced, initiated and/or enhanced inflammations are tuberculosis, leprosy, proprionobacterium, syphilis, Whipple's disease, leptospirosis, brucellosis, and lyme disease.

Parasitic (protozoan or helminthic) caused, induced, initiated and/or enhanced inflammations are toxoplasmosis, acanthameba, toxocariasis, cysticercosis, onchocerciasis.

Examples of inflammatory diseases caused, induced, initiated and/or enhanced by fungi are histoplasmosis, coccidioidomycosis, candidiasis, aspergillosis, sporotrichosis, blastomycosis, and cryptococcosis.

Masquerade syndromes are, for instance, leukemia, lymphoma, retinitis pigmentosa, and retinoblastoma.

Suspected immune-mediated diseases can be selected from the group comprising ankylosing spondylitis, Behcet's disease, Crohn's disease, drug or hypersensitivity reaction, interstitial nephritis, juvenile rheumatoid arthritis, Kawasaki's disease, multiple sclerosis, psoriatic arthritis, Reiter's syndrome, relapsing polychondritis, sarcoidosis, Sjogren's syndrome, systemic lupus erythematosus, ulcerative colitis, vasculitis, vitiligo, Vogt Koyanagi Harada syndrome.

Syndromes confined primarily to the eye are, for instance, acute multifocal placoid pigmentary epitheliopathy, acute retinal necrosis, birdshot choroidopathy, Fuch's heterochromic cyclitis, glaucomatocyclitic crisis, lens-induced uveitis, multifocal choroiditis, pars planitis, serpiginous choroiditis, sympathetic ophthalmia, and trauma.

Examples for inflammatory diseases of the larynx are gastroesophageal (laryngopharyngeal) reflux disease, pediatric laryngitis, acute laryngeal infections of adults, chronic (granulomatous) diseases, allergic, immune, and idiopathic disorders and miscellaneous inflammatory conditions.

Pediatric laryngitis is known as acute (viral or bacterial) infection such as laryngotracheitis (croup), supraglottitis (epiglottitis), diphtheria, and noninfectious causes are for example spasmodic croup and traumatic laryngitis.

Acute laryngeal infections of adults are, for instance, viral laryngitis, common upper respiratory infection, laryngotracheitis, herpes simplex, bacterial laryngitis, supraglottitis, laryngeal abscess, and gonorrhea.

Chronic (granulomatous) diseases can be selected from the group comprising bacterial diseases, tuberculosis, leprosy, scleroma, actinomycosis, tularemia, glanders, spirochetal (syphilis) diseases, mycotic (fungal) diseases, candidiasis, blastomycosis, histoplasmosis, coccidiomycosis, aspergillosis, idiopathic diseases, sarcoidosis, and Wegener's granulomatosis.

Allergic, immune, and idiopathic disorders are, for example, hypersensitivity reactions, angioedema, Stevens-Johnson syndrome, immune and idiopathic disorders, infections of the immunocompromised host, rheuatoid arthritis, systeic lupus erythematosus, cicatricial pemphigoid, relapsing polychondritis, Sjogren's syndrome, and amyloidosis.

Miscellaneous inflammatory conditions are, for instance, parasitic infections, trichinosis, leishmaniasis, schistosomiasis, syngamus laryngeus, inhalation laryngitis, acute (thermal) injury, pollution and inhalant allergy, carcinogens, radiation injury, radiation laryngitis, radionecrosis, vocal abuse, vocal-cord hemorrhage, muscle tension dysphonias, and contact ulcer and granuloma.

Transplant Rejection

Transplant rejection is when a transplant recipient's immune system attacks a transplanted organ or tissue. No two people (except identical twins) have identical tissue antigens. Therefore, in the absence of immunosuppressive drugs, organ and tissue transplantation would almost always cause an immune response against the foreign tissue (rejection), which would result in destruction of the transplant. Though tissue typing ensures that the organ or tissue is as similar as possible to the tissues of the recipient, unless the donor is an identical twin, no match is perfect and the possibility of organ/tissue rejection remains.

The inventive compounds of general formula (I) are used as immunosuppressive drugs and/or anti-rejection drugs in order to prevent transplant rejection.

One example of transplant rejection is the graft-versus-host-disease (GVHD) that can occur following bone marrow transplant. The donor's immune cells in the transplanted marrow make antibodies against the host's (transplant patient's) tissues and attack the patient's vital organs. Transplant rejections (also known as graft rejection or tissue/organ rejection) may commonly occur when tissue or organs, which need blood supply, are transplanted. Said organs comprise especially inner organs such as heart, heart-lungs, lungs, liver, kidney, pancreas, spleen, skin, tissue, bone marrow, spinal marrow, hormone producing glands, gonads and gonadal glands.

Neurodegenerative Diseases

Another aspect of the present invention is directed to the use of at least one compound of the general formula (I) and/or pharmaceutically acceptable salts thereof for prophylaxis and/or treatment of neurodegeneration and neurodegenerative disorders.

Among the hundreds of different neurodegenerative disorders, the attention has been given only to a handful, including Alzheimer disease, Parkinson disease, Huntington disease, and amyotrophic lateral sclerosis.

It is worth to mention that the same neurodegenerative process can affect different areas of the brain, making a given disease appear very different from a symptomatic standpoint.

Neurodegenerative disorders of the central nervous system (CNS) can be grouped into diseases of the cerebral cortex (Alzheimer disease), the basal ganglia (Parkinson disease), the brain-stem and cerebellum, or the spinal cord (amyotrophic lateral sclerosis).

Examples for neurodegeneration and neurodegenerative disorders are Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

According to a still further aspect, the present invention refers to pharmaceutical compositions comprising at least one compound according to the present invention as an active ingredient together with at least one pharmaceutically acceptable (i.e. non-toxic) carrier, excipient and/or diluent. The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical compositions according to the present invention containing at least one compound according to the present invention, e.g. one 4,7-dihydro-5H-thieno[2,3-c]thiopyran-3-carboxylic acid amide and/or one 4,5,6,7-tetrahydro-benzo[b]thiophene-3-carboxylic acid amide, and/or one 4,7-dihydro-5H-thieno[2,3-c]pyran derivative or analogues thereof as set out in general formula (I) in independent claim 1 or claims dependent thereon, and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95 weight % of the heterobicyclic compound and/or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Also included are solid form preparations which are intended to be converted, shortly before use, to liquid form preparations for either oral or parenteral administration. Such liquid forms include solutions, suspensions, and emulsions.

The compounds according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methyl cellulose, polyvinyl alcohols, or denatured gelatins or starch for holding or containing compositions comprising the active ingredient(s). Capsules with hard shells are typically made of blended of relatively high gel strength gelatins from bones or pork skin. The capsule itself may contain small amounts of dyes, opaquing agents, plasticisers and/or preservatives.

Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refers to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75 weight %, and more preferably from about 30 to about 60 weight %.

The term disintegrants refers to materials added to the composition to support break apart (disintegrate) and release the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscaramellose, alginates such as alginic acid and sodium alginate, clays such as bentonites, and effervescent mixtures. The amount of disintegrant in the composition may range from about 2 to about 20 weight % of the composition, more preferably from about 5 to about 10 weight %.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20 weight % of the composition, preferably from about 3 to about 10 weight %, and more preferably from about 3 to about 6 weight %.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5 weight % of the composition, preferably from about 0.5 to about 2 weight %, and more preferably from about 0.3 to about 1.5 weight % of the composition.

Glidents are materials that prevent caking of the components of the pharmaceutical composition and improve the flow characteristics of granulate so that flow is smooth and uniform. Suitable glidents include silicon dioxide and talc. The amount of glident in the composition may range from about 0.1 to about 5 weight % of the final composition, preferably from about 0.5 to about 2 weight %.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5 weight % of the composition, preferably from about 0.1 to about 1 weight %.

Another aspect of the present invention is directed to combination therapies wherein at least one compound according to any formula (I) to (IV) is administered together with a known or commonly used drug against infectious diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke. Especially preferred are combination therapies including systemic combination therapies of at least one compound of the present invention together with known or commonly used HIV drugs, antibiotics or anticancer drugs. Furthermore, the inventive compounds can also be applied in addition to chemotherapy or any other radiotherapy such as hyperthermia for cancer treatment.

In the following section, various reactions are described for the synthesis of the compounds according to general formula (I).

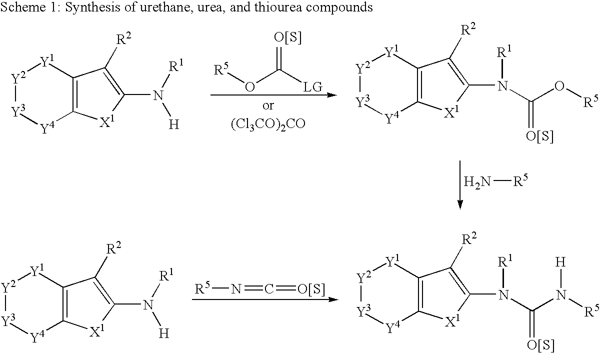

The urethane or thiourethane compounds of the present invention can be obtained by reaction of the corresponding amino compound with an alkyl or aryl formate as represented by the general formula R⁵—O—CO-LG or R⁵—O—CS-LG, wherein LG is a leaving group such as —Cl, —Br, —I, —N₃, —O—CO—C(CH₃)₃ or any other suitable leaving group well known to a person skilled in the art.

The obtained urethane or thiourethane compound can further be converted to an urea or thiourea compound by consumption with a suitable amine such as primary and secondary amines. Said urea or thiourea compounds can also be obtained by conversion of the corresponding amine with an isocyanate or thioisocyanate as shown in Scheme 1.

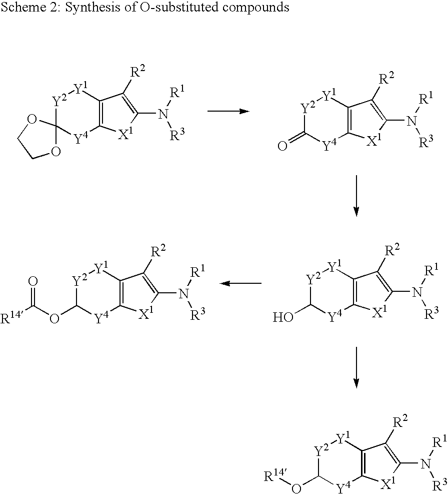

If a residue should be bound to the six-membered ring via an oxygen atom, one suitable route may start from precursors having a keto group protected as a 1,3-dioxolane. As shown in Scheme 2, said compound having a 1,3-dioxolane residues can be converted to the free keto compound by cleavage of the 1,3-dioxolane ring with acid such as hydrochloric acid. Said deprotection reactions are well known to a skilled person. The keto compound can, for instance, further be reduced to the hydroxy compound using e.g. hydrides such as sodium borohydride. The obtained hydroxy compound can be converted to ethers when deprotonated with base such as sodium hydride, MeLi, BuLi, K—OC(CH₃)₃, or any other commonly used base and reacted with an electrophile represented by, for instance R¹⁴′-LG, wherein LG is a leaving group such as —Cl, —Br, —I, —O-mesylate, —O-tosylate, or any other suitable leaving group. Furthermore, the hydroxy group may be esterified by use of carboxylic acid halides, or azides, or activated esters.

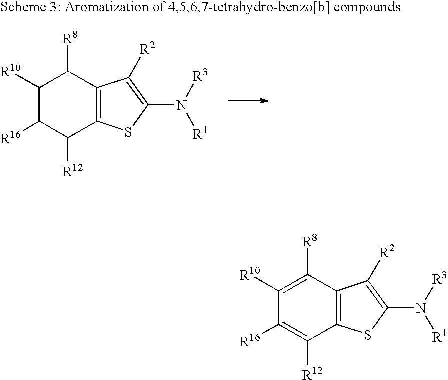

Aromatization of the carbocyclic compounds such as 4,5,6,7-tetrahydro-benzo[b]furan or 4,5,6,7-tetrahydro-benzo[b]thiophene as shown in Scheme 3 can be achieved by use of MoO₃/Al₂O₃, Pt, Pd, DDQ, SeO₂, benzoquinones, or any other dehydrogenation reagents known to a person skilled in the art. Further methods for aromatization of the 6-membered carbocyclic ring are disclosed in Scheme 4.

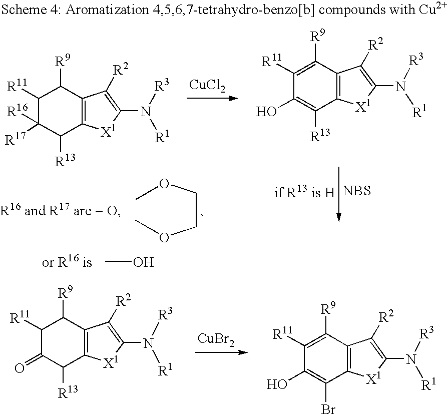

Aromatization of the carbocyclic ring can be achieved by use of copper dichloride starting from a compound of general formula (I) wherein R⁸ and R⁹ or R¹⁰ and R¹¹ or R¹² and R¹³ or R¹⁶ and R¹⁷ represent together an oxygen or a 1,3-dioxolane residue or wherein R⁹ or R¹¹ or R¹³ or R¹⁷ is a hydroxy group. As shown in Scheme 4, for instance, R¹⁶ and R¹⁷ form together a 1,3-dioxolane ring or together with carbon 6 a carbonyl group or R¹⁶ represents a hydroxy group. In this case, aromatization can be achieved with copper dichloride resulting in the corresponding 6-hydroxy-benzo[b] compound. A bromination in ortho position to the hydroxy group can be achieved by means of NBS or Br₂. Said brominated product can also be obtained if the corresponding unsaturated keto compound is reacted with copper dibromide. Depending on the amount of NBS or CuBr₂ used, a mono or dibromination can be obtained. The α,α-dibrominated product can be treated with a base such as potassium carbonate in order to eleminate HBr under aromatization resulting in a compound bearing a bromo and hydroxy substituent (cf. synthesis of compound A15). The bromo residue can further be substituted according to known reaction procedures.

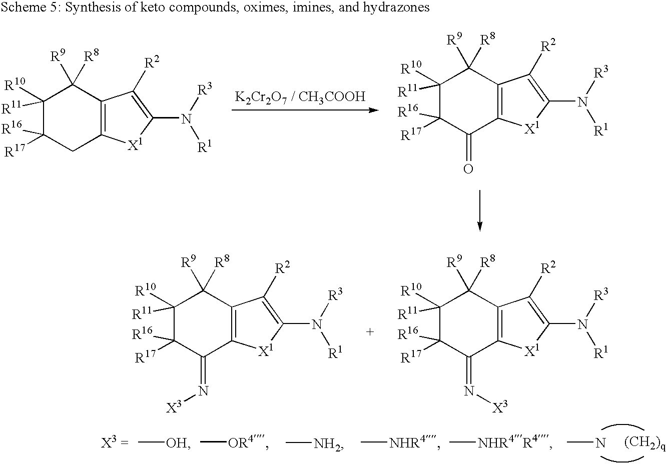

An oxidation of the compounds of general formula (I), especially of general formula (IIa) wherein R¹² and R¹³ represent hydrogen can be performed by the use of Cr₂O₇²⁻ (dochromate) in polar and protic solvents at elevated temperatures. The resulting 7-oxo-compound can then further converted to the corresponding oxime, imine, hydrazone by the use of hydroxylamine, primary amine, O-substituted hydroxylamine, hydrazine, mono or N,N-disubstituted hydrazine according to known procedures. One preferred embodiment of said conversion comprises the use of a microwave for several minutes. Ethanol or propanol or isopropanol was used as solvent and the reaction was carried out at temperature between 70° C. and 160° C., preferably 100° C. and 150° C.

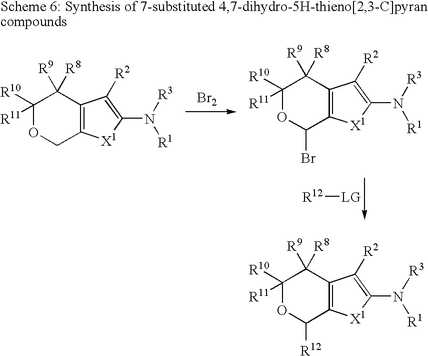

A bromination of the starting material in position 7 as shown in Scheme 6 can be carried out by the use of elemental bromine and sodium acetate in acetic acid. Further substitution of the bromo residues according to known methods leads to a broad spectrum of compounds substituted in position 7.

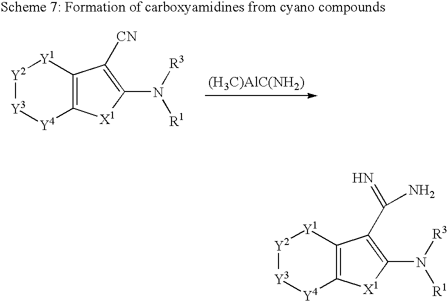

The conversion of a cyano group to a carboxyamidine group can be achieved by the use of ammonium chloride in conjunction with AlMe₃.

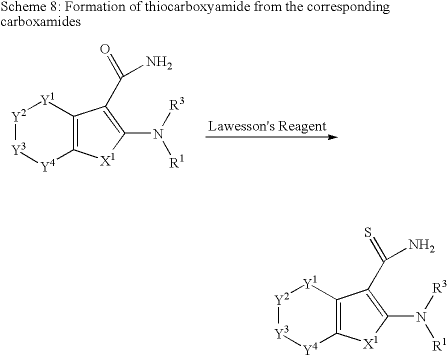

A carboxamide residue can be converted to the corresponding thiocarboxamide residue by the use of the commercially available Lawesson's Reagent.

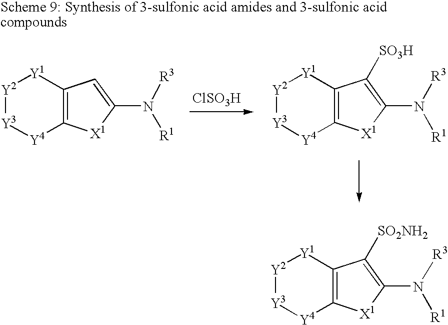

The 3-sulfonic acid compounds of the present invention can be synthesized from the corresponding starting material as shown in Scheme 9 by the use of sulfonic acid chloride in methylene chloride as solvent at lower temperatures. The obtained 3-sulfonic acid compound can further be converted to the 3-sulfonic acid amide by means of, for instance, oxalyl chloride and ammonia.

The 4,7-dihydro-5H-thieno[2,3-c]pyran compounds according to the present invention are obtainable by different synthetic routes. One route, which leads to 4,7-dihydro-5H-thieno[2,3-c]pyran derivatives starts with the reaction of tetrahydro-pyran-4-one or a correspondingly substituted derivative thereof with an cyano-actetate ester under acidic or basic conditions, preferably under acidic conditions, and under elimination of water and subsequent reaction of the reaction product with sulfur in the presence of an organic base to give a corresponding 2-amino-4,7-dihydro-5H-thieno[2,3-c]pyran-3-carboxylic acid ester derivative.

As a next step, the amino group in the thus obtained 2-amino4,7-dihydro-5H-thieno[2,3-c]pyran-3-carboxylic acid ester derivative can be acylated to give a corresponding 2-carbonylamino compound. As an acylation reagent a carboxylic acid chloride is preferably used. This reaction can optionally be carried out in the presence of a base such as an tertiary amide, preferably NEt(ⁱPr)₂.

Other suitable reactions to obtain the secondary carboxylic acid amides can be used, for instance reaction of the amino group with a carboxylic acid and a coupling-agent as used in peptide chemistry, such as HOBT,HOOBT,HBTU or HOAt.

Alternatively, if instead of the acyl group a sulfonyl group is to be attached to the amino group in 2-position, the 2-amino-4,7-dihydro-5H-thieno[2,3-c]pyran-3-carboxylic acid ester derivative can be reacted with a sulfonyl chloride compound to give a corresponding 2-sulfanylamino derivative.

The thus obtained compounds can then optionally be reacted with bromine in the presence of an organic acid, preferably acetic acid, to substitute one hydrogen in 7-position of the heterocyclic nucleus by a hydroxyl group.

The above described 3-carboxylic acid ester derivative compounds can then be reacted in a subsequent reaction step with an alkali metal amide, such as LiNH₂ or NaNH₂, in a polar solvent, which is essentially inert to the alkali metal amide, to give the corresponding 3-carboxylic acid amide derivative. This reaction is preferably carried out under the exclusion of moisture and optionally under an inert atmosphere. The application of lithium amide instead of sodium amide results in higher yields and purer products.

To prepare the corresponding 4,7-dihydro-5H-thieno[2,3-c]pyran derivatives in which a sulfonamide is attached in 3-position, in a first step, 4,7-dihydro-5H-thieno[2,3-c]pyran-2-amine can be acylated, preferably using a carboxylic acid chloride to give the corresponding 2-carbonyl-amino derivative. This compound can then be reacted with sulfurylchloride, preferably under an inert atmosphere and subsequently with ammonia to give the 3-sulfonamide compound.

If the compounds used to synthesise the compounds according to the present invention contain —NH, —SH or —OH functional groups which potentially interfere with the desired reaction, these may of course be protected with suitable protective groups, which can later on be removed from the respective compounds.

To obtain those analogues of the 4,7-dihydro-5H-thieno[2,3-c]pyran derivatives in which the S-atom in the 5-membered ring of the heterocyclic nucleus is substituted either by NR⁴′ or O, the following synthetic approach can be utilized, which is partially based on a method described in Hauser, C. R., Hoffenberg, D. S.; J. Org. Chem. 1955, 20, 1448-1453.

To obtain the O-analogue compounds, the amino group in 2-position of a corresponding 2-amino-3-cyano-4,7-dihydro-5H-furo[2,3c]pyrane derivative can be acylated in a first reaction step, using the acylation reaction described above with reference to the acylation of the 2-amino-4,7-dihydro-5H-thieno[2,3-c]pyran-3-carboxylic acid ester derivatives, i.e. preferably using a carboxylic acid chloride as an acylation agent, obtionally in the presence of a tertiary amine base such as NEt(ⁱPr)₂. Similarly, to obtain the NR⁴′-analogue compounds, a corresponding 2-amino-3-cyano-4,7-dihydro-5H-pyrrolo[2,3-c]pyrane derivative is acylated in the above described manner.

The respective 2-carbonyl-amino derivatives obtained by this acylation can then be reacted with boron trifluoride-acetic acid complex [BF₃.(HOAc)₂] and subsequently treated with an aqueous alkali metal hydroxide solution, such as sodium hydroxide, to convert the cyano group in 3-position of the heterocyclic nucleus into the carboxamide group.

In a further aspect of the present invention, the invention is directed at a method for amidation of an carboxylic acid ester to give the corresponding primary carboxylic acid amide. This amidation comprises the step of reacting an carboxylic acid ester with an alkali metal amide in the presence of a polar solvent, which is essentially inert against the alkali metal amides. Preferably, the molar ratio of carboxylic acid ester to alkali metal amide lies in the range of 1:1 to 1:15, more preferably in the range of 1:5 to 1:14, and most preferably in the range of 1:9 to 1:13.

In a preferred embodiment of the method of the present invention, the alkali metal amide is LiNH₂ or NaNH₂, and preferably is LiNH₂. The solvent is preferably absolute ether or absolute tetrahydrofurane, preferably tetrahydrofurane, and the reaction is preferably carried out under the exclusion of moisture. Preferably, the reaction is carried out at a temperature of 15° C. to 35° C., preferably at 25° C. It is furthermore preferred that the reaction duration lies in the range of from 40 to 80 hours, preferably from 45 to 75 hours.

According to the following inventive procedure various amides can be synthesized:

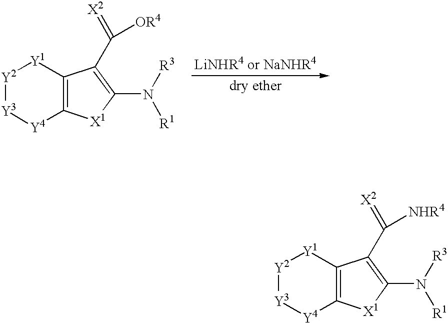

wherein

X² represents O or S, and

R¹, R³, R⁴, Y¹-Y⁴ and X¹ have the meanings as defined in claim 1 or any part of the description.

In a preferred embodiment of said method of the present invention, the carboxylic acid ester is a compound according to the following general formula (D):

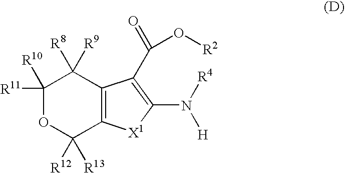

which is amidated to give the primary carboxylic acid amide according to formula (E),

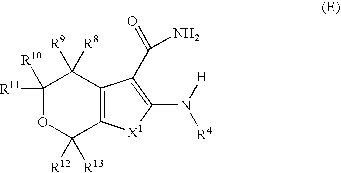

wherein in formulas (D) and (E)

X¹ is selected from S, O, or NR¹, and R¹ is selected from H, substituted or unsubstituted C₁-C₆-alkyl,

R² is linear or branched C₁-C₆ alkyl or aryl and preferably is methyl, ethyl, phenyl or benzyl,

R⁴ is selected from H, —C(═X²)R⁵ and —SO₂R⁵,
wherein X² is O, S or NH and
R⁵ is selected from substituted or unsubstituted C₃-C₁₀-cycloalkyl, C₁-C₆-alkyl, aryl, heteroaryl, heterocycloalkyl, C₂-C₄-alkenyl, C₂-C₄-alkinyl, adamantyl, 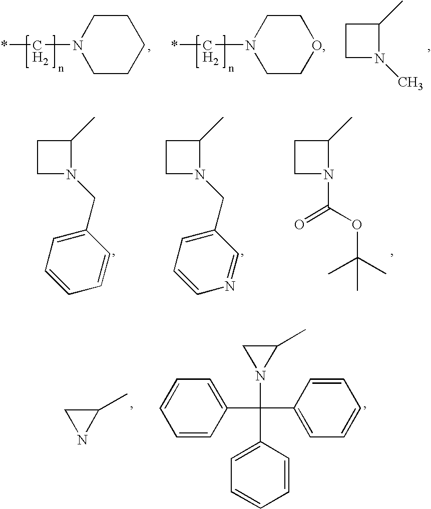
or —(CH₂)ₙ—NR⁶R⁷,
   wherein R⁶ and R⁷ are independently selected from substituted or unsubstituted C₁-C₄-alkyl or C₂-C₄-alkenyl and wherein n=1 to 6,
or NR⁶R⁷,
   wherein
   R⁶ is selected from H, C₁-C₆-alkyl, and
   R⁷ is selected from substituted or unsubstituted C₃-C₁₀-cycloalkyl, C₁-C₆-alkyl, aryl, heteroaryl, heterocycloalkyl, C₂-C₄-alkenyl, C₂-C₄-alkinyl, or adamantyl,

R⁸ is H and R⁹ is selected from H, substituted or unsubstituted C₁-C₆-alkyl

R¹⁰ is selected from H, substituted or unsubstituted C₁-C₆-alkyl, C₁-C₆-alkoxy, or OH

R₁₁ is selected from H and substituted or unsubstituted C₁-C₆-alkyl

R₁₂ is selected from H and substituted or unsubstituted C₁-C₆-alkyl, C₁-C₆-alkoxy, or

OH, and

R¹³ is selected from H or substituted or unsubstituted C₁-C₆-alkyl,

and stereoisomeric and regioisomeric forms and pharmaceutically acceptable salts of these compounds.

In a further preferred embodiment of the method of the present invention, in general formulas (D) and (E)

X¹ is S

R² is methyl or ethyl,

R⁴ is —C(═O)R⁵ and R5 is selected from methyl, ethyl, propyl, butyl, cyclopropyl, cyclobutyl, cyclopentyl, cyclohexyl, C₁-C₁₀-cycloalkyles substituted by at least one methyl or carboxyl group, phenyl, furanyl, thienyl, pyrrolyl, pyridyl, pyrrolidinyl, piperidinyl, tetrahydrofuranyl, ethenyl, cis-prop-1-enyl, trans-prop-1-enyl, cis-prop-2-enyl, trans-prop-2-enyl, but-1-enyl, cis-but-2-enyl, trans-but-2-enyl, but-3-enyl, prop-1-inyl, prop-2-inyl, but-1-inyl, but-2-inyl, but-3-inyl or adamantyl,

R⁸ is H and R⁹ is selected from H, substituted or unsubstituted C₁-C₆-alkyl,

R¹⁰ is selected from H, substituted or unsubstituted C₁-C₆-alkyl, C₁-C₆-alkoxy, or OH,

R₁₁ is selected from H and substituted or unsubstituted C₁-C₆-alkyl,

R₁₂ is selected from H and substituted or unsubstituted C₁-C₆-alkyl, C₁-C₆-alkoxy, or OH, and

R¹³ is selected from H or substituted or unsubstituted C₁-C₆-alkyl.

According to one preferred embodiment of the method of the present invention, the compound according to the general formula (E) is obtained by the following reaction sequence:

Step I:

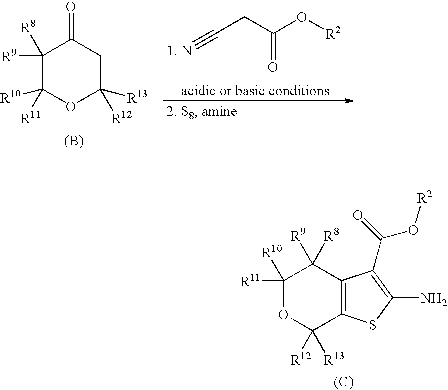

Step II: Acylation of the —NH₂ group in 2-position in compound (C) with R⁵C(═O)LG, wherein LG represents a suitable leaving group, preferably a halogen such as F, Cl, Br or I, most preferably Cl, to give compound (D):

and,

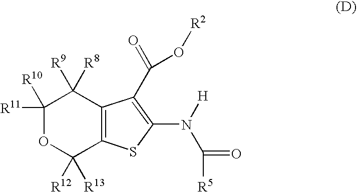

Step III: Amidation of compound (d) as outlined above by means of a sodium or lithium amide, 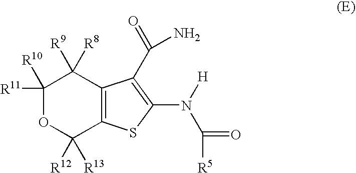
to give compound (E):

It is preferred that in Step I the reaction of compound (B) with the cyano-acetate ester is carried out in a nonpolar solvent, preferably benzene, with the addition of a mixture of ammonium acetate and acetic acid in a molar ratio of greater than 1, preferably in the range from 0.5:1 to 0.8 to 1, and preferably at a temperature in the range of 50 to 100° C., preferably between 70 to 90° C., preferably under removal of water formed in the reaction, and preferably for a duration of 2 to 4 hours.

Furthermore, in a preferred embodiment of the present invention, in Step I the reaction product of the reaction of compound (B) with the cyano-acetate ester is reacted with the S₈ in a protic solvent, preferably EtOH, S₈ being added at least in aquimolar quantities, preferably in an excess of up to 1,5, more preferably of up to 1,2, in the presence of a amine base, preferably morpholine, at reaction temperature of between 25 to 65° C., preferably between 40 and 60° C., and preferably for a duration of 2 to 6 hours.

### DESCRIPTION OF FIGURES

FIG. 1 shows vector, mutant, and wild type after 1 h and 24 h postinfection,

FIG. 2 shows the general formula of claim 1.

FIGS. 3-7 show representative examples of the compounds according to general formula (I).

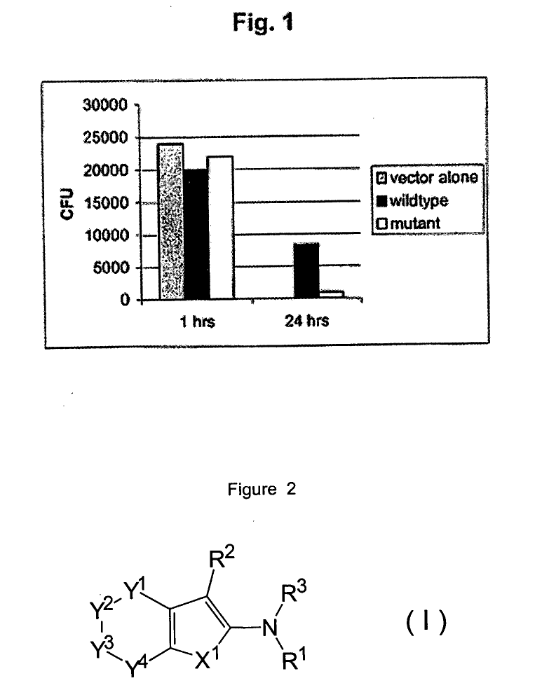

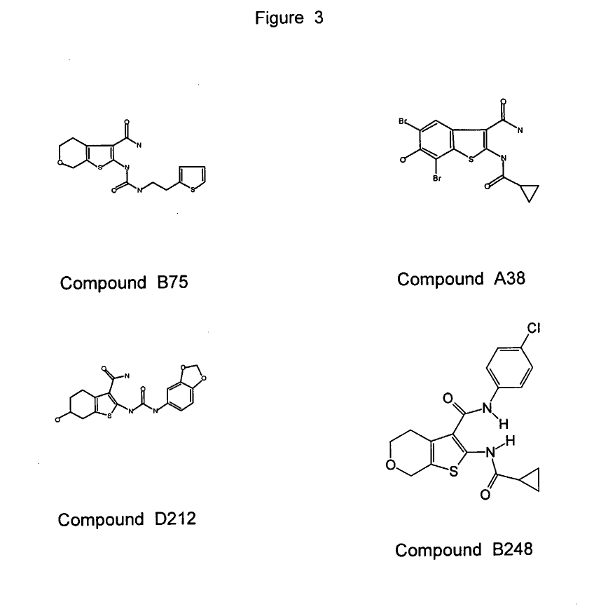

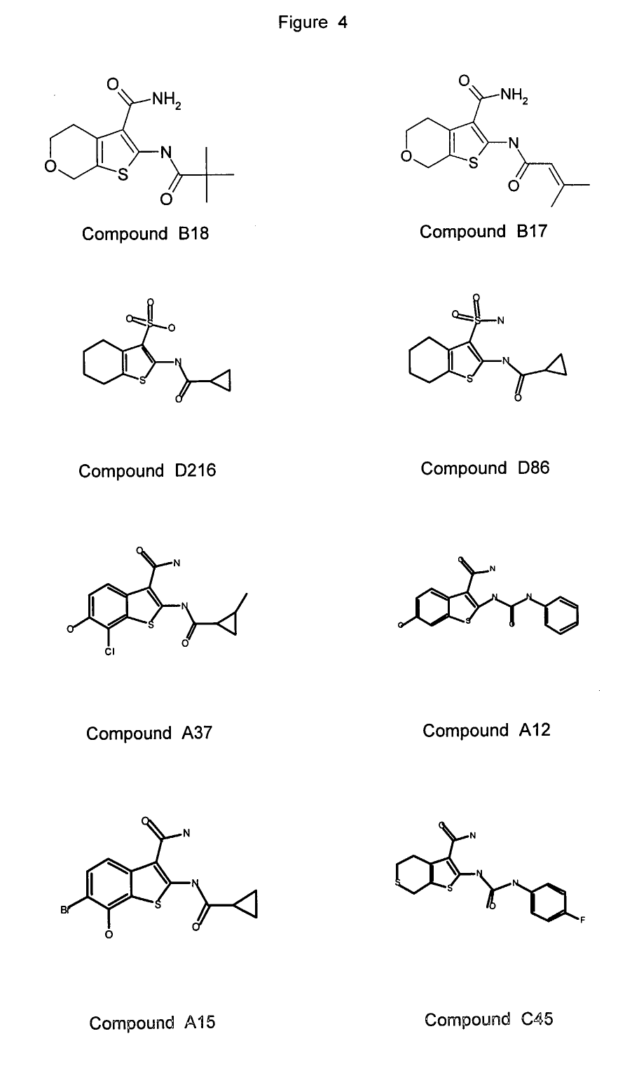

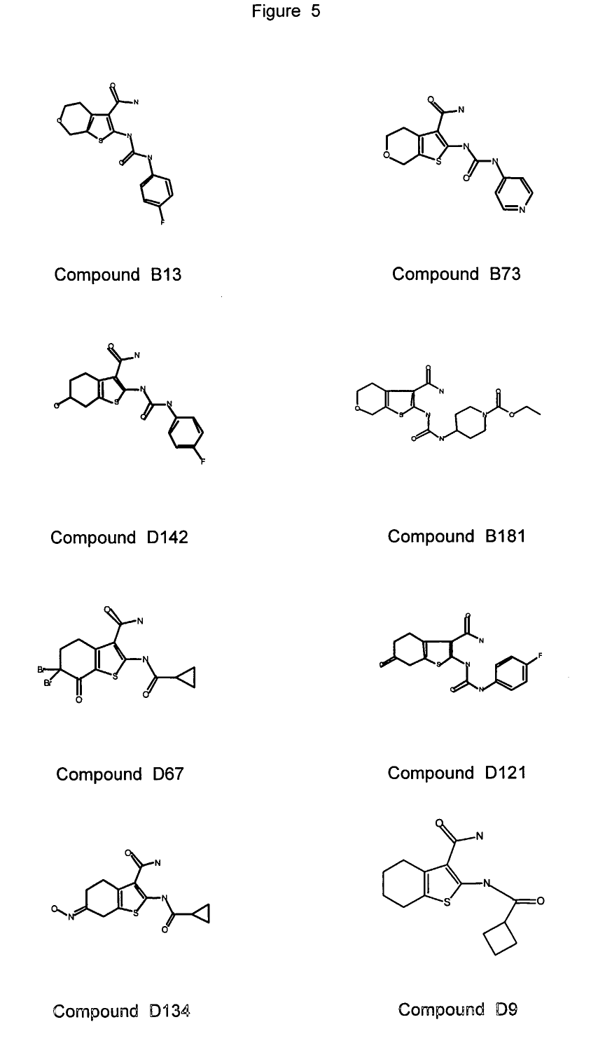

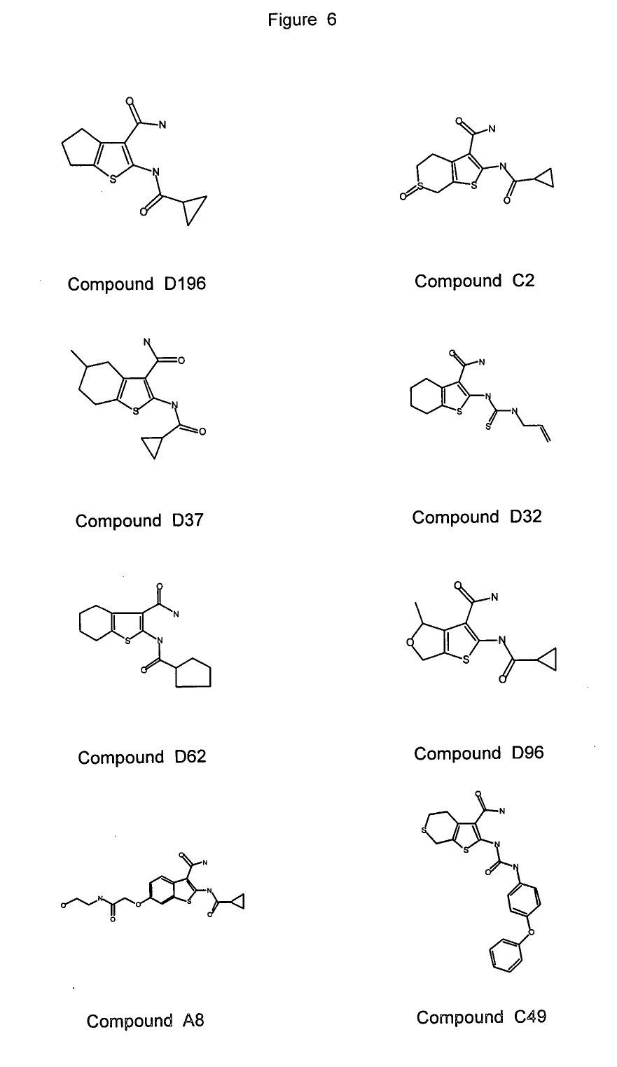

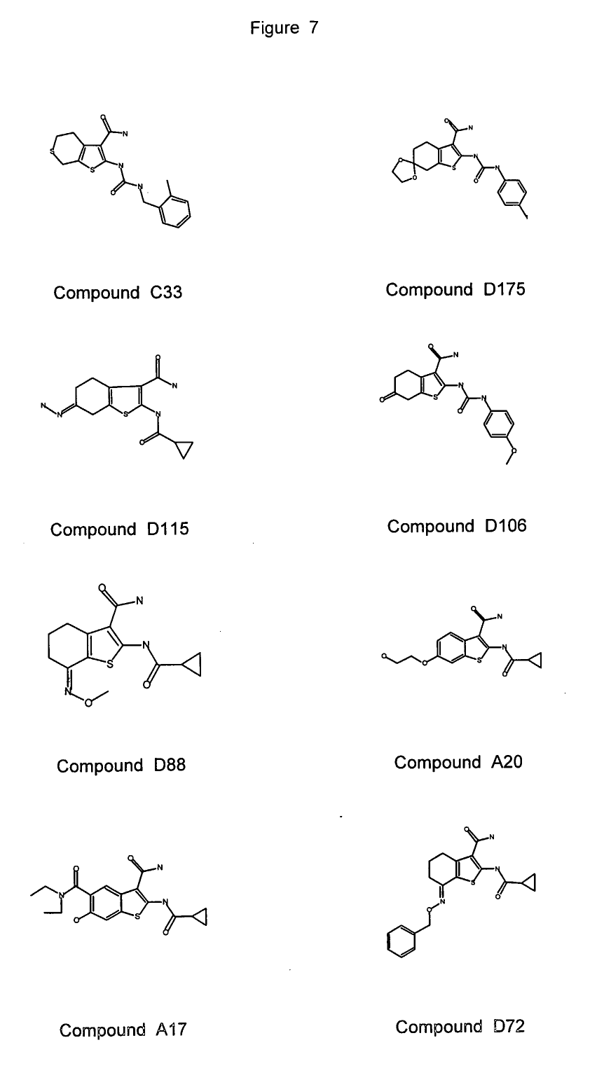

### EXAMPLE

Analytical Methods:

LC/MS data were obtained using a Waters Micromass ZQ instrument with atmospheric pressure chemical ionisation or electrospray ionisation under the conditions described below.

MS Detection:

Scan range for MS Data (m/z)

Start (m/z) 100

End (m/z) 600

With +ve/−ve switching

Ionisation is either electrospray or APCI dependent on compound types.

Standard Prep HPLC Conditions (Method 1)

### HPLC Setup

| | |
|---|---|
| Solvents: | Acetonitrile (Lichrosolv Merck) |
| | Water (deionised) |
| Column: | Waters Xterra MS 5μ C18, 19 × 150 mm. |
| Flow Rate: | 30 ml/min |
| Gradient: | A: Water B: MeCN |

| | | |
|---|---|---|
| Time | A % | B % |
| 0.00 | 100 | 0 |
| 2.50 | 100 | 0 |
| 10.00 | 65 | 35 |
| 10.10 | 0 | 100 |
| 14.00 | 0 | 100 |
| 14.10 | 100 | 0 |
| 15.00 | 100 | 0 |

Standard LC-MS Conditions

### HPLC Method A

| Solvents: | Acetonitrile (Riedel-deHaën; G Chromasolv) | | |
|---|---|---|---|
| | Water (Milli-Q Academic) | | |
| | Formic Acid (Riedel-deHaën; extra pure) | | |
| Column: | Supelco Discovery RP-AmideC16 | | |
| Flow Rate: | 3 ml/min | | |
| Gradient: | A: 10% AcCN/90% Water/0.05% HCOOH B: MeCN | | |
| | Time | A % | B % |
| | 0.00 | 100 | 0 |
| | 0.50 | 100 | 0 |
| | 2.00 | 20 | 80 |
| | 4.00 | 20 | 80 |
| | 4.20 | 100 | 0 |
| | 6.00 | 100 | 0 |

### HPLC Method B

| | |
|---|---|
| Solvents: | Acetonitrile (Riedel-deHaën; G Chromasolv) |
| | Water (Milli-Q Academic) |
| | Formic Acid (Riedel-deHaën; extra pure) |
| Column: | Merck Chromolith C18 |
| Flow Rate: | 2 ml/min |
| Gradient: | A: Water/0.05% HCOOH B: MeCN/0.05% HCOOH |

| | | |
|---|---|---|
| Time | A % | B % |
| 0.00 | 95 | 5 |
| 0.50 | 95 | 5 |
| 5.50 | 5 | 95 |
| 6.00 | 5 | 95 |
| 6.50 | 95 | 5 |
| 7.00 | 95 | 5 |

### HPLC Method C

| | |
|---|---|
| Solvents: | Acetonitrile (Lichrosolv Merck) |
| | Water (Lichrosolv Merck) with 1 mM ammonium |
| | acetate pH 6.8 |
| Column: | Waters Xterra MS 5μ C18, 3.0 × 50 mm. |
| Flow Rate: | 0.8 ml/min |
| Gradient: | A: Water/NH₄OAc B: MeCN |

| | | |
|---|---|---|
| Time | A % | B % |
| 0.00 | 98 | 2 |
| 5.00 | 5 | 95 |
| 6.50 | 5 | 95 |
| 6.60 | 98 | 2 |
| 8.00 | 98 | 2 |

UV Detection via Waters 2996 PDA

For purity assessments the wavelengths at 21 5, 254 and 310 nm were extracted from the PDA data and an average purity was calculated from the peak areas.

All reagents were obtained commercially and used directly. DMF and THF were dried over 4 Å molecular sieves (Fluka). Column chromatography employed Silica Gel 60 (Merck). TLC was carried out using pre-coated aluminium sheets Silica gel 60 F₂₅₄ (Merck).

Standard Conditions for Flash Chromatography

Flash chromatography was done using a SiO₂-column and the following solvents: cyclohexane (cHex), ethyl acetate (EtOAc), dichloromethane (DCM), methanol (MeOH).

### General Synthetic Methods

### General Method 1 for the Preparation of 2-carboxamidothiophene Derivatives

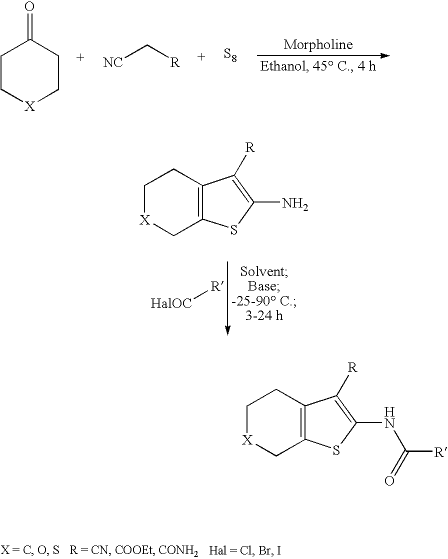

### Synthesis of the Thiophene Scaffold

To a 1:1:1 mixture of the corresponding cyclic ketone, acetonitrile derivative and sulfur in ethanol, morpholine (1.1 eq.) was added dropwise and the mixture heated at 45° C. for 4 h. The sulfur dissolved slowly and after approximately 2 h a yellow precipitate was formed. For complete precipitation the mixture was stored at 4° C. overnight. The solid was filtered, washed with water and re-crystallized from ethanol to give pale-white to yellow crystals of the product.

As an example, the following intermediates were prepared:

### 2-Amino-4,5,6,7-tetrahydro-benzo[b]thiophene-3-carboxamide

Mp.: 186-187° C. (ethanol).

Mass calc. for C₉H₁₂N₂OS: 196.27, found (pos. mode) 197.26.

### 2-Amino-4,7-dihydro-5H-spiro[1-benzothiophene-6,2′-[1,3]dioxolane]-3-carboxamide

Mp.: 157-158° C. (ethanol).

Mass calc. for C₁₁H₁₄N₂O₃S: 254.31, found (pos. mode) 255.4, found (neg. mode) 253.3.

### 2-Amino-5,5-dimethyl-4,7-dihydro-5H-thieno[2,3-c]pyran-3-carboxamide

Mp.: 212-214° C. (ethanol).

Mass calc. for C₁₄H₁₈N₂O₃S: 226.30, found (pos. mode) 249.1 [M+Na⁺], found (neg. mode) 225.2.

### 2-Amino-7,7-dimethyl-4,7-dihydro-5H-thieno[2,3-c]pyran-3-carboxamide

Mass calc. for C₁₄H₁₈N₂O₃S: 226.30, found (pos. mode) 227.1, found (neg. mode) 225.4.

### (4-R,S)-2-Amino-4-methyl-4,6-dihydro-thieno[2,3-c]furan-3-carboxamide

Mass calc. for C₈H₁₀N₂O₂S: 198.25, found (pos. mode) 199.3, found (neg. mode) 197.2.

### 2-Amino-5,5,7,7-tetramethyl-4,5,6,7-tetrahydro-benzo[b]thiophene-3-carboxamide

Mp.: 140-145° C. (ethanol).

Mass calc. for C₁₃H₂₀ON₂OS: 252.38, found (pos. mode) 253.4, found (neg. mode) 251.4.

### (6-R,S)-2-Amino-6-hydroxy-6-methyl-4,5,6,7-tetrahydro-benzo[b]thiophene-3-carboxamide

Mass calc. for C₁₀H₁₄N₂O₂S: 226.30, found (pos. mode) 227.2, found (neg. mode) 225.2.

### (6-R,S)-2-Amino-3-carbamoyl-4,5,6,7-tetrahydro-benzo[b]thiophene-6-carboxylic acid ethyl ester

Mp.: 201-204° C. (ethanol).

Mass calc. for C₁₂H₁₆N₂O₃S: 268.34, found (pos. mode) 269.1, found (neg. mode) 267.2.

### Synthesis of the 2-Carboxamidothiophene Derivatives

### Procedure 1.1. 2-(Cyclopropanecarbonyl-amino)-4,5,6,7-tetrahydro-benzo[b]thiophene-3-carboxamide (D1)

A suspension of 196 mg (1.00 mmol) of 2-amino-4,5,6,7-tetrahydro-benzo[b]thiophene-3-carboxamide, 100 μl (1.10 mmol) of cyclopropanecarboxylic acid chloride and 218 μl (1.25 mmol) of diisopropylethylamine in 3 ml toluene was heated at 90° C. for 4 h. After cooling at room temperature the mixture was diluted with 20 ml EtOAc and washed with 15 ml of 0.5 M HCl-solution. The water phase was extracted twice with 15 ml EtOAc and CH₂Cl₂. The combined organic layers were dried over Na₂SO₄. Evaporation of the solvent and recrystallization of the residue from ethanol gave the product as a light orange powder in 78% yield.

Mp.: 216-217° C. (ethanol).

Mass calc. for C₁₃H₁₆N₂O₂S: 264.34, found (neg. mode) 263.23.

### Procedure 1.2.

0.5 mmol of the corresponding carboxylic acid and 5 drops of abs. DMF were dissolved in 10 ml of abs. THF or abs. DCM under nitrogen atmosphere. 0.56 mmol (1.1 equivalents) of oxalyl chloride were slowly added via syringe at room temperature, and the solution was stirred for 2 h. The solvents were evaporated, and the residue (usually approximately 0.5 ml) was re-dissolved in 5 ml of abs. THF under nitrogen atmosphere. For some compounds the commercially available acid chloride was used instead. 0.56 mmol (1.1 equivalents) of DIPEA were added via syringe, followed by a suspension of 0.5 mmol (1 equivalent) of 2-amino-4,5,6,7-tetrahydro-benzo[b]thiophene-3-carboxamide or 2-amino-4,7-dihydro-5H-thieno[2,3-c]pyran-3-carboxamide in 5 ml of abs. THF. The suspension was stirred at room temperature for 2-24 h. 50 ml of saturated NH₄Cl solution were added, and the mixture was extracted three times with EtOAc. The combined organic layers were washed with water and dried over Na₂SO₄. Filtration and evaporation of the solvent gave the crude product, which was usually purified by recrystallization from hot ethanol. This reaction can be run with pyridine as solvent and base and also in a 1:1 mixture of THF-pyridine or DMSO-pyridine. In the latter two cases a work-up with cold 1N HCl is necessary.

According to this procedure the following compounds were prepared:

D127: Mp.: 198-199° C. (ethanol).

Mass calc. for C₁₆H₂₀N₂O₄S: 336.41, found (pos. mode) 337.2, found (neg. mode) 335.2.

D97: Mp.: 199-200° C. (ethanol).

Mass calc. for C₁₅H₁₈N₂O₄S: 322.39, found (pos. mode) 323.3, found (neg. mode) 321.2.

B156: Mp.: 223-225° C. (ethanol).

Mass calc. for C₁₄H₁₈N₂O₃S: 294.38, found (pos. mode) 317.4 [M+Na⁺], found (neg. mode) 293.4.

B71: Mp.: 195-196° C. (ethanol).

Mass calc. for C₁₄H₁₈N₂O₃S: 294.38, found (pos. mode) 317.4 [M+Na⁺], found (neg. mode) 293.4.

D96: Mp.: 241° C. (ethanol, decomposition).

Mass calc. for C₁₂H₁₄N₂O₃S: 266.32, found (pos. mode) 289.3 [M+Na⁺], found (neg. mode) 265.4.

D95: Mp.: 230-231° C. (ethanol, decomposition).

Mass calc. for C₁₇H₂₄N₂O₂S: 320.46, found (pos. mode) 343.4 [M+Na⁺], found (neg. mode) 319.4.

D201: Mp.: 185° C. (ethanol).

Mass calc. for C₁₄H₁₈N₂O₃S: 294.38, found (pos. mode) 295.1.

D146: Mp.: 211-213° C. (ethanol).

Mass calc. for C₁₆H₂₀N₂O₄S: 336.41, found (pos. mode) 337.1, found (neg. mode) 335.2.

B174: Mp.: 106-108° C.

Mass calc. for C₁₆H₂₀N₂O₄S: 336.41, found (pos. mode) 337.2.

B40: Mass calc. for C₂₀H₂₂N₂O₄S: 386.47, found (pos. mode) 387.1, found (neg. mode) 385.2.

B172: Mp.: 180-181° C.

Mass calc. for C₁₄H₁₈N₂O₄S: 310.37, found (pos. mode) 311.2 found (neg. mode) 309.1.

D94: Mp.: 207-209° C. (EtOH).

Mass calc. for C₁₄H₁₈N₂O₂S: 278.37, found (neg. mode) 277.21.

B221: Mass calc. for C₁₄H₁₈N₂O₃S: 294.37, found (pos. mode) 295.13, found (neg. mode) 293.16.

B175: Mp.: 84-94° C. (flash-column chromatography on silica with cHex/EtOAc 1:1).

Mass calc. for C₂₀H₃₀N₂O₃S: 378.54, found (neg. mode) 377.33.

B42: Mass calc. for C₁₆H₁₈Cl₂N₂O₃S: 388.04, found (pos. mode) 389.05, found (neg. mode) 387.08.

B194: Mp.: 214-217° C. (EtOH; decomposition).

Mass calc. for C₁₄H₁₆Cl₂N₂O₃S: 362.03, found (pos. mode) 363.04, found (neg. mode) 361.09.

### Ethyl 2-(2-furoylamino)-4,5,6,7-tetrahydro-benzo[b]thiophene-3-carboxylate

Mp.: 177-179° C.

Mass calc. for C₁₆H₁₇NO₄S: 319.38, found (pos. mode) 320.24.

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D4 | A | 3.05 | | 279.22 | B112 | A | 1.96 | | 321.2 |
| C1 | A | 2.83 | | 281.15 | A4 | A | 1.92 | | 273.13 |
| D5 | A | 3.01 | 267.21 | | B102 | B | 3.8 | | 379.08 |
| D6 | A | 3.07 | | 263.11 | B226 | B | 3.89 | | 369.15 |
| D7 | A | 3.21 | | 305.1 | B203 | B | 2.55 | | 273.09 |
| D8 | A | 3.11 | 291.08 | | B217 | B | 2.61 | | 316.97 |
| C2 | A | 1.22 | | 297.08 | B183 | B | 2.72 | | 283.25 |
| B1 | A | 1.53 | | 265.16 | B113 | B | 2.49 | | 283.13 |
| C51 | A | 0.39 | | 278.2 | B151 | B | 2.87 | | 306.14 |
| D155 | A | 3.06 | | 292.18 | D116 | B | 4.03 | | 440.18 |
| D44 | A | 1.97 | | 277.21 | D117 | B | 3.23 | | 414.18 |
| D89 | B | 2.5 | | 277.18 | D118 | B | 3.32 | | 378.23 |
| C39 | A | 1.79 | | 281.13 | D151 | B | 3.6 | | 304.16 |
| B83 | A | 1.61 | 267.04 | | D177 | B | 3.73 | | 387.17 |
| C52 | A | 2.12 | | 337.13 | D213 | B | 3.57 | 375.06 | |
| B248 | B | 4.31 | | 375.13 | | | | | |

### Procedure 1.3. 6-Methyl-2-[2-(4-nitro-phenyl)-acetylamino]-4,5,6,7-tetrahydro-benzo[b]thiophene-3-carboxylic acid amide (D190)

To a solution of 210 mg (1 mmol) of 2-Amino-6-methyl-4,5,6,7-tetrahydro-benzo[b]thiophene-3-carboxylic acid amide in 2 mL pyridine, 69 mg (0.5 mmol) of PCl₃ were added at −25° C. After 20 minutes 1 mmol of (4-nitro-phenyl)-acetic acid was added to the solution at −25° C., and the stirring continued at room temperature for 12 h. The reaction mixture was evaporated to dryness, dissolved in ethyl acetate, washed twice with 15 ml water, 15 ml 5% NaHCO₃, and 15 ml saturated NaCl solution. The combined organic layers were dried over Na₂SO₄ and evaporated to dryness. After washing with n-hexane and isopropanol a solid product was obtained in 63% yield.

The following compounds were prepared by this method:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D190 | A | 3.27 | | 373.02 | B107 | A | 1.41 | | 295.16 |
| D189 | A | 3.32 | | 386.11 | | | | | |

### General Method 2 for the Preparation of 2-[3-substituted-(thio)ureido]-thiophene Derivatives

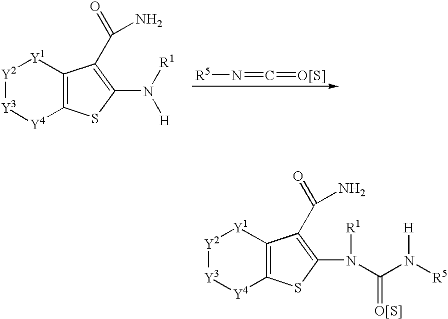

260 mg (1 mmol) 2-amino-4,7-dihydro-5H-spiro[1-benzothiophene-6,2′-[1,3]dioxolane]-3-carboxamide was stirred in 3 ml dry pyridine and 1.1 mmol of the corresponding isocyanate was added at room temperature and stirred overnight. Then 3 ml of distilled water was added and the precipitated crystals were filtered and dried in vacuum.

The following compounds were obtained according to this method:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D237 | B | 3.91 | | 424.00 | D238 | B | 2.39 | | 389.19 |
| D239 | B | 3.16 | | 462.24 | | | | | |

### Procedure 2.1. 2-(3-Cyclopropyl-thioureido)-4,5,6,7-tetrahydro-benzo[b]thiophene-3-carboxamide (D147)

To a suspension of 100 mg (0.51 mmol) 2-amino-4,5,6,7-tetrahydro-benzo[b]thiophene-3-carboxamide in 2.5 ml ethanol, 56 mg (0.56 mmol) of cyclopropylthioisocyanate were added dropwise. The mixture was heated to reflux and became a solution. After approximately 1 h a colourless precipitate was formed. After 2 h of reflux the mixture was cooled and stored at 4° C. overnight. The solid was filtered and washed with cold ethanol to yield the colourless product in 57% yield. ethanol can be replaced by a 1:1 mixture of DMSO abs. and pyridine abs. In this case the reaction is run at 90-100° C.

Mp.: 206-207° C. (decomposition, ethanol).

Mass calc. for C₁₃H₁₇N₃OS₂: 295.42, found (neg. mode) 294.30, (pos. mode) 296.24.

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D188 | A | 3.02 | | 280.11 | B180 | B | 3.47 | | 410.17 |
| D154 | A | 3.13 | | 294.19 | B29 | B | 3.4 | | 360.17 |
| B139 | A | 1.79 | | 298.1 | B28 | B | 3.57 | | 392.18 |
| D205 | B | 4.05 | | 450.09 | B59 | B | 3.38 | | 348.12 |
| B201 | | | | | D200 | B | 3.52 | | 416.26 |
| D198 | B | 3.63 | | 507.23 | B67 | B | 3.55 | | 362.18 |
| D195 | B | 2.3 | 409.2 | | B89 | B | 3.11 | | 358.18 |
| B91 | B | 3.67 | | 394.19 | B90 | B | 3.7 | | 344.21 |
| D109 | B | 4.84 | | 406.08 | B93 | B | 3.59 | | 344.12 |
| D110 | B | 4.93 | | 412.16 | B100 | B | 3.49 | | 364.05 |
| D111 | B | 4.49 | | 398.09 | B101 | B | 3.86 | | 364.05 |
| D132 | B | 3.74 | | 416.2 | B227 | B | 3.61 | | 364.05 |
| B85 | B | 3.81 | | 400.26 | B97 | B | 3.67 | | 380.05 |
| B103 | B | 2.94 | | 310.16 | B237 | B | 3.64 | | 384.98 |
| B179 | B | 3.36 | | 360.23 | B230 | B | 3.05 | | 376.06 |
| B166 | B | 3.44 | | 334.19 | B245 | B | 3.72 | | 418.02 |
| D250 | B | 3.33 | | 418.22 | D251 | B | 3.81 | | 456.2 |
| D252 | B | 3.59 | | 438.22 | D253 | B | 4.33 | 474 | |
| B249 | B | 3.13 | | 362.18 | B250 | B | 3.63 | | 400.18 |
| B251 | B | 3.40 | | 382.18 | B255 | B | 4.17 | | 416.16 |
| B261 | B | 3.17 | | 374.02 | B264 | B | 3.68 | | 388.09 |
| B267 | B | 3.37 | | 344.11 | B268 | B | 3.37 | | 350.04 |
| B269 | B | 3.21 | | 364.05 | B270 | B | 3.85 | | 411.93 |
| B271 | B | 3.91 | | 402.06 | B272 | B | 3.57 | | 370.06 |
| B273 | B | 3.43 | | 489.98 | B274 | B | 3.35 | | 344.06 |
| B275 | B | 3.73 | | 348.17 | B276 | B | 3.65 | | 352.07 |
| B277 | B | 3.68 | | 364.09 | B278 | B | 3.78 | | 344.11 |
| B279 | B | 3.95 | | 442.02 | B280 | B | 3.45 | | 344.11 |
| B281 | B | 4.07 | | 388.14 | B282 | B | 4.01 | | 473.85 |
| B283 | B | 4.34 | | 372 | B284 | B | 3.67 | | 359.4 |
| B285 | B | 4.92 | | 430.12 | B286 | B | 3.61 | | 357.99 |

### Procedure 2.2. 2-[(Anilinocarbonyl)amino]-4,7-dihydro-5H-spiro[1-benzothiophene-6,2′-[1,3]dioxolane]-3-carboxamide (D199)

A solution of 1.0 g (3.9 mmol) 2-amino-4,7-dihydro-5H-spiro[1-benzothiophene-6,2′-[1,3]dioxolane]-3-carboxamide in 20 ml THF was treated dropwise with 425 μl (3.9 mmol) phenyl isocyanate at 0° C. and stirred at 0° C. for 30 min. The mixture was allowed to come to room temperature and stirred for 3 h during which time a colourless precipitate was formed. The solid was filtered and washed with a small amount of cold THF and water. Addition of some water to the mother liquid afforded a second batch of the product as a colourless precipitate, which was filtered and washed with water and methanol. The combined product was obtained in 80% yield. THF can be replaced by a 1:1 mixture of DMSO abs. and pyridine abs. or by 1,4-dioxane (with 0.5 eq of DMAP). If the product does not precipitate, then the reaction mixture is poured onto ice-water and the pH is adjusted to pH 2 with diluted HCl. The precipitate is filtered, washed with water and dried.

Mp.: 212-213° C. (THF).

Mass calc. for C₁₈H₁₉N₃O₄S: 373.43, found (pos. mode) 374.1, found (neg. mode) 372.1.

According to this procedure the following compounds were prepared:

D178: Mp.: 219-220° C. (THF).

Mass calc. for C₁₈H₁₈FN₃O₄S: 391.42, found (pos. mode) 392.1, found (neg. mode) 390.2.

D176: Mp.: 198-199° C. (THF).

Mass calc. for C₁₉H₂₁N₃O₅S: 403.46, found (pos. mode) 404.2, found (neg. mode) 402.2.

D175: Mp.: 182-183° C. (THF).

Mass calc. for C₁₈H₁₈IN₃O₄S: 499.33, found (pos. mode) 500.0, found (neg. mode) 498.1.

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B84 | A | 1.54 | | 282.11 | D149 | A | 2.3 | | 382.09 |
| B8 | B | 3.16 | | 322.13 | D148 | A | 2.29 | | 348.03 |
| B86 | A | 2.03 | | 350.01 | D135 | A | 1.98 | | 328.17 |
| B87 | A | 1.72 | | 330.09 | D136 | A | 2.35 | | 415.93 |
| B88 | A | 2.08 | | 358.11 | D137 | A | 2.47 | | 450.03 |
| C38 | A | 1.96 | | 338.09 | D138 | A | 2.44 | | 416.03 |
| C40 | A | 2.19 | | 366 | D139 | A | 2.1 | | 374.13 |
| C41 | A | 1.91 | | 346.08 | D145 | A | 2.32 | | 392.02 |
| C44 | A | 1.71 | | 298.1 | D153 | A | 2.17 | | 389.09 |
| C14 | A | 2.48 | | 374.1 | D162 | A | 2.1 | | 373.13 |
| B9 | A | 1.83 | | 316.1 | D164 | A | 2.31 | | 366.07 |
| C45 | A | 2.04 | | 350.05 | D165 | A | 2.31 | | 356.19 |
| C11 | A | 2.21 | | 383.98 | D167 | A | 2.19 | | 359.12 |
| C5 | A | 1.95 | | 362.04 | D171 | A | 2.02 | | 344.13 |
| B116 | A | 2.09 | | 384.08 | D179 | A | 2.1 | | 344.12 |
| B117 | A | 2.01 | | 384.02 | D180 | A | 2.09 | | 359.07 |
| B118 | A | 1.79 | | 360.13 | D181 | A | 2.52 | | 381.99 |
| B119 | A | 1.98 | | 361.07 | D182 | A | 1.99 | | 404.09 |
| B120 | A | 1.85 | | 352.09 | D183 | A | 2.1 | | 350.1 |
| B121 | A | 1.74 | | 406.11 | D184 | A | 2.22 | | 359.09 |
| B122 | A | 2.27 | | 384 | D192 | A | 2.01 | | 358.15 |
| B123 | A | 1.84 | | 361.06 | C50 | A | 2.32 | | 399.94 |
| B124 | A | 1.85 | | 346.1 | C49 | A | 2.24 | | 424.01 |
| B125 | A | 1.94 | | 375.09 | C48 | A | 2.05 | | 391.03 |
| B126 | A | 2.06 | | 374.13 | C47 | A | 1.94 | | 374.06 |
| B127 | A | 1.96 | | 361.04 | C46 | A | 2.07 | | 391.05 |
| B128 | A | 1.86 | | 375.08 | C27 | A | 2.07 | | 346.08 |
| B129 | A | 2.07 | | 393.96 | C28 | A | 2.07 | | 346.04 |
| B130 | A | 1.92 | | 330.13 | C29 | A | 1.97 | | 360.08 |
| B11 | A | 1.78 | | 358.12 | C43 | A | 1.98 | | 360.08 |
| B132 | A | 2.25 | | 375.1 | C31 | A | 1.94 | | 346.07 |
| B133 | B | 3.12 | | 346.18 | C32 | A | 1.88 | | 376.05 |
| B134 | A | 2.05 | | 368.05 | C33 | A | 1.97 | | 360.09 |
| B142 | A | 2.2 | | 383.95 | C34 | A | 2.16 | | 399.94 |
| B143 | A | 2.15 | | 408.03 | C35 | A | 2.06 | | 408.05 |
| B144 | A | 2.1 | | 384.02 | C53 | A | 2.21 | | 400.05 |
| B145 | A | 1.81 | | 344.1 | C37 | A | 2.19 | | 409.91 |
| B146 | A | 1.94 | | 392.08 | C36 | A | 2.19 | | 400.05 |
| B147 | A | 1.74 | | 360.08 | C30 | A | 2.23 | | 433.91 |
| B148 | A | 1.85 | | 344.09 | C26 | A | 2.36 | | 467.93 |
| B149 | A | 1.83 | | 344.1 | C25 | A | 2.31 | | 433.98 |
| B150 | A | 1.78 | | 330.09 | C24 | A | 2 | | 392.04 |
| B12 | A | 1.91 | | 330.09 | C23 | A | 2.2 | | 409.92 |
| B74 | A | 2.22 | | 417.95 | C22 | A | 2.06 | | 406.76 |
| B153 | A | 1.87 | | 376.07 | C21 | A | 2.2 | | 410.88 |
| B154 | A | 2.28 | | 451.94 | C20 | A | 2.01 | | 391.03 |
| B155 | A | 2.12 | | 417.96 | C19 | A | 1.99 | | 362.05 |
| B13 | A | 1.88 | | 334.07 | C18 | A | 1.98 | | 377.02 |
| D103 | A | 2.45 | | 382.03 | C17 | A | 2.41 | | 399.95 |
| D83 | A | 2.35 | | 406.09 | C16 | A | 1.89 | | 422.05 |
| D71 | A | 2.17 | | 373.09 | C15 | A | 2 | 370.05 | |
| D75 | A | 2.04 | | 356.11 | C13 | A | 2.12 | | 377.05 |
| D76 | A | 2.16 | | 373.13 | C12 | A | 1.92 | | 376.09 |
| D77 | A | 2.18 | | 328.16 | B82 | B | 3.93 | | 422.14 |
| D70 | A | 2.16 | | 328.19 | B80 | B | 2.76 | | 320.21 |
| D131 | A | 2.03 | | 328.16 | B79 | B | 3.12 | | 360.17 |
| D69 | A | 2.06 | | 342.16 | C10 | B | 2.81 | | 351.06 |
| D68 | A | 2.07 | | 342.17 | B157 | B | 2.05 | | 331.21 |
| D65 | A | 1.97 | | 358.16 | B161 | B | 2.46 | | 333.16 |
| D80 | A | 2.07 | | 342.18 | D114 | B | 4.49 | | 420.15 |
| D64 | A | 2.31 | | 372.13 | C8 | B | 4.27 | 424.16 | |
| B43 | A | 2.06 | | 393.94 | D125 | B | 3.29 | | 416.17 |
| D87 | A | 2.31 | | 382.07 | D152 | B | 2.95 | | 376.18 |
| D107 | A | 2.26 | | 382.01 | D150 | B | 3.04 | 333.08 | |
| D112 | A | 2.15 | | 390.08 | B39 | B | 2.38 | | 388.14 |
| D113 | A | 2.13 | | 332.13 | B38 | B | 3.68 | | 378.09 |
| D124 | A | 2.29 | | 392 | B37 | B | 3.87 | | 410.01 |
| A62 | B | 3.59 | | 394.1 | A77 | B | 4.19 | | 372.06 |
| A64 | B | 3.39 | | 371.2 | A78 | B | 4.61 | | 431.95 |
| A65 | B | 3.00 | | 416.1 | A79 | B | 4.31 | 370.09 | |
| A66 | B | 3.39 | | 340.1 | A80 | B | 4.18 | | 390.04 |
| A100 | B | 4.69 | | 421.95 | A81 | B | 3.87 | | 444.09 |
| A101 | B | 4.32 | | 399.00 | A103 | B | 4.24 | | 398.14 |
| A102 | B | 4.13 | | 354.16 | A104 | B | 4.40 | 385.14 | |
| A121 | B | 3.67 | | 404.05 | D246 | B | 3.46 | | 408.10 |
| D247 | B | 3.21 | | 364.17 | A122 | B | 3.76 | | 394.12 |

### Procedure 2.2: Synthesis of 2-amino-6-hydroxy-benzo[b]thiophene-3-carboxylic acid amide

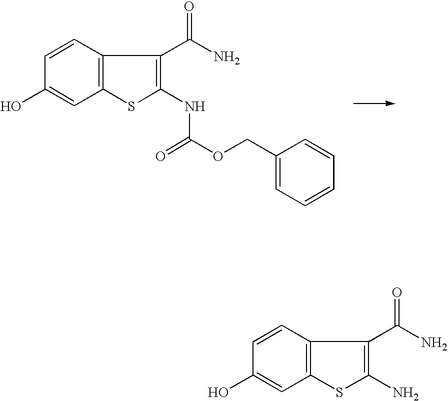

A mixture of 7.1 g (20.73 mmol) of (3-carbamoyl-6-hydroxy-benzo[b]thiophen-2-yl)-carbamic acid benzyl ester, 13.3 g (211 mmol) of ammonium formate, and 7.1 g of 5% palladium on carbon in 900 ml of methanol was stirred under argon for 24 h. The mixture was filtered through Celite and the filtrate concentrated under reduced pressure. The residue was treated with water, the precipitate was filtered off and washed with water and diethyl ether affording 2.15 g (49.9%) of the title compound.

HPLC method B; retention time: 1.83; M⁻: 207.03

### Procedure 2.3: Synthesis of 2-amino-6-ethoxy-benzo[b]thiophene-3-carboxylic acid amide (A76)

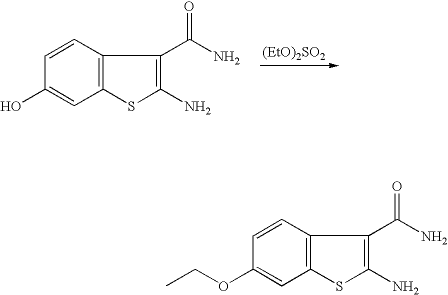

The mixture of 1.66 g (8. mmol) of 2-amino-6-hydroxy-benzo[b]thiophene-3-carboxylic acid amide, 2.2 g (16 mmol) potassium carbonate, 50 ml of acetone and 1.1 ml (1.29 g 8.4 mmol) diethylsulfate was stirred at reflux temperature for 24 hours. The mixture was concentrated under reduced pressure. The residue was treated with 20 ml water and 10 ml of ethylacetate. The insoluble material was filtered off, washed with water, a little ethylacetate and dried affording 1.66 g (yield: 61%) of the title compound as a white solid.

HPLC method B; retention time: 2.84; M⁻: 235.09

NMR (300 MHz, DMSO-d6 ppm): 7.57 (d, 1H), 7.46 (s, 2H), 7.25 (s, 1H), 6.85 (m, 3H), 4.00 (q, 2H), 1.31 (t, 3H)

### Procedure 2.4

The mixture of 14 ml abs. dioxane and 7 ml abs. THF was cooled down to 0° C. and phosgene was bubbled through the solution for 30 min. 2.53 mmol of the amino derivative were dissolved in 5 ml abs. dioxane and 2 ml abs. triethylamine and added slowly to the cold mixture. After stirring for 1 h the mixture was evaporated and 4 ml abs. DMSO was added to the isocyanate compound. 0.5 g (2.53 mmol) 2-Amino-4,7-dihydro-5H-thieno[2,3-c]pyran-3-carboxylic acid amide was dissolved in 8 ml abs. DMSO and 4 ml abs. pyridine and added to the isocyanate-solution. After 2 h stirring at room temperature the mixture was poured on 1N HCl, filtered off, washed with water and dried.

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B81 | B | 3.96 | | 418.27 | B22 | B | 3.93 | 454.27 | |
| B169 | B | 2.4 | 497.18 | | B21 | B | 3.99 | 474.26 | |
| D163 | B | 2.79 | | 493.29 | B51 | B | 3.87 | | 438.27 |
| C6 | B | 2.66 | | 513.23 | B33 | B | 3.9 | | 442.03 |

### Procedure 2.5

Nicotinyl chloride was dissolved in acetone, cooled to −10° C. and an aqueous solution of sodium azide was added. After 30 minutes the reaction mixture was diluted with cold distilled water and extracted 3 times with cold toluene. The organic layer was dried on Na₂SO₄ at 4° C. and filtered. The filtrate was added dropwise into refluxing toluene while stirring and refluxed for one hour. Toluene was evaporated and pyridine-3-isocyanate was obtained as a yellow oil and it was condensed with the corresponding amine as described in Procedure 1. The acyl chloride derivative can be replaced also by a mixed anhydride obtained by reaction of a carboxylic acid with a 10% excess of isobutyl chloroformate and triethylamine.

According to this procedure the following compounds were prepared:

| | HPLC | | | | | HPLC | Retention | | |
|---|---|---|---|---|---|---|---|---|---|
| ID | Method | Retention Time | M⁺ | M⁻ | ID | Method | Time | M⁺ | M⁻ |
| B78 | B | 2.05 | | 317.19 | B76 | B | 3.05 | | 322.1 |

### Procedure 2.6

1 mmol heteroarylamine was dissolved in 2.5 ml THF and 0.33 mmol triphosgene were added at 0° C. to the stirred solution under argon atmosphere. After 5 minutes, 3 mmol triethylamine was added dropwise while keeping the temperature at 0° C. for an additional 5 minutes, then 1 mmol 2-amino-4,7-dihydro-5H-thieno[2,3-c]pyran-3-carboxylic acid amide in 2.5 ml DMSO was added and the reaction mixture was allowed to reach room temperature and stirred for 2 h. The reaction mixture was poured onto ice-water and the precipitated product was filtered off and recrystallized from ethanol-water.

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D166 | B | 4.1 | 436.17 | | B131 | B | 3.18 | 312.02 | |
| B195 | B | 3.55 | | 436.09 | D81 | B | 2.31 | 367.13 | |
| B141 | B | 1.83 | 369.12 | | C9 | B | 2.1 | 385.12 | |
| C42 | B | 3.59 | | 326.14 | B73 | B | 2.05 | | 317.18 |
| D74 | B | 3.8 | 310.18 | | B158 | B | 2.55 | | 331.15 |
| B114 | B | | | | B159 | B | 2.84 | | 347.18 |
| D129 | B | 3.57 | | 352.14 | B160 | B | 2.81 | | 351.13 |
| B106 | B | 2.93 | | 354.15 | B162 | B | 2.37 | 383.18 | |
| C7 | B | 3.32 | | 370.11 | B163 | B | 3.19 | | 377.18 |
| D126 | B | 3.74 | | 386.11 | B164 | B | 3.11 | | 351.14 |
| D208 | B | 3.53 | | 384.13 | B252 | B | 3.58 | | 438.1 |
| B253 | B | 3.17 | | 372.0 | B256 | B | 3.45 | | 408.0 |
| A57 | B | 1.86 | 329.2 | | A59 | B | 1.76 | | 327.2 |
| B254 | B | 2.29 | | 318.1 | A60 | B | 3.30 | | 384.2 |
| A58 | B | 2.83 | 359.1 | | A61 | B | 3.17 | | 382.2 |

### General Method 3 for the Deprotection of 1,3-dioxolanes

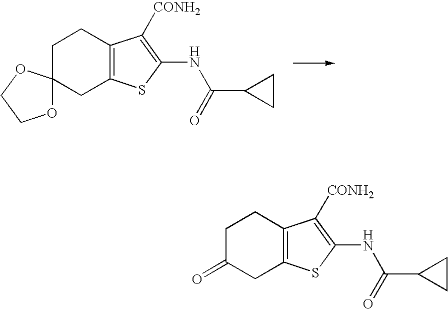

### Procedure 3.1. 2-(Cyclopropanecarbonyl-amino)-6-oxo-4,5,6,7-tetrahydro-benzo[b]thiophene-3-carboxamide (D89)

1.2 g (3.7 mmol) 2-(cyclopropanecarbonyl-amino)-4,7-dihydro-5H-spiro[1-benzothiophene-6,2′-[1,3]dioxolane]-3-carboxamide in 20 ml THF and 10 ml 1 M HCl solution was heated to reflux for 6 h under nitrogen atmosphere. Sat. NaHCO₃ solution was added to give a neutral water phase and the mixture was extracted four times with 50 ml EtOAc. The combined organic layers were washed with brine and dried over Na₂SO₄. Evaporation of the solvent gave the product as a light red powder in 85% yield. Recrystallization from ethanol afforded the product as a colourless solid.

Mp.: 201-202° C. (ethanol).

Mass calc. for C₁₃H₁₄N₂O₃S: 278.33, found (pos. mode) 279.1, found (neg. mode) 277.2.

According to this procedure the following compounds were prepared:

D204:

Mass calc. for C₁₄H₁₆N₂O₃S: 292.36, found (pos. mode) 293.2, found (neg. mode) 291.2.

D106: Mp.: 210-211° C.

Mass calc. for C₁₇H₁₇N₃O₄S: 359.41, found (pos. mode) 360.2, found (neg. mode) 358.2.

D105: Mp.: 221-222° C.

Mass calc. for C₁₆H₁₄IN₃O₃S: 455.28, found (pos. mode) 456.1, found (neg. mode) 454.1.

### Procedure 3.2

The 1,3-dioxolane derivative (2 mmol) was dissolved in abs. dichloromethane (5 ml) and trifluoroacetic acid (2 ml) was added. After stirring at room temperature for 24 h, the solution was concentrated under vacuum, and the solid material was filtered and washed with n-hexane (2×20 ml).

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D119 | B | 3.8 | | 396.15 | D122 | B | 3.1 | | 334.21 |
| D120 | B | 3.02 | | 370.18 | D123 | B | 2.4 | | 295.15 |
| D121 | B | 3.25 | | 346.16 | D240 | B | 3.44 | | 343.20 |

### General Method 4 for the Reduction of 6-oxo-4,5,6,7-tetrahydro-benzo[b]thiophene Derivatives to 6-hydroxy-4,5,6,7-tetrahydro-benzo[b]thiophene Derivatives

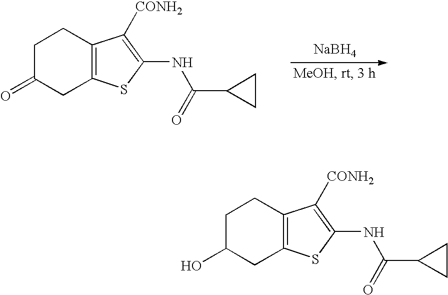

### (6-R,S)-2-(Cyclopropanecarbonyl-amino)-6-hydroxy-4,5,6,7-tetrahydro-benzo[b]thiophene-3-carboxamide (D90)

113 mg (0.41 mmol) of 2-(cyclopropanecarbonyl-amino)-6-oxo-4,5,6,7-tetrahydro-benzo[b]-thiophene-3-carboxamide was suspended in 4 ml methanol, and 19 mg (0.49 mmol) of NaBH₄ were added portion-wise. The mixture was stirred at room temperature for 3 h. After addition of a small amount of water, the solvent was evaporated and the residue taken up in 10 ml sat. NH₄Cl solution and 20 ml EtOAc. Extraction with EtOAc (4×30 ml), washing of the combined organic layers with brine, drying over Na₂SO₄ and evaporation of the solvent gave the crude product, which, upon recrystallization from ethanol, afforded the product as colourless crystals.

Mp.: 238-239° C. (ethanol).

Mass calc. for C₁₃H₁₆N₂O₃S: 280.35, found (pos. mode) 281.29, found (neg. mode) 279.23.

According to this general method the following compounds were prepared:

D202: Mp.: 224-230° C. (ethanol).

Mass calc. for C₁₄H₁₈N₂O₃S: 294.38, found (pos. mode) 295.2, found (neg. mode) 293.2.

D141: Mp.: 180-181° C. (ethanol/methanol, decomposition).

Mass calc. for C₁₇H₁₉N₃O₄S: 361.42, found (pos. mode) 362.1, found (neg. mode) 360.1.

D140: Mp.: 201-202° C. (ethanol/methanol).

Mass calc. for C₁₆H₁₆IN₃O₃S: 457.29, found (pos. mode) 458.0, found (neg. mode) 456.1.

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D90 | A | 1.46 | | 279.14 | D174 | B | 2.88 | | 336.09 |
| D157 | B | 3.21 | | 376.18 | D197 | B | 1.91 | | 269.17 |
| D168 | B | 2.18 | | 294.19 | D207 | B | 3.06 | | 374.1 |
| D169 | B | 3.61 | | 414.19 | D209 | B | 2.5 | | 374.13 |
| D170 | B | 3.41 | | 408.1 | D212 | B | 2.83 | | 374.02 |
| D173 | B | 3.55 | | 398.03 | D211 | B | 2.33 | | 296.11 |
| D244 | B | 3.35 | | 375.95 | D245 | B | 3.33 | | 395.96 |
| D248 | B | 3.52 | | 378.18 | D249 | B | 3.21 | | 378.14 |
| D254 | B | 2.89 | | 375.98 | D256 | B | 3.13 | | 395.98 |
| D255 | B | 3.33 | | 413.98 | D257 | B | 3.84 | | 429.93 |

### General Method 5 for the Preparation of 6-hydroxy-benzo[b]thiophene Derivatives

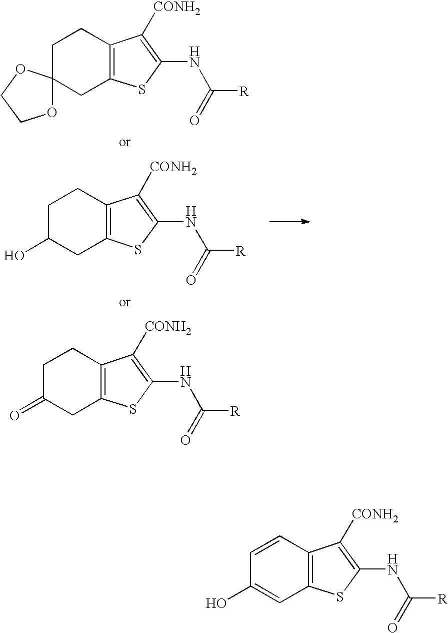

### Procedure 5.1. 2-(Cyclopropanecarbonyl-amino)-6-hydroxy-benzo[b]thiophene-3-carboxamide (A30)

A suspension of 500 mg (1.55 mmol) 2-(cyclopropanecarbonyl-amino)-4,7-dihydro-5H-spiro[1-benzothiophene-6,2′-[1,3]dioxolane]-3-carboxamide in 10 ml CH₃CN was treated with 528 mg (3.10 mmol) CuCl₂.2 H₂O at room temperature. The mixture turned brown immediately and was stirred at room temperature for 2 h. The solvent was evaporated and the residue taken up in 20 ml 0.5 M HCl solution and 40 ml. EtOAc. The mixture was extracted with EtOAc. The combined organic layers were washed with brine and dried over Na₂SO₄. Evaporation of the solvent gave the crude product, which was purified by flash column chromatography on silica gel (eluent cHex/EtOAc 1:2) to afford a slightly purple powder in 79% yield. Further purification by preparative HPLC (Method 1) or recrystallization from ethanol afforded the product as a colourless solid.

For the work-up it is also possible to filter off the purple solid directly after the reaction. After washing the solid with 10 ml acetonitrile, 100 ml 0.3 M HCl solution and finally twice with 20 ml THF, and drying it, a pure product was obtained which could be used in further steps without additional purification.

Mp.: 278-281° C. (CH₃CN/H₂O).

Mass calc. for C₁₃H₁₂N₂O₃S: 276.31, found (pos. mode) 277.2, found (neg. mode) 275.3.

According to this procedure the following compounds were prepared:

A5: Mp.: 220-221° C. (ethanol).

Mass calc. for C₁₄H₁₄N₂O₃S: 290.34, found (pos. mode) 291.2, found (neg. mode) 289.2.

A12: Mp.: >290° C. (CH₃CN/H₂O).

Mass calc. for C₁₆H₁₃N₃O₃S: 327.36, found (pos. mode) 328.1.

A24: Mp.: >280° C. (CH₃CN/H₂O).

Mass calc. for C₁₆H₁₁ClFN₃O₃S: 379.80, found (pos. mode) 380.1 & 382.1, found (neg. mode) 378.1 & 380.1.

A11: Mass calc. for C₁₇H₁₅N₃O₄S: 357.39, found (pos. mode) 358.1, found (neg. mode) 356.1.

A33: Mp.: 226-227° C. (CH₃CN/H₂O).

Mass calc. for C₁₆H₁₂IN₃O₃S: 453.26, found (pos. mode) 454.0 & 456.0, found (neg. mode) 452.1 & 454.1.

| | HPLC | Retention | | |
|---|---|---|---|---|
| ID | Method | Time | M⁺ | M⁻ |
| A22 | B | 3.52 | | 341.19 |
| A98 | B | 2.85 | | 293.09 |

### Procedure 5.2. 2-(Cyclopropanecarbonyl-amino)-6-hydroxy-benzo[b]thiophene-3-carboxamide (A30)

A suspension of 100 mg (0.36 mmol) (6-R,S)-2-(cyclopropanecarbonyl-amino)-6-hydroxy-4,5,6,7-tetrahydro-benzo[b]thiophene-3-carboxamide and 12 mg (0.36 mmol) sulfur in 1.5 ml dimethyl phthalate was heated in the microwave at 245° C. for 20 min. The resulting brown solution was evaporated and the residue purified by flash column chromatography on silica gel (eluent cHex/EtOAc 1:2) afford a colourless solid in 40% yield.

Mass calc. for C₁₃H₁₂N₂O₃S: 276.31, found (pos. mode) 277.2, found (neg. mode) 275.3.

### Procedure 5.3. 6-Hydroxy-2-[3-(4-trifluoromethyl-phenyl)-ureido]-benzo[b]thiophene-3-carboxylic acid amide (A26)

100 mg (0.25 mmol) of 6-Oxo-2-[3-(4-trifluoromethyl-phenyl)-ureido]-4,5,6,7-tetrahydro-benzo[b]thiophene-3-carboxylic acid amide and 80 mg palladium on charcoal (10%) were suspended in the mixture of 18 ml glacial acetic acid and 0.5 ml water. The mixture was stirred at 40° C. for 48 h, then filtered, and the solution was evaporated to dryness to give the title compound in 89% yield.

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| A26 | B | 3.81 | | 394.17 | A1 | B | 3.18 | | 332.05 |
| A31 | B | 3.28 | | 344.03 | A13 | B | 2.22 | | 278.11 |
| A42 | B | 3.78 | | 374.15 | A63 | B | 3.59 | | 410.01 |
| A47 | B | 3.48 | | 374.15 | A82 | B | 3.40 | | 391.97 |
| A48 | B | 3.17 | | 371.98 | A83 | B | 4.07 | | 425.92 |

### Procedure 5.4. 2-[3-(4-Bromo-phenyl)-ureido]-6-hydroxy-benzo[b]thiophene-3-carboxylic acid amide (A39)

Benzoquinone (1.5 mmol) was added to a solution of 2-[3-(4-bromo-phenyl)-ureido]-6-oxo-4,5,6,7-tetrahydro-benzo[b]thiophene-3-carboxylic acid amide in acetic acid. The mixture was stirred overnight at 50° C. The solvent was evaporated, the residue was dissolved in ethyl acetate and washed several times with saturated Na₂SO₃, brine and dried over Na₂SO₄. Evaporation of the solvent gave a solid, which was crystallized from isopropanol.

Yield: 60%

The following compounds were prepared by this method:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| A6 | B | 3.56 | | 273.08 | A41 | B | 3.32 | | 370.06 |
| A39 | B | 3.66 | | 403.97 | A115 | B | 3.80 | | 410.13 |
| A116 | B | 3.48 | | 372.05 | A118 | B | 2.37 | | 290.14 |
| A117 | B | 2.75 | | 292.09 | A119 | B | 3.61 | | 379.91 |

### Procedure 5.5. 3-Carbamoyl-2-(cyclopropanecarbonyl-amino)-benzo[b]thiophene-6-carboxylic acid ethyl ester (A28)

DDQ (2 mmol) and a catalytic amount of p-toluenesulfonic acid were added to a solution of 3-carbamoyl-2-(cyclopropanecarbonyl-amino)-4,5,6,7-tetrahydro-benzo[b]thiophene-6-carboxylic acid ethyl ester in acetic acid and the mixture was stirred for 2 h at 100° C. The solvent was evaporated and the residue was dissolved in ethyl acetate and washed several times with saturated Na₂SO₃ and brine dried over Na₂SO₄. Evaporation of the solvent gave the product (Yield: 70%).

The following compounds were prepared by this method:

| | HPLC | Retention | | |
|---|---|---|---|---|
| ID | Method | Time | M⁺ | M⁻ |
| A27 | B | 4.55 | | 402.06 |
| A28 | B | 3.66 | | 331.15 |

### General Method 6 for the Conversion of 1,3-dioxolane Derivatives to 6-hydroxy-4,5,6,7-tetrahydro-benzo[b]thiophene Derivatives

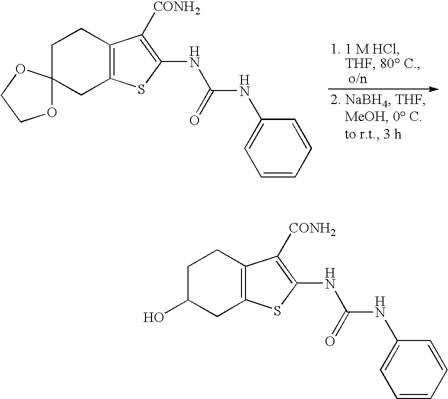

### (6-R,S)-2-[(Anilinocarbonyl)amino]-6-hydroxy-4,5,6,7-tetrahydro-benzo[b]thiophene-3-carboxamide (D143)

300 mg (0.80 mmol) 2-[(anilinocarbonyl)amino]-4,7-dihydro-5H-spiro[1-benzothiophene-6,2′-[1,3]dioxolane]-3-carboxamide was dissolved in 15 ml THF and 5 ml 1 M HCl solution and heated to reflux (oil bath 80° C.) overnight. The mixture was stored in the fridge overnight and the precipitated 6-keto product filtered and washed with water and some THF. After drying of the crude 6-keto compound under fine vacuum overnight (crude yield: 76%), it was reduced as described in general method 4.

Mp.: 153-154° C. (ethanol/methanol).

Mass calc. for C₁₆H₁₇N₃O₃S: 331.40, found (pos. mode) 332.1, found (neg. mode) 330.2.

According to this general method the following compound was prepared:

D142: Mp.: 180-181° C. (ethanol/methanol).

Mass calc. for C₁₆H₁₆FN₃O₃S: 349.39, found (pos. mode) 350.1, found (neg. mode) 348.2.

### General Method 7 for the Preparation of 6-ethoxy-benzo[b]thiophene Derivatives

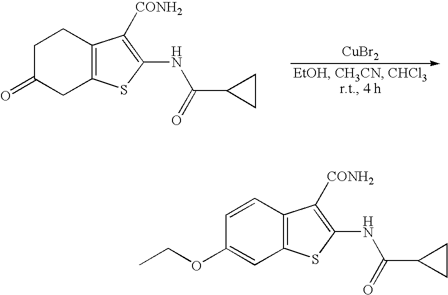

### 2-(Cyclopropanecarbonyl-amino)-6-ethoxy-benzo[b]thiophene-3-carboxamide (A9)

53 mg (0.24 mmol) CuBr₂ was added to a solution of 60 mg (0.22 mmol) 2-(cyclopropanecarbonyl-amino)-6-oxo-4,5,6,7-tetrahydro-benzo[b]thiophene-3-carboxamide in 1 ml CH₃CN, 1 ml CHCl₃ and 20 μl EtOH. The mixture was stirred at room temperature for 4 h. 10 ml water were added and the mixture extracted three times with 20 ml EtOAc. The combined organic layers were washed with brine, dried over Na₂SO₄ and the solvent was evaporated. Separation by preparative HPLC (Method 1) afforded the title compound as a light brown solid in 5% yield.

Mass calc. for C₁₅H₁₆N₂O₃S: 304.36, found (pos. mode) 305.2, found (neg. mode) 303.2.

According to this general method the following compound was prepared:

A23: Mp.: >260° C. (CH₃CN/H₂O).

Mass calc. for C₁₉H₁₉N₃O₄S: 385.44, found (pos. mode) 386.2, found (neg. mode) 384.2.

### General Method 8 for the Preparation of 6-substituted-alkoxy Derivatives

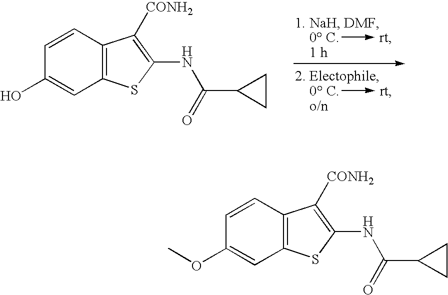

### 2-(Cyclopropanecarbonyl-amino)-6-methoxy-benzo[b]thiophene-3-carboxamide (A2)

To a suspension of 19 mg (0.47 mmol) NaH (60% dispersion in mineral oil) in 2 ml DMF was added a solution of 100 mg (0.36 mmol) 2-(cyclopropanecarbonyl-amino)-6-hydroxy-benzo[b]thiophene-3-carboxamide in 2 ml DMF at 0° C. The mixture was stirred at room temperature for 1 h, cooled again to 0° C. and treated with 30 μl (0.47 mmol) methyl iodide. After stirring overnight at room temperature, the mixture was treated with 20 ml sat. NH₄Cl-solution and extracted three times with 30 ml EtOAc. The combined organic layers were washed with brine and dried over Na₂SO₄. Evaporation of the solvent gave the crude product, which was purified by flash column chromatography on silica gel (eluent cHex/EtOAc 1:1) to afford a reddish foam in 32% yield.

Mass calc. for C₁₄H₁₄N₂O₃S: 290.34, found (pos. mode) 291.1, found (neg. mode) 289.1.

According to this general method the following compounds were prepared:

A34: Mp.: 190-192° C. (CH₃CN/H₂O).

Mass calc. for C₂₀H₂₅N₃O₃S: 387.50, found (pos. mode) 388.2, found (neg. mode) 386.2.

A19: Mp.: 194-195° C. (CH₃CN/H₂O).

Mass calc. for C₁₆H₁₉N₃O₃S: 333.41, found (pos. mode) 334.2.

A25: Mp.: 172-173° C. (ethanol).

Mass calc. for C₁₈H₂₁N₃O₄S: 375.45, found (pos. mode) 376.2, found (neg. mode) 374.2.

A29: Mp.: 200-202° C. (EtOAc).

Mass calc. for C₁₆H₁₆N₂O₅S: 348.38, found (pos. mode) 349.1, found (neg. mode) 347.2.

A21: Mp.: 180-183° C. (H₂O/CH₃CN).

Mass calc. for C₂₀H₂₄N₂O₅S: 404.49, found (pos. mode) 405.2, found (neg. mode) 403.3.

D144: In this example 3.26 equivalents of NaH (60% dispersion in mineral oil) and dimethyl sulfate as the electrophile were used.

Mass calc. for C14H₈N₂O₃S: 294.38, found (pos. mode) 295.2, found (neg. mode) 293.2.

A10: Mass calc. for C₂₀H₂₄ClN₃O₃S: 421.95, found (pos. mode) 422.2 & 424.1, found (neg. mode) 420.2 & 422.2.

### General Method 8A for Bis-Chlorination and Derivatization of the 6-Hydroxy Group

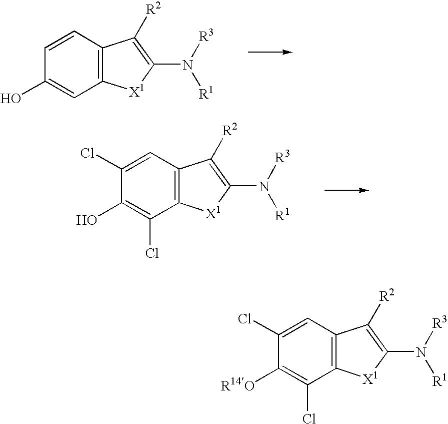

The above outlined reaction scheme is exemplified by the synthesis of A49 and A50.

### Preparation of 5,7-dichloro-2-(cyclopropanecarbonyl-amino)-6-hydroxy benzo[b]thiophene-3-carboxylic acid amide (A43)

0.28 g (0.10 mmol) 2-(Cyclopropanecarbonyl-amino)-6-hydroxy-benzo[b]thiophene-3-carboxylic acid amide and 0.40 g (3.00 mmol) N-chloro-succinimide was stirred at room temperature between 4 to 24 hours in 15 ml acetonitrile. The reaction mixture was diluted with 15 ml water, the solid was filtered off, and was washed three times with 5 ml water. The crude product was refluxed in 10 ml methanol for half an hour, was cooled to room temperature, and was filtered off to give pure product in 80% yield.

Mp.: 240-245° C.

Mass calc. for C13H10Cl2N2O3S: 345.21.

NMR, δ (ppm): 11.83 (broad s, 1H), 10.22 (broad s, 1H), 7.86 (s, 1H), 7.80 (s, 2H), 2.05 (m, 1H), 0.94 (m, 4H).

Compound A120 was prepared according to this reaction procedure.

Yield of A120: 0.22 g (70%)

Mp.: 235-240° C.

NMR, δ (ppm): 11.24 (broad s, 1H), 10.24 (broad s, 1H), 7.88 (s, 1H), 7.78 (broad s, 2H), 4.23 (m, 2H), 1.27 (t, J=7.08 Hz, 3H).

### General Method for the Preparation of 6-alkoxy-5,7-dichloro-2-(cyclopropanecarbonyl-amino)-benzo[b]thiophene-3-carboxylic acid amides

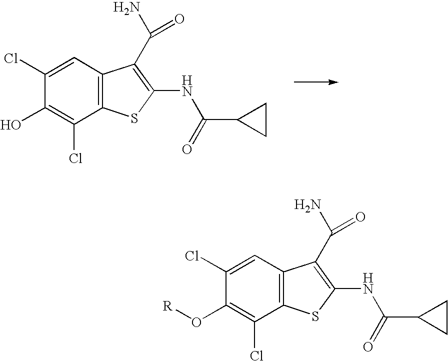

0.18 g (0.50 mmol) 6-hydroxy-5,7-dichloro-2-(cyclopropanecarbonyl-amino)-benzo[b]thiophene-3-carboxylic acid amide was treated with 0.12 g (1.00 mmol) potassium-tert-butoxide at room temperature in 20 ml absolute dimethyl-formamide. After stirring at room temperature for one hour 0.06 mmol alkyl-halide derivative was added into the reaction mixture, and it was heated at 80° C. for four hours. Then the solvent was evaporated under reduced pressure, the residue was titurated with 20 ml water, and extracted three times with 20 ml ethyl-acetate. The combined organics were washed with 20 ml brine and dried over magnesium-sulphate. Evaporation of the solvent gave the crude product, which was recrystallised from ethanol afforded the pure product.

According to this procedure the following compounds were prepared:

| | HPLC | Retention | | |
|---|---|---|---|---|
| ID | Method | Time | M⁺ | M⁻ |
| A44 | B | 4.17 | | 457.1 |
| A45 | B | 4.47 | | 371.0 |
| A46 | B | 4.66 | | 385.0 |

### Preparation of 6-(2-Amino-ethoxy)-5,7-dichloro-2(cyclopropanecarbonyl-amino)-benzo[b]thiophene-3-carboxylic acid amide hydrochloride (A50)

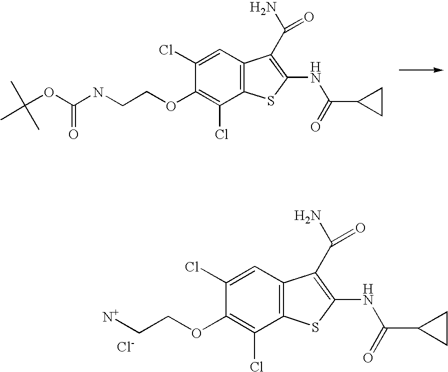

0.17 g (0.34 mmol) {2-[3-Carbamoyl-5,7-dichloro-2-(cyclopropanecarbonyl-amino)-benzo[b]thiophen-6-yloxy]-ethyl}-carbamic acid tert-butyl ester was dissolved in 20 ml methanol, and 1.0 ml ethyl acetate saturated with hydrochloric acid was added into the reaction mixture. After stirring for two hours at room temperature, the solvent was evaporated. The crude product was recrystallised from 5 ml ethanol-(2-propanol) 1-1 mixture to give pure product.

Yield: 65%

Mass calc. for C15H15Cl2N3O3S: 388.28.

NMR, δ (ppm): 11.89 (broad s, 1H), 8.28 (broad s, 3H), 7.97 (s, 1H), 7.88 (s, 2H), 4.24 (t, J=5.22 Hz, 2H), 3.27 (m, 2H), 2.09 (m, 1H), 0.98 (m, 4H).

### General Method 9 for the Preparation of Substituted carbamoyl-(methoxy)-benzo[b]thiophene Derivatives

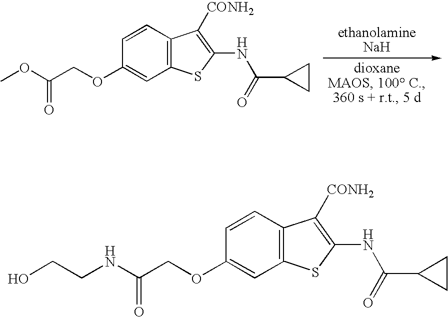

### 2-(Cyclopropanecarbonyl-amino)-6-[(2-hydroxyethylcarbamoyl)-methoxy]-benzo[b]thiophene-3-carboxamide (A8)

60 mg (0.17 mmol) [3-carbamoyl-2-(cyclopropanecarbonyl-amino)-benzo[b]thiophen-6-yloxy]-acetic acid methyl ester was dissolved in 2 ml dioxane and treated with 40 μl (0.66 mmol) ethanolamine. Under stirring 20 mg (0.52 mmol) NaH (60% dispersion in mineral oil) were added. The microwave tube was sealed and heated in the microwave at 100° C. for 360 s. Since the conversion was not complete, the reaction was allowed to stir at room temperature for 5 days. The mixture was treated with 15 ml sat. NH₄Cl solution and extracted four times with 20 ml EtOAc. The combined organic layers were washed with brine and dried over Na₂SO₄. Evaporation of the solvent gave the crude product, which was purified by preparative HPLC (Method 1) to afford the product as a colourless solid in 25% yield.

Mp.: 220-222° C. (H₂O/CH₃CN).

Mass calc. for C₁₇H₁₉N₃O₅S: 377.42, found (pos. mode) 378.2, found (neg. mode) 376.2.

According to this general method the following compounds were prepared:

A7: Mp.: 249-251° C. (H₂O/CH₃CN).

Mass calc. for C₁₆H₁₇N₃O₄S: 347.40, found (pos. mode) 348.2, found (neg. mode) 346.2.

D79: Mp.: 252-254° C. (CH₃CN/H₂O).

Mass calc. for C₁₆H₂₁N₃O₄S: 351.43, found (pos. mode) 352.2, found (neg. mode) 350.2.

According to this procedure the following compounds were prepared:

| | HPLC | Retention | | |
|---|---|---|---|---|
| ID | Method | Time | M⁺ | M⁻ |
| A51 | B | 2.83 | | 402.1 |
| A52 | B | 2.57 | | 390.3 |
| A53 | B | 2.37 | | 420.1 |
| A54 | B | 2.26 | 417.2 | |
| A55 | B | 3.26 | | 482.3 |
| A56 | B | 3.65 | | 436.2 |

### General Method 10 for the Preparation of 7-oxo or 4-oxo-4,5,6,7-tetrahydro-benzo[b]thiophene Derivatives

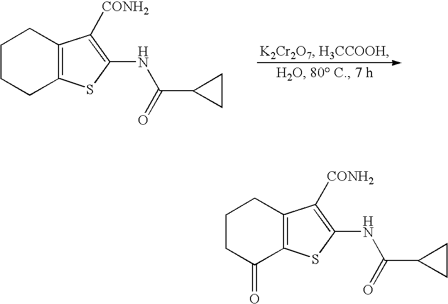

### 2-(Cyclopropylcarbonyl-amino)-7-oxo-4,5,6,7-tetrahydro-benzo[b]thiophen-3-carboxylic acid amide (D172)

A solution of 8.4 g (28.5 mmol) potassium dichromate in 12 ml water at 60° C. was slowly added to 2.5 g (9.5 mmol) 2-(cyclopropanecarbonyl-amino)-4,5,6,7-tetrahydro-benzo[b]thiophene-3-carboxylic acid amide dissolved in 20 ml acetic acid at 60° C. and the reaction was then stirred at 80° C. for 7 h. After the starting material disappeared, the reaction mixture was poured on ice (80 g), and the aqueous layer was extracted three times with EtOAc (250 ml). Evaporation to dryness and recrystallisation of the residue from hot EtOH afforded 1.04 g of the desired product in 40% yield.

Mp.: 203-205° C.

Mass calc. for C₁₃H₁₄N₂O₃S: 278.33, found (pos. mode) 279.1, found (neg. mode) 277.1.

According to this general method the following compounds were prepared:

D161: Mp.: 249-250° C.

Mass calc. for C₁₄H₁₆N₆O₃S: 292.36, found 293.1 (pos. mode), found 291.1 (neg. mode).

D160: Mp.: 188-189° C.

Mass calc. for C₁₄H₁₆N₆O₃S: 292.36, found 293.1 (pos. mode), found 291.1 (neg. mode).

### General Method 11 for the Preparation of 6- or 7-hydrazone-, 6- or 7-hydroxyimino- and 6- or 7-O-substituted hydroxyimino-4,5,6,7-tetrahydro-benzo[b]thiophene Derivatives

### Procedure 11.1

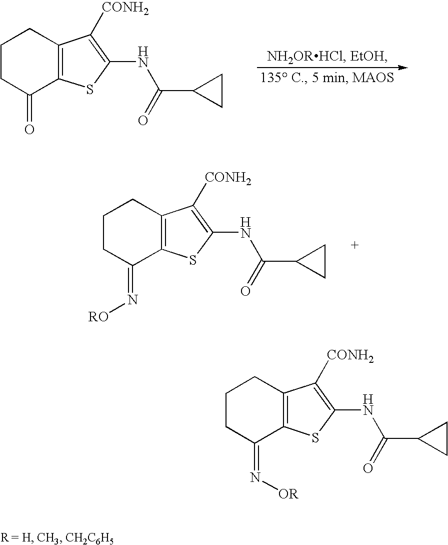

Typically, a mixture of 90 mg (0.32 mmol) 2-(cyclopropylcarbonyl-amino)-7-oxo-4,5,6,7-tetrahydro-benzo[b]-thiophen-3-carboxylic acid amide and hydroxylamine hydrochloride or an O-substituted hydroxylamine derivative hydrochloride salt (0.71 mmol) in 3 ml EtOH was heated in the microwave at 135° C. for 5 min. The reaction was then poured into water (15 ml) and extracted three times with EtOAc (25 ml). Drying of the combined organic layers over Na₂SO₄ and evaporation of the solvent yielded after purification by preparative HPLC (Method 1) the isomerically pure solids (Z and E isomer).

According to this general method the following compounds were prepared:

D84: Mp.: 255-256° C.

Mass calc. for C₁₃H₁₅N₃O₃S: 293.35, found (pos. mode) 294.1.

D104: Mp.: 240-241° C.

Mass calc. for C₁₃H₁₅N₃O₃S: 293.35, found 294.1 (pos. mode).

D85: Mp.: 213-214° C.

Mass calc. for C₁₄H₁₇N₃O₃S: 307.37, found (pos. mode) 308.1.

D88: Mp.: 210-211° C.

Mass calc. for C₁₄H₁₇N₃O₃S: 307.37, found (pos. mode) 308.1.

D72: Mp.: 195-196° C.

Mass calc. for C₂₀H₂₁N₃O₃S: 383.47, found (pos. mode) 384.2, found (neg. mode) 382.3.

D73: Mp.: 176-177° C.

Mass calc. for C₂₀H₂₁N₃O₃S: 383.47, found (pos. mode) 384.4, found (neg. mode) 382.3

D93: Mp.: 265-266° C. (dec.).

Mass calc. for C₁₄H₁₇N₃O₃S: 307.37, found (pos. mode) 308.1.

### Procedure 11.2. 2-(Cyclopropanecarbonyl-amino)-6-hydroxyimino-4,5,6,7-tetrahydro-benzo[b]thiophene-3-carboxylic acid amide (D134)

1 mmol 2-(Cyclopropylcarbonyl-amino)-6-oxo-4,5,6,7-tetrahydro-benzo[b]thiophene-3-carboxylic acid amide was suspended in acetonitrile then 1.5 equivalent K₂CO₃ and 1.5 equivalent hydroxylamine HCl were added to the mixture and stirred at reflux for 5 h. The solvent was removed and extracted with EtOAc two times. The collected organic layer was washed with water and dried over Na₂SO₄, and the solvent was evaporated. The crude product was crystallized from isopropanol. If hydrazine is used, then EtOH is the solvent of choice and the reaction is completed after 24 h at room temperature.

According to this general method the following compounds were prepared:

| | HPLC | Retention | | |
|---|---|---|---|---|
| ID | Method | Time | M⁺ | M⁻ |
| D115 | B | 2.51 | 293.33 | |
| D134 | B | 2.49 | | 292.18 |
| D203 | B | 2.97 | | 373.09 |
| D214 | B | 3.01 | | 306.08 |
| D215 | B | 3.89 | | 382.09 |

### General Method 12 for the Preparation of O-substituted ethyl 2-(hydroxymethyl)cyclo-propanecarboxylates

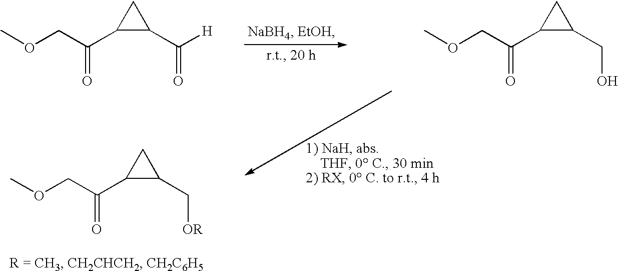

### Ethyl 2-(hydroxymethyl)cyclopropanecarboxylate

To a solution of 1.86 ml ethyl 2-formyl-1-cyclopropane-carboxylate (1.07 mmol, predominantly trans) in 10 ml EtOH at 0° C. were added 692 mg sodium borohydride (18.3 mmol) in three portions, and the mixture was stirred at room temperature for 20 h. After cooling to 0° C., the reaction was quenched by the addition of 1 M HCl solution (10 ml) and the aqueous layer extracted three times with DCM (20 ml). The combined organic layers were dried over Na₂SO₄ and after removal of the solvent 1.99 g of a colourless oil was obtained (Yield: 98%).

Mass calc. for C₇H₁₂O₃: 144.17, found (pos. mode) 145.1

### Ethyl 2-(methoxymethyl)cyclopropanecarboxylate

To a suspension of 72 mg sodium hydride (60% in mineral oil, 1.80 mmol) in 1.5 ml abs. THF were slowly added 200 mg ethyl 2-(hydroxymethyl)cyclopropane-carboxylate (1.39 mmol), and the resulting mixture was stirred for 30 min at 0° C. After dropwise addition of RX (methyliodide, allylbromide or benzylchloride, 1.80 mmol), the reaction was stirred for 3 h at 0° C., then for 1 h at r.t and finally quenched by the addition of water (10 ml). The resulting solution was extracted three times with DCM (10 ml) and the combined organic layers were dried over Na₂SO₄. Evaporation of the solvent and purification by bulb-to-bulb distillation afforded the desired compound as a colourless oil.

Yield: 52%.

Mass calc. for C₈H₁₄O₃: 158.20, found (pos. mode) 159.2.

NMR (200 MHz, CDCl₃): δ 0.85 (m, 1H), 1.23 (m, 1H), 1.26 (t, J=7.3 Hz, 3H), 1.57 (m, 1H), 1.71 (m, 1H), 3.26 (dd, J=10.3, 6.6 Hz, 2H), 3.35 (s, 3H), 3.37 (dd, J=10.3, 6.6 Hz, 1H), 4.12 (q, J=7.3 Hz, 2H).

According to this general method the following compounds were prepared:

### Ethyl 2-[(allyloxy)methyl]cyclopropanecarboxylate

Yield: 79%.

Mass calc. for C₉H₁₄O₃: 184.24.

NMR (200 MHz, CDCl₃): δ 0.88 (m, 1H), 1.24 (m, 1H), 1.26 (t, J=7.3 Hz, 3H), 1.58 (m, 1H), 1.71 (m, 1), 3.38 (m, 2H), 3.99 (m, 2H), 4.12 (q, J=7.3 Hz, 2H), 5.24 (m, 2H), 5.90 (m, 1H).

### Ethyl 2-[(benzyloxy)methyl]cyclopropanecarboxylate

Yield: 43%.

Mass calc. for C₁₄H₁₈O₃: 234.30.

NMR (200 MHz, CDCl₃): δ.0.86 (m, 1H), 1.22 (m, 1H), 1.26 (t, J=7.3 Hz, 3H), 1.52 (m, 1H), 1.73 (m, 1H), 3.41 (m, 2H), 4.12 (q, J=7.3 Hz, 2H), 4.53 (m, 2H), 7.36 (m, 5H).

### General Method 13 for the Hydrolysis of the Ethyl Cyclopropanoate Derivatives to the Corresponding Carboxylic Acids

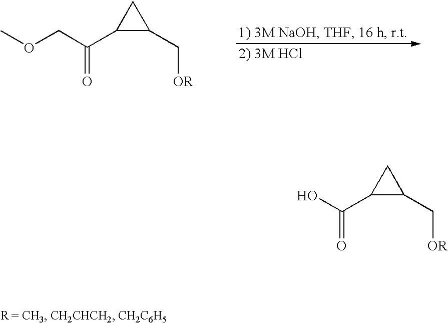

To a solution of ethyl cyclopropanecarboxylate (3.01 mmol) in 6 ml THF were added 6 ml 3M NaOH solution and the reaction was heated to reflux for 6 h. After cooling to room temperature, the reaction was acidified with 3M HCl solution to pH 1 and extracted three times with EtOAc (20 ml). Evaporation of the solvent yielded the title compound.

According to this general method the following compounds were prepared:

2-[(Methyloxy)methyl]cyclopropanecarboxylic acid

Yield: 69%

NMR (200 MHz, CDCl₃): δ.0.91 (m, 2H), 1.58 (m, 1H), 1.76 (m, 1H), 3.34 (dd, J=10.3, 6.6 Hz, 1H), 3.35 (s_{b}, 3H), 3.45 (dd, J=10.3, 5.9 Hz, 1H).

### 2-[(Allyloxy)methyl]cyclopropanecarboxylic acid

Yield: quant.

NMR (200 MHz, CDCl₃): δ.0.91 (m, 2H), 1.57 (m, 1H), 1.77 (m, 1H), 3.33 (dd, J=10.3, 6.6 Hz, 1H), 3.45 (dd, J=10.3, 5.9 Hz, 1H), 3.99 (d, J=5.9 H, 2H), 5.24 (m, 2H), 5.89 (m, 1H), 10.53 (s_{br}, 1H).

### 2-[(Benzyloxy)methyl]cyclopropanecarboxylic acid

Yield: 26%

NMR (200 MHz, CDCl₃): δ.0.93 (m, 1H), 1.26 (m, 1H), 1.56 (m, 1H), 1.79 (m, 1H), 3.34 (dd, J=10.3, 6.6 Hz, 1H), 3.46 (dd, J=10.3, 5.9 Hz, 1H), 4.51 (s, 2H), 7.30 (m, 5H).

### General Method 14 for the Nucleophilic Substitution of the Tosyl-Group with Secondary Amines

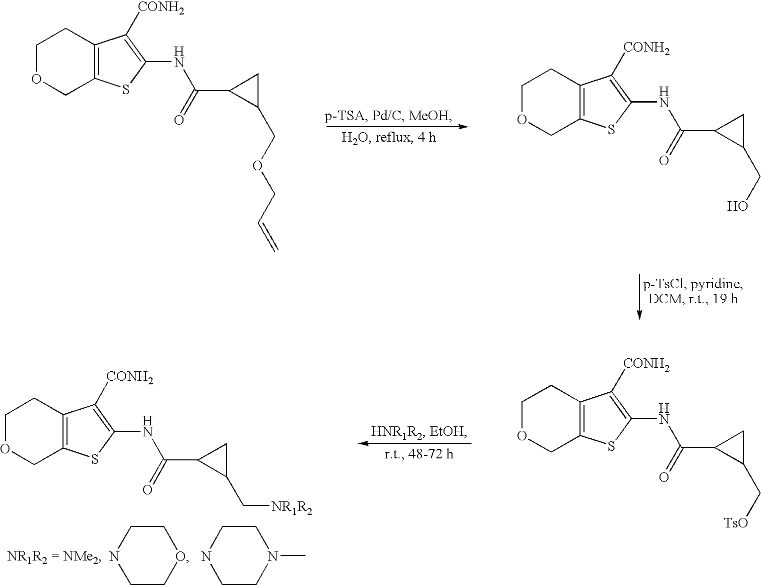

### 2-[(2-Hydroxymethyl-cyclopropanecarbonyl)-amino]-4,7-dihydro-5H-thieno[2,3-c]pyran-3-carboxylic acid amide (B229)

A suspension of 580 mg 2-[(2-allyloxymethyl-cyclopropanecarbonyl)-amino]-4,7-dihydro-5H-thieno[2,3-c]pyran-3-carboxylic acid amide (1.72 mmol), 327 mg p-toluenesulfonic acid (1.72 mmol) and 350 mg palladium on charcoal (10%, 0.33 mmol) in 10 ml methanol and 10 ml water was heated to reflux for 4 h. The reaction mixture was then cooled to room temperature and filtrated through a pad of Celite (solvent EtOAc). The filtrate was washed with brine (30 ml), the organic layer dried over Na₂SO₄ and the solvent evaporated. After purification by flash column chromatography on silica gel (DCM/MeOH 9:1) 208 mg of the title compound were obtained as a powder (Yield: 41%).

Mass calc. for C₁₃H₁₆N₂O₄S: 296.35, found (pos. mode) 297.2 found (neg. mode) 295.1.

### Toluene-4-sulfonic acid 2-(3-carbamoyl-4,7-dihydro-5H-thieno[2,3-c]pyran-2-ylcarbamoyl)-cyclopropylmethyl ester

A solution of 322 mg p-toluenesulfonyl chloride (1.68 mmol) and 208 mg 2-[(2-hydroxymethyl-cyclopropanecarbonyl)-amino]-4,7-dihydro-5H-thieno[2,3-c]pyran-3-carboxylic acid amide in 7 ml abs. DCM was stirred at room temperature and 272 μl pyridine (2.66 mg, 3.36 mmol) were slowly added. After 19 h stirring at room temperature, water (15ml) was added to the reaction mixture and the resulting solution was extracted three times with EtOAc (15 ml). The combined organic layers were dried over Na₂SO₄, the solvent evaporated and purification by flash column chromatography on silica gel (DCM/MeOH 96:4) yielded 210 mg of the desired product (Yield: 66%).

Mass calc. for C₂₀H₂₂N₂O₆S₂: 450.54, found (pos. mode) 451.2 found (neg. mode) 449.2.

The product was directly used in the next reaction step without further analysis.

Nucleophilic Substitution

To a solution of 104 mg toluene-4-sulfonic acid 2-(3-carbamoyl-4,7-dihydro-5H-thieno[2,3-c]pyran-2-ylcarbamoyl)-cyclopropylmethyl ester (0.24 mmol) in 2 ml EtOH and 1 ml DCM were added typically, 20-30 eq. of a secondary amine (neat or as commercially available solutions in THF) and the reaction was stirred for 48-72 h at room temperature. After addition of water (10 ml), the resulting solution was extracted three times with EtOAc (15 ml) and the combined organic layer was dried over Na₂SO₄. Evaporation of the solvent, followed by purification by preparative HPLC (Method 1) afforded the products as off-white solids.

According to this general method the following compounds were prepared:

B197: Mp.: 175-176° C.

Mass calc. for C₁₅H₂₁N₃O₃S: 323.42, found (pos. mode) 324.2 found (neg. mode) 322.2.

B196: Mp.: 193-194° C.

Mass calc. for C₁₇H₂₃N₃O₄S: 365.45, found (pos. mode) 366.1 found (neg. mode) 354.2.

B193:

Mass calc. for C₁₈H₂₆N₄O₃S: 378.50, found (pos. mode) 379.2 found (neg. mode) 377.2.

### General Method 15 for the Conversion of Ethyl 3-carboxylate Derivatives into 3-carboxamide Derivatives

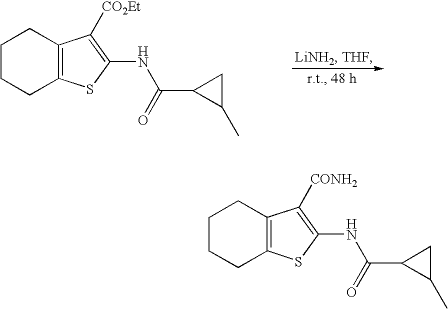

The ethyl ester (1 mmol) was dissolved in 3 ml abs. THF, then LiNH₂ (230 mg, 10 equivalents) was added and the mixture was stirred in a stoppered flask at r.t. for 48 h. The reaction mixture was poured on ice water, and the pH of the solution was adjusted to 5 with 5% HCl. The precipitate was filtered off and washed with cold isopropanol. The reaction can be run also under heterogeneous conditions. Diethyl ether can also be used as solvent.

According to this general method the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D9 | A | 3.08 | 279.13 | | B6 | A | 2.07 | | 359.16 |
| D10 | A | 3.1 | 281.19 | | B7 | A | 1.85 | 309.16 | |
| D11 | A | 3.03 | 279.18 | | B77 | A | 1.56 | | 279.15 |
| D12 | A | 3.36 | | 319.19 | D206 | A | 2.04 | | 291.14 |
| D196 | A | 2.97 | | 249.15 | B95 | A | 1.65 | | 279.18 |
| D13 | A | 3.13 | | 277.09 | B96 | A | 1.98 | | 321.19 |
| D194 | A | 3.24 | | 291.17 | B228 | A | 1.96 | | 321.21 |
| D193 | A | 3.27 | | 339.14 | B98 | A | 1.7 | | 281.18 |
| D187 | A | 3.19 | 293.15 | | B108 | A | 1.98 | 335.12 | |
| D186 | A | 3.26 | 307.15 | | B109 | A | 1.87 | | 307.14 |
| D159 | A | 3.33 | | 305.17 | B110 | A | 1.72 | | 363.16 |
| D158 | A | 3.27 | | 305.13 | B135 | A | 1.93 | 329.11 | |
| D156 | A | 2.07 | | 293.18 | B94 | A | 1.78 | 297.18 | |
| B17 | A | 1.66 | | 279.17 | B137 | A | 1.71 | | 279.1 |
| B18 | A | 1.64 | | 281.19 | B138 | A | 1.9 | 335.04 | |
| B19 | A | 1.91 | | 337.1 | B140 | A | 1.81 | 297.15 | |
| B2 | A | 1.54 | | 267.19 | D102 | A | 2.18 | | 305.18 |
| B10 | A | 1.7 | | 293.19 | B176 | A | 1.16 | | 240.17 |
| B14 | A | 1.71 | | 281.16 | B177 | A | 1.75 | | 293.17 |
| B15 | A | 1.65 | | 279.15 | B225 | A | 1.65 | | 269.11 |
| B16 | A | 1.59 | 267.12 | | B171 | B | 3.32 | | 295.22 |
| B3 | A | 1.91 | | 341.15 | B224 | B | 3.35 | | 295.08 |
| B4 | A | 1.67 | | 279.18 | B173 | B | 3.3 | | 295.22 |
| B5 | A | 1.68 | | 291.12 | B216 | B | 1.88 | | 337.16 |
| D210 | A | 3.46 | | 291.19 | | | | | |

### General Method 16 for the Preparation of Bromo-Substituted 6-hydroxy-benzo[b]thiophene-3-carboxamide Derivatives

### Procedure 16.1. 2-(Cyclopropanecarbonyl-amino)-7-bromo-6-hydroxy-benzo[b]thiophene-3-carboxamide (A32)

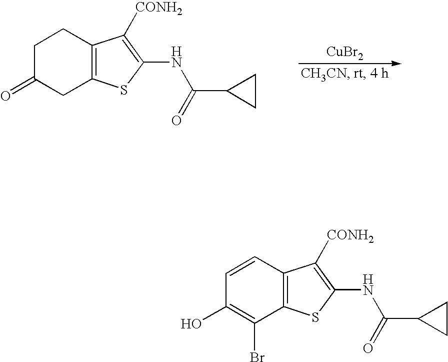

53 mg (0.24 mmol) CuBr₂ were added to a solution of 60 mg (0.22 mmol) 2-(cyclopropanecarbonyl-amino)-6-oxo-4,5,6,7-tetrahydro-benzo[b]thiophene-3-carboxamide in 2 ml CH₃CN. The mixture was stirred at room temperature for 4 h. 10 ml water were added and the mixture extracted three times with 20 ml EtOAc. The combined organic layers were washed with brine, dried over Na₂SO₄ and the solvent was evaporated. Separation by preparative HPLC (Method 1) afforded the title compound as a light brown solid in 10% yield.

### Procedure 16.2.

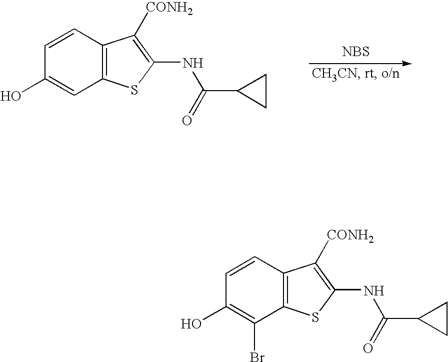

21 mg (0.12 mmol) of N-bromosuccinimide were added to a suspension of 30 mg (0.11 mmol) of 2-(cyclopropanecarbonyl-amino)-6-hydroxy-benzo[b]thiophene-3-carboxamide. The mixture was stirred at room temperature overnight. Water (10 ml) was added and the mixture extracted three times with 15 ml DCM. The combined organic layers were washed with brine, dried over Na₂SO₄ and the solvent was evaporated. Separation by preparative HPLC (Method 1) afforded the title compound as a yellow solid in 45% yield.

Mp.: 236-240° C. (CH₃CN).

Mass calc. for C₁₃H₁₁BrN₂O₃S: 355.21, found (pos. mode) 355.0 & 357.0, found (neg. mode) 353.0 & 355.0.

When using a 2.2 fold excess of N-bromosuccinimide in acetic acid as a solvent and stirring for 48 h at room temperature, the 5-7-di-bromo-derivative is obtained in 77% yield.

According to this procedure the following compound was prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| A14 | B | 3.84 | | 336.97 | A38 | B | 3.42 | | 430.87 |
| A40 | B | 3.33 | | 255.07 | | | | | |

### General Method 17 for the Preparation of Substituted amino- and thio-acetylamino Derivatives in Position 2

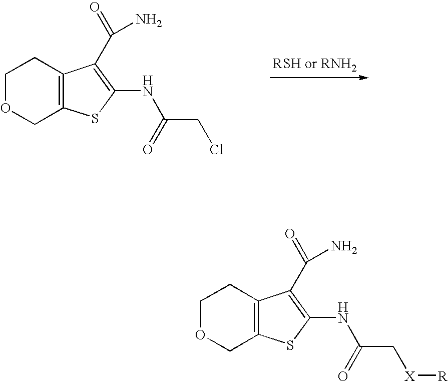

### Procedure 17.1. 2-(2-Cyclopropylamino-acetylamino)-4,7-dihydro-5H-thieno[2,3-c]pyran-3-carboxylic acid amide (B11)

275 mg (1 mmol) of 2-(2-chloro-acetylamino)-4,7-dihydro-5H-thieno[2,3-c]pyran-3-carboxylic acid amide were treated with 2.5 ml cyclopropylamine and refluxed till the product precipitated. The product was filtered, washed with water, cold isopropanol, diethyl ether and dried (Yield: 61%).

The following compounds were prepared by this method:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B111 | B | 1.65 | 296.15 | | B219 | B | 2.03 | | 310.15 |
| B204 | B | 1.68 | | 296.19 | B202 | B | 1.85 | 310.17 | |
| B223 | B | 2.09 | | 324.22 | B218 | B | 1.64 | | 367.19 |
| B64 | B | 1.93 | 326.18 | | B214 | B | 1.7 | 310.1 | |
| B115 | B | 2.3 | | 338.23 | B152 | B | 2.25 | | 364.17 |
| B222 | B | 2.19 | | 324.15 | B92 | B | 1.99 | | 336.16 |
| B220 | B | 2.15 | | 336.17 | | | | | |

### Procedure 17.2. 2-[2-(4-Fluoro-phenylsulfanyl)-acetylamino]-4,7-dihydro-5H-thieno[2,3-c]pyran-3-carboxylic acid amide (B215)

To a suspension of 275 mg (1 mmol) of 2-(2-chloro-acetylamino)-4,7-dihydro-5H-thieno[2,3-c]pyran-3-carboxylic acid amide in EtOH, the corresponding thiol (1.3 eq.) and NaOAc (1.3 eq.) wee added and the mixture was stirred at reflux for 2 h. Upon cooling the product precipitated and it was filtered and washed with water and isopropanol (Yield: 75%).

The following compounds were prepared by this method:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B215 | B | 3.39 | | 365.07 | B170 | B | 3.58 | | 381.05 |
| B213 | B | 3.68 | | 424.92 | B185 | B | 3.78 | 377.11 | |
| B212 | B | 3.33 | | 377.08 | B136 | B | 2.49 | 364.09 | |
| B211 | B | 3.63 | | 381.05 | B200 | B | 3.31 | 379.1 | |
| B210 | B | 3.9 | | 415 | B199 | B | 3.76 | | 375.09 |
| B209 | B | 3.58 | | 419.04 | B198 | B | 3.33 | 407.11 | |
| B208 | B | 3.35 | | 377.1 | B189 | B | 3.9 | | 460.01 |
| B207 | B | 3.92 | | 415.01 | B188 | B | 3.19 | | 429.22 |
| B206 | B | 3.55 | | 361.1 | B187 | B | 3.64 | | 414.95 |
| B205 | B | 3.53 | | 361.09 | B186 | B | 3.06 | | 398.16 |
| B23 | B | 3.62 | | 424.99 | B184 | B | 3.77 | 399.13 | |

### General Method 18 for the Preparation of 7-substituted 2-(cyclopropanecarbonyl-amino)-4,7-dihydro-5H-thieno[2,3-c]pyran-3-carboxylic acid amide Derivatives

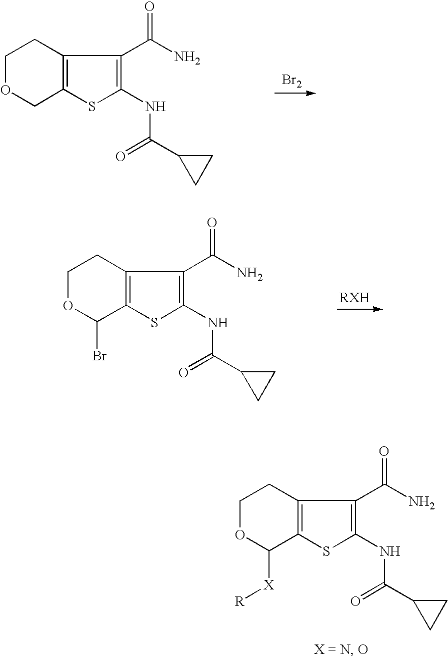

### 7-Bromo-2-(cyclopropanecarbonyl-amino)-4,7-dihydro-5H-thieno[2,3-c]pyran-3-carboxylic acid amide

To a solution of 1.33 g (5.00 mmol) of 2-(cyclopropanecarbonyl-amino)-4,7-dihydro-5H-thieno[2,3-c]pyran-3-carboxylic acid amide and 0.45 g (5.50 mmol) of sodium acetate in 30 ml acetic acid, 0.88 g (5.50 mmol) of bromine was added dropwise at room temperature. After stirring the reaction mixture at room temperature for 1 h the precipitated product was filtered off, washed with 15 ml diisopropyl ether and dried to yield the title compound in 67% yield.

### Synthesis of 2-(Cyclopropanecarbonyl-amino)-7-(2-ethoxy-ethoxy)-4,7-dihydro-5H-thieno[2,3-c]pyran-3-carboxylic acid amide (B240)

170 mg (0.50 mmol) of 7-bromo-2-(cyclopropanecarbonyl-amino)-4,7-dihydro-5H-thieno[2,3-c]pyran-3-carboxylic acid amide, 0.06 g (5.0 mmol) diisopropyl-ethylamine and 5.00 mmol of 2-ethoxy-ethanol were refluxed in 25 ml abs. tetrahydrofuran for 2-4 h. The solvent was evaporated, and the residue was partitioned between ethyl acetate and water (15 ml each). The aqueous phase was extracted two times with 15 ml ethyl acetate, the combined organic phases were washed with 15 ml brine, dried over magnesium sulphate, and the solvent was evaporated. The crude product was crystallized from acetonitrile to give the title compound.

When using N-nucleophiles diisopropyl-ethylamine may be omitted.

According to this general method the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B165 | B | 2.96 | | 309.18 | B65 | B | 1.81 | | 385.23 |
| B49 | B | 2.1 | | 350.19 | B66 | B | 0.56 | | 407.27 |
| B167 | B | 3.93 | | 408.16 | B238 | B | 1.67 | 393.15 | |
| B168 | B | 1.69 | 395.22 | | B239 | B | 1.97 | 373.09 | |
| B99 | B | 3.16 | | 323.18 | B240 | B | 3.01 | | 353.11 |
| B27 | B | 1.52 | | 351.2 | B241 | B | 2.13 | 382.11 | |
| B26 | B | 2.11 | | 365.19 | B235 | B | 2.78 | | 339.08 |
| B25 | B | 2.4 | 456.21 | | B242 | B | 3.11 | | 343.03 |
| B24 | B | 1.64 | 310.18 | | B246 | B | 2.34 | 390.1 | |
| B61 | B | 0.55 | 367.16 | | | | | | |

### Cyclopropanecarboxylic acid(3-carbamimidoyl-4,5,6,7-tetrahydro-benzo[b]-thiophene-2-yl)-amide (D133)

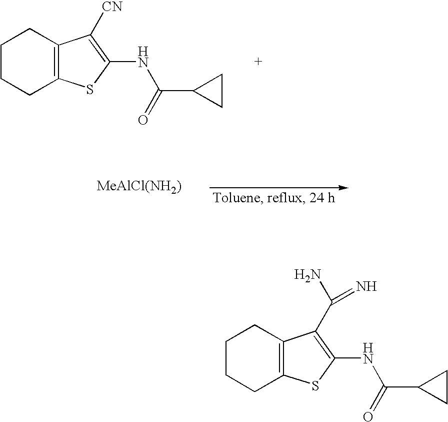

A suspension of 53 mg (1.0 mmol) NH₄Cl in 2.5 ml abs. toluene at 0° C. under argon was treated dropwise with 500 μl (1.0 mmol) AlMe₃ (2 M solution in toluene). The mixture was stirred at room temperature until the evolution of gas stopped. This solution was added dropwise to a suspension of 100 mg (0.41 mmol) cyclopropanecarboxylic acid (3-cyano-4,5,6,7-tetrahydro-benzo[b]thiophene-2-yl)-amide in 1 ml toluene. The mixture was stirred under reflux for 24 h. After cooling to room temperature the mixture was poured into a slurry of 10 g silica gel in CH₂Cl₂ and stirred for 10 min. The silica gel was filtered and washed with CH₂Cl₂. Evaporation of the solvent gave the crude product, which was purified by flash column chromatography on silica gel (eluent DCM/MeOH 10:1 to 4:1) to afford the title product as a colourless solid in 43% yield.

Mp.: >240° C. (decomposition).

Mass calc. for C₁₃H₁₇N₃OS: 263.36, found (pos. mode) 264.25, (neg. mode) 262.29.

### Cyclopropanecarboxylic acid (3-thiocarbamoyl-4,5,6,7-tetrahydro-benzo[b]-thiophene-2-yl)-amide (D99) and 2-(cyclopropanecarbothioyl-amino)-4,5,6,7-tetrahydro-benzo[b]thiophene-3-carbothioic acid amide (D100)

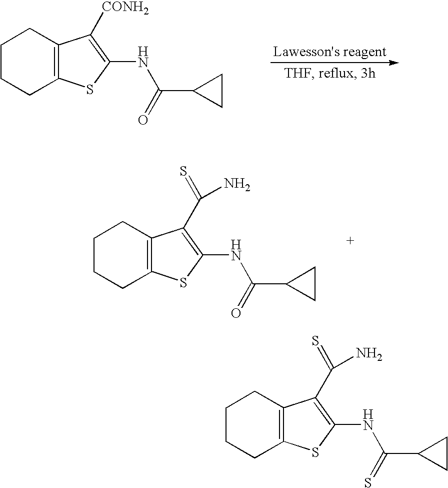

A solution of 243 mg (0.92 mmol) 2-(cyclopropanecarbonyl-amino)-4,5,6,7-tetrahydro-benzo[b]thiophene-3-carboxamide and 557 mg (1.38 mmol) Lawesson's reagent in 6 ml THF was stirred under reflux for 3 h. The crude mixture was directly purified by flash column chromatography on silica gel (eluent cHex/EtOAc 10:1 to 5:1) to yield the title compound (D99) as yellow crystals in 35%.

Mp.: 182-184° C. (cHex/CH₂Cl₂),

Mass calc. for C₁₃H₁₆N₂OS₂: 280.41, found (neg. mode) 279.28; and the title compound (D100) as an orange powder in 18% yield,

Mp.: 169-170° C. (decomposition, cHex/CH₂Cl₂).

### 3-(Aminocarbonyl)-2-(cyclopropanecarbonyl-amino)-1-benzothien-6-yl acetate (A3)

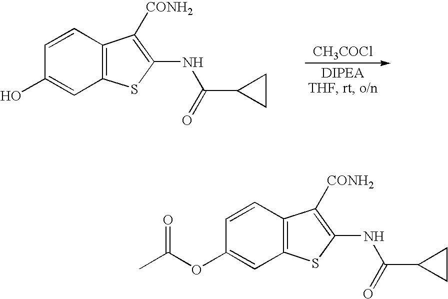

A suspension of 50 mg (0.18 mmol) 2-(cyclopropanecarbonyl-amino)-6-hydroxy-benzo[b]thiophene-3-carboxamide and 35 μl (0.20 mmol) diisopropylethylamine in 2.5 ml THF was treated with 14 μl (0.20 mmol) acetyl chloride and stirred at room temperature overnight. 10 ml 0.5 M HCl solution was added and the mixture extracted three times with 20 ml EtOAc. The combined organic layers were washed with brine and dried over Na₂SO₄. Evaporation of the solvent gave the crude product, which was purified by flash column chromatography on silica gel (eluent cHex/EtOAc 1:1) to afford a colourless solid in 42% yield.

Mp.: 223-224° C. (ethanol).

Mass calc. for C₁₅H₁₄N₂O₄S: 318.35, found (pos. mode) 319.2, found (neg. mode) 317.2.

### 2-(Cyclopropanecarbonyl-amino)-6-hydroxy-benzo[b]thiophene-3,7-dicarboxylic acid 3-amide 7-diethylamide (A18)

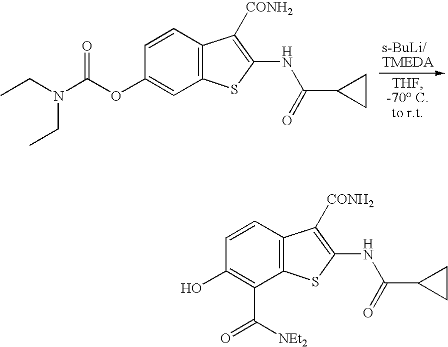

To a solution of 70 mg (0.19 mmol) diethyl-carbamic acid 3-carbamoyl-2-(cyclopropanecarbonyl-amino)-benzo[b]thiophen-6-yl ester and 113 μl (0.75 mmol) N,N,N′,N′-tetramethylethylenediamine in 2 ml THF at −70° C. were added 533 μl (0.75 mmol) s-BuLi (1.4 M solution in cHex) dropwise. The mixture was stirred at −70° C. for 45 min, then slowly heated to room temperature over 2 h and stirred at room temperature for another 2 h. The mixture was treated with 10 ml sat. NH₄Cl solution and extracted three times with 30 ml EtOAc. The combined organic layers were washed with brine and dried over Na₂SO₄. Evaporation of the solvent gave the crude product, which was purified by preparative HPLC (Method 1) to afford the title product as a colourless solid in 15% yield.

Mp.: 233-235° C. (H₂O/CH₃CN).

Mass calc. for C₁₈H₂₁N₃O₄S: 375.45, found (pos. mode) 376.2, found (neg. mode) 374.3.

### 2-(Cyclopropanecarbonyl-amino)-6-hydroxy-benzo[b]thiophene-3,5-dicarboxylic acid 3-amide 5-diethylamide (A17)

In the procedure described for 2-(cyclopropanecarbonyl-amino)-6-hydroxy-benzo[b]thiophene-3,7-dicarboxylic acid 3-amide 7-diethylamide, the title compound was isolated as a second product by preparative HPLC (Method 1) to afford the title product as a colourless solid in 11% yield.

Mp.: 227-228° C. (H₂O/CH₃CN).

Mass calc. for C₁₈H₂₁N₃O₄S: 375.45, found (pos. mode) 376.2, found (neg. mode) 374.2.

### 2-(Cyclopropanecarbonyl-amino)-6-(2-hydroxy-ethoxy)-benzo[b]thiophene-3-carboxamide (A20)

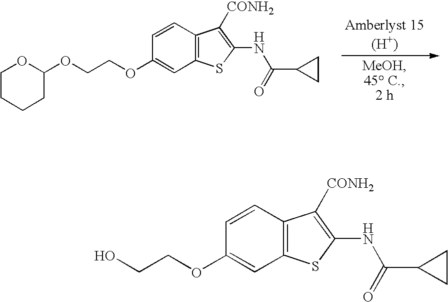

To a solution of 100 mg (0.25 mmol) 2-(cyclopropanecarbonyl-amino)-6-[2-(tetrahydropyran-2-yloxy)-ethoxy]-benzo[b]thiophene-3-carboxamide in 5 ml methanol was added 180 mg Amberlyst 15 (H⁺-form). The mixture was stirred at 45° C. for 2 h, the Amberlyst 15 filtered off and the solvent evaporated. Recrystallization of the residue from ethanol/methanol (2:1) afforded the product as a colourless solid in 91% yield.

Mp.: 203-205° C. (MeOH/EtOH).

Mass calc. for C₁₅H₁₆N₂O₄S: 320.37, found (pos. mode) 321.2.

Compound A49 was prepared according to this method (Yield: 50%).

Mp.: 250-254° C.

NMR, δ (ppm): 11.88 (s, 1H), 7.93 (s, 1H), 7.86 (s, 2H), 4.88 (t, J=5.58 Hz, 1H), 4.05 (t, J=5.13 Hz, 2H), 3.78 (m, 2H), 2.07 (m, 1H), 0.96 (m, 4H).

### 2-(Cyclopropanecarbonyl-amino)-6,6-difluoro-4,5,6,7-tetrahydro-benzo[b]-thiophene-3-carboxamide (D128)

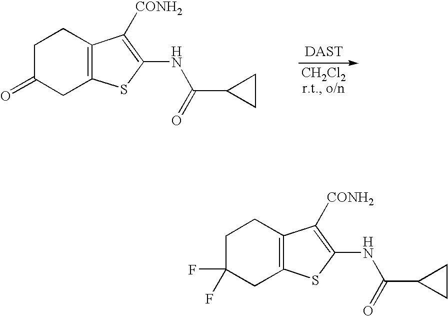

50 mg (0.18 mmol) 2-(cyclopropanecarbonyl-amino)-6-oxo-4,5,6,7-tetrahydro-benzo[b]thio-phene-3-carboxamide was suspended in 2 ml CH₂Cl₂, and 24 μl (0.18 mmol) diethylamino sulfur trifluoride was added dropwise at 0° C. The mixture was stirred at room temperature overnight. 10 ml sat. NH₄Cl-solution was added and the mixture extracted three times with 20 ml EtOAc. The combined organic extracts were washed with brine and dried over Na₂SO₄. Evaporation of the solvent gave the crude product, which was purified by flash column chromatography on silica gel (eluent cHex/EtOAc 1:3). The title compound was isolated as a colourless solid in 12% yield.

Mass calc. for C₁₃H₁₄F₂N₂O₂S: 300.33, found (pos. mode) 301.19, found (neg. mode) 299.20.

Using the same procedure described for compound D128 the following compound was prepared:

D66: Mp.: 207-208° C. (H₂O/CH₃CN).

Mass calc. for C₁₃H₁₅FN₂O₂S: 282.34, found (pos. mode) 283.1, found (neg. mode) 281.1.

### Preparation of Cyclopropanecarboxylic acid (3-sulfamoyl-4,5,6,7-tetrahydro-benzo[b]thiophen-2-yl)-amide (D86)

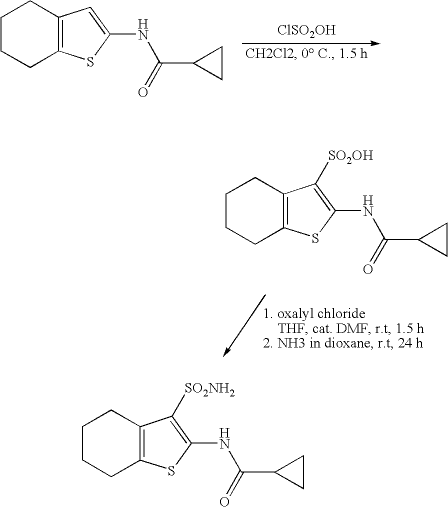

### 2-(Cyclopropanecarbonyl-amino)-4,5,6,7-tetrahydro-benzo[b]thiophene-3-sulfonic acid

40 μl (0.60 mmol) CISO₃H was added dropwise to a suspension of 50 mg (0.23 mmol) cyclopropanecarboxylic acid (4,5,6,7-tetrahydro-benzo[b]thiophen-2-yl)-amide in 1 ml CH₂Cl₂ at 0° C. The resulting solution was stirred at 0° C. for 1.5 h, then 0.5 ml of water were added. Slow evaporation of the solvents under inert atmosphere afforded the title compound as a colourless solid in 85% yield.

Mp.: 225-230° C. (methanol, decomposition).

Mass calc. for C₁₂H₁₅NO₄S₂: 301.39, found (pos. mode) 302.2, found (neg. mode) 300.2.

### Cyclopropanecarboxylic acid (3-sulfamoyl-4,5,6,7-tetrahydro-benzo[b]thiophen-2-yl)-amide (D86)

A suspension of 32 mg (0.11 mmol) 2-(cyclopropanecarbonyl-amino)-4,5,6,7-tetrahydro-benzo[b]thiophene-3-sulfonic acid and 2 drops of DMF in 2 ml THF was treated dropwise with 10 μl (0.12 mmol) oxalyl chloride and stirred for 1.5 h at room temperature. 1 ml (0.5 mmol) ammonia (0.5 M solution in dioxane) was added and the mixture stirred at room temperature for 24 h. The solvent was evaporated and the crude product purified by flash column chromatography on silica gel (eluent cHex/EtOAc 4:1) to afford the title compound as a colourless solid in 25% yield.

Mp.: 235-238° C. (decomposition).

Mass calc. for C₁₂H₁₆N₂O₃S₂: 300.40, found (pos. mode) 301.2, found (neg. mode) 299.2.

### (6-R,S)-3-Carbamoyl-2-(cyclopropanecarbonyl-amino)-4,5,6,7-tetrahydro-benzo[b]thiophene-6-carboxylic acid (D82)

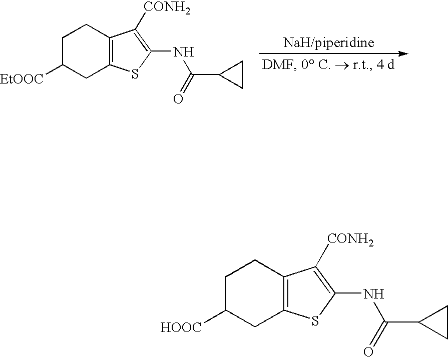

To a suspension of 13 mg (0.31 mmol) NaH (60% dispersion in mineral oil) in 2 ml DMF was added 11 μl (0.14 mmol) piperidine. The mixture was stirred at room temperature for 30 min, cooled to 0° C. and treated with a solution of 35 mg (0.10 mmol) (6-R,S)-2-amino-3-carbamoyl-4,5,6,7-tetrahydro-benzo[b]thiophene-6-carboxylic acid ethyl ester in 2 ml DMF. The mixture was stirred at room temperature for 4 days. 15 ml 0.5 M HCl solution were added and the mixture extracted four times with 40 ml EtOAc. The combined organic extracts were washed with brine and dried over Na₂SO₄. Evaporation of the solvent gave the product as a colourless solid in 90% yield.

Mp.: >230° C. (EtOAc, decomposition).

Mass calc. for C₁₄H₁₆N₂O₄S: 308.36, found (pos. mode) 309.1.

### (6-R,S)-2-(Cyclopropanecarbonyl-amino)-6-hydroxymethyl-4,5,6,7-tetrahydro-benzo[b]thiophene-3-carboxamide (D108)

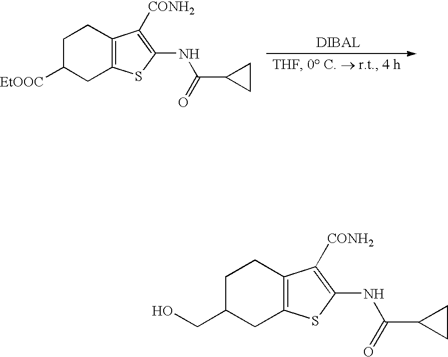

A solution of 50 mg (0.15 mmol) (6-R,S)-2-amino-3-carbamoyl-4,5,6,7-tetrahydro-benzo[b]thiophene-6-carboxylic acid ethyl ester in 1.5 ml THF was treated with 1 ml (1.00 mmol) diisobutylaluminium hydride (1 M solution in DCM) at 0° C. The mixture was stirred at 0° C. for 30 min and then allowed to stir at room temperature for 4 h. The mixture was treated with 10 ml sat. NH₄Cl solution and extracted three times with 30 ml EtOAc. The combined organic extracts were washed with brine and dried over Na₂SO₄. Evaporation of the solvent gave the crude product, which was purified by preparative HPLC (Method 1) to afford the product as a colourless solid in 34% yield.

Mp.: 215-222° C. (CH₃CN/H₂O, decomposition).

Mass calc. for C₁₄H₁₈N₂O₃S: 294.38, found (pos. mode) 295.1.

### (6-R,S)-2-(Cyclopropanecarbonyl-amino)-6-(1-hydroxy-1-methyl-ethyl)-4,5,6,7-tetrahydro-benzo[b]thiophene-3-carboxamide (D101)

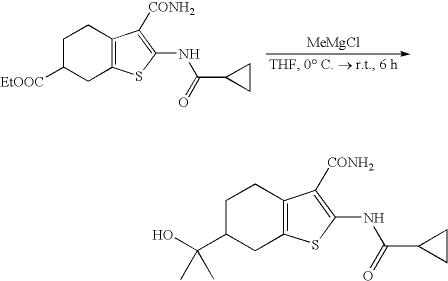

A suspension of 47 mg (0.14 mmol) (6-R,S)-2-amino-3-carbamoyl-4,5,6,7-tetrahydro-benzo[b]thiophene-6-carboxylic acid ethyl ester in 2 ml THF was treated with 233 μl (0.70 mmol) MeMgCl (3 M solution in THF) at 0° C. The mixture was stirred at room temperature for 6 h. The mixture was treated with 10 ml sat. NH₄Cl solution and extracted three times with 30 ml EtOAc. The combined organic extracts were washed with brine and dried over Na₂SO₄. Evaporation of the solvent gave the crude product, which was purified by preparative HPLC (Method 1) to afford the product as a colourless solid in 51% yield.

Mp.: 196-197° C. (CH₃CN/H₂O).

Mass calc. for C₁₆H₂₂N₂O₃S: 322.43, found (pos. mode) 323.1.

### 6,6-Dibromo-2-(cyclopropylcarbonyl-amino)-7-oxo-4,5,6,7-tetrahydro-benzo[b]-thiophen-3-carboxylic acid amide (D67)

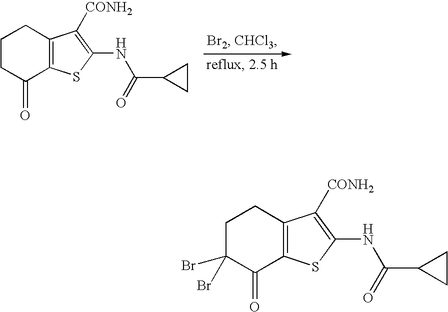

480 mg 2-(cyclopropylcarbonyl-amino)-7-oxo-4,5,6,7-tetrahydro-benzo[b]thiophen-3-carboxylic acid amide (1.73 mmol) and 195 μl bromine (3.79 mmol) in 15 ml chloroform were heated to reflux for 2.5 h. The reaction was then poured into an aqueous sodium thiosulfate solution (20 ml) and extracted three times with EtOAc (20 ml). Drying of the combined organic layers over Na₂SO₄ and evaporation of the solvent gave a brown solid, which was further purified by column chromatography on silica gel (eluent DCM/MeOH 95:5). 585 mg of a brown solid were obtained in 78% yield.

Mp.: 196-197° C. (dec.).

Mass calc. for C₁₃H₁₂Br₂N₂O₃S: 436.12, found (pos. mode) 434.9, 436.9 & 438.8, found (neg. mode) 432.9, 434.9 & 436.9.

### 2-(Cyclopropanecarbonyl-amino)-6-bromo-7-hydroxy-benzo[b]thiophene-3-carboxamide (A15)

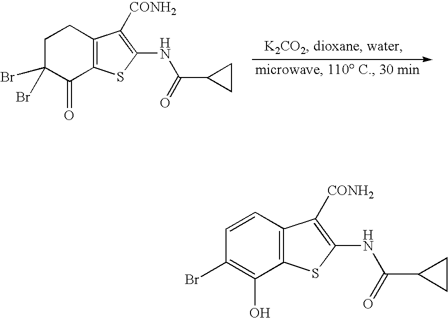

To a mixture of 20 mg 6,6-dibromo-2-(cyclopropylcarbonyl-amino)-7-oxo-4,5,6,7-tetrahydro-benzo[b]thiophen-3-carboxylic acid amide (45.9 μmol) in 1.0 ml dioxane were added 20 mg potassium carbonate (145.0 μmol) dissolved in 600 μl water, and the resulting yellow solution was heated in the microwave at 110° C. for 30 min. The reaction was poured into water (10 ml) and extracted three times with n-butanol (10 ml). The combined organic layers were evaporated to dryness and the resulting brown oil purified by preparative HPLC (Method 1). 6.0 mg of the desired compound were isolated (Yield: 37%).

Mp.: 235-236° C.

Mass calc. for C₁₃H₁₁BrN₂O₃S: 355.21, found (pos. mode) 355.1 & 357.1, found (neg. mode) 353.1 & 355.1.

### Synthesis of 3-(2,2-dichlorovinyl)-2,2-dimethylcyclopropanecarboxylic acid

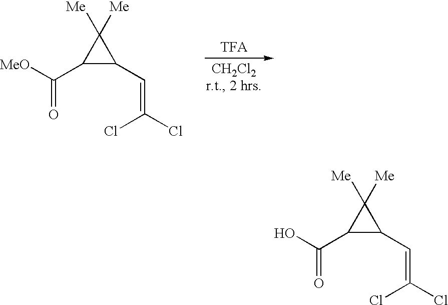

3.8 g (17.1 mmol) of methyl 3-(2,2-dichloroethenyl)-2,2-dimethylcyclopropane carboxylate were dissolved in 20 ml of dichloromethane. 10 ml of trifluoroacetic acid (TFA) were slowly added via syringe at room temperature, and the mixture was stirred for 2 h. After addition of 20 ml of 3N NaOH, keeping the pH below 2, 50 ml of water were added, and the mixture was extracted three times with dichloromethane. The organic layer was dried over Na₂SO₄. Filtration and evaporation of the solvent gave 3.28 g (92%) of a golden liquid, that was used without further purification.

### Synthesis of 2,2-dimethylcyclopropanecarboxylic acid

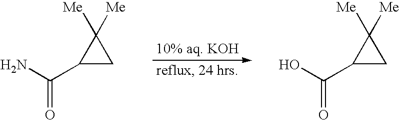

301.6 mg (2.7 mmol) of 2,2-dimethylcyclopropane carboxamide were refluxed for 24 h in 50 ml of 10% aqueous KOH, then stirred at room temperature for another 24 h. The mixture was acidified with concentrated HCl to a pH of 1, then extracted three times with EtOAc. The organic layer was dried over Na₂SO₄. Filtration and evaporation of the solvent gave a pale yellow liquid, that was used without further purification.

Mass calc. for C₆H₁₀O₂: 114.07, found (pos. mode) 115.27.

### Synthesis of 2,2-dichloro-3,3-dimethylcyclopropanecarboxylic acid

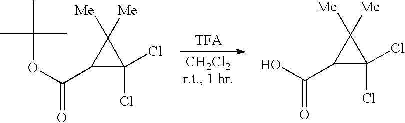

300 mg (1.25 mmol) of tert-butyl 2,2-dichloro-3,3-dimethylcyclopropanecarboxylate were dissolved in 5 ml of dichloromethane. 5 ml of trifluoroacetic acid (TFA) were slowly added via syringe at room temperature, and the mixture was stirred for 1 h. After addition of 3N NaOH, keeping the pH below 2, 20 ml of water were added, and the mixture was extracted three times with dichloromethane. The organic layer was dried over Na₂SO₄. Filtration and evaporation of the solvent gave 215 mg (94%) of a brown oil, that was used without further purification.

NMR (200 MHz, DMSO-d₆): δ 1.35 (s, 6H), 2.48 (s, 1H).

### Synthesis of 2-amino-1-benzyl-4,5,6,7-tetrahydro-1H-indole-3-carboxamide

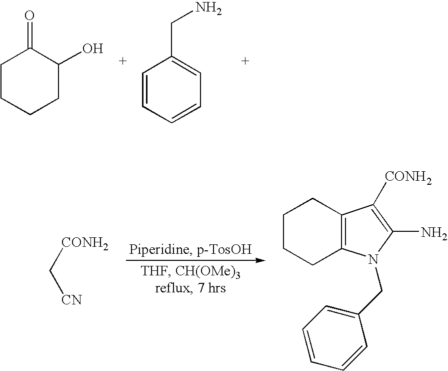

5 g (44 mmol of monomer) of the dimer of 2-hydroxycyclohexanone, 4.8 ml (44 mmol) of benzylamine, and a spatula tip of p-toluenesulfonic acid were dissolved in a mixture of 50 ml of trimethylorthoformate and 50 ml of abs. THF, and heated under reflux for 4 h. After cooling, 2.2 ml of piperidine were added to the solution, followed by dropwise addition of 3.7 g (44 mmol) of cyanoacetamide in 100 ml of abs. methanol. The solution was heated under reflux for 4 h, and subsequently allowed to cool over night. 100 ml of water were added, and the mixture was extracted three times with EtOAc. The organic layer was dried over Na₂SO₄. Filtration and evaporation of the solvents gave a crude material that was recrystallized from aqueous ethanol, giving 591 mg (5%) of a pale white solid.

Mp.: 164-165° C. (aq. EtOH)

Mass calc. for C₁₆H₁₉N₃O: 269.35, found (pos. mode) 270.44.

### Synthesis of 1-benzyl-2-[(cyclopropylcarbonyl)amino]-4,5,6,7-tetrahydro-1H-indole-3-carboxamide (D92)

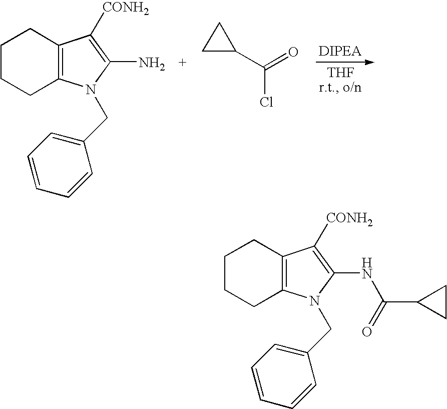

To a solution of 211.4 mg (0.79 mmol) of 2-amino-1-benzyl-4,5,6,7-tetrahydro-1H-indole-3-carboxamide and 300 μl (1.73 mmol; 2.2 equivalents) of diisopropylethylamine (DIPEA) in 20 ml of abs. THF under nitrogen atmosphere were added 85 μl (0.94 mmol; 1.2 equivalents) of cyclopropanecarbonyl chloride via syringe at room temperature. The mixture was stirred over night. The solution was diluted with 20 ml of water and extracted several times with EtOAc. The organic layer was dried over Na₂SO₄. Filtration and evaporation of the solvent gave crude material that was recrystallized from chloroform, giving 17.3 mg (6.5%) of a white solid.

Mp.: >250° C. (decomp.)

Mass calc. for C₂₀H₂₃N₃O₂: 337.42, found (neg. mode) 336.40.

### Synthesis of N-(3-{[(2-hydroxyethyl)amino]carbonyl}-4,5,6,7-tetrahydro-benzo[b]thien-2-yl)-2-furamide (D2)

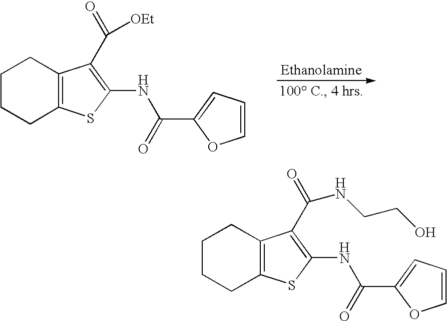

In a distillation setup, 98.1 mg (0.29 mmol) of ethyl 2-(2-furoylamino)-4,5,6,7-tetrahydro-benzo[b]thiophene-3-carboxylate and 2.5 ml of ethanolamine were heated at 100° C. for 4 h. The developing ethanol was continuously distilled from the mixture, additionally applying a slow stream of nitrogen gas. After cooling, the excess of ethanolamine was removed in a Kugelrohr distillation apparatus. The crude residue was recrystallized from ethanol to give 70.2 mg (72%) of fine yellow needles.

Mp.: 194-195° C. (EtOH)

Mass calc. for C₁₆H₁₈N₂O₄S: 334.40, found (neg. mode) 333.28.

### Synthesis of 4-[3-(3-carbamoyl-4,7-dihydro-5H-thieno[2,3-c]pyran-2-yl)-ureido]-1-methyl-pyridinium Iodide (B157)

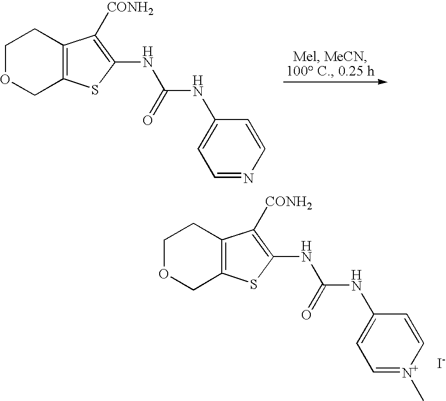

To a solution of 318 mg of 2-(3-Pyridin-4-yl-ureido)-4,7-dihydro-5H-thieno[2,3-c]pyran-3-carboxylic acid amide (1 mmol) in 1 ml acetonitrile, 142 mg of iodomethane (1 mmol) were added. The mixture was heated for 15 minutes at 100° C. in a closed reaction vessel. The precipitate was filtered off, washed with diethyl ether and dried.

HPLC method B; retention time: 2.05; M⁻: 331.21

The following compound was also prepared:

1-Benzyl-4-[3-(3-carbamoyl-4,7-dihydro-5H-thieno[2,3-c]pyran-2-yl)-ureido]-pyridinium Bromide (B72): HPLC method B; retention time: 2.6; M⁺: 410.16

Synthesis of 2-(Cyclopropanecarbonyl-amino)-4,5,6,7-tetrahydro-benzo[b]-thiophene-3-carboxylic acid methylamide (D185)

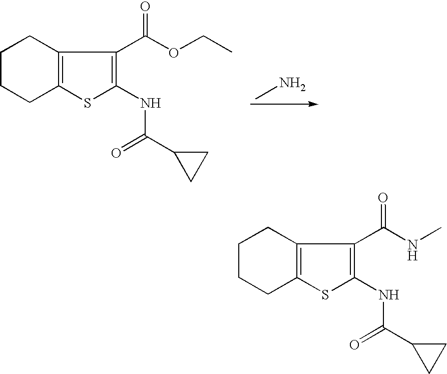

293 mg (1.00 mmol) of 2-(cyclopropanecarbonyl-amino)-4,5,6,7-tetrahydro-benzo[b]thiophene-3-carboxylic acid ethyl ester was stirred in 2 ml of a solution containing 33% of methylamine in ethanol at 0° C. for 48 h. The reaction mixture was evaporated to dryness and the product was purified by column chromatography on silica gel (Yield: 22%).

The following compounds were prepared by this method:

| | HPLC | Retention | | | | | Retention | | |
|---|---|---|---|---|---|---|---|---|---|
| ID | Method | Time | M⁺ | M⁻ | ID | HPLC Method | Time | M⁺ | M⁻ |
| A36 | A | 3.16 | | 273.11 | D185 | A | 3.05 | 279.17 | |
| D191 | A | 3.26 | | 305.18 | | | | | |

### Synthesis of N-(3-carbamoyl 4,7-dihydro-5H-thieno[2,3c]pyran-2-yl)oxalamide (B41)

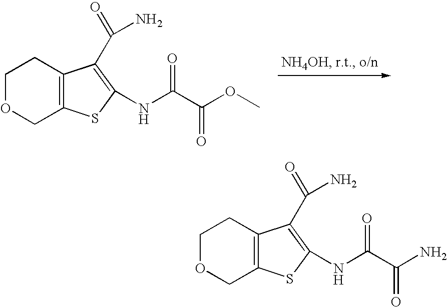

To a mixture of 150 mg (0.53 mmol) of N-(3-carbamoyl 4,7-dihydro-5H-thieno[2,3c]pyran-2-yl)oxalamic acid methylester in 5 ml methanol, 1 ml of conc. ammonium hydroxide was added, and the reaction mixture was stirred overnight in a stoppered flask. The solvent was evaporated and the residue was triturated with 0.5 mL methanol and filtered to obtain the title compound in 96% yield.

HPLC method B; retention time: 2.19; M⁻: 268.11

### General Method 19 for the Preparation of Carbamic Acid Esters and Ureas Derived Therefrom

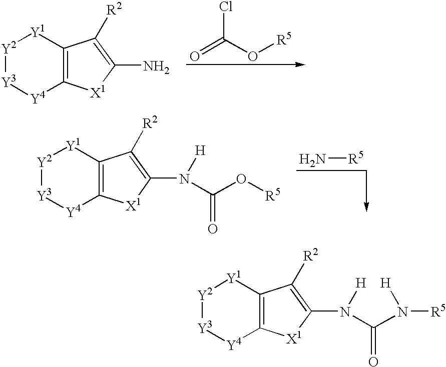

The above outlined general method 19 is exemplified by the synthesis of A67.

### 19.1A Synthesis of (3-carbamoyl-6,6-ethylenedioxy-4,5,6,7-tetrahydro-benzo[b]thiophen-2-yl)-carbamic acid phenyl ester (D235)

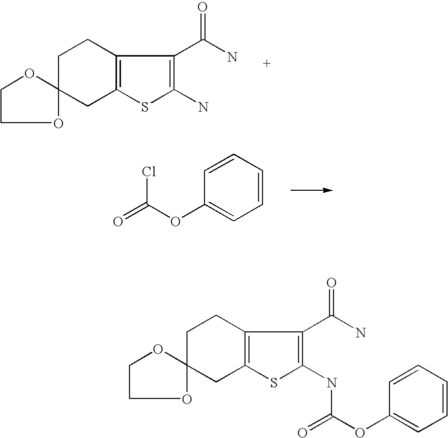

D235 also named as phenyl 7′-(aminocarbonyl)spiro[1,3-dioxolane-2,3′-[9]thiabicyclo[4.3.1]deca[6(10),7]dien]-8′-ylcarbamate was prepared according to procedure 19.1C. After stirring the reaction mixture, the reaction mixture was diluted with water (60 ml), and the mixture was extracted with EtOAc (3×30 ml). The combined organic layers were washed with brine and dried over Na₂SO₄. Evaporation of the solvent in vacuum at 20° C., the residue was treated with diethyl ether, the precipitate was filtered off and washed with diethyl ether affording 3.53 g (94%) of the title compound.

HPLC method B; retention time: 3.57; M⁻: 373.42

19.1B Preparation of (3-carbamoyl-6,6-ethylenedioxy-4,5,6,7tetrahydro-benzo[b]thiophen-2-yl)-carbamic acid ethyl ester

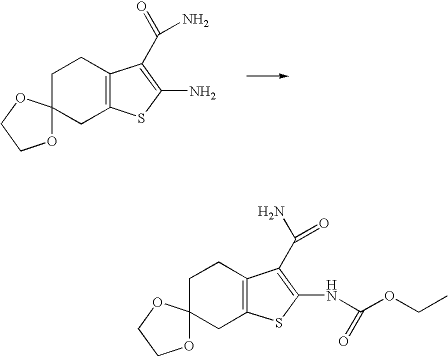

This compound was prepared according to 19.1A or 19.1C using dry pyridine as solvent (Yield: 0.27=85%)

NMR, δ (ppm): 10.92 (s, 1H), 7.20 (broad s, 2H), 4.15 (m, 2H), 3.93 (s, 4H), 2.82 (m, 4H), 1.82 (m, 2H), 1.23 (t, J=7.08 Hz, 3H).

### 19.1C Preparation of (3-carbamoyl-4,7-dihydro-5H-thieno[2,3-c]pyran-2-yl)-carbamic acid phenyl ester

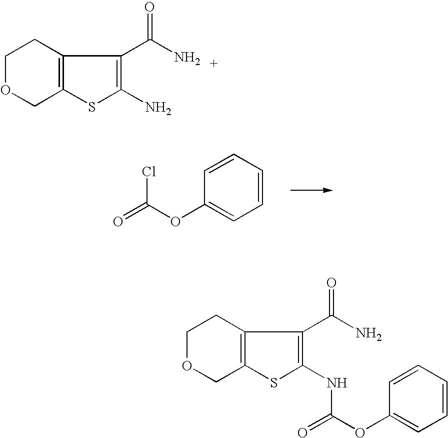

To a solution of 2-amino-4,7-dihydro-5H-thieno[2,3-c]pyran-3-carboxylic acid amide (4.45 g, 22.44 mmol) in 90 ml of tetrahydrofuran, pyridine (2.3 ml, 28.4 mmol) was added and stirred at 5° C. To the reaction mixture phenyl chloroformate (3.4 ml 24.58 mmol) was added dropwise at 5° C. After stirring for 3 h the mixture was diluted with water (50 ml), the solid was collected, washed with water, and dried in vacuum, affording 5.76 g (yield: 81%) of the title compound as a white solid.

### 19.2 Synthesis of (3-carbamoyl-6-hydroxy-benzo[b]thiophen-2-yl)-carbamic acid phenyl ester

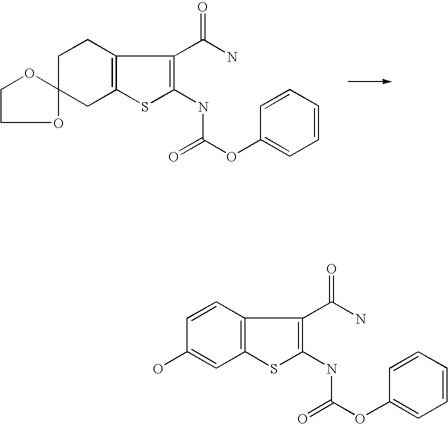

A suspension of 3.07 g (8.2 mmol) (3-carbamoyl-6,6-ethylenedioxy-4,5,6,7-tetrahydro-benzo[b]thiophen-2-yl)-carbamic acid phenyl ester in 65 ml CH₃CN was treated with 2.93 g (17.1 mmol) CuCl₂.2 H₂O at room temperature. The mixture turned brown immediately and was stirred at room temperature for 1 h. The solvent was evaporated in vacuum at 20° C. and the residue taken up in 100 ml EtOAc and extracted with 6×100 ml 0.5 M HCl solution. The organic layer was washed with brine and dried over Na₂SO₄. Evaporation of the solvent, the residue was treated with diethyl ether, the precipitate was filtered off and washed with diethyl ether affording 2.16 g (80.5%) of the title compound.

HPLC method B; retention time: 3.38; M⁻: 327.35

According to this Method, (3-carbamoyl-6-hydroxy-benzo[b]thiophen-2-yl)-carbamic acid ethyl ester was Prepared.

Yield: 0.18 (65%)

NMR, δ (ppm): 11.24 (s, 1H), 9.51 (s, 1H), 7.72 (d, J=8.79 Hz, 1H), 7.24 (broad s, 2H), 7.23 (d, J=2.01 Hz, 1H), 6.87 (dd, J¹=2.13 Hz, J²=8.76 Hz, 1H ), 4.21 (m, 2H), 1.27 (t, J=7.08 Hz, 3H).

### 19.3A 2-(3-cyclopentyl-ureido)-4,7-dihydro-5H-thieno[2,3-c]pyran-3-carboxylic acid amide

To a mixture of (3-carbamoyl-4,7-dihydro-5H-thieno[2,3-c]pyran-2-yl)-carbamic acid phenyl ester (95.5 mg, 0.30 mmol) and 2 ml of THF, cyclopentylamine (0.75 mmol) was added. After stirring at ambient temperature until the reaction was completed (2-6 h), the mixture was poured into 1M NaOH, extracted twice with EtOAc, and the combined organic layers were dried over Na₂SO₄, filtered and concentrated. Purification of the residue by chromatogrqnhy (1 mm plate, eluent:chloroform-EtOAc 3:2), followed by concentration and trituration of the residue with ether afforded 2-(3-cyclopentyl-ureido)-4,7-dihydro-5H-thieno[2,3-c]pyran-3-carboxylic acid amide as a colourless solid.

Yield: 81%

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| B181 | B | 2.84 | | 395.19 | B75 | B | 3.11 | | 350.17 |
| B182 | B | 2.31 | 270.1 | | B47 | B | 3.48 | | 336.29 |
| B52 | B | 2.37 | | 310.13 | B48 | B | 2.89 | | 308.21 |
| B58 | B | 2.74 | | 326.2 | B50 | B | 3.17 | | 374.26 |
| B53 | B | 1.77 | 325.21 | | B70 | B | 11.6 | | 313.18 |
| B32 | B | 3.33 | | 385.18 | B69 | B | 1.88 | 341.22 | |
| B31 | B | 2.79 | | 381.23 | B68 | B | 1.92 | | 353.27 |
| B30 | B | 2.69 | | 296.18 | B54 | B | 2.86 | | 308.24 |
| B57 | B | 3.17 | | 324.15 | B55 | B | 3.37 | | 336.25 |
| B190 | B | 3.91 | | 352.2 | B56 | B | 3.25 | | 334.25 |
| B191 | B | 1.99 | | 284.19 | B63 | B | 4 | | 376.29 |
| B192 | B | 2.09 | 300.15 | | B231 | B | 1.84 | | 314.1 |
| B60 | B | 2.29 | | 312.14 | B232 | B | 2.04 | 383.2 | |
| B62 | B | 3.56 | | 348.28 | B233 | B | 1.84 | 347.11 | |
| B34 | B | 1.85 | 350.18 | | B234 | B | 3.64 | | 350.17 |
| B35 | B | 3.03 | | 320.18 | B247 | B | 1.48 | 354.15 | |
| B36 | B | 3.32 | 349.15 | | B236 | B | 3.5 | | 370.13 |
| B46 | B | 2.01 | | 328.21 | B243 | B | 3 | | 310.08 |
| B44 | B | 2.24 | | 324.21 | B244 | B | 1.8 | 257.05 | |
| B45 | B | 3 | | 334.21 | B257 | B | 2.60 | 325.13 | |
| B258 | B | 1.75 | 340.13 | | B260 | B | 3.87 | | 376.25 |
| B259 | B | 4.17 | | 378.28 | B261 | B | 1.95 | | 332.18 |
| B264 | B | 3.64 | | 467.11 | B262 | B | 2.86 | | 332.02 |
| B249 | B | 3.87 | | 364.16 | D233 | B | 3.75 | | 430.24 |
| D231 | B | 4.03 | | 420.19 | D234 | B | 4.12 | | 432.22 |
| D232 | B | 3.66 | | 420.2 | | | | | |

The following compounds were prepared by this method using 25 mg 4-dimethylaminopyridine as catalyst:

| | | | | |
|---|---|---|---|---|
| | HPLC | Retention | | |
| ID | Method | Time | M⁺ | M⁻ |

| | | | |
|---|---|---|---|
| B249 | B | 2.55 | 332.18 |
| B250 | B | 3.64 | 467.11 |

### 19.3B Synthesis of 6-hydroxy-2-[3-(2-thiophen-2-yl-ethyl)-ureido]-benzo[b]thio-phene-3-carboxylic acid amide (A67)

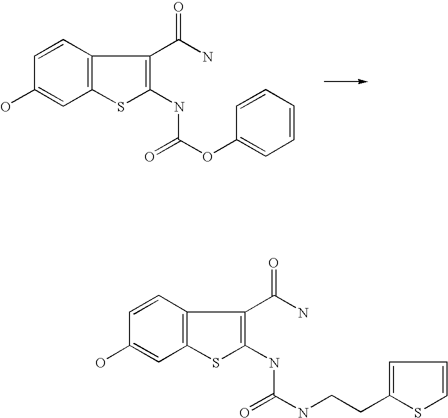

To a mixture of (3-carbamoyl-6-hydroxy-benzo[b]thiophen-2-yl)-carbamic acid phenyl ester (131 mg, 0.40 mmol), 0.2 ml DMSO and 2.5 ml of THF, 2-thiopheneethylamine (0.117 ml 1.0 mmol) was added. After stirring at ambient temperature until the reaction was completed (24-48 h), the mixture was poured into 10 ml of 1N HCl solution and extracted twice with EtOAc (20 ml). The combined organic layers were dried over Na₂SO₄, filtered and concentrated. Purification of the residue by chromatography (1 mm plate, eluent: chloroform-methanol 9:1), followed by concentration and trituration of the residue with ether afforded the title compound as a white solid.

Yield: 39%.

NMR (300 MHz, DMSO-d6 ppm): 10.77 (s, 1H), 9.34 (s, 1H), 7.96 (s, 1H), 7.66 (d, 1H), 7.41 (s, 2H), 7.34 (d, 1H), 7.14 (s, 1H), 6.96 (m, 2H), 6.82 (d, 1H), 3.38 (t, 2H), 2.98 (t, 2H)

According to this general method the following compounds were prepared:

| | HPLC | Retention | | |
|---|---|---|---|---|
| ID | Method | Time | M⁺ | M⁻ |
| A68 | B | 3.48 | | 346.04 |
| A69 | B | 2.96 | | 318.03 |
| A70 | B | 3.58 | | 358.11 |
| A71 | B | 3.29 | | 384.06 |
| A72 | B | 2.12 | | 294.07 |
| A73 | B | 2.14 | | 308.08 |
| A74 | B | 2.50 | | 333.11 |
| A75 | B | 3.22 | | 344.07 |

The following compounds were prepared by this method using (3-carbamoyl-6-oxo-ethylenylketal-4,5,6,7-tetrahydro-benzo[b]thiophen-2-yl)-carbamic acid phenyl ester (m.p. 175° C.) as starting material:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| D218 | B | 4.33 | | 434.18 | D224 | B | 3.38 | | 406.06 |
| D219 | B | 3.18 | | 451.09 | D225 | B | 2.14 | 403.09 | |
| D220 | B | 2.27 | | 469.17 | D226 | B | 2.10 | | 423.11 |
| D221 | B | 2.22 | | 340.10 | D227 | B | 3.50 | | 390.13 |
| D222 | B | 2.24 | | 370.05 | D228 | B | 4.23 | | 432.16 |
| D223 | B | 3.90 | 408.13 | | D229 | B | 4.23 | | 432.19 |
| D236 | B | 3.90 | 408.13 | | D230 | B | 3.81 | | 404.16 |

### General Method 20 for the Synthesis of the 6-hydroxy-7chloro Derivatives

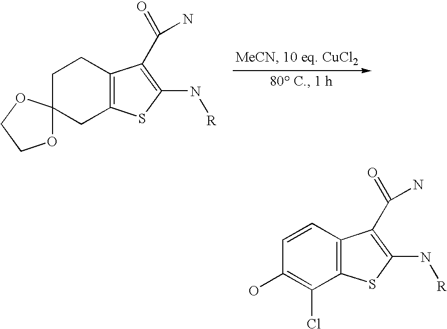

To a solution of the corresponding ketal derivative (1 mmol) in 10 ml acetonitrile 1.7 g (10 mM) fine powdered CuCl₂ was added with stirring. The reaction mixture was heated to 80° C. and stirred for 1 hour. Then the solvent was evaporated 10 ml 1N hydrochloric acid was added to the residue and extracted with 3×10 ml ethyl acetate.

Organic phase was dried over sodium sulfate and evaporated to dryness. The resulted dark crystalline material was washed with 0.5 ml cold ethylacetate and the formed crystals were isolated. The crude products may be recrystallized from acetonitrile if necessary.

According to this general method the following compounds were prepared:

| | HPLC | Retention | | | | | Retention | | |
|---|---|---|---|---|---|---|---|---|---|
| ID | Method | Time | M⁺ | M⁻ | ID | HPLC Method | Time | M⁺ | M⁻ |
| A35 | B | 3.14 | | 309.03 | A91 | B | 4.26 | | 402.00 |
| A84 | B | 3.99 | | 411.91 | A92 | B | 4.52 | | 496.14 |
| A85 | B | 4.51 | | 427.80 | A93 | B | 3.60 | | 394.08 |
| A37 | B | 3.42 | | 323.02 | A94 | B | 4.10 | | 428.13 |
| A87 | B | 3.63 | | 384.91 | A95 | B | 3.59 | | 396.11 |
| A88 | B | 4.06 | | 427.87 | A96 | B | 3.91 | | 378.12 |
| A89 | B | 3.91 | | 405.92 | A97 | B | 3.41 | | 449.96 |
| A90 | B | 4.00 | | 387.92 | A112 | B | 3.86 | | 425.00 |

### N-(3-Carbamoyl-4,5,6,7-tetrahydro-benzo[b]thiophen-2-yl)-succinamic acid (D130)

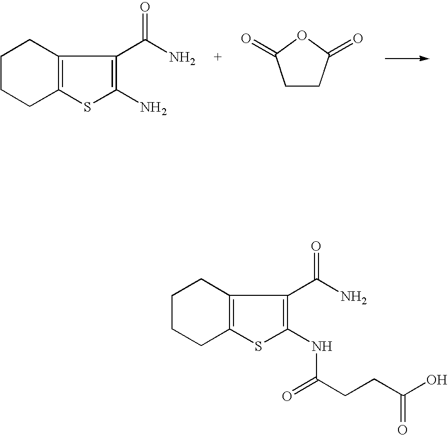

To a solution of 196 mg (1 mmol) of 2-amino-4,5,6,7-tetrahydro-benzo[b]thiophene-3-carboxylic acid amide in 5 ml abs. dioxane, 120 mg (1.2 mmol) of succinic anhydride were added. The mixture was stirred at 100° C. for 10 h. The reaction mixture was poured into cold water, the resulting precipitate was collected, washed with water and dried (Yield: 70%).

HPLC method B; retention time: 2.94; M⁻: 295.17

The following compounds were prepared by this method:

### N-(3-Carbamoyl-4,7-dihydro-5H-thieno[2,3-c]pyran-2-yl)-succinamic acid (B104)

HPLC method B; retention time: 2.17; M⁻: 297.08

### 3-(3-Carbamoyl-4,7-dihydro-5H-thieno[2,3-c]pyran-2-ylcarbamoyl)-acrylic acid (B105)

HPLC method B; retention time: 2.17; M⁻: 295.06

### 3-(3-Carbamoyl-4,5,6,7-tetrahydro-benzo[b]thiophen-2-ylcarbamoyl)-acrylic acid (D78)

HPLC method B; retention time: 2.89; M⁻: 293.08

### Synthesis of 2-(3-phenylpropionyl-amino)-4,7-dihydro-5H-thienopyrane-3-carboxamide (B178)

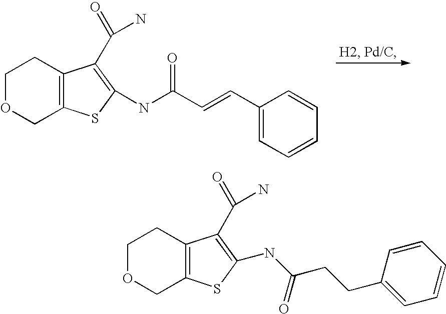

To a solution of 240 mg of 2-{[(2E)-3-phenylprop-2-enoyl]amino}-4,7-dihydro-5H-thieno[2,3-c]pyran-3-carboxamide in 10 ml of ethanol, 50 mg of 10% palladium/charcoal were added and the mixture hydrogenated at room temperature and normal pressure. The catalyst was removed by filtration over Celite and the solvent was evaporated (Yield: 93%).

HPLC method A; retention time: 1.86; M⁺: 331.13

### I. Preparation of Ethyl 2-amino-4,7-dihydro-5H-thieno[2,3-c]pyran-3-carboxylate

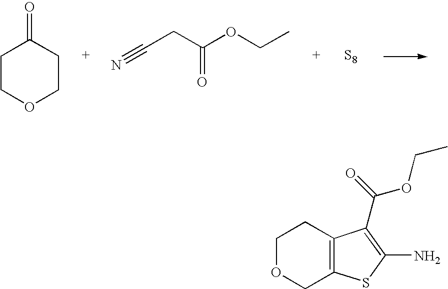

1 mM tetrahydro-pyran-4-one, 106 μl (1 mM) ethyl-cyano-acetate, 0.15 mM ammonium-acetate, and 0.2 mM acetic acid where dissolved in 3 ml benzene and stirred at reflux temperature in a round-bottomed flask equipped with water-remover trap, for 3 hours. The reaction mixture was washed with 2 ml 10% K₂CO₃ solution, dried, and evaporated to dryness. The solid material was dissolved in 1.5 ml EtOH and was stirred with 1.05 mM sulphur and 0.575 mM morpholine at 45-50° C., for 4 hours. The reaction mixture was evaporated to dryness, washed with n-hexane and isopropylalcohol. This reaction step was developed starting from a procedure described by Gewald, K; Schinke, E; Böttcher, H; Chem. Ber. 1966, 99, 974.

Yield: 57%

NMR: 7.28 (s, 2H), 4.43 (s, 2H), 4.16 (q, 2H), 3.79 (t, 2H), 3.67 (t, 2H), 1.25 (t, 3H)

### II. Preparation of 2-(cyclopropanecarbonyl-amino)-4,7-dihydro-5H-thieno[2,3-c]pyran-3-carboxylic acid ethyl ester

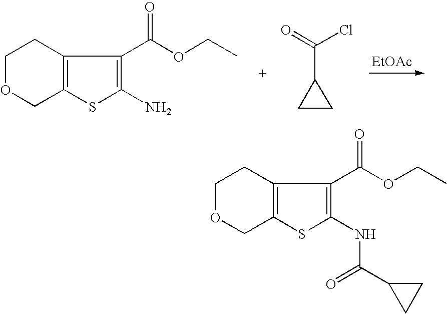

1 mM cyclopropanecarbonyl chloride was added dropwise to a well stirred, 15 ml ethylacetate solution of 301 mg (1.00 mM) 2-amino-4,7-dihydro-5H-thieno[2,3-c]pyran-3-carboxylic acid ethyl ester. The reaction mixture was stirred for 3 hours, then diluted to 50 ml, washed two times with water, dried with MgSO₄, and evaporated to dryness. The product was washed with n-hexane and isopropanol.

Yield: 42%

NMR: 11.19 (s, 1H), 4.60 (s, 2H), 4.30 (q, 2H), 3.84 (t, 2H), 2.78 (t, 2H), 2.03 (m, 1H), 1.33 (t, 3H), 0.93 (m, 4H)

Analogous to this method the following compounds were also synthesized:

2-[(Furan-2-carbonyl)-amino]-4,7-dihydro-5H-thieno[2,3-c]pyran-3-carboxylic acid ethyl ester (Yield 57%), NMR: 11.90 (s, 1H), 8.06 (s, 1H), 7.39 (d, 1H), 6.79 (dd, 1H), 4.66 (s, 2H), 4.35 (q, 2H), 3.86 (t, 2H), 2.82 (t, 2H), 1.35 (t, 3H);

2-[(Adamantane-1-carbonyl)-amino]-4,7-dihydro-5H-thieno[2,3-c]pyran-3-carboxylic acid ethyl ester (Yield 67%), NMR: 11.36 (s, 1H), 4.62 (s, 2H), 4.32 (q, 2H), 3.84 (t, 2H), 2.79 (t, 2H), 2.06 (bs, 2H), 1.90 (s, 8H), 1.72 (s, 6H), 1.33 (t, 3H);

2-(Cyclohexanecarbonyl-amino)-4,7-dihydro-5H-thieno[2,3-c]pyran-3-carboxylic acid ethyl ester (Yield 70%), NMR: 11.10 (s, 1H), 4.61 (s, 2H), 4.30 (q, 2H), 3.84 (t, 2H), 2.78 (t, 2H), 1.90 (d, 2H), 1.69 (m, 3H), 1.43-1.18-(m, 9H);

2-[(2-Methyl-cyclopropanecarbonyl)-amino]-4,7-dihydro-5H-thieno[2,3-c]pyran-3-carboxylic acid ethyl ester (Yield 57%), NMR: 11.15 (s, 1H), 4.60 (s, 2H), 4.25 (q, 2H), 3.64 (t, 2H), 2.78 (t, 2H), 1.80 (m, 1H), 1.33 (t, 3H), 1.11 (d, 3H), 0.79 (m, 1H);

2-(Cyclobutanecarbonyl-amino)-4,7-dihydro-5H-thieno[2,3-c]pyran-3-carboxylic acid ethyl ester (Yield 76%), NMR: 10.91 (s, 1H), 4.61 (s, 2H), 4.28 (q, 2H), 3.83 (t, 2H), 3.44 (m, 1H), 2.78 (bs, 2H), 2.23 (m, 4H), 1.97 (m, 1H), 1.83 (m, 1H), 1.31 (t, 3H);

2-Acetylamino-4,7-dihydro-5H-thieno[2,3-c]pyran-3-carboxylic acid ethyl ester (Yield 85%), NMR: 10.93 (s, 1H), 4.61 (s, 2H), 4.29 (q, 2H), 3.84 (t, 2H), 2.77 (t, 2H), 2.24 (s, 3H), 1.32 (t, 3H);

### 2-(3-Methyl-but-2enoylamino)-4,7-dihydro-5H-thieno[2,3-c]pyran-3-carboxylic acid ethyl ester (Yield 64%), NMR: 10.93 (s, 1H), 4.61 (s, 2H), 4.29 (q, 2H), 3.84 (t, 2H), 2.77 (t, 2H), 2.24 (s, 3H), 1.32 (t, 3H);

2-But-2-enoylamino-4,7-dihydro-5H-thieno[2,3-c]pyran-3-carboxylic acid ethyl ester (Yield 76%), NMR: 11.02 (s, 1H), 6.90 (m, 1H), 6.35 (dd, 1H), 4.63 (s, 2H), 4.30 (q, 2H), 3.84 (t, 2H), 2.79 (t, 2H), 1.92 (s, 3H), 1.89 (s, 3H), 1.32 (t, 3H);

2-(2-Methyl-butyrylamino)-4,7-dihydro-5H-thieno[2,3-c]pyran-3-carboxylic acid ethyl ester (Yield 69%), 11.05(s, 1H),4.62(s,2H),4.30(q,2H), 3.84(t,2H), 2.80(t,2H), 2.59 (m,1H), 1.64(m, 1H), 1.50(m, 1H), 1.32(t,3H), 1.14(d,3H), 0.87(t,3H);

2-(2,2-Dimethyl-propionylamino)-4,7-dihydro-5H-thieno[2,3-c]pyran-3-carboxylic acid ethyl ester (yield 76%), NMR: 11.05(s, 1H),4.62(s,2H),4.30 (q,2H), 3.84(t,2H), 2.80(t,2H), 2.59(m, 1H), 1.64(m,1H), 1.50(m, 1H), 1.32(t,3H), 1.14(d,3H), 0.87 (t,3H);

2-(2-Chloro-acetylamino)-4,7-dihydro-5H-thieno[2,3-c]pyran-3-carboxylic acid ethyl ester (Yield 82%), NMR: 11.64 (s, 1H), 4.64 (s, 2H), 4.61 (s, 2H), 4.32 (q, 2H), 3.85 (t, 2H), 2.80 (t, 2H), 1.32 (t, 3H);

2-(3,4-Difluoro-benzoylamino)-4,7-dihydro-5H-thieno[2,3-c]pyran-3-carboxylic acid ethyl ester (Yield 79%), NMR: 11.89 (s, 1H), 7.95 (m, 1H), 7.76 (m, 2H), 4.67 (s, 2H), 4.34 (q, 2H), 3.87 (t, 2H), 2.82 (t, 2H), 1.34 (t, 3H);

2-Isobutyrylamino-4,7-dihydro-5H-thieno[2,3-c]pyran-3-carboxylic acid ethyl ester (Yield 80%), NMR: 11.08 (s, 1H), 4.62 (s, 2H), 4.30 (q, 2H), 3.84 (t, 2H), 2.78 (m, 3H), 1.32 (t, 3H), 1.17 (d, 6H);

2-(Cyclopentanecarbonyl-amino)-4,7-dihydro-5H-thieno[2,3-c]pyran-3-carboxylic acid ethyl ester (Yield 77%), NMR: 11.04 (s, 1H), 4.61 (s, 2H), 4.29 (q, 2H), 3.84 (t, 2H), 2.99 (m, 1H), 2.78 (t, 2H), 1.91 (m, 2H), 1.65 (m, 6H), 1.31 (t, 3H).

### III. Preparation of 2-methanesulfonylamino-4,7-dihydro-5H-thieno[2,3-c]pyran-3-carboxylic acid ethyl ester

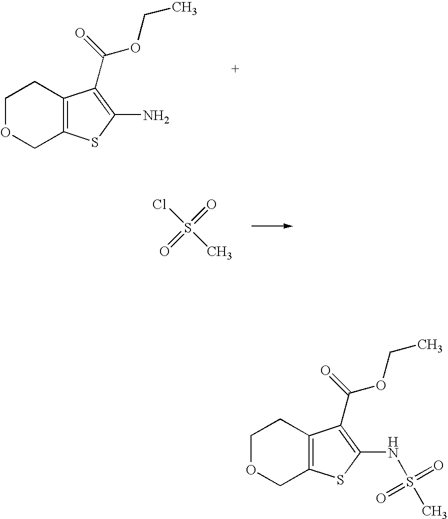

1 mmol 2-Amino-4,7-dihydro-5H-thieno[2,3-c]pyran-3-carboxylic acid ethyl ester was dissolved in 10 ml benzene and 348 μl (2.5 equivalent) triethylamine, 195 μl (2.5 equiv.) methanesulfonyl chloride was added. The reaction mixture was refluxed for 8 hours. The mixture was extracted with 1×15 ml water, 1×15 ml NaHCO₃, then 1×15 ml water, 1×15 ml 1N HCl and saturated NaCl solution. The organic layer was dried above MgSO₄, the solvent was evaporated to vacuo and the residue was crystallized from hexane-isopropanol. (TLC-Eluent:Hexan-Ethylacetate: 2:1)

Yield: 65%, NMR: 11.03 (s, 1H), 4.73 (s, 2H), 4.28 (q, 2H), 3.89 (t, 2H), 3.53 (s, 3H), 2.83 (t, 2H), 1.29 (t, 3H).

### IV. Preparation of 2-acetamino-7-hydroxy-4,7-dihidro-5H-thieno[2,3-c]pyran-3-carboxylic acid ethyl ester

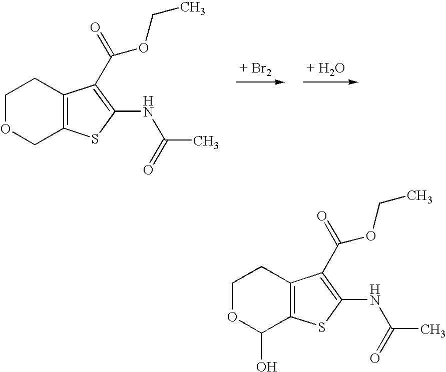

269 mg (1 mmol) 2-Acetylamino-4,7-dihydro-5H-thieno[2,3-c]pyran-3-carboxylic acid ethyl ester was dissolved in 15 ml acetic acid and 82 mg (1 mmol) sodium-acetate was added to the mixture, then heated to 55° C. 159 mg bromine in 15 ml acetic acid was added slowly to the mixture. After one hour stirring it was evaporated under reduced pressure and extracted three times with ethyl acetate and 15 ml water. The organic layer was washed with 10 ml NaHCO₃ solution and dried with MgSO₄. The solution was evaporated under reduced pressure and the product was crystallized from hexane. The product was washed with IPA, and recrystallized with diisopropyl-ether. Yield: 39% NMR: 10.92 (s, 1H), 4.83 (d, 1H), 4.73 (d, 1H), 4.49 (d, 1H), 4.29 (q, 2H), 3.90 (d, 1H), 3.65 (d, 1H), 2.24 (s, 3H), 1.32 (t, 3H).

The compound 2-(cyclopropanecarbonyl-amino)-7-hydroxy-4,7-dihydro-5H-thieno[2,3-c]pyran-3-carboxylic acid ethyl ester was synthesized in a analogous reaction. Yield: 62%, NMR: 11.20 (s, 1H), 5.65 (s, 1H), 4.93-4.65 (m, 2H), 4.34 (q, 2H), 4.24-4.03 (m, 3H), 2.10 (m, 1H), 1.38 (t, 3H), 0.93 (m, 4H).

### V. Preparation of 2-(cyclopropanecarbonyl-amino)-4,5,6,7-tetrahydro-benzo[b]thiophene-3-carboxylic acid amide (Compound B1)

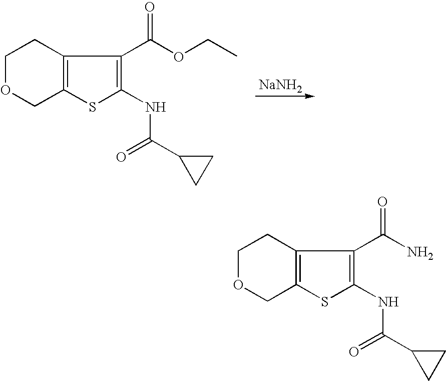

470 mg (12.00 mM) sodium amide was added to the solution of 293 mg (1.00 mM) 2-(cyclopropanecarbonyl-amino)-4,5,6,7-tetrahydro-benzo[b]thiophene-3-carboxylic acid ethyl ester in 8 ml abs. tetrahydrofurane. The air-tightly closed reaction mixture was stirred at room temperature for 72 hours.

After the starting material disappeared, the pH of the reaction mixture was set to 5-6 with ice cold, 1 N HCl, the precipitated product was filtered off, washed twice with 5 ml n-hexane and dried.

Yield: 89% white, or off-white crystals; NMR: 11.75 (s, 1H), 4.62 (s, 2H), 3.83 (t, 2h), 2.79 (t, 2H), 1.89 (m, 1H), 0.87 (m, 4H)

### VI. Preparation of 2-(cyclopentanecarbonyl-amino)-4,7-dihydro-5H-thieno[2,3-c]pyran-3-carboxylic acid amide (Compound B10).

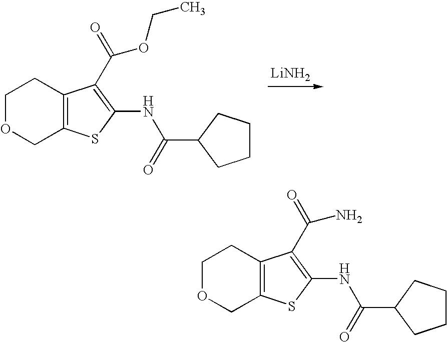

1 mmol 2-(Cyclopentanecarbonyl-amino)-4,7-dihydro-5H-thieno[2,3-c]pyran-3-carboxylic acid ethyl ester was dissolved in 3 ml abs. THF, then 230 mg (10 equivalent) LiNH₂ was added and the mixture was stirred in a stoppered flask at r.t. for 48 hours. The reaction mixture was poured on ice water, the pH of the solution was adjusted to 5 with 5% HCl. The precipitated crystals were filtered out and washed with cold isopropanol. (TLC Eluent:chloroform-MeOH 10:1)

Yield: 79%, NMR: 11.73 (s, 1H), 7.2 (bd, 2H), 4.63 (s, 2H), 3.83 (t, 2H), 2.88 (m, 1H), 2.80 (t, 2H), 1.89 (m, 2H), 1.64 (m, 6H).

The following compounds were also prepared by this method:

2-(2-Methyl-butyrylamino)-4,7-dihydro-5H-thieno[2,3-c]pyran-3-carboxylic acid amide (Compound B14), Yield: 67%, NMR: 11.77 (s, 1H), 7.2 (bs, 2H), 4.63 (s, 2H), 3.83 (t, 2H), 2.80 (t, 2H), 2.46 (m, 1H), 1.60 (m, 1H), 1.47 (m, 1H), 1.12 (d, 3H), 0.85 (t, 3H);

2-(Cyclobutanecarbonyl-amino)-4,7-dihydro-5H-thieno[2,3-c]pyran-3-carboxylic acid amide (Compound B15), Yield: 74%, NMR: 11.63 (s, 1H), 7.2 (bs, 2H), 4.63 (s, 2H), 3.83 (t, 2H), 3.33 (m, 1H), 2.81 (t, 2H), 2.20 (m, 4H), 1.97 (m, 1H), 1.83 (m, 1H);

2-[(2-Phenyl-cyclopropanecarbonyl)-amino]-4,7-dihydro-5H-thieno[2,3-c]pyran-3-carboxylic acid amide (Compound B3), Yield: 73%, NMR: 11.26 (s, 1H), 7.32-7.19 (m, 5H), 4.62 (s, 2H), 4.28 (q, 2H), 3.84 (t, 2H), 2.78 (t, 2H), 1.55 (m, 1H), 1.43 (m, 1H), 1.30 (t, 3H);

2-But-2-enoylamino-4,7-dihydro-5H-thieno[2,3-c]pyran-3-carboxylic acid amide (Compound B16), Yield: 49%, NMR: 11.64 (s, 1H), 7.3 (bd, 2H), 6.83 (m, 1H), 6.23 (dd, 1H), 4.64 (s, 2H), 3.83 (t, 2H), 2.80 (t, 2H), 1.89 (d, 3H);

2-(3-Methyl-but-2-enoylamino)-4,7-dihydro-5H-thieno[2,3-c]pyran-3-carboxylic acid amide (Compound B17), Yield 31%, NMR: 11.56 (s, 1H), 7.2 (bs, 2H), 5.93 (s, 1H), 4.64 (s, 2H), 3.83 (t, 2H), 2.80 (t, 2H), 2.16 (s, 3H), 1.90 (s, 3H);

2-(2,2-Dimethyl-propionylamino)-4,7-dihydro-5H-thieno[2,3-c]pyran-3-carboxylic acid amide (Compound B18), Yield 32%, NMR: 12.33 (s, 1H), 7.2 (bs, 2H), 4.64 (s, 2H), 3.83 (t, 2H), 2.83 (t, 2H), 1.22 (s, 9H);

2-(3,4-Difluoro-benzoylamino)-4,7-dihydro-5H-thieno[2,3-c]pyran-3-carboxylic acid amide (Compound B19), Yield: 70%; NMR: 13.01 (s, 1H), 7.88 (m, 1H), 7.70 (m, 2H), 7.30 (bs, 2H), 4.69 (s, 2H), 3.86 (t, 2H), 2.86 (t, 2H);

2-Isobutyrylamino-4,7-dihydro-5H-thieno[2,3-c]pyran-3-carboxylic acid amide (Compound B2), Yield: 61%, NMR: 11.81 (s, 1H), 7.2 (bd, 2H), 4.63 (s, 2H), 3.83 (t, 2H), 2.81 (t, 2H), 2.67 (m, 1H), 1.14 (d, 6H);

2-[(2-Methyl-cyclopropanecarbonyl)-amino]-4,7-dihydro-5H-thieno[2,3-c]pyran-3-carboxylic acid amide (Compound B4), Yield: 53%, NMR: 11.71 (s, 1H), 7.5 (bs, 1H), 7.0 (bs, 1H), 4.61 (s, 2H), 3.82 (t, 2H), 2.79 (t, 2H), 1.64 (m, 1H), 1.09 (d, 3H), 0.73 (m, 1H);

2-[(Furan-2-carbonyl)-amino]-4,7-dihydro-5H-thieno[2,3-c]pyran-3-carboxylic acid amide (Compound B5), Yield: 34%, NMR: 12.72 (s, 1H), 8.02 (d, 1H), 7.7 (bs, 1H), 7.31 (d, 1H), 7.2 (bs, 1H), 6.76 (dd, 1H), 4.67 (s, 2H), 3.85 (t, 2H), 2.86 (t, 2H);

2-[(Adamantane-1-carbonyl)-amino]-4,7-dihydro-5H-thieno[2,3-c]pyran-3-carboxylic acid amide (Compound B6), Yield: 61%, NMR: 12.23 (s, 1H), 7.3 (b, 2H0, 4.63 (s, 2H), 3.83 (t, 2H), 2.83 (t, 2H), 2.03 (s, 3H), 1.86 (s, 5H), 1.70 (s, 5H);

2-(Cyclohexanecarbonyl-amino)-4,7-dihydro-5H -thieno[2,3-c]pyran-3-carboxylic acid amide (Compound B7), Yield: 63%, NMR: 11.79 (s, 1H), 7.2 (bd, 2H), 4.63 (s, 2H), 3.82 (t, 2H), 2.80 (t, 2H), 2.41 (m, 1H), 1.89-1.62 (m, 5H), 1.40-1.17 (m, 5H).

### Preparation of 2-(cyclopropanecarbonyl-amino)-4,7-dihydro-5H-thieno[2,3-c]pyran-3-sulfonamide

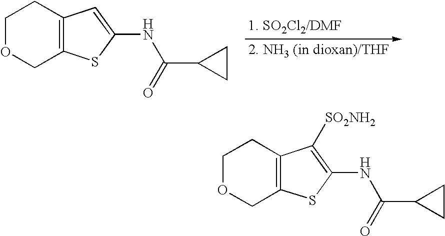

Sulfurylchloride (13 mmol) was added dropwise to DMF (13 mmol) at 0° C. under Argon. The mixture was stirred for 30 min at 0° C. and 2-(cyclopropanecarbonyl-amino)-4,7-dihydro-5H-thieno[2,3-c]pyrane (10 mmol) in 2 ml DCM added. The mixture was stirred for 1 h at r.t., diluted with 2 ml of THF and treated with an excess of NH₃ (2 M solution in dioxane, 10 ml, 20 mmol). The mixture was stirred at room temperature overnight. Evaporation of the solvent and recrystallization afforded the title compound.

### Preparation of 2-[(cyclopropanecarbonyl)-amino]-3-cyano-4,7-dihydro-5H-furo[2,3-c]pyrane

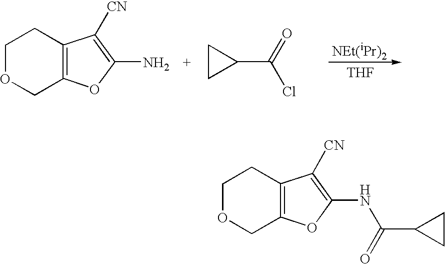

2-Amino-3-cyano-4,7-dihydro-5H-furo[2,3-c]pyrane (0.66 mmol) and cyclopropylcarbonyl chloride (0.8 mmol) were dissolved in 10 mL of abs. THF. Diisopropylethylamine (0.8 mmol) was added via syringe, and the mixture was stirred overnight at room temperature. After dilution with 20 ml of water, the aqueous phase was extracted four times with ethylacetate, the organic layer washed once with water, dried over sodium sulfate and the solvents evaporated. Recrystallization of the crude material from hot ethanol gave the desired product.

### Preparation of 1-benzyl-2-[(cyclopropanecarbonyl)-amino]-3-cyano-4,7-dihydro-5H-pyrrolo[2,3-c]pyrane

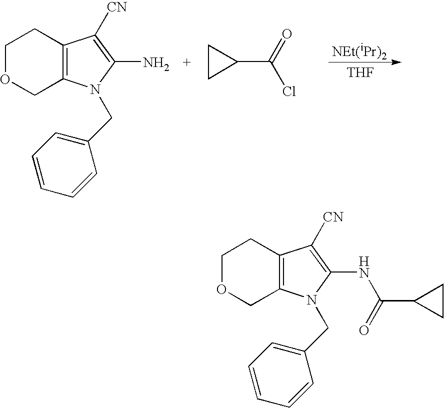

1-Benzyl-2-amino-3-cyano-4,7-dihydro-5H-pyrrolo[2,3-c]pyrane (4.1 mmol) and cyclopropylcarbonyl chloride (4.9 mmol) were dissolved in 10 ml of abs. THF. 1.5 ml of diisopropylethylamine were added via syringe, and the mixture was stirred overnight at room temperature. After dilution with 20 ml of water, the aqueous phase was extracted four times with ethylacetate, the organic layer washed once with water, dried over sodium sulfate and the solvents evaporated. Recrystallization of the crude material from hot ethanol gave the desired product.

### Preparation of 2-[(cyclopropanecarbonyl)amino]-4,7-dihydro-5H-furo[2,3-c]pyrane-3-carboxamide

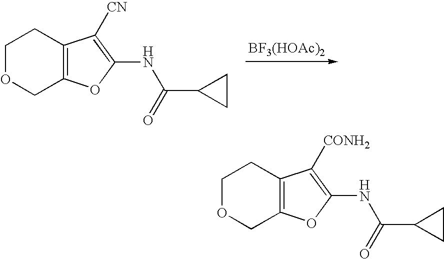

2-(Cyclopropanecarbonyl-amino)-3-cyano-4,7-dihydro-5H-furo[2,3-c]pyrane (4.3 mmol), 1 ml of water, and 7 ml of boron trifluoride-acetic acid complex are heated at 120° C. for 10 minutes. After cooling, the reaction mixture is treated with 50 ml of 6 N sodium hydroxide solution, the aqueous mixture is extracted with ethylacetate, dried over sodium sulfate and the solvents evaporated. The crude material can be recrystallized from hot ethanol.

### Preparation of 1-benzyl-2-[(cyclopropanecarbonyl)amino]-4,7-dihydro-5H-pyrrolo[2,3-c]pyrane-3-carboxamide

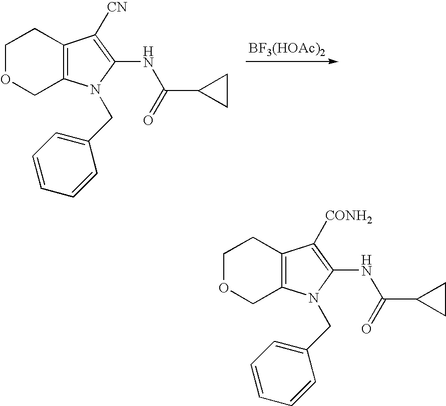

1-Benzyl-2-(cyclopropanecarbonyl-amino)-3-cyano-4,7-dihydro-5H-pyrrolo[2,3-c]pyrane (4 mmol), 1 ml of water, and 7 ml of boron trifluoride-acetic acid complex are heated at 120° C. for 10 minutes. After cooling, the reaction mixture is treated with 50 ml of 6 N sodium hydroxide solution, the aqueous mixture is extracted with ethylacetate, dried over sodium sulfate and the solvents evaporated. The crude material is recrystallized from hot ethanol.

### General Method 21 for the Synthesis of the 6-urethane Derivatives

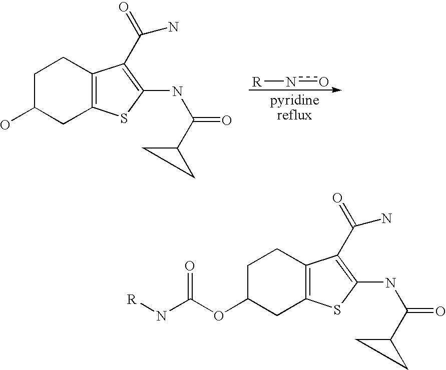

A solution of the hydroxy derivative (1 mmol) and corresponding isocyanate (1.1 mmol) in 10 ml abs. pyridine was refluxed and stirred for 1 hour. Then the solvent was evaporated, 10 ml 1N hydrochloric acid was added to the residue and extracted with 3×10 ml ethyl acetate. Organic phase was dried over sodium sulphate and evaporated to dryness and crystallized from isopropanol or cleaned by chromatography on silica column using ethyl acetate.

| | HPLC | Retention | | |
|---|---|---|---|---|
| ID | Method | Time | M⁺ | M⁻ |
| D205 | B | 4.05 | | 450.09 |
| D198 | B | 3.63 | | 507.23 |

### General Method 22 for Deprotection of Boc-Protected Amino Groups

The protected derivative D198 (0.5 mmol) was dissolved in 5 ml ethyl acetate and 1 ml ethyl acetate saturated with hydrochloric acid was added with stirring. After one hour the formed crystals were filtered and washed carefully with ethyl acetate and dried in vacuum.

| | HPLC | Retention | | |
|---|---|---|---|---|
| ID | Method | Time | M⁺ | M⁻ |
| D234 | B | 2.30 | 409.20 | |

### General Method 23 for the Synthesis of Oxalic Derivatives

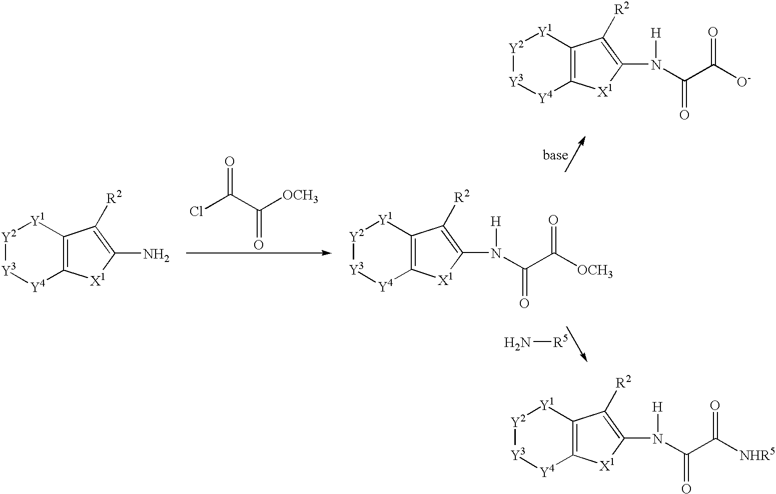

wherein

the substituents X¹, Y¹, Y², Y³, Y⁴, R², and R⁵ have the meaning as defined in claim 1.

10 mmol of the corresponding 2-amino derivative (ketal derivative for 6-hydroxy compound) was dissolved in 10 dry pyridine and 11 mmol chloro-oxo-acetic acid methyl ester was added dropwise with stirring at room temperature. After two hours the reaction mixture was diluted with 10 ml of water and the crystalline product was filtered. Methyl ester was removed by equivalent amount of sodium hydroxide in 50% aqueous methanol (stirring at room temperature overnight, and isolated in crystalline form when the reaction mixture was acidified with 1 N hydrochloric acid. Ketal protecting group was removed with cupric chloride as given elsewhere. 2-hydroxyethylamide was obtained from the methyl ester and aminoethanol as representative for H₂N—R⁵ in ethanolic solution or any other suitable solvent. The product crystallizes after 2 days at room temperature.

The following compounds were synthesized according to this procedure:

| | HPLC | Retention | | |
|---|---|---|---|---|
| ID | Method | Time | M⁺ | M⁻ |
| A99 | B | 1.86 | | 279.09 |
| B263 | B | 2.16 | | 312.02 |

### General Method 24 for the Synthesis of Substituted-phenyl-5-ureido-benzo[b]thiophene Derivatives

### Procedure 24.1. 2-(Cyclopropanecarbonyl-amino)-5-nitro-benzo[b]thiophene-3-carboxylic acid amide (A113)

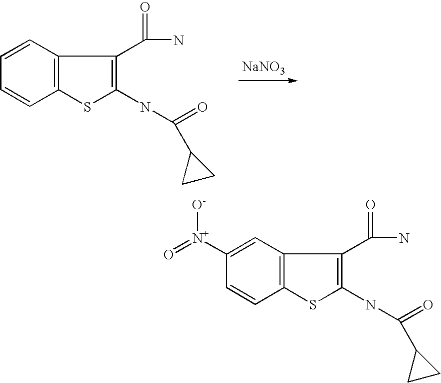

To a solution of 0.427 g (1.64 mmol) 2-(Cyclopropanecarbonyl-amino)-benzo[b]thiophene-3-carboxylic acid amide in 35 ml acetic acid was added 0.195 g (2.3 mmol) NaNO₃ at room temperature. It was stirring for two days. The precipitate was collected, washed with water. The yield is 65%.

| | HPLC | Retention | | |
|---|---|---|---|---|
| ID | Method | Time | M⁺ | M⁻ |
| A113 | B | 3.46 | | 304.13 |

### Procedure 24.2. 5-Amino-2-(cyclopropanecarbonyl-amino)-benzo[b]thiophene-3-carboxylic acid amide (A114)

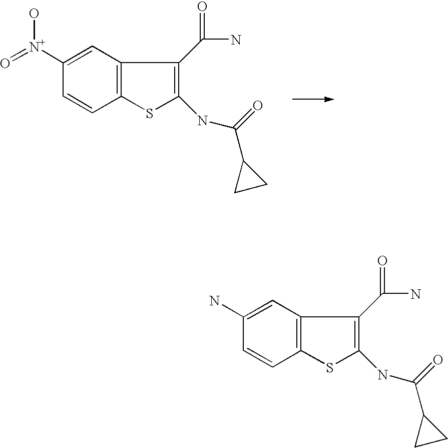

0.305 g (1 mmol) 2-(Cyclopropanecarbonyl-amino)-5-nitro-benzo[b]thiophene-3-carboxylic acid amide was hydrogenated in 40 ml abs. ethanol with catalytic amount of Pd/C (atmospheric pressure). The mixture was stirred overnight at 25° C., then filtered, and the solution was evaporated to dryness to give the title compound in 90% yield.

| | HPLC | Retention | | |
|---|---|---|---|---|
| ID | Method | Time | M⁺ | M⁻ |
| A114 | B | 1.91 | | 274.16 |

### Procedure 24.3. 5-[3-(4-Bromo-phenyl)-ureido]-2-(cyclopropanecarbonyl-amino)-benzo[b]thiophene-3-carboxylic acid amide (A106)

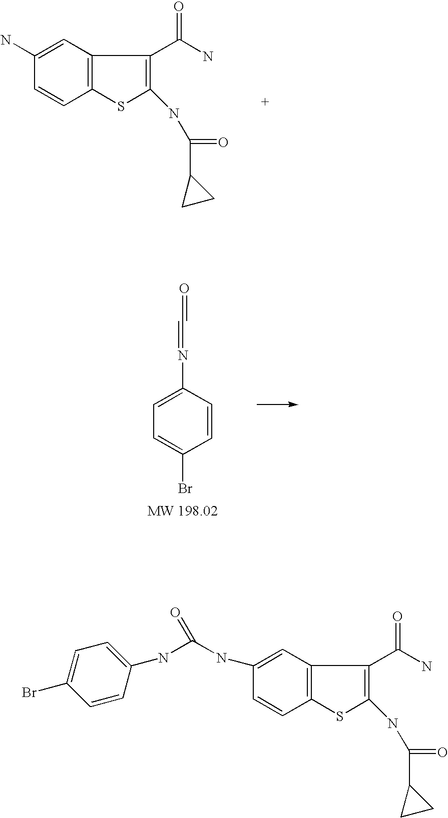

0.195 g (0.71 mmol) 5-Amino-2-(cyclopropanecarbonyl-amino)-benzo[b]thiophene-3-carboxylic acid amide was dissolved in 5 ml abs. dichloroethane. 0.141 g (0.71 mmol) 4-bromo-phenylisocyanate was added. The mixture was stirred at 100° C. for 3 h. The reaction was followed by TLC (EtOAc:Hexan 1:1). The solution was cooled and hexane was added, the resulted precipitate was filtered off and washed with hexane. The yield is 80%

According to this procedure the following compounds were prepared:

| | HPLC | Retention | | | | HPLC | Retention | | |
|---|---|---|---|---|---|---|---|---|---|
| ID | Method | Time | M⁺ | M⁻ | ID | Method | Time | M⁺ | M⁻ |
| A105 | B | 3.83 | | 439.23 | A107 | B | 4.12 | | 518.77 |
| A106 | B | 4.00 | | 470.84 | A108 | B | 3.58 | | 393.02 |
| B265 | B | 4.13 | | 362.19 | B266 | B | 4.09 | | 416.16 |
| D241 | B | 3.71 | | 418.03 | D242 | B | 4.26 | | 472.03 |
| D243 | B | 3.68 | | 438.03 | | | | | |

### General Method 25 for the Synthesis of 7-bromo-6-hydroxy-benzo[b]thiophene Derivatives

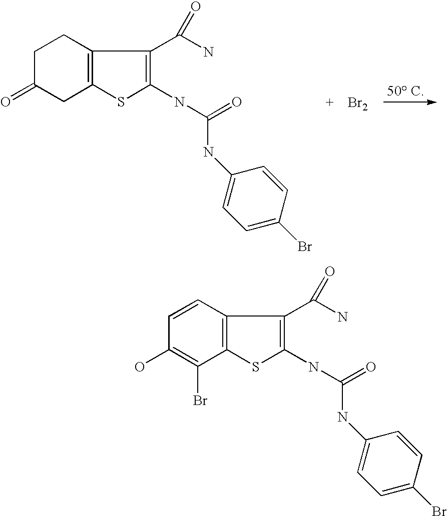

To a solution of 1 mmol 2-[3-(substituted-phenyl)-ureido]-6-oxo-4,5,6,7-tetrahydro-benzo[b]thiophene-3-carboxylic acid amide in acetic acid (150 ml) was added 2.4 mmol bromine in 24 ml acetic acid at 50° C. The progress of the reaction was followed by TLC.(CHCl₃:MeOH 10:1). When the reaction was completed, acetic acid was evaporated. The product was crystallized from isopropanol.

According to this general method the following compounds were prepared:

| | HPLC | Retention | | |
|---|---|---|---|---|
| ID | Method | Time | M⁺ | M⁻ |
| A109 | B | 4.03 | | 482.00 |
| A110 | B | 4.15 | | 488.08 |
| A111 | B | 3.83 | | 450.07 |

Biochemical Methods and Experiments

In the following documents, background information is given with regard to the methods, micoorganisms and enzymes used according to the present invention: Peirs et al., A serine/threonine protein kinase from Mycobacterium tuberculosis, Eur. J. Biochem., March 1, 244(2), 604-612 (1997); Arruda et al., Cloning of an M. tuberculosis DNA fragment associated with entry and survival inside cells, Science 261, 1454-1457 (1993); Wieles et al., Increased intracellular survival of Mycobacterium smegmatis containing the Mycobacterium leprae thioredoxin-thioredoxin reductase gene, Infect Immun. 65(7), 2537-2541 (1997); Zahrt, Mycobacterium tuberculosis signal transduction system required for persistent infections, Proc. Natl. Acad. Sci. 98 (22), 12706-12711 (2001); and Mundayoor et al., Identification of genes involved in the resistance of mycobacteria to killing by macrophages, Ann. N. Y. Acad. Sci. 730, 26-36 (1994).

Bacterial Strains and Culture Conditions

Mycobacterium smegmatis was grown in Middlebrook 7H9 medium (supplier: Difco), supplemented with 10% ADC (Difco), 0.05% Tween-80 and 0.5% glycerol. E. coli was cultivated in LB- or TB-broth without any additional ingredients. Cloning, mutagenesis and expression of PknG and other mycobacterial kinases was done as described by Koul et. al. (Serine/threonine kinases, PknG and PknF of Mycobacterium tuberculosis: characterisation and localisation. Microbiology, 147, 2001).

GST-Fusion Protein Purification

Purification of GST-fusion proteins was done as described previously by Koul et. al. (Serine/threonine kinases, PknG and PknF of Mycobacterium tuberculosis: characterisation and localisation. Microbiology, 147, 2001). E. coli BL21 cultures containing the respective plasmids were grown overnight in TB-broth. After IPTG induction, the suspensions were incubated for another 16 hours at room temperature. The bacteria were harvested by centrifugation, resuspended in 1× PBS and lysed by sonification. After addition of Triton X-100 (1% final concentration) and subsequent clarifying of the lysates the GST-fusion proteins were purified by addition of GST-sepharose following PBS washes. The proteins were eluted with a buffer containing 50 mM glutathion, 20 mM Tris (pH 8.0), 0.1 M NaCl, 0.1 M Triton X-100 and 1 mM DTT. Thereafter, the eluates were dialysed in 20 mM HEPES (pH 7.5) and 30% glycerol.

Determination of Protein Kinase Activity

The activity of all protein serine threonine kinases from Mycobacterium tuberculosis was determined by addition of myelin basic protein as a substrate in an in vitro kinase assay. The buffer conditions were as follows: 20 mM HEPES (pH 7.5), 20 mM MgCl₂, and 5 mM MnCl₂, for all kinases except PknG, PknI, PknJ, and PknL. These protein kinases required lower salt concentrations, namely 1 mM MgCl₂, and 1 mM MnCl₂. The optimum ATP concentration for each kinase was determined by titration of ATP in a range between 0.0033 μM and 100 μM. The inhibitor studies were performed with ATP concentrations similar to the Michaelis constant (Kₘ) for ATP. We further analysed the role of PknG in pathogenesis of mycobacteria in cellular infection model.

Infection of Macrophage Cells with Recombinant Mycobacterium smegmatis

Mycobacterium smegmatis, electroporated with either vector alone or mycobacterial expression vector containing PknG (wild type) or PknG-K181M (Mutant), was cultured for 2 days in Middlebrook 7H9 medium containing 0.05% Tween-80 and 0.5% glycerol. Bacteria were pelleted at 1500×g for 3 minutes by centrifugation and resuspended by vigorous agitating (Vortex) in Dulbecco's modified Eagle's medium (DMEM, GIBCO-BRL, Gaithersburg, USA)) containing 5% fetal bovine serum (FBS) for infecting murine macrophage cell line RAW (American Type Culture Collection No. 91B-71). This yielded a bacterial supernatant consisting mostly of single mycobacterial cells as observed by acid fast staining. Under the assumption that an optical density (O.D.) of 0.1 at 650 nm equals to 10⁸ CFU/ml (see in this respect Wei et al., “Identification of a Mycobacterium tuberculosis Gene that enhances survival of M. smegmatis in Macrophages”, J. Bacteriol. 182, 377-384 (2000)), the O.D. of Mycobacterium smegmatis cell suspension was measured and diluted to 5×10⁶ CFU/ml in DMEM containing 5% FBS. Viable counts were performed on Middlebrook 7H10 medium.

The RAW cell line was maintained in DMEM medium supplemented with 10% FBS. The survival assay for recombinant Mycobacterium smegmatis was performed as described by Wei et al., cited above. RAW cells were plated in a 24 well tissue culture plate (4×10⁵ cells/well) and incubated overnight in 5% CO₂ at 37° C. The inoculum (1 ml) containing 5×10⁶ recombinant Mycobacterium smegmatis was added to achieve multiplicities of infection (moi) of 10. The plate was incubated for 2 hours at 37° C. in 5% CO₂. The infected monolayers were washed twice with warm DMEM and treated with 2% FCS containing 200 μg of amikacin/mI for 1 hour at 37° C. to kill extracellular M. smegmatis. The cells were again washed twice with warm DMEM and further incubated in DMEM containing 20 μg of amikacin. This time point was considered 0 hours of infection. The 24 hours infected monolayer was incubated with 20 μg of amikacin/ml to prevent extracellular growth of any bacteria released by premature lysis of infected RAW cells. Cells were washed twice with warm DMEM before lysis was effected by addition of a 0.1% SDS solution and vigorously pipeting several times to ensure lysis of cells and release of surviving bacteria. The lysates were diluted in 7H9 broth and plated onto 7H10 agar plates and CFU were counted after incubation at 37° C. for 4 to 5 days.

Validation of Mycobacterial Kinase as a Mycobacterial Virulence Gene

Mycobacterium smegmatis was electroporated either with wildtype or mutant kinase (which exerts no kinase activity) or vector control. Mouse macrophage cell line (RAW) was infected with the various recombinant M. smegmatis expressing either pknG wild type or PknG K/M mutant or vector alone. After infection, the cells were lysed at different time points and the amount of intracellular bacteria was analysed. As can be seen from FIG. 1, after one hour postinfection the amount of bacteria recovered from macrophages infected with M. smegmatis expressing PknG wild type or K/M mutant or vector alone was the same. This shows that the recombinant M. smegmatis strains were internalised with equal efficiency. However, after 24 hour postinfection the amount of M. smegmatis transformed with the vector control or the mutant kinase was substantially decreased within macrophages. This shows an efficient clearance or degradation of the the M. smegmatis expressing vector alone or PknG K/M mutant by the lysosomal degradation pathway with in the macrophages. But in contrast, after 24 hrs an approximately tenfold increased amount of M. smegmatis survived within the cells expressing wildtype PknG compared to the mutant. This clearly demonstrates that the kinase activity of PknG increases the intracellular survival of M. smegmatis within macrophages and as such makes PknG an important virulence factor of mycobacteria. Consequently, the kinase is a promising target for recognising, monitoring, and controlling therapy of various diseases.

### General Assay Protocol for all Kinases

| | |
|---|---|
| Reaction Volume: | 40 μl |
| Reaction Time: | 60 min |
| Reaction Temperature: | room temperature |
| Assay Plate: | 96 well U bottom plate (Greiner, 650161) |
| MultiScreen-PH Plate: | 96 well MAPH Filter Plates |
| | (Millipore, MAPHNOB50) |
| Filter Washing Solution: | 0.75% H₃PO₄ |
| Szintilation Liquid: | Supermix Liquid Szintillator |
| | (PerkinElmer, 1200-439) |

Controls:

| | |
|---|---|
| Negative Control (C−): | 100 mM EDTA, no Inhibitor |
| Positive Control (C+): | no Inhibitor |

Reaction Buffer:

20 mM Tris-HCl, pH 7.5

10 mM MgCl2

1 mM DTT

Final Assay Concentrations:

| | |
|---|---|
| Kinase: | Use kinase conc. yielding 10% |
| | ATP turn over. |
| ATP: | 1 μM |
| Adenosine 5′-[γ-³³P]triphosphate: | 12.5 μCi/ml (Amersham Biosciences, |
| | BF1000) |
| Myelin Basic Protein (MBP): | 10 μM (Invitrogen, 13228-010) |

Pipetting Sequence:
1) Add 8 μl 50 μM MBP in Reaction Buffer to each well of Assay Plate
2) Add 10 μl 500 mM EDTA in H2O to C− wells
3) Add 8 μl 62.5 μCi/ml Adenosine 5′-[γ-³³P]triphosphate+5 μM ATP in Reaction Buffer to each well
4) Add 8 μl 5 fold concentrated inhibitor in 5% DMSO in Reaction Buffer to each well except to C− and C+ wells
5) Add 8 μl 5% DMSO in Reaction Buffer to C− and C+ wells
6) Add 8 μl 5 fold concentrated kinase in Reaction Buffer to each well
7) Incubate 1 hr at room temperature
8) Add 10 μl 50 mM EDTA in H2O to each well except to C− wells
9) Prepare MAPH plates by adding 200 μl 0.75% H₃PO₄ to each well
10) Exhaust 0.75% H₃PO₄ using Millipore vacuum station
11) Add 60 μl 0.75% H₃PO₄ to each well of MAPH Filter Plate
12) Transfer 30 μl sample per well from Assay Plate to corresponding well of MAPH Filter Plate
13) Incubate 30 min at room temperature
14) Wash each well of MAPH Filter Plates 3× with 200 μl 0.75% H₃PO₄ using Millipore vacuum station
15) Add 20 μl Szintilation Liquid to each well of MAPH Filter Plate
16) Seal MAPH Filter Plate
17) Store MAPH Filter Plate 30 min in darkness
18) Quantify radioactivity

Determination of RNA Polymerase II C-terminal Domain Phosphorylation:

The phosphorylation status of RNA polymerase II C-terminal domain was determined by western blot techniques. PM1 cells were seeded in 6-well plates at a density of 5×10⁵ per well. After over night incubation cells were treated with compound as indicated in the respective experiments. Cells were pelleted and lysed with 300 μl 3× Laemmli buffer followed by 30 min denaturing at 65° C. After separation of equal lysate volumes by SDS-PAGE the proteins were transferred to nitrocellulose membranes (Schleicher&Schuell) and probed with anti-SER2 (H5), anti-SER5 (H14) or RNA Poll II-antibodies purchased from Eurogentec and Santa Cruz, respectively. The amount of reactive protein was visualized by ECL detection methods (Amersham).

Growth Assay Using Alamar Blue™:

PM1 cells were seeded in 12-well plates at a density of 1.5×10⁵ per well with RPMI 1640 containing 10% FCS (fetal calf serum), 1% L-Glutamine and 1% Na-Pyruvate (Sigma). Cells were incubated with compound for 2-3 days (37° C., 6% CO₂) followed by subsequent splitting and renewing of compound-containing medium. At each of these time points an aliquot of cells served as data point for relative growth (given in % of the DMSO control [=100%]). The cell number was determined by addition of 10 μL Alamar Blue™ (Biozol) to 100 μl cell aliquots following the manufacturer's instructions.

HIV Replication Assay in PM1 Cells:

PM1 cells were seeded in 12-well plates at a density of 1.5×10⁵ per well with RPMI 1640 containing 10% FCS, 1% L-Glutamine and 1% Na-Pyruvate (Sigma). Cells were previously infected with HIV-1 BaL for 3 h at a concentration of about 5×10⁸ μg p24/cell. After addition of the respective compounds cells were incubated for 6 to 10 days. During this incubation the cells were passaged and compound-containing medium was renewed. The concentration of p24 in the cellular supernatants was determined at each of this time points using a previously described ELISA assay (Bevec et al., Proceedings of the National Academy of Sciences U.S.A. 1992, 89(20), 9870-9874).

NFκB-Dependent Transcriptional Activity:

The used NIH 3T3 75E11/300D8 cell line is described elsewhere (J. Eickhoff et al., Journal of Biological Chemistry, 2004, 279(10), 9642-9652).

HBV-Replication:

To test anti-HBV-activity of compounds the HBV-producing cell line HepG2-2.2.15 (M. A. Sells, PNAS 1987, 84, 1005-1009) was used. 1.0×10⁴ cells were seeded in 96-well microtiter plates in DMEM medium supplemented with 10% FCS. After incubation at 37° C. in 5% CO₂ atmosphere for 24 hours the medium was replaced with fresh medium containing the appropriately diluted compound. 3 days later medium was replaced by freshly prepared inhibitor-containing medium and the cells were incubated for further 3 days. Subsequently 200 μl lysis buffer (50 mM Tris-Cl 7.5; 1 mM EDTA 8.0; 0.5% NP40) per well was added. To remove cell debris and nucleic acids, lysate was centrifuged (15000 rpm, 10 min, 4° C.). Cellular and viral RNA was removed by addition of 2 μl of RNase. 100 μl of the samples were spotted onto an uncharged nylon membrane pre-wetted with PBS (phosphate-buffered saline) using a 96 well-blotting chamber (MINIfold Dot-Blot, Schleicher&Schüll). After further washing with 200 μl PBS per well the membrane was treated twice with 0.5M NaOH, 1.5M NaCl (2 min) and 4 times with 0.5M Tris 7.5, 3M NaCl (1 min). The nucleic acids were fixed by UV-treatment and used for hybridisation with a radioactive HBV-fragment prepared from the overgenome-length HBV-plasmid pT-HBV1.3 (L. G. Guidotti et al., Journal of Virology 1995., 69(10), 6158-6169).

The fixed membrane was pre-hybridized in a standard hybridisation buffer (50% formamide, 5×SSPE, 10×Denhards, 1% SDS, 100 μg/ml salmon sperm DNA) for at least 3 hours at 42° C. and hybridised overnight against the labelled HBV-fragment. The preparation of the HBV-fragment with the “Random primers DNA labelling system” (Invitrogen) was done according to the manufacturer's instructions. Hybridized filter were washed at room temperature with 2×SSC, at 62° C. with 2×SSC, 0.5% SDS and at 62° C. with 0.5×SSC, 0.5% SDS. Each washing step was carried out twice. The intensity of the HBV-DNA was quantified using a phosphoimager (Fuji). To test the cell viability 0.5×10⁴ HepG2-2.2.15-cells were seeded in 96-well-microtiter plates in DMEM medium supplemented with 10% fetal bovine serum. After incubation at 37° C. for 24 hours the medium was replaced by fresh compound-containing medium. 3 days later medium was replaced again by freshly prepared medium containing the inhibitor and the cells were incubated for further 3 days at 37° C. After the incubation period 1/10 volume of Alamar Blue (Serotec) solution containing a growth dependant indicator was added and the cells were incubated for 3 h at 37° C. Absorbance was monitored at 570 nm and 600 nm wavelength.

HCMV Replication:

Human foreskin fibroblasts (HFF) cell culture were grown in DMEM containing 10% FCS. For HCMV-replication assays, HFF cells were infected with HCMV strain AD169 producing EGFP (HCMV AD169-GFP; 27). 1 h post infection, medium was changed with medium containing the indicated compound concentration (0.3 μM, 1 μM and 3 μM, respectively) After incubation of 7 days cells were lysed (in 25 mM Tris, pH 7.5, 2 mM DTT, 1% Triton X-100 and 10% glycerol) and analysed for EGFP content in a Wallac Victor fluorescence detector.

HCV Replicon Assays:

Compounds were tested for activity in the HCV replicon system described by Bartenschlager and coworkers (Lohmann et al, Replication of subgenomic hepatitis C virus RNAs in a hepatoma cell line. Science 285, 110. 1999).

TABLE I
Inhibitory effect on mycobacterial protein kinase G (PknG) of selected
compounds according the present invention

| | Inhibition of PknG |
|---|---|
| Compound No. | (IC₅₀, [μM]) |
| Compounds: | <0.1 |
| A38, D37, D72, D88, D196, | |
| Compounds: | 0.1-1.0 |
| A8, A20, B1, B75, C45, D67, D106, | |
| D115, D175, D212 | |
| Compounds: | 1.1-10 |
| A17, B10, B15, B16, B17, B181, C49, | |
| D9, D32, D62, D96 | |
| Compounds: | >10 |
| B2, B14, B18, C2, C33 | |

TABLE II
Inhibitory effect of the compounds of the present invention on different
targets (+ = >50% inhibition at concentration 10 μM)

| \textcircled{1} | \textcircled{2} | \textcircled{3} | \textcircled{4} | \textcircled{5} | \textcircled{6} | \textcircled{7} | \textcircled{8} | \textcircled{9} | \textcircled{10} | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|---|---|
| A12 | | | + | + | + | + | | | + | + | + | + | + | + | + | + | | + |
| A15 | | | | + | + | + | | | + | | + | + | | | + | | + | + |
| A37 | + | | + | + | + | + | + | + | + | + | + | + | | + | + | | | + |
| B13 | | + | | | | | | | | + | + | | | | + | | | |
| B73 | | | | + | | | | | | | | + | | | + | | | |
| C45 | | | + | | | + | | | | | + | + | | | + | | | |
| D67 | | | + | + | + | + | | | | | | + | | | + | | + | |
| D86 | | | | | | | | | | | | | | | | | | |
| D115 | | | + | + | + | + | | | | + | + | + | | | | | | |
| D121 | | | + | + | + | + | | | | | | + | | | + | + | | |
| D134 | | | | + | | + | | | | | | + | | | | | | |
| D142 | | | + | + | | | + | | | | | + | | | + | + | | |

\textcircled{1} Compound Number
\textcircled{2} Target AKT1/PKBa act. HIS
\textcircled{3} Target CDK1/CycB act.GST-HIS
\textcircled{4} Target CDK2/CycA
\textcircled{5} Target CK1-alpha GST
\textcircled{6} Target EGFR GST-HIS
\textcircled{7} Target GSK-3beta HIS
\textcircled{8} Target IKKbeta HIS
\textcircled{9} Target InsR GST
\textcircled{10} Target Jnk1a1 HIS [symbol]
Target Kit human GST [symbol]
Target PDGFRbeta GST [symbol]
Target RICK-STREP 1 [symbol]
Target ROCK2 human HIS [symbol]
Target RSK1 act. HIS [symbol]
Target SRPK1 GST [symbol]
Target c-Src HIS [symbol]
Target cMet GST-HIS [symbol]
Target p70S6K HIS

Table I shows the half-maximal inhibition concentration (IC₅₀) values of representative compound according to general formula (I). Table II shows inhibition rates greater than 50% of various kinases. The results exhibited in both tables prove that the compounds of the present invention are potent pharmaceutically active agents against various diseases that can be treated and/or prohibited by inhibition of the targets \textcircled{1}-\textcircled{10}, [symbol] - [symbol] .

1. Compounds having the general formula (I) 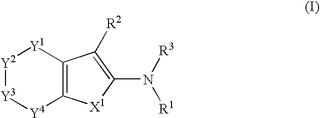
wherein
X¹ is selected from S, O, NH, NR⁴;
Y¹-Y²-Y³-Y⁴ represent the following residues: 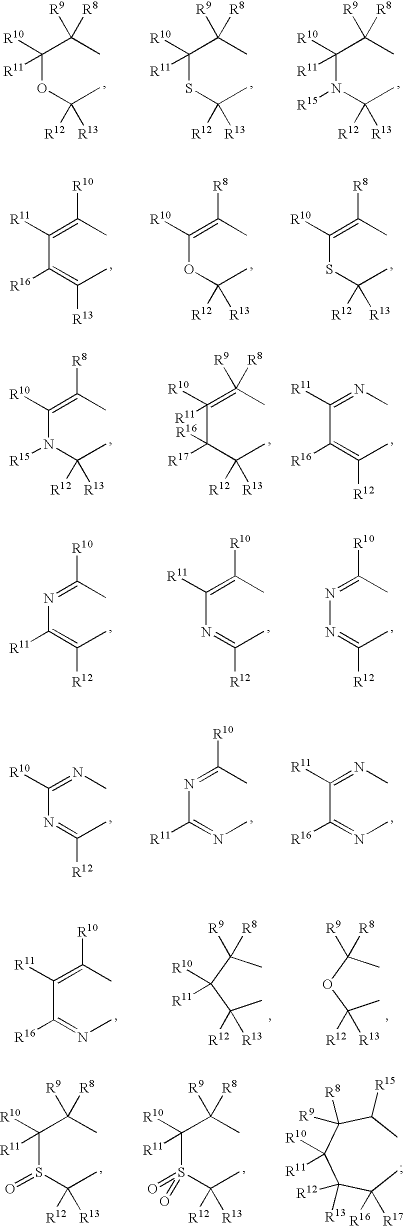
R¹, R⁴, R⁴′, R⁴″, R⁴′″, R⁴″″, R⁸, R⁹ and R¹⁵ are independently of each other selected from —H, substituted or unsubstituted C₁-C₈-alkyl, substituted or unsubstituted C₃-C₁₀-cycloalkyl, substituted or unsubstituted C₁-C₆-heterocyclyl, substituted or unsubstituted C₂-C₆-alkenyl, substituted or unsubstituted C₂-C₆-alkinyl, -Ph, —CH₂Ph, substituted phenyl, substituted benzyl, substituted or unsubstituted C₁-C₈-acyl;
R² represents the residue —CO—NH—R⁴, —CS—NH—R⁴, —SO₂—NH—R⁴, —C(═NH)—NH—R⁴, —COOR⁴, —SO₃—R⁴;
R³ is selected from —H, —CO—R⁵, —CS—R⁵, —C(═NR⁴″)—R⁵, —SO₂R⁵, —CO—R⁵′; —CS—R⁵′, —C(═NR⁴″)—R⁵′, —SO₂R⁵′, —CO—NR⁴″—R⁵, —CS—NR⁴″—R⁵, —CO—CO—NR⁴″—R⁵, —CO—CO—O—R⁵, —CO—OR⁵, —CS—OR⁵, —C(═NH)—NR⁴″—R⁵;
R⁵ is selected from substituted or unsubstituted C₃-C₁₀-cycloalkyl, substituted or unsubstituted C₁-C₈-alkyl, substituted or unsubstituted aryl, substituted or unsubstituted heteroaryl, substituted or unsubstituted C₁-C₆-heterocyclyl, substituted or unsubstituted C₂-C₆-alkenyl, substituted or unsubstituted alkylaryl, substituted or unsubstituted C₂-C₆-alkinyl, adamantyl, —H, —R¹⁴, —R¹⁸, —(CH₂)ₙ—C₁-C₆-heterocyclyl, —(CH₂)ₙ—C₃-C₁₀-cycloalkyl, 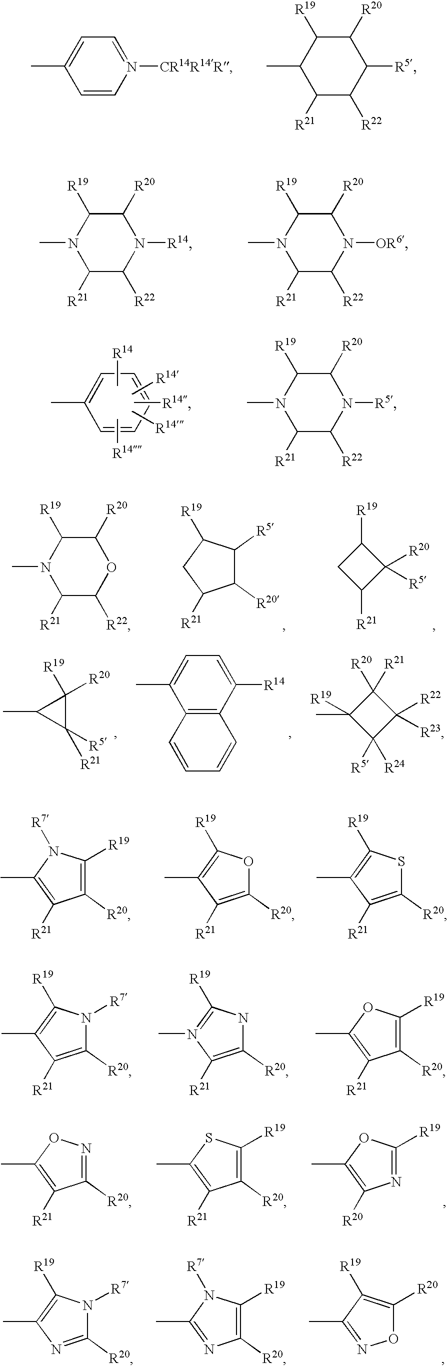 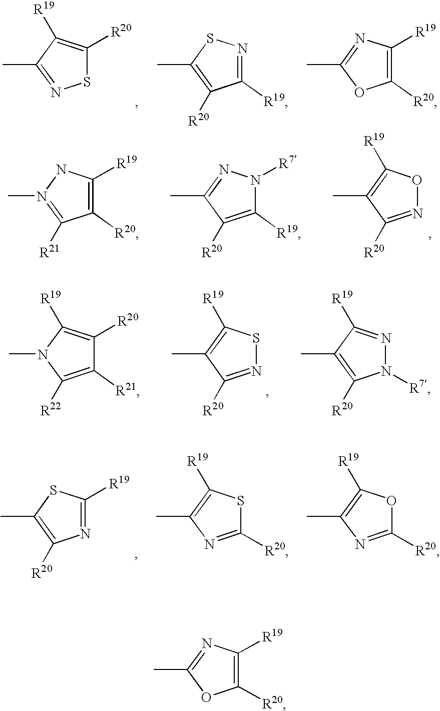
R⁶, R⁶′, R⁷ and R⁷′ are independently of each other selected from —H, substituted or unsubstituted C₃-C₁₀-cycloalkyl, substituted or unsubstituted C₁-C₈-alkyl, substituted or unsubstituted aryl, -Ph, —CH₂Ph, substituted phenyl, substituted benzyl, substituted or unsubstituted heteroaryl, substituted or unsubstituted C₁-C₆-heterocyclyl, substituted or unsubstituted C₂-C₆-alkenyl, substituted or unsubstituted C₂-C₆-alkinyl, adamantyl;
—R⁵′, R¹⁰, R¹¹, R¹², R¹³, R¹⁶ and R¹⁷ are independently of each other selected from —H, —OH, —NH₂, —R¹⁴, —R¹⁴′, —R¹⁴″, —R¹⁴′″, —R¹⁴″″, —R¹⁸, —R¹⁸′, —R¹⁸″, —R¹⁸′″, —R¹⁸″″, -Ph, —CH₂Ph, substituted phenyl, substituted benzyl, substituted or unsubstituted C₁-C₈-alkyl, substituted or unsubstituted C₃-C₁₀-cycloalkyl, substituted or unsubstituted C₁-C₆-heterocyclyl, substituted or unsubstituted C₂-C₆-alkenyl, substituted or unsubstituted C₂-C₆-alkinyl, adamantyl, substituted or unsubstituted aryl, substituted or unsubstituted heteroaryl, substituted or unsubstituted C₁-C₆-alkoxyl, substituted or unsubstituted —0—C₃-C₁₀-cycloalkyl, substituted or unsubstituted —O—C₁-C₆-heterocyclyl, substituted or unsubstituted —O-aryl, substituted or unsubstituted —O-heteroaryl, —OOC—O—R¹⁴′, —O—CO—R¹⁴′, —COO—R¹⁴40 , —O—CO—NR⁷R¹⁴, —NR¹⁴″″—CO—NR⁷R¹⁴, —NR¹⁴″″—CO—R¹⁴, —NR¹⁴″″—CS—NR⁷R¹⁴, —NR¹⁴″″—CS—OR¹⁴, —NR¹⁴″″—C(═NR¹⁴′″)—NR⁷R¹⁴, —O—CS—NR⁷R¹⁴, —CO—NR⁷R¹⁴, —NR⁷R¹⁴, —CH₂—SR¹⁴″″, —O—R¹⁴′, —OCR¹⁴′R¹⁴″R¹⁴′″, —OCH₂—R¹⁴′, —O—(CH₂)ₘ—R¹⁴′, —OCH₂—CR¹⁴′R¹⁴″R¹⁴′″, —O—(CH₂)ₘ—CR¹⁴′R¹⁴″R¹⁴′″, —O—CR¹⁹R²⁰—CR¹⁴′R¹⁴″R¹⁴′″, —OCH₂—OR¹⁴′, —O—(CH₂)ₚ—OR¹⁴′, —OCH₂—OCR¹⁴′R¹⁴″R¹⁴′″, —O—(CH₂)ₚ—OCR¹⁴′R¹⁴″R¹⁴′″, —O—CR¹⁹R²⁰—OCR¹⁴′R¹⁴″R¹⁴′″, —OCH₂—NR¹⁴″R¹⁴′″, —O—(CH₂)ₚ—NR¹⁴″R¹⁴′″, —O—CR¹⁹R²⁰—NR¹⁴″R¹⁴′″, —OCH₂—CO—R¹⁴, —O—(CH₂)ₘ—CO—R¹⁴′, —OCH₂—CO—CR¹⁴′R¹⁴″R¹⁴′″, —O—(CH₂)ₘ—CO—CR¹⁴′R¹⁴″R¹⁴′″, —O—CR¹⁹R²⁰—CO—CR¹⁴′R¹⁴″R¹⁴′″, —OCH₂—O—CO—R¹⁴′, —O—(CH₂)ₚ—CO—CO—R¹⁴′, —OCH₂—O—CO—CR¹⁴′R¹⁴″R¹⁴′″, —O—(CH₂)ₚ—O—CO—CR¹⁴′R¹⁴″R¹⁴′″, —O—CR¹⁹R²⁰—O—CO—CR¹⁴′R¹⁴″R¹⁴′″, —OCH₂—O—CO—OR¹⁴′, —O—(CH₂)ₚ—O—CO—OR¹⁴′, —OCH₂—O—CO—OCR¹⁴′R¹⁴″R¹⁴′″, —O—(CH₂)ₚ—O—CO—OCR¹⁴′R¹⁴″R¹⁴′″, —O—CR¹⁹R²⁰—O—CO—OCR¹⁴′R¹⁴″R¹⁴′″, —OCH₂—NR¹⁴′—CO—R¹⁴″, —O—(CH₂)ₚ—NR¹⁴′—CO—R¹⁴″, —OCH₂—NR¹⁴′—CO—CR¹⁴″R¹⁴′″R¹⁴″″, —O—(CH₂)ₚ—NR¹⁴′—CO—CR¹⁴″R¹⁴′″R¹⁴″″, —O—CR¹⁹R²⁰—NR¹⁴′—CO—CR¹⁴″R¹⁴′″R¹⁴″″, —OCH₂—NR¹⁴′—CO—OR¹⁴″, —O—(CH₂)ₚ—NR¹⁴′—CO—OR¹⁴″, —OCH₂—NR¹⁴′—CO—OCR¹⁴″R¹⁴′″R¹⁴″″, —O—(CH₂)ₚ—NR¹⁴′—CO—OCR¹⁴″R¹⁴′″R¹⁴″″, —O—CR¹⁹R²⁰—NR¹⁴′—CO—OCR¹⁴″R¹⁴′″R¹⁴″″, —OCH₂—CO—OR¹⁴′, —OCH₂—CO—OCR¹⁴′R¹⁴″R¹⁴′″, —O—(CH₂)ₘ—CO—OR¹⁴′, —O—(CH₂)ₘ—CO—OCR¹⁴′R¹⁴″R¹⁴′″, —O—CR¹⁹R²⁰—CO—OCR¹⁴′R¹⁴″R¹⁴40 ″, —OCH₂—CO—NR¹⁴″R¹⁴′″, —O—(CH₂)ₘ—CO—NR¹⁴″R¹⁴′″, —OCH₂—O—CO—NR¹⁴″R¹⁴′″, —O—(CH₂)ₚ—O—CO—NR¹⁴″R¹⁴′″, —O—CR¹⁹R²⁰—O—CO—NR¹⁴″R¹⁴′″, —OCH₂—NR¹⁴″—CO—NR¹⁴′″R¹⁴″″, —O—(CH₂)ₚ—NR¹⁴″—CO—NR¹⁴′″R¹⁴″″, —O—CR¹⁹R²⁰—NR¹⁴″—CO—NR¹⁴′″R¹⁴″″, —OCH₂—NR¹⁴″—C(═NR¹⁴′)—NR¹⁴′″R¹⁴″″, —O—(CH₂)ₚ—NR¹⁴″—C(═NR¹⁴′)—NR¹⁴′″R¹⁴″″, —CR¹⁴′R¹⁴″R¹⁴′″, —CH₂—R¹⁴′, —(CH₂)ₘ—R¹⁴′, —CH₂—CR¹⁴′R¹⁴″R¹⁴′″, —CH₂)ₘ—CR¹⁴′R¹⁴″R¹⁴′″, —CR¹⁹R²⁰—CR¹⁴′R¹⁴″R¹⁴′″, —CH₂—OR¹⁴′, —(CH₂)ₘ—OR¹⁴′, —CH₂—OCR¹⁴′R¹⁴″R¹⁴′″—(CH₂)ₘ—OCR¹⁴′R¹⁴″R¹⁴′″, —CR¹⁹R²⁰—OCR¹⁴′R¹⁴″R¹⁴′″, —CH₂—NR¹⁴″R¹⁴′″, —(CH₂)ₘ—NR¹⁴″R¹⁴′″, —CR¹⁹R²⁰—NR¹⁴″R¹⁴′″, —CH₂—CO—R¹⁴′, —(CH₂)ₘ—CO—R¹⁴′, —CH₂—CO—CR¹⁴′R¹⁴″R¹⁴′″, —(CH₂)ₘ—CO—CR¹⁴′R¹⁴″R¹⁴′″, —CR¹⁹R²⁰—CO—CR¹⁴′R¹⁴″R¹⁴′″, —CH₂—O—CO—R¹⁴′, —(CH₂ₘ—O—CO—R¹⁴′, —CH₂—O—CO—CR¹⁴′R¹⁴″R¹⁴′″, —(CH₂)ₘ—O—CO—CR¹⁴′R¹⁴″R¹⁴′″, —CR¹⁹R²⁰—O—CO—CR¹⁴′R¹⁴″R¹⁴′″, —CH₂—O—CO—OR¹⁴′, —(CH₂)ₘ—O—CO—OR¹⁴′, —CH₂—O—CO—OCR¹⁴′R¹⁴″R¹⁴′″, —(CH₂)ₘ—O—CO—OCR¹⁴′R¹⁴″R¹⁴′″, —CR¹⁹R²⁰—O—CO—OCR¹⁴′R¹⁴″R¹⁴′″, —CH₂—NR¹⁴′—CO—R¹⁴″, —(CH₂)ₘ—NR¹⁴′—CO—R¹⁴″, —CH₂—NR¹⁴′—CO—CR¹⁴″R¹⁴′″R¹⁴″″, —(CH₂)ₘ—NR¹⁴′—CO—CR¹⁴″R¹⁴′″R¹⁴″″, —CR¹⁹R²⁰—NR¹⁴′—CO—CR¹⁴″R¹⁴′″R¹⁴″″, —CH₂—NR¹⁴′—CO—OR¹⁴″, —(CH₂)ₘ—NR¹⁴′—CO—OR¹⁴″, —CH₂—NR¹⁴′—CO—OCR¹⁴″R¹⁴′″R¹⁴″″, —(CH₂)ₘ—NR¹⁴′—CO—OCR¹⁴″R¹⁴′″R¹⁴″″, —CR¹⁹R²⁰—NR¹⁴′—CO—OCR¹⁴″R¹⁴′″R¹⁴″″, —CH₂—CO—OR¹⁴′, —CH₂—CO—OCR¹⁴′R¹⁴″R¹⁴′″, —(CH₂)ₘ—CO—OR¹⁴′, —(CH₂)ₘ—CO—OCR¹⁴′R¹⁴″R¹⁴′″, —CR¹⁹R²⁰—CO—OCR¹⁴′R¹⁴″R¹⁴′″, —CH₂—CO—NR¹⁴″R¹⁴′″, —(CH₂)ₘ—CO—NR¹⁴″R¹⁴′″, —CR¹⁹R²⁰—CO—NR¹⁴″R¹⁴′″, —CH₂—O—CO—NR¹⁴″R¹⁴′″, —(CH₂)ₘ—O—CO—NR¹⁴′″R¹⁴″″, —CR¹⁹R²⁰—O—CO—NR¹⁴″R¹⁴′″, —CH₂—NR¹⁴″—CO—NR¹⁴′″R¹⁴″″, —(CH₂)ₘ—NR¹⁴″—CO—NR¹⁴′″R¹⁴″″, —CR¹⁹R²⁰—NR¹⁴″—CO—NR¹⁴′″R¹⁴″″, —CH₂—NR¹⁴″—C(═NR¹⁴′)—NR¹⁴′″R¹⁴″″, —(CH₂)ₘ—NR¹⁴″—C(═NR¹⁴′)—NR¹⁴′″R¹⁴″″, —CR¹⁹R²⁰—NR¹⁴″—C(═NR¹⁴′)—NR¹⁴′″R¹⁴″″, —NR¹⁴—CR¹⁴′R¹⁴″R¹⁴′″, —NR¹⁴—CH₂—R¹⁴′, —NR¹⁴—(CH₂)ₘ—R¹⁴′, —NR¹⁴—CH₂—CR¹⁴′R¹⁴″R¹⁴′″, —NR¹⁴—(CH₂)ₘ—CR¹⁴′R¹⁴″R¹⁴′″, —NR¹⁴—CR¹⁹R²⁰—CR¹⁴′R¹⁴″R¹⁴′″, —NR¹⁴—CH₂—OR¹⁴′, —NR¹⁴—(CH₂)ₚ—OR¹⁴′, —NR¹⁴—CH₂—OCR¹⁴′R¹⁴″R¹⁴′″, —NR¹⁴—(CH₂)ₚ—OCR¹⁴′R¹⁴″R¹⁴′″, —NR¹⁴—CR¹⁹R²⁰—OCR¹⁴′R¹⁴″R¹⁴′″, —NR¹⁴—CH₂—NR¹⁴″R¹⁴′″, —NR¹⁴—(CH₂)ₚ—NR¹⁴″R¹⁴′″, —NR¹⁴—CR¹⁹R²⁰—NR¹⁴″R¹⁴′″, —NR¹⁴—CH₂—CO—R¹⁴′, —NR¹⁴—(CH₂)ₘ—CO—R¹⁴′, —NR¹⁴—CH₂—CO—CR¹⁴′R¹⁴″R¹⁴′″, —NR¹⁴—(CH₂)ₘ—CO—CR¹⁴′R¹⁴″R¹⁴′″, —NR¹⁴—CR¹⁹R²⁰—CO—CR¹⁴′R¹⁴″R¹⁴′″, —NR¹⁴—CH₂—O—CO—R¹⁴′, —NR¹⁴—(CH₂)ₚ—O—CO—R¹⁴′, —NR¹⁴—CH₂—O—CO—CR¹⁴′R¹⁴″R¹⁴′″, —NR¹⁴—(CH₂)ₚ—O—CO—CR¹⁴′R¹⁴″R¹⁴′″, —NR¹⁴—CR¹⁹R²⁰—O—CO—CR¹⁴′R¹⁴″R¹⁴′″, —NR¹⁴—CH₂—O—CO—OR¹⁴′, —NR¹⁴—(CH₂)ₚ—O—CO—OR¹⁴′, —NR¹⁴—CH₂—O—CO—OCR¹⁴′R¹⁴″R¹⁴′″, —NR¹⁴—(CH₂)ₚ—O—CO—OCR¹⁴′R¹⁴″R¹⁴′″, —NR¹⁴—CR¹⁹R²⁰—O—CO—OCR¹⁴′R¹⁴″R¹⁴′″, —NR¹⁴—CH₂—NR¹⁴′—CO—R¹⁴″, —NR¹⁴—(CH₂)ₚ—NR¹⁴′—CO—R¹⁴″, —NR¹⁴—CH₂—NR¹⁴′—CO—CR¹⁴″R¹⁴′″R¹⁴″″, —NR¹⁴—(CH₂)ₚ—NR¹⁴′—CO—CR¹⁴″R¹⁴′″R^{41 ″, —NR}¹⁴—CR¹⁹R²⁰—NR¹⁴′—CO—CR¹⁴″R¹⁴′″R¹⁴″″, —NR¹⁴—CH₂—NR¹⁴′—CO—OR¹⁴″, —NR¹⁴—(CH₂)ₚ—NR¹⁴′—CO—OR¹⁴″, —NR¹⁴—CH₂—NR¹⁴′—CO—OCR¹⁴″R¹⁴′″R¹⁴″″, —NR¹⁴—(CH₂)ₚ—NR¹⁴′—CO—OCR¹⁴″R¹⁴′″R¹⁴″″, —NR¹⁴—CR¹⁹R²⁰—NR¹⁴′—CO—OCR¹⁴″R¹⁴′″R¹⁴″″, —NR¹⁴—CH₂—CO—OR¹⁴′, —NR¹⁴—CH₂—CO—OCR¹⁴′R¹⁴″R¹⁴′″, —NR¹⁴—(CH₂)ₘ—CO—OR¹⁴′, —NR¹⁴—(CH₂)ₘ—CO—OCR¹⁴′R¹⁴″R¹⁴′″, —NR¹⁴—CR¹⁹R²⁰—CO—OCR¹⁴′R¹⁴″R¹⁴′″, —NR¹⁴—CH₂—CO—NR¹⁴″R¹⁴′″, —NR¹⁴—(CH₂)ₘ—CO—NR¹⁴″R¹⁴′″, —NR¹⁴—CR¹⁹R²⁰—CO—NR¹⁴″R¹⁴′″, —NR¹⁴—CH₂—O—CO—NR¹⁴″R¹⁴′″, —NR¹⁴—(CH₂)ₚ—O—CO—NR¹⁴″R¹⁴′″, —NR¹⁴—CR¹⁹R²⁰—O—CO—NR¹⁴″R¹⁴′″, —NR¹⁴—Ch₂—NR¹⁴″—CO—NR¹⁴′″R¹⁴″″, —NR¹⁴—(CH₂)ₚ—NR¹⁴″—CO—NR¹⁴′″R¹⁴″″, —NR¹⁴—CR¹⁹R²⁰—NR¹⁴″—CO—NR¹⁴′″R¹⁴″″, —NR¹⁴—CH₂—NR¹⁴″—C(═NR¹⁴′)—NR¹⁴′″R¹⁴″″, —NR¹⁴—(CH₂)ₚ-NR¹⁴″—C(═NR¹⁴′)—NR¹⁴′″R¹⁴″″, —NR¹⁴—CR¹⁹R²⁰—NR¹⁴″—C(═NR¹⁴′)—NR¹⁴′″R¹⁴″″, —NR⁶R⁷, —(CH₂)ₙ—NR⁶R¹⁴, —(CH₂)ₙ—OR¹⁴, —(CH₂)ₙ—R¹⁴, —(CH₂)ₙ—SR¹⁴, —(CH₂)ₙ—SO—R¹⁴, —(CH₂)ₙ—SO₂—R¹⁴, —(CH₂)ₙ—SO₃—R¹⁴, —(CH₂)ₙ—CO—R¹⁴, —(CH₂)ₙ—COO—R¹⁴, —(CH₂)ₙ—O—CO—R¹⁴, —(CH₂)ₙ—NH—CO—R¹⁴, —(CH₂)ₙ—CO—NR⁶R¹⁴, —(CH)ₙ—O—CO—OR¹⁴, —(CH₂)ₙ—NH—CO—OR¹⁴, —(CH₂)ₙ—O—CO—NR⁶R¹⁴, —(CH₂)ₙ—NH—CO—NR⁶R¹⁴, —(CH₂)ₙ—NH—CS—NR⁶R¹⁴, —(CH₂)ₙ—NH—C(═NH)—NR⁶R¹⁴, —(CH₂)ₙ—aryl, —(CH₂)ₙ-heteroaryl, —CR¹⁹R²⁰—NR⁶R¹⁴, —CR¹⁹R²⁰—OR¹⁴, —CR¹⁹R²⁰R¹⁴, —CR¹⁹R²⁰—SR¹⁴, —CR¹⁹R²⁰—SO—R¹⁴, —CR¹⁹R²⁰—SO₂—R¹⁴, —CR¹⁹R²⁰—SO₃—R¹⁴, —CR¹⁹R²⁰—CO—R¹⁴, —CR¹⁹R²⁰—COO—R¹⁴, —CR¹⁹R²⁰—O—CO—R¹⁴, —CR¹⁹R²⁰—NH—CO—R¹⁴, —CR¹⁹R²⁰—CO—NR⁶R¹⁴, —CR¹⁹R²⁰—O—CO—OR¹⁴, —CR¹⁹R²⁰—NH—CO—OR¹⁴, —CR¹⁹R²⁰—O—CO—NR⁶R¹⁴, —CR¹⁹R²⁰—NH—CO—NR⁶R¹⁴, —CR¹⁹R²⁰—NH—CS—NR⁶R¹⁴, —CR¹⁹R²⁰—NH—C(═NH)—NR⁶R¹⁴, —CR¹⁹R²⁰-aryl, —CR¹⁹R²⁰-heteroaryl, —CH═CH—R¹⁸, —CH═CH—R¹⁴, —CR¹⁸′═CR¹⁸″R¹⁸′″, —CR¹⁴′═CR¹⁴″R¹⁴′″;
R¹¹ and R¹⁶ can form together with Y² and Y³ a fife-membered or six-membered saturated substituted or unsubstituted carbocyclic ring;
R⁸ and R⁹ represent together ═O, ═N—OH, ═N—OR⁴″″, ═N—R⁴″″, ═N—NH₂, ═N—NHR⁴″″, ═N—NR⁴′″R⁴″″, 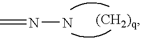
thus forming a carbonyl group, or an oxime, or a hydrazone, together with the carbon atom Y¹, or R⁸ and R⁹ form together a carbocyclic or heterocyclic ring;
R¹⁰ and R¹¹ if bound to the same carbon atom Y² represent together ═O, ═N—OH, ═N—OR⁴″″, ═N—R⁴″″, ═N—NH₂, ═N—NHR⁴″″, ═N—NR⁴′″R⁴″″, 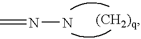
thus forming a carbonyl group, or an oxime, or a hydrazone, together with the carbon atom Y², or R¹⁰ and R¹¹ form together a carbocyclic or heterocyclic ring;
R¹² and R¹³ represent together ═O, ═N—OH, ═N—OR⁴″″, ═N—R⁴″″, ═N—NH₂, ═N—NHR⁴″″, 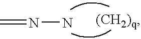
thus forming a carbonyl group, or an oxime, or a hydrazone, together with the carbon atom Y⁴, or R¹² and R¹³ form together a carbocyclic or heterocyclic ring;
R¹⁶ and R¹⁷ represent together ═O, ═N—OH, ═N—OR⁴″″, ═N—R⁴″″, ═N—NH₂, ═N—NHR⁴″″, ═N—NR⁴40 ″R⁴″″, 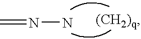
thus forming a carbonyl group, or an oxime, or a hydrazone, together with the carbon atom Y³, or R¹⁶ and R¹⁷ form together a carbocyclic or heterocyclic ring;
R¹⁴, R¹⁴′, R¹⁴″, R¹⁴′″, and R¹⁴″″ are independently of each other selected from —H, substituted or unsubstituted C₃-C₁₀-cycloalkyl, substituted or unsubstituted C₁-C₈-alkyl, substituted or unsubstituted aryl, substituted or unsubstituted heteroaryl, substituted or unsubstituted C₁-C₆-heterocyclyl, substituted or unsubstituted C₂-C₆-alkenyl, substituted or unsubstituted C₂-C₆-alkinyl, adamantyl, substituted or unsubstituted alkylaryl, —R¹⁸, —R¹⁸′, —R¹⁸″, —R¹⁸′″, R¹⁸″″, —R¹⁹, —R²⁰, —R²¹, —R²², —R²³, —(CH₂)ₙ—O—CO—R¹⁸″″, —(CH₂)ₙ—NH—CO—R¹⁸″″, —(CH₂)ₙ—NH—CO—R¹⁸″″, —(CH₂)ₙ—R¹⁸′, —(CH₂)ₙ—R¹⁸″, —(CH₂)ₙ—R¹⁸′″, —(CH₂)ₙ—SO—R¹⁸′″, —(CH₂)ₙ—SO₂—R¹⁸′″, —(CH₂)ₙ—CO—R¹⁸″″, —(CH₂)ₙ—NH—CO—R¹⁸″″, —(CH₂)ₙ—CO—NH—NH₂, —(CH₂)ₙ—SR¹⁸″, —C≡C—R¹⁸″″, —CR¹⁸′═CR¹⁸″R¹⁸′″, 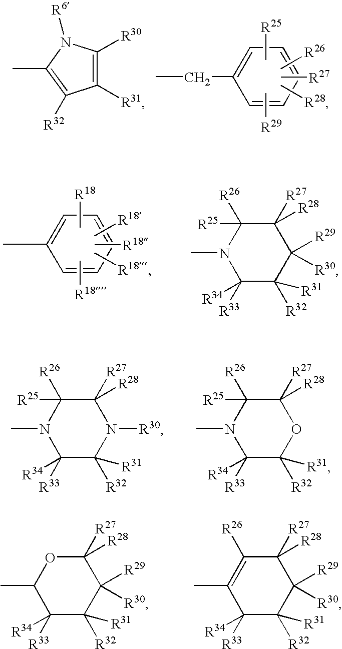 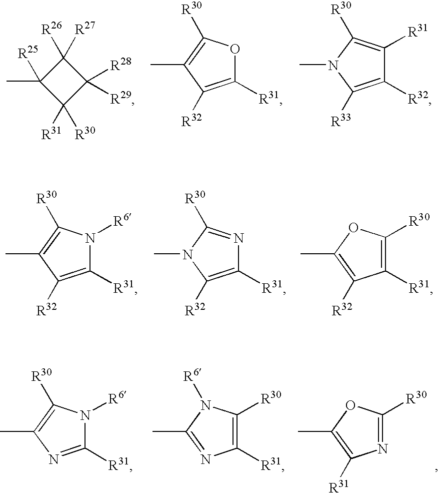
R¹⁸, R¹⁸′, R¹⁸″, R¹⁸′″, R¹⁸″″, R¹⁹-R³³ are independently of each other selected from —H, —OH, —OCH₃, —OC₂H₅, —OC₃H₇, —O-cyclo-C₃H₅, —OCH(CH₃)₂, —OC(CH₃)₃, —OPh, —OCH₂-Ph, —OCPh₃, —OR⁶, —OR⁷, —SH, —SCH₃, —SC₂H₅, —SC₃H₇, —S-cyclo-C₃H₅, —SCH(CH₃)₂, —SC(CH₃)₃, —SR⁶, —SR⁷, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —COOR⁶, —COOR⁷, —OOC—CH₃, —OOC—C₂H₅, —OOC—C₃H₇, —OOC-Cyclo-C₃H₅, —OOC—CH(CH₃)₂, —OOC—C(CH₃)₃, —OOC—R⁶, —OOC—R⁷, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —CONR⁶R⁷, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —NR⁶R⁷, —SOCH₃, —SOC₂H₅, —SOC₃H₇, —SO-cyclo-C₃H₅, —SOCH(CH₃)₂, —SOC(CH₃)₃, —SO—R⁶, —SO—R⁷, —SO₂CH₃, —SO₂C₂H₅, —SO₂C₃H₇, —SO₂-cyclo-C₃H₅, —SO₂CH(CH₃)₂, —SO₂C(CH₃)₃, —SO₂—R⁶, —SO₂—R⁷, —SO₃H, —SO₃CH₃, —SO₃C₂H₅, —SO₂C₃H₇, —SO₃-cyclo-C₃H₅, —SO₃CH(CH₃)₂, —SO₃C(CH₃)₃, —SO₃—R⁶, —SO₃—R⁷, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —O—COOC—R⁶, —O—COOC—R⁷, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CO—N(R⁶)₂, —NH—CO—N(R⁷)₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—CS—N(R⁶)₂, —NH—CS—N(R⁷)₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N[CH(CH₃)₂]₂, —NH—C(═NH)—N[C(CH₃)₃]₂, —NH—C(═NH)—N(R⁶)₂, —NH—C(═NH)—N(R⁷)₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—N(R⁶)₂, —O—CO—N(R⁷)₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —O—CO—OR⁶, —O—CO—OR⁷, —CH₂F, —CHF₂, —CF₃, —CH₂Cl, —CHCl₂, —CCl₃, —CH₂Br, —CHBr₂, —CBr₃, —CH₂, —CHl₂, —Cl₃, —CH₂—CH₂F, —CH₂—CHF₂, —CH₂—CF₃, —CH₂—CH₂Cl, —CH₂—CHCl₂, —CH₂—CCl₃, —CH₂—CH₂Br, —CH₂—CHBr₂, —CH₂—CBr₃, —CH₂—CH₂, —CH₂—CHI₂, —CH₂—Cl₃, —CH₂OH, —CH₂SH, —CH₂NH₂, —CH₂N(CH₃)₂, —CH₂N(C₂H₅)₂, —C₂H₄—OH, —C₂H₄—SH, —C₂H₄—NH₂, —C₂H₄—N(CH₃)₂, —C₂H₄—N(C₂H₅)₂, —CH₂OCH₃, —CH₂SCH₃, —CH₂OC₂H₅, —CH₂SC₂H₅, —C₂H₄—OCH₃, —C₂H₄—SCH₃, —C₂H₄—OC₂H₅, —C₂H₄—SC₂H₅, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, —Ph, —CH₂-Ph, —CH═CH₂, —CH═CHF, —CH═CF₂, —CF═CF₂, —CH═CHCl, —CH═CCl₂, —CCl═CCl₂, —CH═CHBr, —CH═CBr₂, —CBr═CBr₂, —CH₂—CH═CH₂, —C(CH₃)═CH₂, —CH═CH—CH₃, —CH═CH—CH₂—OH, —CH═CH—COOH, —CH═CH—COOCH₃, —CH═CH—COOC₂H₅, —CH═CH-Ph, —C(CH₃)═CH—CH₃, —C₂H₄—CH═CH₂, —CH═C(CH₃)₂, —C≡CH, —C≡C—CH₃, —CH₂—C≡CH, —CPh₃; 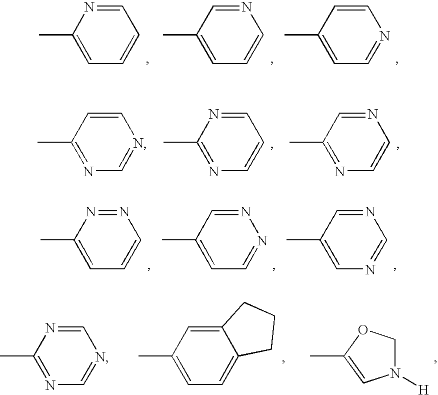 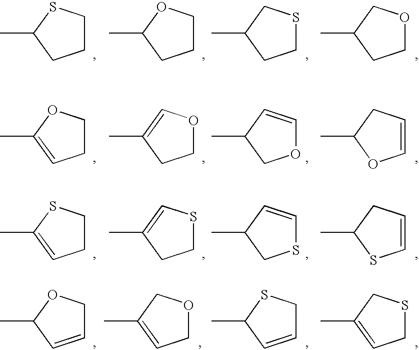 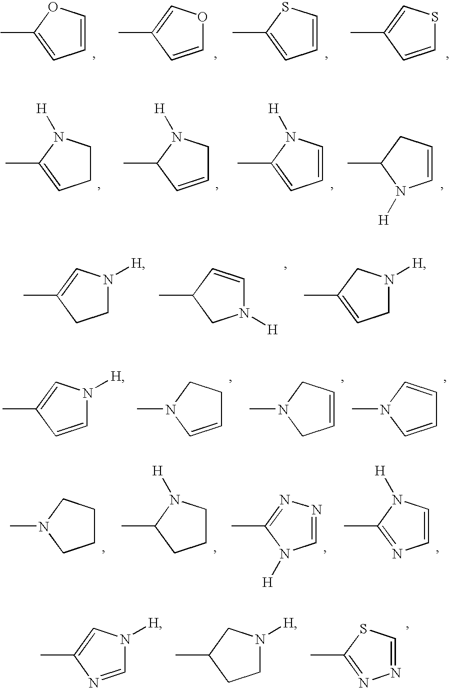 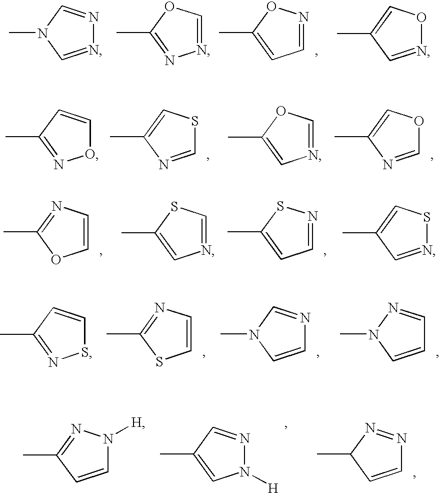 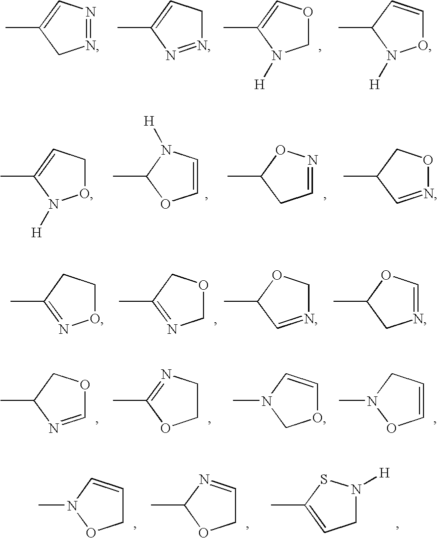 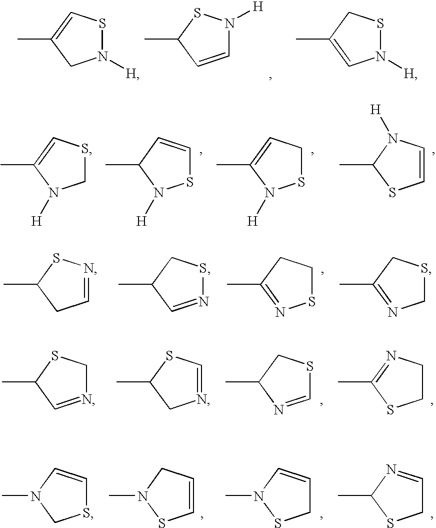 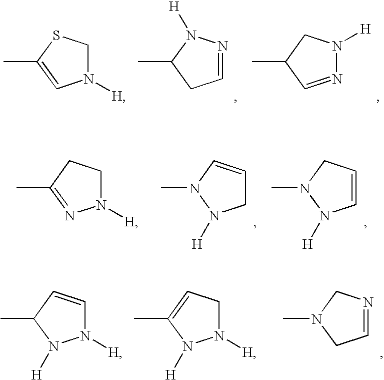 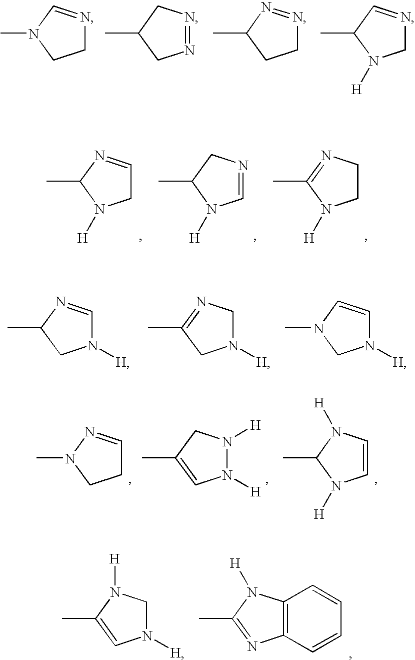 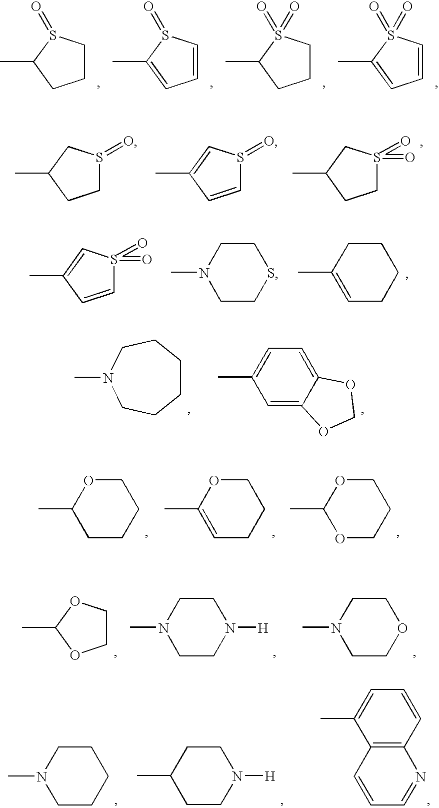 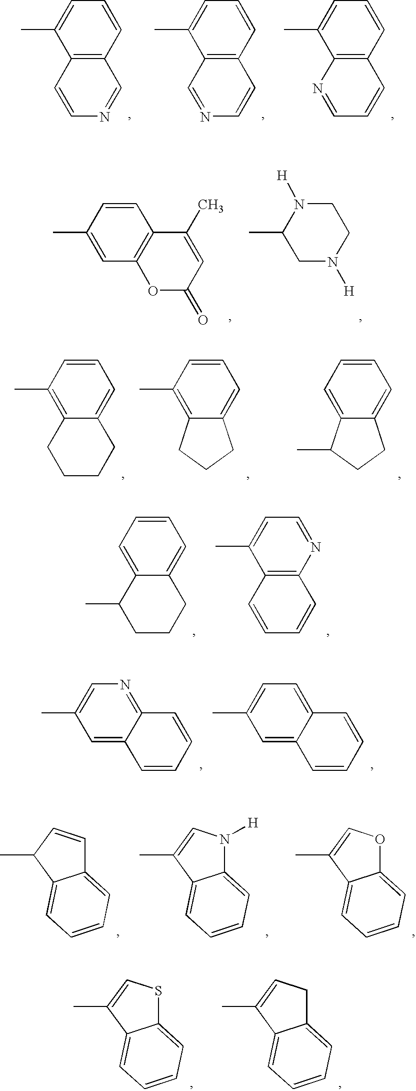 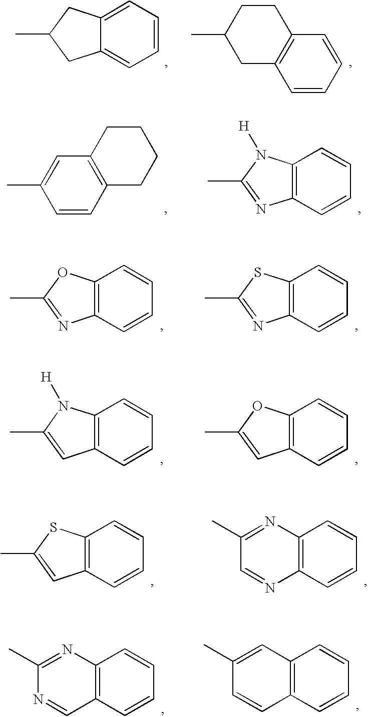 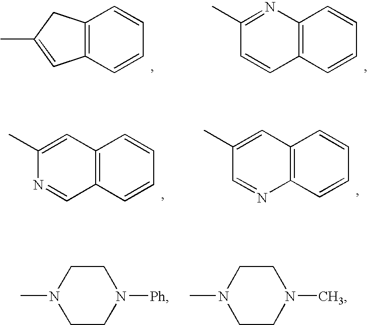
substituted or unsubstituted C₁-C₈-alkyl, substituted or unsubstituted C₂-C₆-alkenyl, substituted or unsubstituted C₂-C₆-alkinyl, substituted or unsubstituted C₃-C₁₀-cycloalkyl, substituted or unsubstituted aryl, -Ph, —CH₂Ph, substituted phenyl, substituted benzyl, substituted or unsubstituted heteroaryl, substituted or unsubstituted C₁-C₆-heterocyclyl;
m and n are independently of each other integers from 0 - 6;
p is an integer from 1-6;
q is an integer from 2-6;
and stereoisomeric and regioisomeric forms and pharmaceutically acceptable salts of these compounds;
with the proviso that if Y¹-Y²-Y³-Y⁴ represent 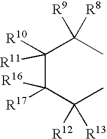
R² does not represent —COOR⁴ and with the proviso that the following compounds are excluded from the scope of the present invention:
2-(Toluene-4-sulfonylamino)-4,5,6,7-tetrahydro-benzo[b]thiophene-3-carboxylic acid amide;
2-[(5-Bromo-2-hydroxy-benzylidene)-amino]-4,5,6,7-tetrahydro-benzo[b]thiophene-3-carboxylic acid amide;
2-[(2-Chloro-benzylidene)-amino]-4,5,6,7-tetrahydro-benzo[b]thiophene-3-carboxylic acid amide;
Furan-2-carboxylic acid [3-(4-methoxy-phenylcarbamoyl)-4,5,6,7-tetrahydro-benzo[b]thiophen-2-yl]-amide;
2-[3-(4-Methoxy-phenyl)-3-(2,2,2-trifluoro-acetylamino)-propionyl-amino]-4,5,6,7-tetrahydro-benzo[b]thiophene-3-carboxylic acid amide;
2-(7-Ethyl-4-oxo-3-phenyl-3,4,5,6,7,8-hexahydro-benzo)[4,5]thieno[2,3-d]pyrimidin-2-ylsulfanyl)-N-(2-isopropoxy-phenyl)-acetamide;
N,N-Diethyl-2-(7-ethyl-4-oxo-3-phenyl-3,4,5,6,7,8-hexahydro-benzo)[4,5]thieno[2,3-d]pyrimidin-2-ylsulfanyl)-acetamide;
2-(2,2,3,3-Tetrafluro-propionylamino)-4,5,6,7-tetrahydro-benzo[b]thiophene-3-carboxylic acid amide;
2-[3-(2,2,2-Trichloro-1-propionylamino-ethyl)-thioureido]-4,5,6,7-tetrahydro-benzo[b]thiophene-3-carboxylic acid amide;
2-(2,4-Dichlorophenoxy)-N-(4-oxo-2-propyl-5,6,7,8-tetrahydro-4H-benzo[4,5]thieno[2,3-d]pyrimidin-3-yl)-acetamide;
6-Methyl-2-[(thiophene-2-carbonyl)-amino]-4,5,6,7-tetrahydro-benzo[b]thiophene-3-carboxylic acid amide;
2-[3-(3,4-Dichloro-phenyl-ureido]-4,5,6,7-tetrahydro-benzo[b]thiophene-3-carboxylic acid amide;
2-(2-Piperidin-1-yl-acetylamino)-4,5,6,7-tetrahydro-benzo[b]thiophene-3-carboxylic acid amide;
2-[2-(4-Methyl-piperazin-1-yl)-acetylamino]-4,5,6,7-tetrahydro-benzo[b]thiophene-3-carboxylic acid amide;
2-(3-Furan-2-yl-acryloylamino)-4,5,6,7-tetrahydro-benzo[b]thiophene-3-carboxylic acid amide;
2-[(2-Ethoxy-benzylidene)-amino]-4,5,6,7-tetrahydro-benzo[b]thiophene-3-carboxylic acid amide;
6-Methyl-2-(3-phenyl-propionylamino)-4,5,6,7-tetrahydro-benzo[b]thiophene-3-carboxylic acid amide;
Tetrahydro-furan-2-carboxylic acid (3-carbamoyl-6-methyl-4,5,6,7-tetrahydro-benzo[b]thiophen-2-yl)-amide;
2-(4-Fluoro-benzenesulfonylamino)-4,5,6,7-tetrahydro-benzo[b]thiophene-3-carboxylic acid amide;
6-tert-Butyl-2-[2-(5-methyl-3-trifluoromethyl-pyrazol-1-yl)-acetyl-amino]-4,5,6,7-tetrahydro-benzo[b]thiophene-3-carboxylic acid amide;
6-tert-Butyl-2-[2-(3,5-dimethyl-4-nitro-pyrazol-1-yl)-acetylamino]-4,5,6,7-tetrahydro-benzo[b]thiophene-3-carboxylic acid amide;
2-Amino-6-tert-butyl-4,5,6,7-tetrahydro-benzo[b]thiophene-3-carboxylic acid amide;
2-[2-(3,5-Dimethyl-4-nitro-pyrazol-1-yl)-acetylamino]-4,5,6,7-tetrahydro-benzo[b]thiophene-3-carboxylic acid amide;
2-(3-Carboxy-acryloylamino)-6-(1,1-dimethyl-propyl)-4,5,6,7-tetrahydro-benzo[b]thiophene-3-carboxylic acid amide;
6-(1,1-Dimethyl-propyl)-2-[(5-methyl-furan-2-carbonyl)-amino]-4,5,6,7-tetrahydro-benzo[b]thiophene-3-carboxylic acid amide;
2-(Cyclopropanecarbonyl-amino)-4,5,6,7-tetrahydro-benzo[b]thiophene-3-carboxylic acid amide;
2-[2-(4-Nitrophenyl)-acetylamino]-4,5,6,7-tetrahydro-benzo[b]thiophene-3-carboxylic acid amide;
6-Methyl-2-[2-(3-nitro-[1,2,4]triazol-1-yl)-acetylamino]-4,5,6,7-tetrahydro-benzo[b]thiophene-3-carboxylic acid amide;
Furan-2-carboxylic acid [3-(2-hydroxy-ethylcarbamoyl)-4,5,6,7-tetrahydro-benzo[b]thiophen-2-yl]-amide;
2-Acetylamino-4,5,6,7-tetrahydrobenzo[b]thiophene-3-carboxylic acid amide;
2-(2,2,-Dimethyl-propionylamino)-4,5,6,7-tetrahydrobenzo[b]thiophene-3-carboxylic acid amide;
2-Isobutyrylamino-4,5,6,7-tetrahydrobenzo[b]thiophene-3-carboxylic acid amide;
2-(2-Methyl-acryloylamino)-4,5,6,7-tetrahydrobenzo[b]thiophene-3-carboxylic acid amide;
2-[(Thiophene-2-carbonyl)-amino]-4,5,6,7-tetrahydrobenzo[b]thiophene-3-carboxylic acid amide;
Furan-2-carboxylic acid (3-carbamoyl-4,5,6,7-tetrahydro-benzo[b]thiophen-2-yl)-amide;
2-(Cyclobutanecarbonyl-amino)-4,5,6,7-tetrahydrobenzo[b]thiophene-3-carboxylic acid amide;
2-(2-Methyl-butyrylamino)-4,5,6,7-tetrahydrobenzo[b]thiophene-3-carboxylic acid amide;
2-(Cyclopropanecarbonyl-amino)-4-methyl-4,5,6,7-tetrahydrobenzo[b]thiophene-3-carboxylic acid amide;
6-tert-Butyl2-(cyclopropanecarbonyl-amino)-4,5,6,7-tetrahydrobenzo[b]thiophene-3-carboxylic acid amide;
2-(Cyclopropanecarbonyl-amino)-6-methyl-4,5,6,7-tetrahydrobenzo[b]thiophene-3-carboxylic acid amide;
2-(Cyclopropylmethyl-amino)-4,5,6,7-tetrahydrobenzo[b]thiophene-3-carboxylic acid amide;
2-(Cyclohexanecarbonyl-amino)4,5,6,7-tetrahydrobenzo[b]thiophene-3-carboxylic acid amide;
2-Acetylamino-6-methyl-4,5,6,7-tetrahydrobenzo[b]thiophene-3-carboxylic acid amide;
2-Amino-4,7-dihydro-5H-thieno[2,3-c]-thiopyran-3-carboxylic acid amide;
2-(Cyclopropanecarbonyl-amino)-4,7-dihydro-5H-thieno[2,3-c]-thiopyran-3-carboxylic acid amide;
2-(Cyclopropanecarbonyl-amino)-6λ⁴-oxo-4,5,6,7-tetrahydro-thieno[2,3-c]thiopyran-3-carboxylic acid amide;
N-[3-carbamoyl-4,5,6,7-tetrahydrobenzo[b]thien-2-yl]-acetamide;
N-[3-carbamoyl-4,5,6,7-tetrahydrobenzo[b]thien-2-yl]-propionamide;
N-[3-carbamoyl-4,5,6,7-tetrahydrobenzo[b]thien-2-yl]-2-butynoic amide;
N-[3-carbamoyl-4,5,6,7-tetrahydrobenzo[b]thien-2-yl]-cyanoacetamide;
N-[3-carbamoyl-4,5,6,7-tetrahydrobenzo[b]thien-2-yl]-cyclopropanecarboxamide;
N-[3-carbamoyl-4,5,6,7-tetrahydrobenzo[b]thien-2-yl]-isobutyramide;
N-[3-carbamoyl-4,5,6,7-tetrahydrobenzo[b]thien-2-yl]-3,3-dimethylacrylic amide;
N-[3-carbamoyl-4,5,6,7-tetrahydrobenzo[b]thien-2-yl]-2-ketobutyramide;
N-[3-carbamoyl-4,5,6,7-tetrahydrobenzo[b]thien-2-yl]-N,N-dimethylglycinamide;
N-[3-carbamoyl-4,5,6,7-tetrahydrobenzo[b]thien-2-yl]-3-chloropropionamide;
N-[3-carbamoyl-4,5,6,7-tetrahydrobenzo[b]thien-2-yl]-imidazol-4-carboxamide;
N-[3-carbamoyl-4,5,6,7-tetrahydrobenzo[b]thien-2-yl]-pyrrole-2-carboxamide;
N-[3-carbamoyl-4,5,6,7-tetrahydrobenzo[b]thien-2-yl]-cyclopentanecarboxamide;
N-[3-carbamoyl-4,5,6,7-tetrahydrobenzo[b]thien-2-yl]-1-cyanocyclopropane-carboxamide;
N-[3-carbamoyl-4,5,6,7-tetrahydrobenzo[b]thien-2-yl]-N-acetylglycinamide;
N-[3-carbamoyl-4,5,6,7-tetrahydrobenzo[b]thien-2-yl]-pyrrole-3-carboxamide;
N-[3-carbamoyl-4,5,6,7-tetrahydrobenzo[b]thien-2-yl]-benzamide;
N-[3-carbamoyl-4,5,6,7-tetrahydrobenzo[b]thien-2-yl]-4-pyrazolecarboxamide;
N-[3-carbamoyl-4,5,6,7-tetrahydrobenzo[b]thien-2-yl]-picolinic amide;
N-[3-carbamoyl-4,5,6,7-tetrahydrobenzo[b]thien-2-yl]-nicotinic amide;
N-[3-carbamoyl-4,5,6,7-tetrahydrobenzo[b]thien-2-yl]-isonicotinic amide;
N-[3-carbamoyl-4,5,6,7-tetrahydrobenzo[b]thien-2-yl]-2-pyrazinecarboxamide;
N-[3-carbamoyl-4,5,6,7-tetrahydrobenzo[b]thien-2-yl]-1-methylpyrrole-2-carboxamide;
N-[3-carbamoyl-4,5,6,7-tetrahydrobenzo[b]thien-2-yl]-3-methyl-2-furoic amide;
N-[3-carbamoyl-4,5,6,7-tetrahydrobenzo[b]thien-2-yl]-5-methylisoxazole-4-carboxamide;
N-[3-carbamoyl-4,5,6,7-tetrahydrobenzo[b]thien-2-yl]-3-methylisoxazole-4-carboxylic
N-[3-carbamoyl-4,5,6,7-tetrahydrobenzo[b]thien-2-yl]-thiophene-2-carboxamide;
N-[3-carbamoyl-4,5,6,7-tetrahydrobenzo[b]thien-2-yl]-thiophene-3-carboxamide;
N-[3-carbamoyl-4,5,6,7-tetrahydrobenzo[b]thien-2-yl]-dl-pyroglutamic amide;
N-[3-carbamoyl-4,5,6,7-tetrahydrobenzo[b]thien-2-yl]-1-(aminocarbonyl)-1-cyclopropanecarboxamide;
N-[3-carbamoyl-4,5,6,7-tetrahydrobenzo[b]thien-2-yl]-o-toluic amide;
N-[3-carbamoyl-4,5,6,7-tetrahydrobenzo[b]thien-2-yl]-5-methylisoxazole-3-carboxamide;
N-[3-carbamoyl-4,5,6,7-tetrahydrobenzo[b]thien-2-yl]-m-toluic amide;
N-[3-carbamoyl-4,5,6,7-tetrahydrobenzo[b]thien-2-yl]-3-aminopyrazole-4-carboxamide;
N-[3-carbamoyl-4,5,6,7-tetrahydrobenzo[b]thien-2-yl]-p-toluic amide;
N-[3-carbamoyl-4,5,6,7-tetrahydrobenzo[b]thien-2-yl]-salicylic amide;
N-[3-carbamoyl-4,5,6,7-tetrahydrobenzo[b]thien-2-yl]-3-hydroxybenzamide;
N-[3-carbamoyl-6-methyl-4,5,6,7-tetrahydrothieno[2,3-c]pyridin-2-yl]-3,4,5-trimethoxybenzamide;
N-[3-carbamoyl-6-methyl-4,5,6,7-tetrahydrothieno[2,3-c]pyridin-2-yl]-2,4,6-trimethoxybenzamide;
N-[3-carbamoyl-6-methyl-4,5,6,7-tetrahydrothieno[2,3-c]pyridin-2-yl]-3-chlorobenzo[b]thiophene-2-carboxamide;
N-[3-carbamoyl-6-methyl-4,5,6,7-tetrahydrothieno[2,3-c]pyridin-2-yl]-3-(phenylsulfonyl)propionamide;
N-[3-carbamoyl-6-methyl-4,5,6,7-tetrahydrothieno[2,3-c]pyridin-2-yl]-4-toluenesulfonylacetamide;
N-[3-carbamoyl-6-methyl-4,5,6,7-tetrahydrothieno[2,3-c]pyridin-2-yl]-4-methylsulfonylphenylacetamide;
N-[3-carbamoyl-6-methyl-4,5,6,7-tetrahydrothieno[2,3-c]pyridin-2-yl]-5-fluoroindole-3-acetamide;
N-[3-carbamoyl-6-methyl-4,5,6,7-tetrahydrothieno[2,3-c]pyridin-2-yl]-3-phthalimido-propionamide;
N-[3-carbamoyl-6-methyl-4,5,6,7-tetrahydrothieno[2,3-c]pyridin-2-yl]-5-methoxy-2-methyl-3-indoleacetamide;
N-[3-carbamoyl-6-methyl-4,5,6,7-tetrahydrothieno[2,3-c]pyridin-2-yl]-5-methoxy-1-indanone-3-acetamide;
N-[3-carbamoyl-6-methyl-4,5,6,7-tetrahydrothieno[2,3-c]pyridin-2-yl]-5-(4-chlorophenyl)-2-furoic amide;
N-[3-carbamoyl-6-methyl-4,5,6,7-tetrahydrothieno[2,3-c]pyridin-2-yl]-6-chlorokynurenic amide;
N-[3-carbamoyl-6-methyl-4,5,6,7-tetrahydrothieno[2,3-c]pyridin-2-yl]-N′-(4-chlorophenyl)maleamic amide;
N-[3-carbamoyl-6-methyl-4,5,6,7-tetrahydrothieno[2,3-c]pyridin-2-yl]-N′-p-tosylglycinamide;
N-[3-carbamoyl-6-methyl-4,5,6,7-tetrahydrothieno[2,3-c]pyridin-2-yl]-5-chloroindole-2-carboxamide;
N-[3-carbamoyl-6-methyl-4,5,6,7-tetrahydrothieno[2,3-c]pyridin-2-yl]-N′-(1-naphthyl)maleamic amide;
N-[3-carbamoyl-6-methyl-4,5,6,7-tetrahydrothieno[2,3-c]pyridin-2-yl]-3-iodobenzamide;
N-[3-carbamoyl-6-methyl-4,5,6,7-tetrahydrothieno[2,3-c]pyridin-2-yl]-4-iodobenzamide;
N-[3-carbamoyl-6-methyl-4,5,6,7-tetrahydrothieno[2,3-c]pyridin-2-yl]-N-m-tolylphthalamic amide;
N-[3-carbamoyl-6-methyl-4,5,6,7-tetrahydrothieno[2,3-c]pyridin-2-yl]-N′-acetyl-dl-histidine;
N-[3-carbamoyl-6-methyl-4,5,6,7-tetrahydrothieno[2,3-c]pyridin-2-yl]-3-acetamino-6-bromobenzamide;
N-[3-carbamoyl-6-methyl-4,5,6,7-tetrahydrothieno[2,3-c]pyridin-2-yl]-2-acetamido-5-bromobenzamide;
N-[3-carbamoyl-6-methyl-4,5,6,7-tetrahydrothieno[2,3-c]pyridin-2-yl]-2-iodophenylacetamide;
N-[3-carbamoyl-6-methyl-4,5,6,7-tetrahydrothieno[2,3-c]pyridin-2-yl]-4-iodophenylacetamide;
N-[3-carbamoyl-6-methyl-4,5,6,7-tetrahydrothieno[2,3-c]pyridin-2-yl]-8-(3-carboxamidopropyl)-1,3-dimethylxanthine;
N-[3-carbamoyl-6-methyl-4,5,6,7-tetrahydrothieno[2,3-c]pyridin-2-yl]-7-bromokynurenic amide;
N-[3-carbamoyl-6-methyl-4,5,6,7-tetrahydrothieno[2,3-c]pyridin-2-yl]-N′-benzoyl-dl-phenylalaninamide;
N-[3-carbamoyl-6-methyl-4,5,6,7-tetrahydrothieno[2,3-c]pyridin-2-yl]-indole-3-butyramide;
N-[3-carbamoyl-6-methyl-4,5,6,7-tetrahydrothieno[2,3-c]pyridin-2-yl]-4-chloroindole-3-acetamide;
N-[3-carbamoyl-6-methyl-4,5,6,7-tetrahydrothieno[2,3-c]pyridin-2-yl]-dl-desthiobiotin;
N-[3-carbamoyl-6-methyl-4,5,6,7-tetrahydrothieno[2,3-c]pyridin-2-yl]-4,6-dichloroindole-2-carboxamide;
N-[3-carbamoyl-6-methyl-4,5,6,7-tetrahydrothieno[2,3-c]pyridin-2-yl]-N′-benzoyl-histidinamid;
2-Amino-6-(4-fluoro-phenyl)-benzo[b]thiophene-3-carboxylic acid amide;
2-Ureido-benzo[b]thiophene-3-carboxylic acid amide.

2. Compounds according to claim 1, wherein X¹ is S.

3. Compounds according to claim 1, wherein R⁸ and R⁹ represent hydrogen.

4. Compounds according to claim 1, represented by the general formula (IId): 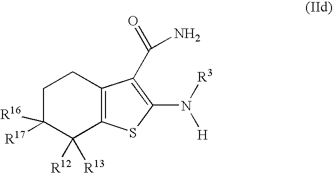
wherein
R³, R¹², R¹³, R¹⁶, and R¹⁷ have the meanings as defined in claim 1.

5. Compounds according to claim 1, represented by the general formula (IIIa): 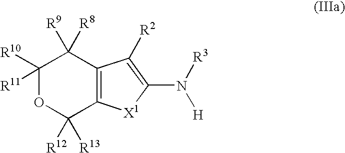
wherein
X¹ is selected from S, O, NR⁴′,
R⁴′ is selected from H, substituted or unsubstituted C₁-C₆-alkyl,
R² is selected from —CO—NH—R⁴, —CS—NH—R⁴, —SO₂—NH—R⁴;
wherein R⁴ is selected from —H, HO-substituted, H₂N-substituted or HS-substituted C₁-C₆-alkyl,
R³ is selected from H, —C(═O)R⁵, —C(═S)R⁵, —C(═NH)R⁵ and —SO₂R⁵, wherein
R⁵ is selected from substituted or unsubstituted C₃-C₆-cycloalkyl, C₁-C₆-alkyl, aryl, heteroaryl, heterocycloalkyl, C₂-C₄-alkenyl, C₂-C₄-alkinyl, adamantyl, 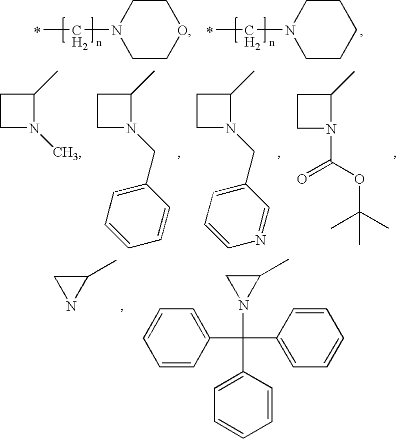
or —(CH₂)ₙ—NR⁶R⁷
wherein R⁶ and R⁷ are independently selected from substituted or unsubstituted C₁-C₄-alkyl or C₂-C₄-alkenyl and wherein n=1 to 6,
or NR⁶R⁷,
wherein
R⁶ is selected from H, C₁-C₆-alkyl, and
R⁷ is selected from substituted or unsubstituted C₃-C₆-cycloalkyl, C₁-C₆-alkyl,
aryl, heteroaryl, heterocycloalkyl, C₂-C₄-alkenyl, C₂-C₄-alkinyl, or adamantyl,
R⁸ is H and R⁹ is selected from H, substituted or unsubstituted C₁-C₆-alkyl
R¹⁰ is selected from H, substituted or unsubstituted C₁-C₆-alkyl, C₁-C₆-alkoxy, or OH
R₁₁ is selected from H and substituted or unsubstituted C₁-C₆-alkyl
R₁₂ is selected from H and substituted or unsubstituted C₁-C₆-alkyl, C₁-C₆-alkoxy, or OH, and
R¹³ is selected from H or substituted or unsubstituted C₁-C₆-alkyl,
and stereoisomeric and regioisomeric forms and pharmaceutically acceptable salts of these compounds.

6. Compounds according to any claim 1, wherein R² is —CO—NH₂.

7. Compounds of general formula (I) according to claim 1, selected from the group comprising:
(Comp. A1) 2-(3-Cyclohexyl-ureido)-6-hydroxy-benzo[b]thiophene-3-carboxylic acid amide,
(Comp. A2) 2-(Cyclopropanecarbonyl-amino)-6-methoxy-benzo[b]thiophene-3-carboxylic acid amide,
(Comp. A3) Acetic acid 3-carbamoyl-2-(cyclopropanecarbonyl-amino)-benzo[b]thiophen-6-yl ester,
(Comp. A4) 2-[(2-Methyl-cyclopropanecarbonyl)-amino]-benzo[b]thiophene-3-carboxylic acid amide,
(Comp. A5) 6-Hydroxy-2-[(2-methyl-cyclopropanecarbonyl)-amino]-benzo[b]thiophene-3-carboxylic acid amide,
(Comp. A6) 2-(cyclopropanecarbonyl-amino)-6-methyl-benzo[b]thiophene-3-carboxylic acid amide,
(Comp. A7) 2-(Cyclopropanecarbonyl-amino)-6-methylcarbamoylmethoxy-benzo[b]thiophene-3-carboxylic acid amide,
(Comp. A8) 2-(Cyclopropanecarbonyl-amino)-6-[(2-hydroxy-ethylcarbamoyl)-methoxy]-benzo[b]thiophene-3-carboxylic acid amide,
(Comp. A9) 2-(Cyclopropanecarbonyl-amino)-6-ethoxy-benzo[b]thiophene-3-carboxylic acid amide,
(Comp. A10) 7-Chloro-2-(cyclopropanecarbonyl-amino)-6-(2-piperidin-1-yl-ethoxy)-benzo[b]thiophene-3-carboxylic acid amide,
(Comp. A11) 6-Hydroxy-2-[3-(4-methoxy-phenyl)-ureido]-benzo[b]thiophene-3-carboxylic acid amide,
(Comp. A12) 6-Hydroxy-2-(3-phenyl-ureido)-benzo[b]thiophene-3-carboxylic acid amide,
(Comp. A13) N-(3-Carbamoyl-6-hydroxy-benzo[b]thiophen-2-yl)-oxalamide,
(Comp. A14) 5-Bromo-2-(cyclopropanecarbonyl-amino)-benzo[b]thiophene-3-carboxylic acid amide,
(Comp. A15) 6-Bromo-2-(cyclopropanecarbonyl-amino)-7-hydroxy-benzo[b]thiophene-3-carboxylic acid amide,
(Comp. A16) 2-(Cyclopropanecarbonyl-amino)-benzo[b]thiophene-3-carboxylic acid amide,
(Comp. A17) 2-(Cyclopropanecarbonyl-amino)-6-hydroxy-benzo[b]thiophene-3,5-dicarboxylic acid 3-amide-5-diethylamid,
(Comp. A18) 2-(Cyclopropanecarbonyl-amino)-6-hydroxy-benzo[b]thiophene-3,7-dicarboxylic acid 3-amide-7-diethylamid,
(Comp. A19) 6-(3-Amino-propoxy)-2-(cyclopropanecarbonyl-amino)-benzo[b]thiophene-3-carboxylic acid amide,
(Comp. A20) 2-(Cyclopropanecarbonyl-amino)-6-(2-hydroxy-ethoxy)-benzo[b]thiophene-3-carboxylic acid amide,
(Comp. A21) 2-(Cyclopropanecarbonyl-amino)-6-[2-(tetrahydro-pyran-2-yloxy)-ethoxy]-benzo[b]thiophene-3-carboxylic acid amide,
(Comp. A22) (3-Carbamoyl-6-hydroxy-benzo[b]thiophen-2-yl)-carbamic acid benzyl ester,
(Comp. A23) 6-Ethoxy-2-[3-(4-methoxy-phenyl)-ureido]-benzo[b]thiophene-3-carboxylic acid amide,
(Comp. A24) 7-Chloro-2-[3-(4-fluoro-phenyl)-ureido]-6-hydroxy-benzo[b]thiophene-3-carboxylic acid amide,
(Comp. A25) Diethyl-carbamicacid-3-carbamoyl-2-(cyclopropane-carbonyl-amino)-benzo[b]thiophen-6-yl ester,
(Comp. A26) 6-Hydroxy-2-[3-(4-trifluoromethyl-phenyl)-ureido]-benzo[b]thiophene-3-carboxylic acid amid,
(Comp. A27) 2-[3-(4-Bromo-phenyl)-ureido]-6-methyl-benzo[b]thiophene-3-carboxylic acid amide,
(Comp. A28) 3-Carbamoyl-2-(cyclopropanecarbonyl-amino)-benzo[b]thiophen-6-carboxylic acid ethyl ester,
(Comp. A29) [3-Carbamoyl-2-(cyclopropanecarbonyl-amino)-benzo[b]thiophen-6-yloxy]-acetic acid methyl ester
(Comp. A30) 2-(Cyclopropanecarbonyl-amino)-6-hydroxy-benzo[b]thiophene-3-carboxylic acid amide,
(Comp. A31) 2-[3-(4-Fluoro-phenyl)-ureido]-6-hydroxy-benzo[b]thiophene-3-carboxylic acid amid,
(Comp. A32) 7-Bromo-2-(cyclopropanecarbonyl-amino)-6-hydroxy-benzo[b]thiophene-3-carboxylic acid amide,
(Comp. A33) 6-Hydroxy-2-[3-(4-iodo-phenyl)-ureido]-benzo[b]thiophene-3-carboxylic acid amide,
(Comp. A34) 2-(Cyclopropanecarbonyl-amino)-6-(2-piperidin-1-yl-ethoxy)-benzo[b]thiophene-3-carboxylic acid amide,
(Comp. A35) 7-Chloro-2-(cyclopropanecarbonyl-amino)-6-hydroxy-benzo[b]thiophene-3-carboxylic acid amide,
(Comp. A36) 2-(Cyclopropanecarbonyl-amino)-benzo[b]thiophene-3-carboxylic acid methylamide,
(Comp. A37) 7-Chloro-6-hydroxy-2-[(2-methyl-cyclopropanecarbonyl)-amino]-benzo[b]thiophene-3-carboxylic acid amide,
(Comp. A38) 5,7-Dibromo-2-(cyclopropanecarbonyl-amino)-6-hydroxy-benzo[b]thiophene-3-carboxylic acid amide,
(Comp. A39) 2-[3-(4-Bromo-phenyl)-ureido]-6-hydroxy-benzo[b]thiophene-3-carboxylic acid amide,
(Comp. A40) 2-Cyclopropyl-7-methyl-3H-benzo[4,5]thieno[2,3-d]pyrimidin-4-one;
(Comp. A41) 2-[3-(4-Ethoxy-phenyl)-ureido]-6-hydroxy-benzo[b]thiophene-3-carboxylic acid amide,
(Comp. A42) 2-[3-(3-Chloro-4-methyl-phenyl)-ureido]-6-hydroxy-benzo[b]thiophene-3-carboxylic acid amide,
(Comp. A43) 5,7-dichloro-2-(cyclopropanecarbonyl-amino)-6-hydroxy-benzo[b]thiophene-3-carboxylic acid amide,
(Comp. A44) 5,7-Dichloro-2-(cyclopropanecarbonyl-amino)-6-methoxy-benzo[b]thiophene-3-carboxylic acid amide,
(Comp. A45) 5,7-Dichloro-2-(cyclopropanecarbonyl-amino)-6-ethoxy-benzo[b]thiophene-3-carboxylic acid amide,
(Comp. A46) 5,7-Dichloro-2-(cyclopropanecarbonyl-amino)-6-isopropoxy-benzo[b]thiophene-3-carboxylic acid amide,
(Comp. A47) 2-[3-(3-Chloro-2-methyl-phenyl)-ureido]-6-hydroxy-benzo[b]thiophene-3-carboxylic acid amide,
(Comp. A48) 6-Hydroxy-2-[3-(2-methylsulfanyl-phenyl)-ureido]-benzo[b]thiophene-3-carboxylic acid amide,
(Comp. A49) 5,7-Dichloro-2-(cyclopropanecarbonyl-amino)-6-(2-hydroxyethoxy)-benzo[b]thiophene-3-carboxylic acid amide,
(Comp. A50) 6-(2-Amino-ethoxy)-5,7-dichloro-2(cyclopropanecarbonyl-amino)-benzo[b]thiophene-3-carboxylic acid amide hydrochloride,
(Comp. A51) 2-(Cyclopropanecarbonyl-amino)-6-(2-morpholin-4-yl-2-oxo-ethoxy)-benzo[b]thiophene-3-carboxamide,
(Comp. A52) 2-(Cyclopropanecarbonyl-amino)-6-[(3-hydroxy-propyl-carbamoyl)-methoxy]-benzo[b]thiophene-3-carboxamide,
(Comp. A53) 6-{[Bis-(2-hydroxy-ethyl)-carbamoyl]-methoxy}-2-(cyclopropanecarbonyl-amino)-benzo[b]thiophene-3-carboxamide,
(Comp. A54) 2-(Cyclopropanecarbonyl-amino)-6-[2-(4-methyl-piperazin-1-yl)-2-oxo-ethoxy]-benzo[b]thiophene-3-carboxamide,
(Comp. A55) 2-(Cyclopropanecarbonyl-amino)-6-[(3,4-dimethoxy-benzylcarbamoyl)-(methoxy]-benzo[b]thiophene-3-carboxamide,
(Comp. A56) 2-(Cyclopropanecarbonyl-amino)-6-[(1-phenyl-ethylcarbamoyl)-methoxy]-benzo[b]thiophene-3-carboxamide,
(Comp. A57) 6-Hydroxy-2-(3-pyridin-3-yl-ureido)-benzo[b]thiophene-3-carboxylic acid amide,
(Comp. A58) 6-Hydroxy-2-[3-(6-methoxy-pyridin-3-yl)-ureido]-benzo[b]thiophene-3-carboxylic acid amide,
(Comp. A59) 6-Hydroxy-2-(3-pyridin-4-yl-ureido)-benzo[b]thiophene-3-carboxylic acid amide,
(Comp. A60) 2-[3-(4-Cyano-3-isopropyl-isoxazol-5-yl)-ureido]-6-hydroxy-benzo[b]thiophene-3-carboxylic acid amide,
(Comp. A61) 2-[3-(4-Cyano-3-cyclopropyl-isoxazol-5-yl)-ureido]-6-hydroxy-benzo[b]thiophene-3-carboxylic acid amide,
(Comp. A62) 2-[3-(2,3-Dichloro-phenyl)-ureido]-6-hydroxy-benzo[b]thiophene-3-carboxylic acid amide,
(Comp. A63) 6-Hydroxy-2-[3-(2-trifluoromethoxy-phenyl)-ureido]-benzo[b]thiophene-3-carboxylic acid amide,
(Comp. A64) 6-Hydroxy-2-[3-(4-nitro-phenyl)-ureido]-benzo[b]thiophene-3-carboxylic acid amide,
(Comp. A65) 6-Hydroxy-2-[3-(3,4,5-trimethoxy-phenyl)-ureido]-benzo[b]thiophene-3-carboxylic acid amide,
(Comp. A66) 6-Hydroxy-2-(3-p-tolyl-ureido)-benzo[b]thiophene-3-carboxylic acid amide,
(Comp. A67) 6-Hydroxy-2-[3-(2-thiophen-2-yl-ethyl)-ureido]-benzo[b]thiophene-3-carboxylic acid amide,
(Comp. A68) 6-Hydroxy-2-[3-(4-methyl-cyclohexyl)-ureido]-benzo[b]thiophene-3-carboxylic acid amide,
(Comp. A69) 2-(3-Cyclopentyl-ureido)-6-hydroxy-benzo[b]thiophene-3-carboxylic acid amide,
(Comp. A70) 2-[3-(2-Cyclohex-1-enyl-ethyl)-ureido]-6-hydroxy-benzo[b]thiophene-3-carboxylic acid amide,
(Comp. A71) 6-Hydroxy-2-{3-[2-(4-methoxy-phenyl)-ethyl]-ureido}-benzo[b]thiophene-3-carboxylic acid amide,
(Comp. A72) 6-Hydroxy-2-[3-(2-hydroxy-ethyl)-ureido]-benzo[b]thiophene-3-carboxylic acid amide,
(Comp. A73) 6-Hydroxy-2-[3-(3-hydroxy-propyl)-ureido]-benzo[b]thiophene-3-carboxylic acid amide,
(Comp. A74) 6-Hydroxy-2-(3-piperidin-1-yl-ureido)-benzo[b]thiophene-3-carboxylic acid amide,
(Comp. A75) 2-(3-Bicyclo[2.2.1]hept-2-yl-ureido)-6-hydroxy-benzo[b]thiophene-3-carboxylic acid amide,
(Comp. A76) 2-Amino-6-ethoxy-benzo[b]thiophene-3-carboxylic acid amide,
(Comp. A77) 6-Ethoxy-2-[3-(4-fluoro-phenyl)-ureido]-benzo[b]thiophene-3-carboxylic acid amide,
(Comp. A78) 2-[3-(4-Bromo-phenyl)-ureido]-6-ethoxy-benzo[b]thiophene-3-carboxylic acid amide,
(Comp. A79) 6-Ethoxy-2-(3-p-tolyl-ureido)-benzo[b]thiophene-3-carboxylic acid amide,
(Comp. A80) 2-[3-(2,4-Difluoro-phenyl)-ureido]-6-ethoxy-benzo[b]thiophene-3-carboxylic acid amide,
(Comp. A81) 6-Ethoxy-2-[3-(3,4,5-trimethoxy-phenyl)-ureido]-benzo[b]thiophene-3-carboxylic acid amide,
(Comp. A82) 2-[3-(2-Difluoromethoxy-phenyl)-ureido]-6-hydroxy-benzo[b]thiophene-3-carboxylic acid amide,
(Comp. A83) 6-Hydroxy-2-[3-(4-trifluoromethylsulfanyl-phenyl)-ureido]-benzo[b]thiophene-3-carboxylic acid amide,
(Comp. A84) 7-Chloro-2-(4-fluoro-3-chlorobenzoyl-amino)-6-hydroxy-benzo[b]thiophene-3-carboxylic acid amide,
(Comp. A85) 7-Chloro-2-(3,5-dichlorobenzoyl-amino)-6-hydroxy-benzo[b]thiophene-3-carboxylic acid amide,
(Comp. A86) 7-Chloro-6-hydroxy-2-[3-(2-methylsulfanyl-phenyl)-ureido]-benzo[b]thiophene-3-carboxylic acid amide,
(Comp. A87) 7-Chloro-2-(4-cyanobenzoyl-amino)-6-hydroxy-benzo[b]thiophene-3-carboxylic acid amide,
(Comp. A88) 7-Chloro-2-(2,3-dichlorobenzoyl-amino)-6-hydroxy-benzo[b]thiophene-3-carboxylic acid amide,
(Comp. A89) 7-Chloro-2-(4-thiomethylbenzoyl-amino)-6-hydroxy-benzo[b]thiophene-3-carboxylic acid amide,
(Comp. A90) 7-Chloro-2-(3,4-dimethylbenzoyl-amino)-6-hydroxy-benzo[b]thiophene-3-carboxylic acid amide,
(Comp. A91) 7-Chloro-2-(4-isopropylbenzoyl-amino)-6-hydroxy-benzo[b]thiophene-3-carboxylic acid amide,
(Comp. A92) 7-Chloro-2-(3,5-bistrifluoromethylbenzoyl-amino)-6-hydroxy-benzo[b]thiophene-3-carboxylic acid amide,
(Comp. A93) 7-Chloro-2-(4-chlorobenzoyl-amino)-6-hydroxy-benzo[b]thiophene-3-carboxylic acid amide,
(Comp. A94) 7-Chloro-2-(4-trifluoromethylbenzoyl-amino)-6-hydroxy-benzo[b]thiophene-3-carboxylic acid amide,
(Comp. A95) 7-Chloro-2-(2,4-difluorobenzoyl-amino)-6-hydroxy-benzo[b]thiophene-3-carboxylic acid amide,
(Comp. A96) 7-Chloro-2-(3-fluorobenzoyl-amino)-6-hydroxy-benzo[b]thiophene-3-carboxylic acid amide,
(Comp. A97) 7-Chloro-2-(3,4,5-trimethoxybenzoyl-amino)-6-hydroxy-benzo[b]thiophene-3-carboxylic acid amide,
(Comp. A98) 2-(3-benzo[1,3]dioxol-5yl-ureido)-6-hydroxy-benzo[b]thiophene-3-carboxylic acid amide,
(Comp. A99) N-(3-carbamoyl-6-hydroxy-benzo[b]thiophen-2-yl)-oxalamic acid,
(Comp. A100) 6-Ethoxy-2-[3-(4-trifluoromethyl-phenyl)-ureido]-benzo[b]thiophene-3-carboxylic acid amide,
(Comp. A101) 6-Ethoxy-2-[3-(4-nitro-phenyl)-ureido]-benzo[b]thiophene-3-carboxylic acid amide,
(Comp. A102) 6-Ethoxy-2-(3-phenyl-ureido)-benzo[b]thiophene-3-carboxylic acid amide,
(Comp. A103) 6-Ethoxy-2-[3-(4-ethoxy-phenyl)-ureido]-benzo[b]thiophene-3-carboxylic acid amide,
(Comp. A104) 6-Ethoxy-2-[3-(4-methylsulfanyl-phenyl)-ureido]-benzo[b]thiophene-3-carboxylic acid amide,
(Comp. A105) 2-(Cyclopropanecarbonyl-amino)-5-[3-(4-methylsulfanyl-phenyl)-ureido]-benzo[b]thiophene-3-carboxylic acid amide,
(Comp. A106) 5-[3-(4-Bromo-phenyl)-ureido]-2-(cyclopropanecarbonyl-amino)-benzo[b]thiophene-3-carboxylic acid amide,
(Comp. A107) 2-(Cyclopropanecarbonyl-amino)-5-[3-(4-iodo-phenyl)-ureido]-benzo[b]thiophene-3-carboxylic acid amide,
(Comp. A108) 2-(Cyclopropanecarbonyl-amino)-5-(3-phenyl-ureido)-benzo[b]thiophene-3-carboxylic acid amide,
(Comp. A109) 7-Bromo-2-[3-(4-bromo-phenyl)-ureido]-6-hydroxy-benzo[b]thiophene-3-carboxylic acid amide,
(Comp. A110) 7-Bromo-6-hydroxy-2-[3-(4-trifluoromethoxy-phenyl)-ureido]-benzo[b]thiophene-3-carboxylic acid amide,
(Comp. A111) 7-Bromo-6-hydroxy-2-[3-(4-methylsulfanyl-phenyl)-ureido]-benzo[b]thiophene-3-carboxylic acid amide,
(Comp. A112) 7-Chloro-2-[3-(4-difluoromethoxy-phenyl)-ureido]-6-hydroxy-benzo[b]thiophene-3-carboxylic acid amide,
(Comp. A113) 2-(Cyclopropanecarbonyl-amino)-5-nitro-benzo[b]thiophene-3-carboxylic acid amide,
(Comp. A114) 5-Amino-2-(cyclopropanecarbonyl-amino)-benzo[b]thiophene-3-carboxylic acid amide,
(Comp. A115) 6-Hydroxy-2-[3-(4-trifluoromethoxy-phenyl)-ureido]-benzo[b]thiophene-3-carboxylic acid amide,
(Comp. A116) 6-Hydroxy-2-[3-(4-methylsulfanyl-phenyl)-ureido]-benzo[b]thiophene-3-carboxylic acid amide,
(Comp. A117) 2-(3,3-Dimethyl-ureido)-6-hydroxy-benzo[b]thiophene-3-carboxylic acid amide,
(Comp. A118) 2-(3-Cyclopropyl-ureido)-6-hydroxy-benzo[b]thiophene-3-carboxylic acid amide,
(Comp. A119) 6-Hydroxy-2-[3-(3-methylsulfanyl-phenyl)-ureido]-benzo[b]thiophene-3-carboxylic acid amide,
(Comp. A120) (3-Carbamoyl-5,7-dichloro-6-hydroxy-benzo[b]thiophen-2-yl)-carbamic acid ethyl ester,
(Comp. A121) 2-[3-(3-Bromo-phenyl)-ureido]-6-hydroxy-benzo[b]thiophene-3-carboxylic acid amide,
(Comp. A122) 6-Hydroxy-2-[3-(3-trifluoromethyl-phenyl)-ureido]-benzo[b]thiophene-3-carboxylic acid amide,
(Comp. B1) 2-(Cyclopropanecarbonyl-amino)-4,7-dihydro-5H-thieno[2,3-c]pyran-3-carboxylic acid amide,
(Comp. B2) 2-Isobutyrylamino-4,7-dihydro-5H-thieno[2,3-c]pyran-3-carboxylic acid amide,
(Comp. B3) 2-[(2-Phenyl-cyclopropanecarbonyl)-amino]-4,7-dihydro-5H-thieno[2,3-c]pyran-3-carboxylic acid amide,
(Comp. B4) 2-[(2-Methyl-cyclopropanecarbonyl)-amino]-4,7-dihydro-5H-thieno[2,3-c]pyran-3-carboxylic acid amide,
(Comp. B5) 2-[(Furan-2-carbonyl)-amino]-4,7-dihydro-5H-thieno[2,3-c]pyran-3-carboxylic acid amide,
(Comp. B6) 2-[(Adamantane-1-carbonyl)-amino]-4,7-dihydro-5H-thieno[2,3-c]pyran-3-carboxylic acid amide,
(Comp. B7) 2-(Cyclohexanecarbonyl-amino)-4,7-dihydro-5H-thieno[2,3-c]pyran-3-carboxylic acid amide,
(Comp. B8) 2-(3-Cyclohexyl-ureido)-4,7-dihydro-5H-thieno[2,3-c]pyran-3-carboxylic acid amide,
(Comp. B9) 2-(3-Phenyl-ureido)-4,7-dihydro-5H-thieno[2,3-c]pyran-3-carboxylic acid amide,
(Comp. B10) 2-(Cyclopentanecarbonyl-amino)-4,7-dihydro-5H-thieno[2,3-c]pyran-3-carboxylic acid amide,
(Comp. B11) 2-[3-(4-Acetyl-phenyl)-ureido]-4,7-dihydro-5H-thieno[2,3-c]pyran-3-carboxylic acid amide,
(Comp. B12) 2-[3-(4-Methyl-phenyl)-ureido]-4,7-dihydro-5H-thieno[2,3-c]pyran-3-carboxylic acid amide,
(Comp. B13) 2-[3-(4-Fluoro-phenyl)-ureido]-4,7-dihydro-5H-thieno[2,3-c]pyran-3-carboxylic acid amide,
(Comp. B14) 2-(2-Methyl-butyrylamino)-4,7-dihydro-5H-thieno[2,3-c]pyran-3-carboxylic acid amide,
(Comp. B15) 2-(Cyclobutanecarbonyl-amino)-4,7-dihydro-5H-thieno[2,3-c]pyran-3-carboxylic acid amide,
(Comp. B16) 2-But-2-enoylamino-4,7-dihydro-5H-thieno[2,3-c]pyran-3-carboxylic acid amide,
(Comp. B17) 2-(3-Methyl-but-2-enoylamino)-4,7-dihydro-5H-thieno[2,3-c]pyran-3-carboxylic acid amide,
(Comp. B18) 2-(2,2-Dimethyl-propionylamino)-4,7-dihydro-5H-thieno[2,3-c]pyran-3-carboxylic acid amide,
(Comp. B19) 2-(3,4-Difluoro-benzoylamino)-4,7-dihydro-5H-thieno[2,3-c]pyran-3-carboxylic acid amide,
(Comp. B20) 5,5-Dimethyl-2-(3-phenyl-ureido)-4,7-dihydro-5H-thieno[2,3-c]pyran-3-carboxylic acid amide,
(Comp. B21) 2-[3-(5-Phenyl-2-p-tolyl-2H-pyrazol-3-yl)-ureido]-4,7-dihydro-5H-thieno[2,3-c]pyran-3-carboxylic acid amide,
(Comp. B22) 2-[3-(5-tert-Butyl-2-p-tolyl-2H-pyrazol-3-yl)-ureido]-4,7-dihydro-5H-thieno[2,3-c]pyran-3-carboxylic acid amide,
(Comp. B23) 2-[2-(2-Bromo-phenylsulfanyl)-acetylamino]-4,7-dihydro-5H-thieno[2,3-c]pyran-3-carboxylic acid amide,
(Comp. B24) 2-(Cyclopropanecarbonyl-amino)-7-dimethylamino-4,7-dihydro-5H-thieno[2,3-c]pyran-3-carboxylic acid amide,
(Comp. B25) 2-(Cyclopropanecarbonyl-amino)-7-[4-(4-methyl-piperazin-1-yl)-phenylamino]-4,7-dihydro-5H-thieno[2,3-c]pyran-3-carboxylic acid amide,
(Comp. B26) 2-(Cyclopropanecarbonyl-amino)-7-(4-methyl-piperazin-1-yl)-4,7-dihydro-5H-thieno[2,3-c]pyran-3-carboxylic acid amide,
(Comp. B27) 2-(Cyclopropanecarbonyl-amino)-7-(2-dimethylamino-ethylamino)-4,7-dihydro-5H-thieno[2,3-c]pyran-3-carboxylic acid amide,
(Comp. B28) 2-(3-Biphenyl-2-yl-ureido)-4,7-dihydro-5H-thieno[2,3-c]pyran-3-carboxylic acid amide,
(Comp. B29) 2-[3-(2-Ethoxy-phenyl)-ureido]-4,7-dihydro-5H-thieno[2,3-c]pyran-3-carboxylic acid amide,
(Comp. B30) 2-(3,3-Diethyl-ureido)-4,7-dihydro-5H-thieno[2,3-c]pyran-3-carboxylic acid amide,
(Comp. B31) 4-(3-Carbamoyl-4,7-dihydro-5H-thieno[2,3-c]pyran-2-ylcarbamoyl)-piperazine-1-carboxylic acid ethyl ester,
(Comp. B32) 4-Phenyl-piperazine-1-carboxylic acid (3-carbamoyl-4,7-dihydro-5H-thieno[2,3-c]pyran-2-yl)-amide,
(Comp. B33) 2-{3-[3-(4-Chloro-phenyl)-4-cyano-isoxazol-5-yl]-ureido}-4,7-dihydro-5H-thieno[2,3-c]pyran-3-carboxylic acid amide,
(Comp. B34) 2-[3-(3-Imidazol-1-yl-propyl)-ureido]-4,7-dihydro-5H-thieno[2,3-c]pyran-3-carboxylic acid amide,
(Comp. B35) 2-(3,3-Diallyl-ureido]-4,7-dihydro-5H-thieno[2,3-c]pyran-3-carboxylic acid amide,
(Comp. B36) (3-Carbamoyl-4,7-dihydro-5H-thieno[2,3-c]pyran-2-yl)-carbamic acid 2-methoxy-phenyl ester,
(Comp. B37) 2-[3-(4-Bromo-phenyl)-thioureido]-4,7-dihydro-5H-thieno[2,3-c]pyran-3-carboxylic acid amide,
(Comp. B38) 2-[3-(4-Methylsulfanyl-phenyl)-thioureido]-4,7-dihydro-5H-thieno[2,3-c]pyran-3-carboxylic acid amide,
(Comp. B39) 2-[3-(Hydrazinocarbonyl-phenyl-methyl)-ureido]-4,7-dihydro-5H-thieno[2,3-c]pyran-3-carboxylic acid amide,
(Comp. B40) 2-[(2-Benzyloxymethyl-cyclopropanecarbonyl)-amino]-4,7-dihydro-5H-thieno[2,3-c]pyran-3-carboxylic acid amide,
(Comp. B41) N-(3-Carbamoyl-4,7-dihydro-5H-thieno[2,3-c]pyran-2-yl)-oxalamide,
(Comp. B42) 2-{[3-(2,2-Dichloro-vinyl)-2,2-dimethyl-cyclopropanecarbonyl]-amino}-4,7-dihydro-5H-thieno[2,3-c]pyran-3-carboxylic acid amide,
(Comp. B43) 2-[3-(4-Bromo-phenyl)-ureido]-4,7-dihydro-5H-thieno[2,3-c]pyran-3-carboxylic acid amide,
(Comp. B44) 4-Hydroxy-piperidine-1-carboxylic-acid (3-carbamoyl-4,7-dihydro-5H-thieno[2,3-c]pyran-2-yl)-amide
(Comp. B45) 2-[3-(5-Methyl-furan-2-ylmethyl)-ureido]-4,7-dihydro-5H-thieno[2,3-c]pyran-3-carboxylic acid amide,
(Comp. B46) 2-[3,3-Bis-(2-hydroxy-ethyl)-ureido]-4,7-dihydro-5H-thieno[2,3-c]pyran-3-carboxylic acid amide,
(Comp. B47) 2-[3-(4-Methyl-cyclohexyl)-ureido]-4,7-dihydro-5H-thieno[2,3-c]pyran-3-carboxylic acid amide,
(Comp. B48) 2-(3-Cyclopentyl-ureido)-4,7-dihydro-5H-thieno[2,3-c]pyran-3-carboxylic acid amide,
(Comp. B49) 2-(Cyclopropanecarbonyl-amino)-7-morpholyn-4-yl-4,7-dihydro-5H-thieno[2,3-c]pyran-3-carboxylic acid amide,
(Comp. B50) 2-{3-[2-(4-Methoxy-phenyl)-ethyl]-ureido}-4,7-dihydro-5H-thieno[2,3-c]pyran-3-carboxylic acid amide,
(Comp. B51) 2-[3-(2-tert-Butyl-5-phenyl-2H-pyrazol-3-yl)-ureido]-4,7-dihydro-5H-thieno[2,3-c]pyran-3-carboxylic acid amide,
(Comp. B52) Morpholine-4-carboxylic-acid(3-carbamoyl-4,7-dihydro-5H-thieno[2,3-c]pyran-2-yl)-amide,
(Comp. B53) 4-Methyl-piperazine-1-carboxylic-acid (3-carbamoyl-4,7-dihydro-5H-thieno[2,3-c]pyran-2-yl)-amide,
(Comp. B54) Piperidine-1-carboxylic-acid (3-carbamoyl-4,7-dihydro-5H-thieno[2,3-c]pyran-2-yl)-amide,
(Comp. B55) 2-[3-(2-Methyl-cyclohexyl)-ureido]-4,7-dihydro-5H-thieno[2,3-c]pyran-3-carboxylic acid amide,
(Comp. B56) 2-[3-Bicyclo[2.2.1]hept-2-yl)-ureido]-4,7-dihydro-5H-thieno[2,3-c]pyran-3-carboxylic acid amide,
(Comp. B57) 2-(3,3-Diisopropyl-ureido)-4,7-dihydro-5H-thieno[2,3-c]pyran-3-carboxylic acid amide,
(Comp. B58) Thiomorpholine-4-carboxylic-acid(3-carbamoyl-4,7-dihydro-5H-thieno[2,3-c]pyran-2-yl)-amide,
(Comp. B59) 2-[3-(5-Fluoro-2-methyl-phenyl)-ureido]-4,7-dihydro-5H-thieno[2,3-c]pyran-3-carboxylic acid amide,
(Comp. B60) 2-[3-(2-Hydroxy-butyl)-ureido]-4,7-dihydro-5H-thieno[2,3-c]pyran-3-carboxylic acid amide,
(Comp. B61) 2-(Cyclopropanecarbonyl-amino)-7-(3-dimethylamino-propylamino)-4,7-dihydro-5H-thieno[2,3-c]pyran-3-carboxylic acid amide,
(Comp. B62) 2-[3-(2-Cyclohex-1-enyl-ethyl)-ureido]-4,7-dihydro-5H-thieno[2,3-c]pyran-3-carboxylic acid amide,
(Comp. B63) 2-[3-(2,6,6-Trimethyl-bicyclo[3.1.1]hept-3-yl)-ureido]-4,7-dihydro-5H-thieno[2,3-c]pyran-3-carboxylic acid amide,
(Comp. B64) 2-(2-Ethylamino-acetylamino)-4,7-dihydro-5H-thieno[2,3-c]pyran-3-carboxylic acid amide,
(Comp. B65) 2-(Cyclopropanecarbonyl-amino)-7-(2-pyridin-2-yl-ethylamino)-4,7-dihydro-5H-thieno[2,3-c]pyran-3-carboxylic acid amide,
(Comp. B66) 2-(Cyclopropanecarbonyl-amino)-7-(3-morpholin-4-yl-propylamino)-4,7-dihydro-5H-thieno[2,3-c]pyran-3-carboxylic acid amide,
(Comp. B67) 2-[3-(4-Methylsulfanyl-phenyl)-ureido]-4,7-dihydro-5H-thieno[2,3-c]pyran-3-carboxylic acid amide,
(Comp. B68) 2-[3-(3-Diethylamino-propyl)-ureido]-4,7-dihydro-5H-thieno[2,3-c]pyran-3-carboxylic acid amide,
(Comp. B69) 2-[3-(2-Diethylamino-ethyl)-ureido]-4,7-dihydro-5H-thieno[2,3-c]pyran-3-carboxylic acid amide,
(Comp. B70) 2-[3-(2-Dimethylamino-ethyl)-ureido]-4,7-dihydro-5H-thieno[2,3-c]c]pyran-3-carboxylic acid amide,
(Comp. B71) 2-(Cyclopropanecarbonyl-amino)-7,7-dimethyl-4,7-dihydro-5H-thieno[2,3-c]pyran-3-carboxylic acid amide,
(Comp. B72) 2-[3-(1-Benzyl-pyridin-4-yl)-ureido]-4,7-dihydro-5H-thieno[2,3-c]pyran-3-carboxylic acid amide,
(Comp. B73) 2-(3-Pyridin-4-yl-ureido]-4,7-dihydro-5H-thieno[2,3-c]pyran-3-carboxylic acid amide,
(Comp. B74) 2-[3-(4-Chloro-3-trifluoromethyl-phenyl)-ureido]-4,7-dihydro-5H-thieno[2,3-c]pyran-3-carboxylic acid amide,
(Comp. B75) 2-[3-(2-Thiophen-2-yl-ethyl)-ureido]-4,7-dihydro-5H-thieno[2,3-c]pyran-3-carboxylic acid amide,
(Comp. B76) 2-(3-Thiophen-2-yl-ureido)-4,7-dihydro-5H-thieno[2,3-c]pyran-3-carboxylic acid amide,
(Comp. B77) 2-(2-Cyclopropyl-acetylamino)-4,7-dihydro-5H-thieno[2,3-c]pyran-3-carboxylic acid amide,
(Comp. B78) 2-(3-Pyridin-3-yl-ureido)-4,7-dihydro-5H-thieno[2,3-c]pyran-3-carboxylic acid amide,
(Comp. B79) 2-(3-Benzo[1,3]dioxol-5-yl-ureido)-4,7-dihydro-5H-thieno[2,3-c]pyran-3-carboxylic acid amide,
(Comp. B80) 2-(3-Furan-2-ylmethyl-ureido)-4,7-dihydro-5H-thieno[2,3-c]pyran-3-carboxylic acid amide,
(Comp. B81) 2-[3-(2,5-Di-tert-butyl-2H-pyrazol-3-yl)-ureido]-4,7-dihydro-5H-thieno[2,3-c]pyran-3-carboxylic acid amide,
(Comp. B82) 2-[3-(4-Benzyloxy-phenyl)-ureido]-4,7-dihydro-5H-thieno[2,3-c]pyran-3-carboxylic acid amide,
(Comp. B83) 2-(2-Methyl-acryloylamino)-4,7-dihydro-5H-thieno[2,3-c]pyran-3-carboxylic acid amide,
(Comp. B84) 2-(3-Isopropyl-ureido)-4,7-dihydro-5H-thieno[2,3-c]pyran-3-carboxylic acid amide,
(Comp. B85) 2-[3-(2,6-Diisopropyl-phenyl)-ureido]-4,7-dihydro-5H-thieno[2,3-c]pyran-3-carboxylic acid amide,
(Comp. B86) 2-[3-(3-Chloro-phenyl)-ureido]-4,7-dihydro-5H-thieno[2,3-c]pyran-3-carboxylic acid amide,
(Comp. B87) 2-(3-Benzyl-ureido)-4,7-dihydro-5H-thieno[2,3-c]pyran-3-carboxylic acid amide,
(Comp. B88) 2-[3-(4-Isopropyl-phenyl)-ureido]-4,7-dihydro-5H-thieno[2,3-c]pyran-3-carboxylic acid amide,
(Comp. B89) 2-[3-(3-Acetyl-phenyl)-ureido]-4,7-dihydro-5H-thieno[2,3-c]pyran-3-carboxylic acid amide,
(Comp. B90) 2-[3-(3-Ethyl-phenyl)-ureido]-4,7-dihydro-5H-thieno[2,3-c]pyran-3-carboxylic acid amide,
(Comp. B91) 2-[3-(1-Naphthalen-1-yl-ethyl)-ureido]-4,7-dihydro-5H-thieno[2,3-c]pyran-3-carboxylic acid amide,
(Comp. B92) 2-(2-Azepan-1-yl-acetylamino)-4,7-dihydro-5H-thieno[2,3-c]pyran-3-carboxylic acid amide,
(Comp. B93) 2-[3-(3,4-Dimethyl-phenyl)-ureido]-4,7-dihydro-5H-thieno[2,3-c]pyran-3-carboxylic acid amide,
(Comp. B94) 2-(2-Ethyl-butyrylamino)-4,7-dihydro-5H-thieno[2,3-c]pyran-3-carboxylic acid amide,
(Comp. B95) 2-[(1-Methyl-cyclopropanecarbonyl)-amino]-4,7-dihydro-5H-thieno[2,3-c]pyran-3-carboxylic acid amide,
(Comp. B96) 2-[(3-Methyl-cyclohexanecarbonyl)-amino]-4,7-dihydro-5H-thieno[2,3-c]pyran-3-carboxylic acid amide,
(Comp. B97) 2-[3-(5-Chloro-2-methoxy-phenyl)-ureido]-4,7-dihydro-5H-thieno[2,3-c]pyran-3-carboxylic acid amide,
(Comp. B98) 2-(3-Methyl-butyrylamino)-4,7-dihydro-5H-thieno[2,3-c]pyran-3-carboxylic acid amide,
(Comp. B99) 2-(Cyclopropanecarbonyl)-amino)-7-isopropoxy-4,7-dihydro-5H-thieno[2,3-c]pyran-3-carboxylic acid amide,
(Comp. B100) 2-[3-(3-Chloro-2-methyl-phenyl)-ureido]-4,7-dihydro-5H-thieno[2,3-c]pyran-3-carboxylic acid amide,
(Comp. B101) 2-[3-(2-Chloro-3-methyl-phenyl)-ureido]-4,7-dihydro-5H-thieno[2,3-c]pyran-3-carboxylic acid amide,
(Comp. B102) 2-(4-Bromo-benzoylamino)-4,7-dihydro-5H-thieno[2,3-c]pyran-3-carboxylic acid amide,
(Comp. B103) 2-[3-(1-Methyl-cyclopropyl)-thioureido]-4,7-dihydro-5H-thieno[2,3-c]pyran-3-carboxylic acid amide,
(Comp. B104) N-(3-Carbamoyl-4,7-dihydro-5H-thieno[2,3-c]pyran-2-yl)-succinamic acid,
(Comp. B105) 3-(3-Carbamoyl-4,7-dihydro-5H-thieno[2,3-c]pyran-2-ylcarbamoyl)-acrylic acid,
(Comp. B106) 2-[3-(3-Carbamoyl-4,7-dihydro-5H-thieno[2,3-c]pyran-2-yl)-ureido]-3-methyl-butyric acid methyl ester,
(Comp. B107) 2-[(Tetrahydro-furan-3-carbonyl)-amino]-4,7-dihydro-5H-thieno[2,3-c]pyran-3-carboxylic acid amide,
(Comp. B108) 2-(2-Bicyclo[2.2.1]hept-2-yl-acetylamino)-4,7-dihydro-5H-thieno[2,3-c]pyran-3-carboxylic acid amide,
(Comp. B109) 2-(2-Cyclopentyl-acetylamino)-4,7-dihydro-5H-thieno[2,3-c]pyran-3-carboxylic acid amide,
(Comp. B110) 2-[2-(3-Acetyl-2,2-dimethyl-cyclobutyl)-acetylamino]-4,7-dihydro-5H-thieno[2,3-c]pyran-3-carboxylic acid amide,
(Comp. B111) 2-(2-Cyclopropylamino-acetylamino)-4,7-dihydro-5H-thieno[2,3-c]pyran-3-carboxylic acid amide,
(Comp. B112) 2-[(2,2,3,3-Tetramethyl-cyclopropanecarbonyl)-amino]-4,7-dihydro-5H-thieno[2,3-c]pyran-3-carboxylic acid amide,
(Comp. B113) N-(3-Carbamoyl-4,7-dihydro-5H-thieno[2,3-c]pyran-2-yl)-oxalamic acid methyl ester,
(Comp. B114) 2-[3-(3-Carbamoyl-4,7-dihydro-5H-thieno[2,3-c]pyran-2-yl)-ureido]-3-methyl-butyric acid allyl ester,
(Comp. B115) 2-[2-(1,3-Dimethyl-butylamino)-acetylamino]-4,7-dihydro-5H-thieno[2,3-c]pyran-3-carboxylic acid amide,
(Comp. B116) 2-[3-(3-Trifluoromethyl-phenyl)-ureido]-4,7-dihydro-5H-thieno[2,3-c]pyran-3-carboxylic acid amide,
(Comp. B117) 2-[3-(2,3-Dichloro-phenyl)-ureido]-4,7-dihydro-5H-thieno[2,3-c]pyran-3-carboxylic acid amide,
(Comp. B118) 2-[3-(2-Methoxy-benzyl)-ureido]-4,7-dihydro-5H-thieno[2,3-c]pyran-3-carboxylic acid amide,
(Comp. B119) 2-[3-(4-Nitro-phenyl)-ureido]-4,7-dihydro-5H-thieno[2,3-c]pyran-3-carboxylic acid amide,
(Comp. B120) 2-[3-(2,4-Difluoro-phenyl)-ureido]-4,7-dihydro-5H-thieno[2,3-c]pyran-3-carboxylic acid amide,
(Comp. B121) 2-[3-(3,4,5-Trimethoxy-phenyl)-ureido]-4,7-dihydro-5H-thieno[2,3-c]pyran-3-carboxylic acid amide,
(Comp. B122) 2-[3-(3,5-Dichloro-phenyl)-ureido]-4,7-dihydro-5H-thieno[2,3-c]pyran-3-carboxylic acid amide,
(Comp. B123) 2-[3-(2-Nitro-phenyl)-ureido]-4,7-dihydro-5H-thieno[2,3-c]pyran-3-carboxylic acid amide,
(Comp. B124) 2-[3-(3-Methoxy-phenyl)-ureido]-4,7-dihydro-5H-thieno[2,3-c]pyran-3-carboxylic acid amide,
(Comp. B125) 2-[3-(2-Methyl-5-nitro-phenyl)-ureido]-4,7-dihydro-5H-thieno[2,3-c]pyran-3-carboxylic acid amide,
(Comp. B126) 2-(3-Adamantan-1-yl-ureido)-4,7-dihydro-5H-thieno[2,3-c]pyran-3-carboxylic acid amide,
(Comp. B127) 2-[3-(3-Nitro-phenyl)-ureido]-4,7-dihydro-5H-thieno[2,3-c]pyran-3-carboxylic acid amide,
(Comp. B128) 2-[3-(2-Methyl-3-nitro-phenyl)-ureido]-4,7-dihydro-5H-thieno[2,3-c]pyran-3-carboxylic acid amide,
(Comp. B129) 2-[3-(3-Bromo-phenyl)-ureido]-4,7-dihydro-5H-thieno[2,3-c]pyran-3-carboxylic acid amide,
(Comp. B1 30) 2-(3-m-Tolyl-ureido)-4,7-dihydro-5H-thieno[2,3-c]pyran-3-carboxylic acid amide,
(Comp. B131) 2-[3-(3-Methyl-butyl)-ureido]-4,7-dihydro-5H-thieno[2,3-c]pyran-3-carboxylic acid amide,
(Comp. B132) 2-[3-(4-Methyl-3-nitro-phenyl)-ureido]-4,7-dihydro-5H-thieno[2,3-c]pyran-3-carboxylic acid amide,
(Comp. B133) 2-[3-(4-Methoxy-phenyl)-ureido]-4,7-dihydro-5H-thieno[2,3-c]pyran-3-carboxylic acid amide,
(Comp. B134) 2-[3-(3-Chloro-4-fluoro-phenyl)-ureido]-4,7-dihydro-5H-thieno[2,3-c]pyran-3-carboxylic acid amide,
(Comp. B135) 2-(3-Phenyl-acryloylamino)-4,7-dihydro-5H-thieno[2,3-c]pyran-3-carboxylic acid amide,
(Comp. B136) 2-[3-(3-Amino-phenylsulfanyl)-acetylamino]-4,7-dihydro-5H-thieno[2,3-c]pyran-3-carboxylic acid amide,
(Comp. B137) 2-(2-Methyl-but-2-enoylamino)-4,7-dihydro-5H-thieno[2,3-c]pyran-3-carboxylic acid amide,
(Comp. B138) 2-(4,4,4-Trifluoro-3-methyl-but-2-enoylamino)-4,7-dihydro-5H-thieno[2,3-c]pyran-3-carboxylic acid amide,
(Comp. B139) 2-(3-Isopropyl-thioureido)-4,7-dihydro-5H-thieno[2,3-c]pyran-3-carboxylic acid amide,
(Comp. B140) 2-(3,3-Dimethyl-butyrylamino)-4,7-dihydro-5H-thieno[2,3-c]pyran-3-carboxylic acid amide,
(Comp. B141) 2-[3-(3-Morpholyn-4-yl-propyl)-ureido]-4,7-dihydro-5H-thieno[2,3-c]pyran-3-carboxylic acid amide,
(Comp. B142) 2-[3-(3,4-Dichloro-phenyl)-ureido]-4,7-dihydro-5H-thieno[2,3-c]pyran-3-carboxylic acid amide,
(Comp. B143) 2-[3-(4-Phenoxy-phenyl)-ureido]-4,7-dihydro-5H-thieno[2,3-c]pyran-3-carboxylic acid amide,
(Comp. B144) 2-[3-(4-Trifluoromethyl-phenyl)-ureido]-4,7-dihydro-5H-thieno[2,3-c]pyran-3-carboxylic acid amide,
(Comp. B145) 2-[3-(2-Methyl-benzyl)-ureido]-4,7-dihydro-5H-thieno[2,3-c]pyran-3-carboxylic acid amide,
(Comp. B146) 2-(3-Biphenyl-4-yl-ureido)-4,7-dihydro-5H-thieno[2,3-c]pyran-3-carboxylic acid amide,
(Comp. B147) 2-[3-(4-Methoxy-benzyl)-ureido]-4,7-dihydro-5H-thieno[2,3-c]pyran-3-carboxylic acid amide,
(Comp. B148) 2-[3-(4-Methyl-benzyl)-ureido]-4,7-dihydro-5H-thieno[2,3-c]pyran-3-carboxylic acid amide, (Comp. B149) 2-[3-(3-Methyl-benzyl)-ureido]-4,7-dihydro-5H-thieno[2,3-c]pyran-3-carboxylic acid amide,
(Comp. B150) 2-(3-o-Tolyl-ureido)-4,7-dihydro-5H-thieno[2,3-c]pyran-3-carboxylic acid amide,
(Comp. B151) 5-Methyl-isoxazole-4-carboxylic acid (3-carbamoyl-4,7-dihydro-5H-thieno[2,3-c]pyran-2-yl) amide,
(Comp. B152) 2-[2-(2-Thiophen-2-yl-ethylamino)-acetylamino]-4,7-dihydro-5H-thieno[2,3-c]pyran-3-carboxylic acid amide,
(Comp. B153) 2-[2-(3,5-Dimethoxy-phenylamino)-acetylamino]-4,7-dihydro-5H-thieno[2,3-c]pyran-3-carboxylic acid amide,
(Comp. B154) 2-[3-(3,5-Bis-trifluoromethyl-phenyl)-ureido]-4,7-dihydro-5H-thieno[2,3-c]pyran-3-carboxylic acid amide,
(Comp. B155) 2-[3-(2-Chloro-5-trifluoromethyl-phenyl)-ureido]-4,7-dihydro-5H-thieno[2,3-c]pyran-3-carboxylic acid amide,
(Comp. B156) 2-(Cyclopropanecarbonyl-amino)-5,5-dimethyl-4,7-dihydro-5H-thieno[2,3-c]pyran-3-carboxylic acid amide,
(Comp. B157) 4-[3-(3-Carbamoyl-4,7-dihydro-5H-thieno[2,3-c]pyran-2-yl)-ureido]-1-methyl-pyridinium iodide,
(Comp. B158) 2-[3-(6-Methyl-pyridin-2-yl)-ureido]-4,7-dihydro-5H-thieno[2,3-c]pyran-3-carboxylic acid amide,
(Comp. B159) 2-[3-(6-Methoxy-pyridin-3-yl)-ureido]-4,7-dihydro-5H-thieno[2,3-c]pyran-3-carboxylic acid amide,
(Comp. B160) 2-[3-(2-Chloro-pyridin-3-yl)-ureido]-4,7-dihydro-5H-thieno[2,3-c]pyran-3-carboxylic acid amide,
(Comp. B161) 2-(3-Pyridin-3-yl-thioureido)-4,7-dihydro-5H-thieno[2,3-c]pyran-3-carboxylic acid amide,
(Comp. B162) 2-[3-(2-Methyl-quinolin-4-yl)-ureido]-4,7-dihydro-5H-thieno[2,3-c]pyran-3-carboxylic acid amide,
(Comp. B163) 2-[3-(2,6-Dimethoxy-pyridin-3-yl)-ureido]-4,7-dihydro-5H-thieno[2,3-c]pyran-3-carboxylic acid amide,
(Comp. B164) 2-[3-(6-Chloro-pyridin-3-yl)-ureido]-4,7-dihydro-5H-thieno[2,3-c]pyran-3-carboxylic acid amide,
(Comp. B165) 2-(Cyclopropanecarbonyl-amino)-7-ethoxy-4,7-dihydro-5H-thieno[2,3-c]pyran-3-carboxylic acid amide,
(Comp. B166) 2-[3-(3-Fluoro-phenyl)-ureido]-4,7-dihydro-5H-thieno[2,3-c]pyran-3-carboxylic acid amide,
(Comp. B167) 7-(3-Chloro-4-fluoro-phenylamino)-2-(cyclopropanecarbonyl-amino)-4,7-dihydro-5H-thieno[2,3-c]pyran-3-carboxylic acid amide, (Comp. B168) 2-(Cyclopropanecarbonyl-amino)-7-(2-morpholin-4-yl-ethylamino)-4,7-dihydro-5H-thieno[2,3-c]pyran-3-carboxylic acid amide,
(Comp. B169) 2-{3-[4-(2-Morpholin-4-yl-ethoxy)-naphthalen-1 -yl]-ureido}-4,7-dihydro-5H-thieno[2,3-c]pyran-3-carboxylic acid amide,
(Comp. B170) 2-[2-(2-Chloro-phenylsulfanyl)-acetylamino]-4,7-dihydro-5H-thieno[2,3-c]pyran-3-carboxylic acid amide,
(Comp. B171) 2-(3-Methyl-pentanoylamino)-4,7-dihydro-5H-thieno[2,3-c]pyran-3-carboxylic acid amide,
(Comp. B172) 2-[(2-Methoxymethyl-cyclopropanecarbonyl)-amino]-4,7-dihydro-5H-thieno[2,3-c]pyran-3-carboxylic acid amide,
(Comp. B173) 2-(2-Methyl-pentanoylamino)-4,7-dihydro-5H-thieno[2,3-c]pyran-3-carboxylic acid amide,
(Comp. B174) 2-[(2-Allyloxymethyl-cyclopropanecarbonyl)-amino]-4,7-dihydro-5H-thieno[2,3-c]pyran-3-carboxylic acid amide,
(Comp. B 175) 2-[(2-Octyl-cyclopropanecarbonyl)-amino]-4,7-dihydro-5H-thieno[2,3-c]pyran-3-carboxylic acid amide,
(Comp. B176) 2-Ureido-4,7-dihydro-5H-thieno[2,3-c]pyran-3-carboxylic acid amide,
(Comp. B177) 2-(3-Methyl-pent-4-enoylamino)-4,7-dihydro-5H-thieno[2,3-c]pyran-3-carboxylic acid amide,
(Comp. B178) 2-(3-Phenyl-propionylamino)-4,7-dihydro-5H-thieno[2,3-c]pyran-3-carboxylic acid amide,
(Comp. B179) 2-[3-(4-Ethoxy-phenyl)-ureido]-4,7-dihydro-5H-thieno[2,3-c]pyran-3-carboxylic acid amide,
(Comp. B180) 2-[3-(5-Chloro-2,4-dimethoxy-phenyl)-ureido]-4,7-dihydro-5H-thieno[2,3-c]pyran-3-carboxylic acid amide,
(Comp. B181) 4-[3-(3-Carbamoyl-4,7-dihydro-5H-thieno[2,3-c]pyran-2-yl)-ureido]-piperidine-1-carboxylic-acid ethyl ester,
(Comp. B182) 2-(3,3-Dimethyl-ureido)-4,7-dihydro-5H-thieno[2,3-c]pyran-3-carboxylic acid amide,
(Comp. B183) 2-[(2-Fluoro-cyclopropanecarbonyl)-amino]-4,7-dihydro-5H-thieno[2,3-c]pyran-3-carboxylic acid amide,
(Comp. B184) 2-[2-(Naphthalen-2-ylsulfanyl)-acetylamino]-4,7-dihydro-5H-thieno[2,3-c]pyran-3-carboxylic acid amide,
(Comp. B185) 2-[2-(2,4-Dimethyl-phenylsulfanyl)-acetylamino]-4,7-dihydro-5H-thieno[2,3-c]pyran-3-carboxylic acid amide,
(Comp. B 186) 2-[2-(Quinolin-8-ylsulfanyl)-acetylamino]-4,7-dihydro-5H-thieno[2,3-c]pyran-3-carboxylic acid amide,
(Comp. B187) 2-[2-(2,6-Dichloro-phenylsulfanyl)-acetylamino]-4,7-dihydro-5H-thieno[2,3-c]pyran-3-carboxylic acid amide,
(Comp. B188) 2-[2-(4-Methyl-2-oxo-2H-chromen-7-ylsulfanyl)-acetylamino]-4,7-dihydro-5H-thieno[2,3-c]pyran-3-carboxylic acid amide,
(Comp. B189) 2-[2-(2-Nitro-4-trifluoromethyl-phenylsulfanyl)-acetylamino]-4,7-dihydro-5H-thieno[2,3-c]pyran-3-carboxylic acid amide,
(Comp. B190) 2-(3,3-Dibutyl-ureido)-4,7-dihydro-5H-thieno[2,3-c]pyran-3-carboxylic acid amide,
(Comp. B191) 2-[3-(2-Hydroxy-ethyl)-ureido]-4,7-dihydro-5H-thieno[2,3-c]pyran-3-carboxylic acid amide,
(Comp. B192) 2-[3-(3-Hydroxy-propyl)-ureido]-4,7-dihydro-5H-thieno[2,3-c]pyran-3-carboxylic acid amide,
(Comp. B193) 2-{[2-(4-Methyl-piperazin-1 -ylmethyl)-cyclopropanecarbonyl]-amino}-4,7-dihydro-5H-thieno[2,3-c]pyran-3-carboxylic acid amide,
(Comp. B194) 2-[(2,2-Dichloro-3,3-dimethyl-cyclopropanecarbonyl)-amino]-4,7-dihydro-5H-thieno[2,3-c]pyran-3-carboxylic acid amide,
(Comp. B195) 2-[3-(3-Carbamoyl-4,7-dihydro-5H-thieno[2,3-c]pyran-2-yl)-ureido]-3-(4-chloro-phenyl)-propionic-acid methyl ester,
(Comp. B196) 2-[(2-Morpholin-4-ylmethyl-cyclopropanecarbonyl)-amino]-4,7-dihydro-5H-thieno[2,3-c]pyran-3-carboxylic acid amide,
(Comp. B197) 2-[(2-Dimethylaminomethyl-cyclopropanecarbonyl)-amino]-4,7-dihydro-5H-thieno[2,3-c]pyran-3-carboxylic acid amide,
(Comp. B198) 2-[(3-Carbamoyl-4,7-dihydro-5H-thieno[2,3-c]pyran-2-ylcarbamoyl)-methylsulfanyl]-benzoic-acid methyl ester,
(Comp. B199) 2-[2-(2,5-Dimethyl-phenylsulfanyl)-acetylamino]-4,7-dihydro-5H-thieno[2,3-c]pyran-3-carboxylic acid amide,
(Comp. B200) 2-[2-(2-Methoxy-phenylsulfanyl)-acetylamino]-4,7-dihydro-5H-thieno[2,3-c]pyran-3-carboxylic acid amide,
(Comp. B201) 2-(3-Cyclopropyl-thioureido)-4,7-dihydro-5H-thieno[2,3-c]pyran-3-carboxylic acid amide,
(Comp. B202) 2-[2-(Cyclopropylmethyl-amino)-acetylamino]-4,7-dihydro-5H-thieno[2,3-c]pyran-3-carboxylic acid amide,
(Comp. B203) 2-(2-Chloro-acetylamino)-4,7-dihydro-5H-thieno[2,3-c]pyran-3-carboxylic acid amide,
(Comp. B204) 2-(2-lsopropylamino-acetylamino)-4,7-dihydro-5H-thieno[2,3-c]pyran-3-carboxylic acid amide,
(Comp. B205) 2-(2-m-Tolylsulfanyl-acetylamino)-4,7-dihydro-5H-thieno[2,3-c]pyran-3-carboxylic acid amide,
(Comp. B206) 2-(2-o-Tolylsulfanyl-acetylamino)-4,7-dihydro-5H-thieno[2,3-c]pyran-3-carboxylic acid amide,
(Comp. B207) 2-[2-(3,4-Dichloro-phenylsulfanyl)-acetylamino]-4,7-dihydro-5H-thieno[2,3-c]pyran-3-carboxylic acid amide,
(Comp. B208) 2-[2-(3-Methoxy-phenylsulfanyl)-acetylamino]-4,7-dihydro-5H-thieno[2,3-c]pyran-3-carboxylic acid amide,
(Comp. B209) 2-[2-(2,3,5,6-Tetrafluoro-phenylsulfanyl)-acetylamino]-4,7-dihydro-5H-thieno[2,3-c]pyran-3-carboxylic acid amide,
(Comp. B210) 2-[2-(2,5-Dichloro-phenylsulfanyl)-acetylamino]-4,7-dihydro-5H-thieno[2,3-c]pyran-3-carboxylic acid amide,
(Comp. B211) 2-[2-(3-Chloro-phenylsulfanyl)-acetylamino]-4,7-dihydro-5H-thieno[2,3-c]pyran-3-carboxylic acid amide,
(Comp. B212) 2-[2-(4-Methoxy-phenylsulfanyl)-acetylamino]-4,7-dihydro-5H-thieno[2,3-c]pyran-3-carboxylic acid amide,
(Comp. B213) 2-[2-(3-Bromo-phenylsulfanyl)-acetylamino]-4,7-dihydro-5H-thieno[2,3-c]pyran-3-carboxylic acid amide,
(Comp. B214) 2-(2-Pyrrolidin-1-yl-acetylamino)-4,7-dihydro-5H-thieno[2,3-c]pyran-3-carboxylic acid amide,
(Comp. B215) 2-[2-(4-Fluoro-phenylsulfanyl)-acetylamino]-4,7-dihydro-5H-thieno[2,3-c]pyran-3-carboxylic acid amide,
(Comp. B216) 2-[2-(4-Methyl-piperazin-1-yl))-acetylamino]-4,7-dihydro-5H-thieno[2,3-c]pyran-3-carboxylic acid amide,
(Comp. B217) 2-(2-Bromo-acetylamino)-4,7-dihydro-5H-thieno[2,3-c]pyran-3-carboxylic acid amide,
(Comp. B218) 2-[2-(2-Morpholin-4-yl-ethylamino))-acetylamino]-4,7-dihydro-5H-thieno[2,3-c]pyran-3-carboxylic acid amide,
(Comp. B219) 2-(2-Butylamino-acetylamino)-4,7-dihydro-5H-thieno[2,3-c]pyran-3-carboxylic acid amide,
(Comp. B220) 2-(2-Cyclohexylamino-acetylamino)-4,7-dihydro-5H-thieno[2,3-c]pyran-3-carboxylic acid amide,
(Comp. B221) 2-[(2,2-Dimethyl-cyclopropanecarbonyl)-amino]-4,7-dihydro-5H-thieno[2,3-c]pyran-3-carboxylic acid amide,
(Comp. B222) 2-[2-(2-Methyl-butylamino)-acetylamino]-4,7-dihydro-5H-thieno[2,3-c]pyran-3-carboxylic acid amide,
(Comp. B223) 2-[2-(1,2-Dimethyl-propylamino)-acetylamino]-4,7-dihydro-5H-thieno[2,3-c]pyran-3-carboxylic acid amide,
(Comp. B224) 2-(4-Methyl-pentanoylamino)-4,7-dihydro-5H-thieno[2,3-c]pyran-3-carboxylic acid amide,
(Comp. B225) (3-Carbamoyl-4,7-dihydro-5H-thieno[2,3-c]pyran-2-yl)-carbamic acid ethyl ester,
(Comp. B226) 2-(3-Trifluoromethyl-benzoylamino)-4,7-dihydro-5H-thieno[2,3-c]pyran-3-carboxylic acid amide,
(Comp. B227) 2-[3-(5-Chloro-2-methyl-phenyl)-ureido]-4,7-dihydro-5H-thieno[2,3-c]pyran-3-carboxylic acid amide,
(Comp. B228) 2-[(2-Methyl-cyclohexanecarbonyl)-amino]-4,7-dihydro-5H-thieno[2,3-c]pyran-3-carboxylic acid amide,
(Comp. B229) 2-[(2-Hydroxymethyl-cyclopropanecarbonyl)-amino]-4,7-dihydro-5H-thieno[2,3-c]pyran-3-carboxylic acid amide,
(Comp. B230) 2-[3-(2,4-Dimethoxy-phenyl)-ureido]-4,7-dihydro-5H-thieno[2,3-c]pyran-3-carboxylic acid amide,
(Comp. B231) 2-[3-(2-Hydroxy-1-hydroxymethyl-ethyl)-ureido]-4,7-dihydro-5H-thieno[2,3-c]pyran-3-carboxylic acid amide,
(Comp. B232) 2-[3-(4-Diethylamino-1-methyl-butyl)-ureido]-4,7-dihydro-5H-thieno[2,3-c]pyran-3-carboxylic acid amide,
(Comp. B233) 2-[3-(2-Pyridin-2-yl-ethyl)-ureido]-4,7-dihydro-5H-thieno[2,3-c]pyran-3-carboxylic acid amide,
(Comp. B234) 2-[3-(1-Cyclohexyl-ethyl)-ureido]-4,7-dihydro-5H-thieno[2,3-c]pyran-3-carboxylic acid amide,
(Comp. B235) 2-(Cyclopropanecarbonyl-amino)-7-(2-methoxy-ethoxy)-4,7-dihydro-5H-thieno[2,3-c]pyran-3-carboxylic acid amide,
(Comp. B236) 2-[3-(1,2,3,4-Tetrahydro-naphthalen-1-yl)-ureido]-4,7-dihydro-5H-thieno[2,3-c]pyran-3-carboxylic acid amide,
(Comp. B237) 2-[3-(5,6-Dichloro-pyridin-3-yl)-ureido]-4,7-dihydro-5H-thieno[2,3-c]pyran-3-carboxylic acid amide,
(Comp. B238) 2-(Cyclopropanecarbonyl-amino)-7-(2-piperidin-1-yl-ethylamino)-4,7-dihydro-5H-thieno[2,3-c]pyran-3-carboxylic acid amide,
(Comp. B239) 2-(Cyclopropanecarbonyl-amino)-7-[(pyridin-2-ylmethyl)-amino]-4,7-dihydro-5H-thieno[2,3-c]pyran-3-carboxylic acid amide,
(Comp. B240) 2-(Cyclopropanecarbonyl-amino)-7-(2-ethoxy-ethoxy)-4,7-dihydro-5H-thieno[2,3-c]pyran-3-carboxylic acid amide,
(Comp. B241) 2-(Cyclopropanecarbonyl-amino)-7-(2-diethylamino-ethoxy)-4,7-dihydro-5H-thieno[2,3-c]pyran-3-carboxylic acid amide,
(Comp. B242) 2-(Cyclopropanecarbonyl-amino)-7-(2-chloro-ethoxy)-4,7-dihydro-5H-thieno[2,3-c]pyran-3-carboxylic acid amide,
(Comp. B243) 2-[3-(1,2-Dimethyl-propyl)-ureido]-4,7-dihydro-5H-thieno[2,3-c]pyran-3-carboxylic acid amide,
(Comp. B244) 2-(Hydrazino-carbonyl-amino)-4,7-dihydro-5H-thieno[2,3-c]pyran-3-carboxylic acid amide,
(Comp. B245) 2-[3-(4-Chloro-2-trifluoromethyl-phenyl)-ureido]-4,7-dihydro-5H-thieno[2,3-c]pyran-3-carboxylic acid amide,
(Comp. B246) 2-(Cyclopropanecarbonyl-amino)-7-(4-fluoro-benzylamino)-4,7-dihydro-5H-thieno[2,3-c]pyran-3-carboxylic acid amide,
(Comp. B247) 2-[3-(2-Piperazin-2-yl-ethyl)-ureido]-4,7-dihydro-5H-thieno[2,3-c]pyran-3-carboxylic acid amide,
(Comp. B248) 2-(Cyclopropanecarbonyl-amino)-4,7-dihydro-5H-thieno[2,3-c]pyran-3-carboxylic acid (4-chloro-phenyl)-amide,
(Comp. B249) 2-[3-(2-Methylsulfanyl-phenyl)-ureido]-4,7-dihydro-5H-thieno[2,3-c]pyran-3-carboxylic acid amide,
(Comp. B250) 2-[3-(2-Trifluoromethoxy-phenyl)-ureido]-4,7-dihydro-5H-thieno[2,3-c]pyran-3-carboxylic acid amide,
(Comp. B251) 2-[3-(2-Difluoromethoxy-phenyl)-ureido]-4,7-dihydro-5H-thieno[2,3-c]pyran-3-carboxylic acid amide,
(Comp. B252) 2-{3-[4-Cyano-3-(4-methoxy-phenyl)-isoxazol-5-yl]-ureido}-4,7-dihydro-5H-thieno[2,3-c]pyran-3-carboxylic acid amide,
(Comp. B253) 2-[3-(4-Cyano-3-cyclopropyl-isoxazol-5-yl)-ureido]-4,7-dihydro-5H-thieno[2,3-c]pyran-3-carboxylic acid amide,
(Comp. B254) 2-(3-Pyrimidin-2-yl-ureido)-4,7-dihydro-5H-thieno[2,3-c]pyran-3-carboxylic acid amide,
(Comp. B255) 2-[3-(4-Trifluoromethylsulfanyl-phenyl)-ureido]-4,7-dihydro-5H-thieno[2,3-c]pyran-3-carboxylic acid amide,
(Comp. B256) 2-[3-(4-Cyano-3-phenyl-isoxazol-5-yl)-ureido]-4,7-dihydro-5H-thieno[2,3-c]pyran-3-carboxylic acid amide,
(Comp. B257) 2-(3-Piperidin-1-yl-ureido)-4,7-dihydro-5H-thieno[2,3-c]pyran-3-carboxylic acid amide,
(Comp. B258) 2-[3-(4-Methyl-piperazin-1-yl)-ureido]-4,7-dihydro-5H-thieno[2,3-c]pyran-3-carboxylic acid amide,
(Comp. B259) 2-[3-(4-tert-Butyl-cyclohexyl)-ureido]-4,7-dihydro-5H-thieno[2,3-c]pyran-3-carboxylic acid amide,
(Comp. B260) 2-[3-(1S,2S,3S,5R-2,6,6-Trimethyl-bicyclo[3.1.1]hept-3-yl)-ureido]-4,7-dihydro-5H-thieno[2,3-c]pyran-3-carboxylic acid amide,
(Comp. B261) 2-[3-(2,3-Dihydro-benzo[1,4]dioxin-6-yl)-ureido]-4,7-dihydro-5H-thieno[2,3-c]pyran-3-carboxylic acid amide,
(Comp. B262) 2-[3-(2-Hydroxy-phenyl)-ureido]-4,7-dihydro-5H-thieno[2,3-c]pyran-3-carboxylic acid amide,
(Comp. B263) N-(3-carbamoyl-4,7-dihydro-5H-thieno[2,3]pyran-2-yl)-N′-(2-hydroxyethyl)-oxalamide,
(Comp. B264) 4-[3-(3-Carbamoyl-4,7-dihydro-5H-thieno[2,3-c]pyran-2-yl)-ureido]-benzoic acid ethyl ester,
(Comp. B265) 2-[3-(3-Methylsulfanyl-phenyl)-ureido]-4,7-dihydro-5H-thieno[2,3-c]pyran-3-carboxylic acid amide,
(Comp. B266) 2-[3-(3-Methylsulfanyl-phenyl)-ureido]-4,7-dihydro-5H-thieno[2,3-c]pyran-3-carboxylic acid amide,
(Comp. B267) 2-[3-(2-Ethyl-phenyl)-ureido]-4,7-dihydro-5H-thieno[2,3-c]pyran-3-carboxylic acid amide,
(Comp. B268) 2-[3-(2-Chloro-phenyl)-ureido]-4,7-dihydro-5H-thieno[2,3-c]pyran-3-carboxylic acid amide,
(Comp. B269) 2-[3-(2-Chloro-6-methyl-phenyl)-ureido]-4,7-dihydro-5H-thieno[2,3-c]pyran-3-carboxylic acid amide,
(Comp. B270) 2-[3-(4-Bromo-2-fluoro-phenyl)-ureido]-4,7-dihydro-5H-thieno[2,3-c]pyran-3-carboxylic acid amide,
(Comp. B271) 2-[3-(2-Fluoro-3-trifluoromethyl-phenyl)-ureido]-4,7-dihydro-5H-thieno[2,3-c]pyran-3-carboxylic acid amide,
(Comp. B272) 2-[3-(2,3,4-Trifluoro-phenyl)-ureido]-4,7-dihydro-5H-thieno[2,3-c]pyran-3-carboxylic acid amide,
(Comp. B273) 2-[3-(2,6-Dibromo-4-fluoro-phenyl)-ureido]-4,7-dihydro-5H-thieno[2,3-c]pyran-3-carboxylic acid amide,
(Comp. B274) 2-[3-(2,3-Dimethyl-phenyl)-ureido]-4,7-dihydro-5H-thieno[2,3-c]pyran-3-carboxylic acid amide,
(Comp. B275) 2-[3-(3-Fluoro-4-methyl-phenyl)-ureido]-4,7-dihydro-5H-thieno[2,3-c]pyran-3-carboxylic acid amide,
(Comp. B276) 2-[3-(3,4-Difluoro-phenyl)-ureido]-4,7-dihydro-5H-thieno[2,3-c]pyran-3-carboxylic acid amide,
(Comp. B277) 2-[3-(4-Chloro-2-methyl-phenyl)-ureido]-4,7-dihydro-5H-thieno[2,3-c]pyran-3-carboxylic acid amide,
(Comp. B278) 2-[3-(4-Ethyl-phenyl)-ureido]-4,7-dihydro-5H-thieno[2,3-c]pyran-3-carboxylic acid amide,
(Comp. B279) 2-[3-(3-lodo-phenyl)-ureido]-4,7-dihydro-5H-thieno[2,3-c]pyran-3-carboxylic acid amide,
(Comp. B280) 2-[3-(2,5-Dimethyl-phenyl)-ureido]-4,7-dihydro-5H-thieno[2,3-c]pyran-3-carboxylic acid amide,
(Comp. B281) 2-[3-(4-Butoxy-phenyl)-ureido]-4,7-dihydro-5H-thieno[2,3-c]pyran-3-carboxylic acid amide,
(Comp. B282) 2-[3-(2,4-Dibromo-phenyl)-ureido]-4,7-dihydro-5H-thieno[2,3-c]pyran-3-carboxylic acid amide,
(Comp. B283) 2-[3-(4-Butyl-phenyl)-ureido]-4,7-dihydro-5H-thieno[2,3-c]pyran-3-carboxylic acid amide,
(Comp. B284) 2-[3-(4-Dimethylamino-phenyl)-ureido]-4,7-dihydro-5H-thieno[2,3-c]pyran-3-carboxylic acid amide,
(Comp. B285) 2-[3-(4-Heptyloxy-phenyl)-ureido]-4,7-dihydro-5H-thieno[2,3-c]pyran-3-carboxylic acid amide,
(Comp. B286) 2-[3-(2-Propyl-phenyl)-ureido]-4,7-dihydro-5H-thieno[2,3-c]pyran-3-carboxylic acid amide.
(Comp. C1) 2-(Cyclopropanecarbonyl-amino)-4,7-dihydro-5H-thieno[2,3-c]thiopyran-3-carboxylic acid amide,
(Comp. C2) 2-(Cyclopropanecarbonyl-amino)-6-oxo-4,5,6,7-tetrahydro-6λ⁴-thieno[2,3-c]thiopyran-3-carboxylic acid amide,
(Comp. C3) 3-Carbamoyl-2-(cyclopropanecarbonyl-amino)-4,7-dihydro-5H-thieno[2,3-c]pyridine-6-carboxylic acid ethyl ester,
(Comp. C4) 6-Acetyl-2-(cyclopropanecarbonyl-amino)-4,5,6,7-tetrahydro-thieno[2,3-c]pyridine-3-carboxylic acid amide,
(Comp. C5) 2-[3-(4-Methoxy-phenyl)-ureido]-4,7-dihydro-5H-thieno[2,3-c]thiopyran-3-carboxylic acid amide,
(Comp. C6) 2-{3-[4-(2-Morpholin-4-yl-ethoxy)-naphthalen-1-yl]-ureido}-4,7-dihydro-5H-thieno[2,3-c]thiopyran-3-carboxylic acid amide,
(Comp. C7) 2-[3-(3-Carbamoyl-4,7-dihydro-5H-thieno[2,3-c]thiopyran-2-yl)-ureido]-3-methyl-butyric acid methyl ester,
(Comp. C8) 2-[3-(4-Benzyloxy-phenyl)-ureido]-4,7-dihydro-5H-thieno[2,3-c]thiopyran-3-carboxylic acid amide,
(Comp. C9) 2-[3-(3-Morpholin-4-yl-propyl)-ureido]-4,7-dihydro-5H-thieno[2,3-c]thiopyran-3-carboxylic acid amide,
(Comp. C10) 2-(3-Pyridin-3-yl-ureido)-4,7-dihydro-5H-thieno[2,3-c]thiopyran-3-carboxylic acid amide,
(Comp. C11) 2-[3-(3-Chloro-4-fluoro-phenyl)-ureido]-4,7-dihydro-5H-thieno[2,3-c]thiopyran-3-carboxylic acid amide,
(Comp. C12) 2-[3-(2-Methoxy-benzyl)-ureido)-4,7-dihydro-5H-thieno[2,3-c]thiopyran-3-carboxylic acid amide,
(Comp. C13) 2-[3-(4-Nitro-phenyl)-ureido]-4,7-dihydro-5H-thieno[2,3-c]thiopyran-3-carboxylic acid amide,
(Comp. C14) 2-[3-(4-Isopropyl-phenyl)-ureido]-4,7-dihydro-5H-thieno[2,3-c]thiopyran-3-carboxylic acid amide,
(Comp. C15) 2-[3-(2,4-Difluoro-phenyl)-ureido]-4,7-dihydro-5H-thieno[2,3-c]thiopyran-3-carboxylic acid amide,
(Comp. C16) 2-[3-(3,4,5-Trimethoxy-phenyl)-ureido]-4,7-dihydro-5H-thieno[2,3-c]thiopyran-3-carboxylic acid amide,
(Comp. C17) 2-[3-(3,5-Dichloro-phenyl)-ureido]-4,7-dihydro-5H-thieno[2,3-c]thiopyran-3-carboxylic acid amide,
(Comp. C18) 2-[3-(2-Nitro-phenyl)-ureido]-4,7-dihydro-5H-thieno[2,3-c]thiopyran-3-carboxylic acid amide,
(Comp. C19) 2-[3-(3-Methoxy-phenyl)-ureido]-4,7-dihydro-5H-thieno[2,3-c]thiopyran-3-carboxylic acid amide,
(Comp. C20) 2-[3-(2-Methyl-3-nitro-phenyl)-ureido]-4,7-dihydro-5H-thieno[2,3-c]thiopyran-3-carboxylic acid amide,
(Comp. C21) 2-[3-(4-Chloro-3-nitro-phenyl)-ureido]-4,7-dihydro-5H-thieno[2,3-c]thiopyran-3-carboxylic acid amide,
(Comp. C22) 2-[3-(2-Methoxy-5-nitro-phenyl)-ureido]-4,7-dihydro-5H-thieno[2,3-c]thiopyran-3-carboxylic acid amide,
(Comp. C23) 2-[3-(3-Bromo-phenyl)-ureido]-4,7-dihydro-5H-thieno[2,3-c]thiopyran-3-carboxylic acid amide,
(Comp. C24) 2-[3-(3,5-Dimethoxy-phenyl)-ureido]-4,7-dihydro-5H-thieno[2,3-c]thiopyran-3-carboxylic acid amide,
(Comp. C25) 2-[3-(4-Chloro-3-trifluoromethyl-phenyl)-ureido]-4,7-dihydro-5H-thieno[2,3-c]thiopyran-3-carboxylic acid amide,
(Comp. C26) 2-[3-(3,5-Bis-trifluoromethyl-phenyl)-ureido]-4,7-dihydro-5H-thieno[2,3-c]thiopyran-3-carboxylic acid amide,
(Comp. C27) 2-(3-m-Tolyl-ureido)-4,7-dihydro-5H-thieno[2,3-c]thiopyran-3-carboxylic acid amide,
(Comp. C28) 2-(3-p-Tolyl-ureido)-4,7-dihydro-5H-thieno[2,3-c]thiopyran-3-carboxylic acid amide,
(Comp. C29) 2-[3-(3-Methyl-benzyl)-ureido]-4,7-dihydro-5H-thieno[2,3-c]thiopyran-3-carboxylic acid amide,
(Comp. C30) 2-[3-(2-Chloro-5-trifluoromethyl-phenyl)-ureido]-4,7-dihydro-5H-thieno[2,3-c]thiopyran-3-carboxylic acid amide,
(Comp. C31) 2-(3-o-Tolyl-ureido)-4,7-dihydro-5H-thieno[2,3-c]thiopyran-3-carboxylic acid amide,
(Comp. C32) 2-[3-(4-Methoxy-benzyl)-ureido]-4,7-dihydro-5H-thieno[2,3-c]thiopyran-3-carboxylic acid amide,
(Comp. C33) 2-[3-(2-Methyl-benzyl)-ureido]-4,7-dihydro-5H-thieno[2,3-c]thiopyran-3-carboxylic acid amide,
(Comp. C34) 2-[3-(2,3-Dichloro-phenyl)-ureido]-4,7-dihydro-5H-thieno[2,3-c]thiopyran-3-carboxylic acid amide,
(Comp. C35) 2-(3-Biphenyl-4-yl-ureido)-4,7-dihydro-5H-thieno[2,3-c]thiopyran-3-carboxylic acid amide,
(Comp. C36) 2-[3-(3-Trifluoromethyl-phenyl)-ureido]-4,7-dihydro-5H-thieno[2,3-c]thiopyran-3-carboxylic acid amide,
(Comp. C37) 2-[3-(4-Bromo-phenyl)-ureido]-4,7-dihydro-5H-thieno[2,3-c]thiopyran-3-carboxylic acid amide,
(Comp. C38) 2-(3-Cyclohexyl-ureido)-4,7-dihydro-5H-thieno[2,3-c]thiopyran-3-carboxylic acid amide,
(Comp. C39) 2-(2-Methyl-acryloylamino)-4,7-dihydro-5H-thieno[2,3-c]thiopyran-3-carboxylic acid amide,
(Comp. C40) 2-[3-(3-Chloro-phenyl)-ureido]-4,7-dihydro-5H-thieno[2,3-c]thiopyran-3-carboxylic acid amide,
(Comp. C41) 2-(3-Benzyl-ureido)-4,7-dihydro-5H-thieno[2,3-c]thiopyran-3-carboxylic acid amide,
(Comp. C42) 2-[3-(3-Methyl-butyl)-ureido]-4,7-dihydro-5H-thieno[2,3-c]thiopyran-3-carboxylic acid amide,
(Comp. C43) 2-[3-(4-Methyl-benzyl)-ureido]-4,7-dihydro-5H-thieno[2,3-c]thiopyran-3-carboxylic acid amide,
(Comp. C44) 2-(3-Isopropyl-ureido)-4,7-dihydro-5H-thieno[2,3-c]thiopyran-3-carboxylic acid amide,
(Comp. C45) 2-[3-(4-Fluoro-phenyl)-ureido]-4,7-dihydro-5H-thieno[2,3-c]thiopyran-3-carboxylic acid amide,
(Comp. C46) 2-[3-(2-Methyl-5-nitro-phenyl)-ureido]-4,7-dihydro-5H-thieno[2,3-c]thiopyran-3-carboxylic acid amide,
(Comp. C47) 2-[3-(4-Acetyl-phenyl)-ureido]-4,7-dihydro-5H-thieno[2,3-c]thiopyran-3-carboxylic acid amide,
(Comp. C48) 2-[3-(4-Methyl-2-nitro-phenyl)-ureido]-4,7-dihydro-5H-thieno[2,3-c]thiopyran-3-carboxylic acid amide,
(Comp. C49) 2-[3-(4-Phenoxy-phenyl)-ureido]-4,7-dihydro-5H-thieno[2,3-c]thiopyran-3-carboxylic acid amide,
(Comp. C50) 2-[3-(3,4-Dichloro-phenyl)-ureido]-4,7-dihydro-5H-thieno[2,3-c]thiopyran-3-carboxylic acid amide,
(Comp. C51) 2-(Cyclopropanecarbonyl-amino)-6-methyl-4,5,6,7-tetrahydro-thieno[2,3-c]pyridine-3-carboxylic acid amide,
(Comp. C52) 2-[(2,2,3,3-Tetramethyl-cyclopropanecarbonyl)-amino]-4,7-dihydro-5H-thieno[2,3-c]thiopyran-3-carboxylic acid amide,
(Comp. C53) 2-[3-(4-Trifluoromethyl-phenyl)-ureido]-4,7-dihydro-5H-thieno[2,3-c]thiopyran-3-carboxylic acid amide,
(Comp. D1) 2-(Cyclopropanecarbonyl-amino)-4,5,6,7-tetrahydro-benzo[b]thiophene-3-carboxylic acid amide,
(Comp. D2) Furan-2-carboxylic acid2-[3-(2-hydroxy-ethylcarbamoyl)-4,5,6,7-tetrahydro-benzo[b]thiophen-2-yl]-amide,
(Comp. D3) 2-Acetylamino-4,5,6,7-tetrahydro-benzo[b]thiophene-3-carboxylic acid amide,
(Comp. D4) 2-(2,2-Dimethyl-propionylamino)-4,5,6,7-tetrahydro-benzo[b]thiophene-3-carboxylic acid amide,
(Comp. D5) 2-Isobutyrylamino)-4,5,6,7-tetrahydro-benzo[b]thiophene-3-carboxylic acid amide,
(Comp. D6) 2-(2-Methyl-acryloylamino)-4,5,6,7-tetrahydro-benzo[b]thiophene-3-carboxylic acid amide,
(Comp. D7) 2-[(Thiophene-2-carbonyl)-amino]-4,5,6,7-tetrahydro-benzo[b]thiophene-3-carboxylic acid amide,
(Comp. D8) Furan-2-carboxylic acid (3-carbamoyl-4,5,6,7-tetrahydro-benzo[b]thiophen-2-yl]-amide,
(Comp. D9) 2-(Cyclobutanecarbonylamino)-4,5,6,7-tetrahydro-benzo[b]thiophene-3-carboxylic acid amide,
(Comp. D10) 2-(2-Methyl-butyrylamino)-4,5,6,7-tetrahydro-benzo[b]thiophene-3-carboxylic acid amide,
(Comp. D11) 2-(Cyclopropanecarbonyl-amino)-4-methyl-4,5,6,7-tetrahydro-benzo[b]thiophene-3-carboxylic acid amide,
(Comp. D12) 6-tert-Butyl-2-(cyclopropanecarbonyl-amino)-4,5,6,7-tetrahydro-benzo[b]thiophene-3-carboxylic acid amide,
(Comp. D13) 2-(Cyclopropanecarbonyl-amino)-6-methyl-4,5,6,7-tetrahydro-benzo[b]thiophene-3-carboxylic acid amide,
(Comp. D14) 2-(Cyclohexanecarbonyl-amino)-4,5,6,7-tetrahydro-benzo[b]thiophene-3-carboxylic acid amide,
(Comp. D15) 2-Acetylamino-6-methyl-4,5,6,7-tetrahydro-benzo[b]thiophene-3-carboxylic acid amide,
(Comp. D16) N-(3-Carbamoyl-4,5,6,7-tetrahydro-benzo[b]thiophen-2-yl)-succinamic acid methyl ester,
(Comp. D17) 6-Methyl-2-propyonylamino-4,5,6,7-tetrahydro-benzo[b]thiophene-3-carboxylic acid amide,
(Comp. D18) 2-lsobutyrylamino-6-methyl-4,5,6,7-tetrahydro-benzo[b]thiophene-3-carboxylic acid amide,
(Comp. D19) 2-Butyrylamino-4,5,6,7-tetrahydro-benzo[b]thiophene-3-carboxylic acid amide,
(Comp. D20) 3-(3-Carbamoyl-4,5,6,7-tetrahydro-benzo[b]thiophen-2-ylcarbamoyl)-acrylic acid,
(Comp. D21) N-(3-Carbamoyl-4,5,6,7-tetrahydro-benzo[b]thiophen-2-yl)-succinamic acid,
(Comp. D22) 2-Benzoylamino-5,6-dihydro-4H-cyclopenta[b]thiophene-3-carboxylic acid amide,
(Comp. D23) Furan-2-carboxylic acid (3-carbamoyl-5,6-dihydro-4H-cyclopenta[b]thiophen-2-yl]-amide,
(Comp. D24) 2-(2-Cyano-acetylamino)-4,5,6,7-tetrahydro-benzo[b]thiophene-3-carboxylic acid amide,
(Comp. D25) 2-(2-Phenoxy-acetylamino)-4,5,6,7-tetrahydro-benzo[b]thiophene-3-carboxylic acid amide,
(Comp. D26) 2-(3-Phenyl-ureido)-4,5,6,7-tetrahydro-benzo[b]thiophene-3-carboxylic acid amide,
(Comp. D27) 2-(2-Piperidin-1-yl-acetylamino)-4,5,6,7-tetrahydro-benzo[b]thiophene-3-carboxylic acid amide,
(Comp. D28) 2-(2-Dialkylamino-acetylamino)-4,5,6,7-tetrahydro-benzo[b]thiophene-3-carboxylic acid amide,
(Comp. D29) 2-(2-Morpholin-4-yl-acetylamino)-4,5,6,7-tetrahydro-benzo[b]thiophene-3-carboxylic acid amide,
(Comp. D30) 4-Ethyl-piperazine-1-carboxylic acid (3-carbamoyl-4,5,6,7-tetrahydro-benzo[b]thiophen-2-yl]-amide,
(Comp. D31) 2-(2-Diethylamino-acetylamino)-4,5,6,7-tetrahydro-benzo[b]thiophene-3-carboxylic acid amide,
(Comp. D32) 2-(3-Allyl-thioureido)-4,5,6,7-tetrahydro-benzo[b]thiophene-3-carboxylic acid amide,
(Comp. D33) 2-Pentanoylamino-4,5,6,7-tetrahydro-benzo[b]thiophene-3-carboxylic acid amide,
(Comp. D34) 6-Methyl-2-(3-thiophen-2-yl-acryloylamino)-4,5,6,7-tetrahydro-benzo[b]thiophene-3-carboxylic acid amide,
(Comp. D35) 2-[(Adamantane-1-carbonyl)-amino]-4,5,6,7-tetrahydro-benzo[b]thiophene-3-carboxylic acid amide,
(Comp. D36) 5,6-Dihydro-[1,4]-dioxine-2-carboxylic acid (3-carbamoyl-4,5,6,7-tetrahydro-benzo[b]thiophen-2-yl]-amide,
(Comp. D37) 2-(Cyclopropanecarbonyl-amino)-5-methyl-4,5,6,7-tetrahydro-benzo[b]thiophene-3-carboxylic acid amide,
(Comp. D38) 2-lsobutyrylamino-5-methyl-4,5,6,7-tetrahydro-benzo[b]thiophene-3-carboxylic acid amide,
(Comp. D39) 2-Phenylacetylamino-4,5,6,7-tetrahydro-benzo[b]thiophene-3-carboxylic acid amide,
(Comp. D40) (3-Carbamoyl-4,5,6,7-tetrahydro-benzo[b]thiophen-2-yl)-carbamic acid butyl ester,
(Comp. D41) 6-Methyl-2-(3-methyl-but-2-enoylamino)-4,5,6,7-tetrahydro-benzo[b]thiophene-3-carboxylic acid amide,
(Comp. D42) (3-Carbamoyl-4,5,6,7-tetrahydro-benzo[b]thiophen-2-yl)-carbamic acid ethyl ester,
(Comp. D43) 2-(2-Cyano-3-furan-2yl-acryloylamino)-4,5,6,7-tetrahydro-benzo[b]thiophene-3-carboxylic acid amide,
(Comp. D44) 2-(3-Methyl-but-2-enoylamino)-4,5,6,7-tetrahydro-benzo[b]thiophene-3-carboxylic acid amide,
(Comp. D45) 2-(3-Phenyl-acryloylamino)-4,5,6,7-tetrahydro-benzo[b]thiophene-3-carboxylic acid amide,
(Comp. D46) 2-(2-Methoxy-acetylamino)-4,5,6,7-tetrahydro-benzo[b]thiophene-3-carboxylic acid amide,
(Comp. D47) 2-[2-(1,1-Dioxo-tetrahydro-1λ⁶-thiophen-3-yl)-acetylamino]-4,5,6,7-tetrahydro-benzo[b]thiophene-3-carboxylic acid amide,
(Comp. D48) 2-[2-(4-Methoxyphenyl)-acetylamino]-4,5,6,7-tetrahydro-benzo[b]thiophene-3-carboxylic acid amide,
(Comp. D49) (3-Carbamoyl-6-methyl-4,5,6,7-tetrahydro-benzo[b]thiophen-2-yl)-carbamic acid phenyl ester,
(Comp. D50) 2-(4-Phenoxy-butyrylamino)-4,5,6,7-tetrahydro-benzo[b]thiophene-3-carboxylic acid amide,
(Comp. D51) (3-Carbarmoyl-4,5,6,7-tetrahydro-benzo[b]thiophen-2-y1)-carbamic acid propyl ester,
(Comp. D52) Tetrahydro-furan-2-carboxylic-acid(3-Carbamoyl-4,5,6,7-tetrahydro-benzo[b]thiophen-2-yl)-amide,
(Comp. D53) 2-(4-Phenyl-butyrylamino]-4,5,6,7-tetrahydro-benzo[b]thiophene-3-carboxylic acid amide,
(Comp. D54) 2-(3-Pyrrolidin-1-yl-propionylamino)-4,5,6,7-tetrahydro-benzo[b]thiophene-3-carboxylic acid amide,
(Comp. D55) 2-(2-Ethyl-hexanoylamino)-4,5,6,7-tetrahydro-benzo[b]thiophene-3-carboxylic acid amide,
(Comp. D56) 2-(3-Cyclopentyl-propionylamino)-4,5,6,7-tetrahydro-benzo[b]thiophene-3-carboxylic acid amide,
(Comp. D57) 2-(3-Cyclohexyl-propionylamino)-4,5,6,7-tetrahydro-benzo[b]thiophene-3-carboxylic acid amide,
(Comp. D58) 2-(3-Methyl-butyrylamino)-4,5,6,7-tetrahydro-benzo[b]thiophene-3-carboxylic acid amide,
(Comp. D59) 2-(2,2,2-Trifluoro-acetylamino)-4,5,6,7-tetrahydro-benzo[b]thiophene-3-carboxylic acid amide,
(Comp. D60) 2-(3-Furan-2-yl-acryloylamino)-4,5,6,7-tetrahydro-benzo[b]thiophene-3-carboxylic acid amide,
(Comp. D61) 2-[(2-Phenyl-cyclopropanecarbonyl)-amino]-4,5,6,7-tetrahydro-benzo[b]thiophene-3-carboxylic acid amide,
(Comp. D62) 2-(Cyclopentanecarbonyl-amino)-6-4,5,6,7-tetrahydro-benzo[b]thiophene-3-carboxylic acid amide,
(Comp. D63) 2-[(Bicyclo[2.2.1 ]heptane-2-carbonyl)-amino]-4,5,6,7-tetrahydro-benzo[b]thiophene-3-carboxylic acid amide,
(Comp. D64) 2-(3-Adamantan-1-yl-ureido)-4,5,6,7-tetrahydro-benzo[b]thiophene-3-carboxylic acid amide,
(Comp. D65) 2-[3-(4-Methoxy-benzyl)-ureido]-4,5,6,7-tetrahydro-benzo[b]thiophene-3-carboxylic acid amide,
(Comp. D66) 2-(Cyclopropanecarbonyl-amino)-6-fluoro-4,5,6,7-tetrahydro-benzo[b]thiophene-3-carboxylic acid amide,
(Comp. D67) 6,6-Dibromo-2-(cyclopropanecarbonyl-amino)-7-oxo-4,5,6,7-tetrahydro-benzo[b]thiophene-3-carboxylic acid amide,
(Comp. D68) 2-[3-(4-Methyl-benzyl)-ureido]-4,5,6,7-tetrahydro-benzo[b]thiophene-3-carboxylic acid amide,
(Comp. D69) 2-[3-(3-Methyl-benzyl)-ureido]-4,5,6,7-tetrahydro-benzo[b]thiophene-3-carboxylic acid amide,
(Comp. D70) 2-(3-p-Tolyl-ureido)-4,5,6,7-tetrahydro-benzo[b]thiophene-3-carboxylic acid amide,
(Comp. D71) 2-[3-(4-Methyl-2-nitro-phenyl)-ureido]-4,5,6,7-tetrahydro-benzo[b]thiophene-3-carboxylic acid amide,
(Comp. D72) E-7-Benzyloxyimino-2-(cyclopropanecarbonyl-amino)-4,5,6,7-tetrahydro-benzo[b]thiophene-3-carboxylic acid amide,
(Comp. D73) Z-7-Benzyloxyamino-2-(cyclopropanecarbonyl-amino)-4,5,6,7-tetrahydro-benzo[b]thiophene-3-carboxylic acid amide,
(Comp. D74) 2-[3-(3-Methyl-butyl)-ureido]-4,5,6,7-tetrahydro-benzo[b]thiophene-3-carboxylic acid amide,
(Comp. D75) 2-[3-(4-Acetyl-phenyl)-ureido]-4,5,6,7-tetrahydro-benzo[b]thiophene-3-carboxylic acid amide,
(Comp. D76) 2-[3-(2-Methyl-5-nitro-phenyl)-ureido]-4,5,6,7-tetrahydro-benzo[b]thiophene-3-carboxylic acid amide,
(Comp. D77) 2-(3-m-Tolyl-ureido)-4,5,6,7-tetrahydro-benzo[b]thiophene-3-carboxylic acid amide,
(Comp. D78) 3-(3-Carbamoyl-4,5,6,7-tetrahydro-benzo[b]thiophen-2-ylcarbamoyl)-acrylic acid,
(Comp. D79) 2-(Cyclopropanecarbonyl-amino)-4,5,6,7-tetrahydro-benzo[b]thiophene-3,6-dicarboxylic acid 3-amide 6-[(2-hydroxy-ethyl)-amide],
(Comp. D80) 2-[3-(2-Methyl-benzyl)-ureido]-4,5,6,7-tetrahydro-benzo[b]thiophene-3-carboxylic acid amide,
(Comp. D81) 2-[3-(3-Morpholin-4-yl-propyl)-ureido]-4,5,6,7-tetrahydro-benzo[b]thiophene-3-carboxylic acid amide,
(Comp. D82) 3-Carbamoyl-2-(cyclopropanecarbonyl-amino)-4,5,6,7-tetrahydro-benzo[b]thiophene-6-carboxylic acid,
(Comp. D83) 2-[3-(4-Phenoxy-phenyl)-ureido]-4,5,6,7-tetrahydro-benzo[b]thiophene-3-carboxylic acid amide,
(Comp. D84) E-2-(Cyclopropanecarbonyl-amino)-7-hydroxyimino-4,5,6,7-tetrahydro-benzo[b]thiophene-3-carboxylic acid amide,
(Comp. D85) E-2-(Cyclopropanecarbonyl-amino)-7-methoxyimino-4,5,6,7-tetrahydro-benzo[b]thiophene-3-carboxylic acid amide,
(Comp. D86) Cyclopropanecarboxylic acid (3-sulfamoyl-4,5,6,7-tetrahydro-benzo[b]thiophen-2-yl)-amide,
(Comp. D87) 2-[3-(4-Trifluoromethyl-phenyl)-ureido]-4,5,6,7-tetrahydro-benzo[b]thiophene-3-carboxylic acid amide,
(Comp. D88) Z-2-(Cyclopropanecarbonyl-amino)-7-methoxyimino-4,5,6,7-tetrahydro-benzo[b]thiophene-3-carboxylic acid amide,
(Comp. D89) 2-(cyclopropanecarbonyl-amino)-6-oxo-4,5,6,7-tetrahydro-benzo[b]thiophene-3-carboxylic acid amide,
(Comp. D90) 2-(cyclopropanecarbonyl-amino)-6-hydroxy-4,5,6,7-tetrahydro-benzo[b]thiophene-3-carboxylic acid amide,
(Comp. D91) 2-Phenylmethanesulfonylamino-4,5,6,7-tetrahydro-benzo[b]thiophene-3-carboxylic acid amide,
(Comp. D92) 1-Benzyl-2-(cyclopropanecarbonyl-amino)-4,5,6,7-tetrahydro-1H-indole-3-carboxylic acid amide,
(Comp. D93) 2-(Cyclopropanecarbonyl-amino)-7-hydroxyimino-6-methyl-4,5,6,7-tetrahydro-benzo[b]thiophene-3-carboxylic acid amide,
(Comp. D94) 2-[(2-Methyl-cyclopropanecarbonyl)-amino]-4,5,6,7-tetrahydro-benzo[b]thiophene-3-carboxylic acid amide,
(Comp. D95) 2-(Cyclopropanecarbonyl-amino)-5,5,7,7-tetramethyl-4,5,6,7-tetrahydro-benzo[b]thiophene-3-carboxylic acid amide,
(Comp. D96) 2-(Cyclopropanecarbonyl-amino)-4-methyl-4,6-dihydro-thieno[2,3-c]furan-3-carboxylic acid amide,
(Comp. D97) 2-[(Cyclopropylcarbonyl)amino]-4,7-dihydro-5H-spiro[1-benzothiophene-6,2′-[1,3]dioxolane]-3-carboxamide,
(Comp. D98) 2-(Cyclopropanecarbonyl-amino)-4,4a,5,6,7,8,8a,9-octahydro-naphtho[2,3-b]thiophene-3-carboxylic acid amide,
(Comp. D99) Cyclopropanecarboxylic-acid(3-thiocarbamoyl-4,5,6,7-tetrahydro-benzo[b]thiophen-2-yl)-amide,
(Comp. D100) 2-(Cyclopropanecarbothioyl-amino)-4,5,6,7-tetrahydro-benzo[b]thiophene-3-carbothioic acid amide,
(Comp. D101) 2-(Cyclopropanecarbonyl-amino)-6-(1-hydroxy-1-methyl-ethyl)-4,5,6,7-tetrahydro-benzo[b]thiophene-3-carboxylic acid amide,
(Comp. D102) 6-Ethyl-2-[(2-methyl-cyclopropanecarbonyl)-amino]-4,5,6,7-tetrahydro-benzo[b]thiophene-3-carboxylic acid amide,
(Comp. D103) 2-[3-(3,4-Dichloro-phenyl)-ureido]-4,5,6,7-tetrahydro-benzo[b]thiophene-3-carboxylic acid amide,
(Comp. D104) Z-2-(Cyclopropanecarbonyl-amino)-7-hydroxyimino-4,5,6,7-tetrahydro-benzo[b]thiophene-3-carboxylic acid amide,
(Comp. D105) 2-[3-(4-lodo-phenyl)-ureido]-6-oxo-4,5,6,7-tetrahydro-benzo[b]thiophene-3-carboxylic acid amide,
(Comp. D106) 2-[3-(4-Methoxy-phenyl)-ureido]-6-oxo-4,5,6,7-tetrahydro-benzo[b]thiophene-3-carboxylic acid amide,
(Comp. D107) 2-[3-(2,3-Dichloro-phenyl)-ureido]-4,5,6,7-tetrahydro-benzo[b]thiophene-3-carboxylic acid amide,
(Comp. D108) 2-(Cyclopropanecarbonyl-amino)-6-hydroxymethyl-4,5,6,7-tetrahydro-benzo[b]thiophene-3-carboxylic acid amide,
(Comp. D109) 2-[3-(4-Bromo-phenyl)-ureido]-6-methyl-4,5,6,7-tetrahydro-benzo[b]thiophene-3-carboxylic acid amide,
(Comp. D110) 6-Methyl-2-[3-(4-trifluoromethoxy-phenyl)-ureido]-4,5,6,7-tetrahydro-benzo[b]thiophene-3-carboxylic acid amide,
(Comp. D111) 2-[3-(4-trifluoromethoxy-phenyl)-ureido]-4,5,6,7-tetrahydro-benzo[b]thiophene-3-carboxylic acid amide,
(Comp. D 112) 2-(3-Biphenyl-4-yl-ureido)-4,5,6,7-tetrahydro-benzo[b]thiophene-3-carboxylic acid amide,
(Comp. D113) 2-[3-(4-Fluoro-phenyl)-ureido]-4,5,6,7-tetrahydro-benzo[b]thiophene-3-carboxylic acid amide,
(Comp. D114) 2-[3-(4-Benzyloxy-phenyl)-ureido]-4,5,6,7-tetrahydro-benzo[b]thiophene-3-carboxylic acid amide,
(Comp. D115) 2-(Cyclopropanecarbonyl-amino)-6-hydrazono-4,5,6,7-tetrahydro-benzo[b]thiophene-3-carboxylic acid amide,
(Comp. D116) 2-[({[4-(Trifluoromethyl)phenyl]amino}carbonyl)amino]-4,7-dihydro-5H-spiro[1-benzothiophene-6,2′-[1,3]dioxolane]-3-carboxamide,
(Comp. D117) 2-({[(4-Acetylphenyl)amino]carbonyl}amino)-4,7-dihydro-5H-spiro[1-benzothiophene-6,2′-[1,3]dioxolane]-3-carboxamide,
(Comp. D118) 2-(3-Cyclohexyl-ureido]-(6-oxo-ethylenylketal)-4,5,6,7-tetrahydro-benzo[b]thiophene-3-carboxylic acid amide,
(Comp. D119) 6-Oxo-2-[3-(4-trifluoromethyl-phenyl)-ureido]-4,5,6,7-tetrahydro-benzo[b]thiophene-3-carboxylic acid amide,
(Comp. D120) 2-[3-(4-Acetyl-phenyl)-ureido]-6-oxo-4,5,6,7-tetrahydro-benzo[b]thiophene-3-carboxylic acid amide,
(Comp. D121) 2-[3-(4-Fluoro-phenyl)-ureido]-6-oxo-4,5,6,7-tetrahydro-benzo[b]thiophene-3-carboxylic acid amide,
(Comp. D122) 2-(3-Cyclohexyl-ureido]-6-oxo-4,5,6,7-tetrahydro-benzo[b]thiophene-3-carboxylic acid amide,
(Comp. D123) N-(3-Carbamoyl-6-oxo-4,5,6,7-tetrahydro-benzo[b]thiophen-2-yl)-oxalamic acid methyl ester,
(Comp. D124) 2-[3-(4-Bromo-phenyl)-ureido]-4,5,6,7-tetrahydro-benzo[b]thiophene-3-carboxylic acid amide,
(Comp. D125) 2-{[(1,3-Benzodioxol-5-ylamino)carbonyl]amino}-4,7-dihydro-5H-spiro[1-benzothiophene-6,2′-[1,3]dioxolane]-3-carboxamide,
(Comp. D126) 2-{[(2-Methylcyclopropyl)carbonyl]amino}-4,7-dihydro-5H-spiro[1-benzothiophene-6,2′-[1,3]dioxolane]-3-carboxamide,
(Comp. D127) 2-[(2-Methyl-cyclopropanecarbonyl)-amino]-(6-oxo-ethylenylketal)-4,5,6,7-tetrahydro-benzo[b]thiophene-3-carboxylic acid amide,
(Comp. D128) 2-(Cyclopropanecarbonyl-amino)-6,6-difluoro-4,5,6,7-tetrahydro-benzo[b]thiophene-3-carboxylic acid amide,
(Comp. D129) 2-[3-(3-Carbamoyl-4,5,6,7-tetrahydro-benzo[b]thiophen-2-yl)-ureido]-3-methyl-butyric acid methyl ester,
(Comp. D130) N-(3-Carbamoyl-4,5,6,7-tetrahydro-benzo[b]thiophen-2-yl)-succinamic acid,
(Comp. D131) 2-(3-o-Tolyl-ureido)-4,5,6,7-tetrahydro-benzo[b]thiophene-3-carboxylic acid amide,
(Comp. D132) 3-Carbamoyl-2-[3-(4-methoxy-phenyl)-ureido]-4,5,6,7-tetrahydro-benzo[b]thiophene-6-carboxylic acid ethyl ester,
(Comp. D133) Cyclopropanecarboxylic acid (3-carbamimidoyl-4,5,6,7-tetrahydro-benzo[b]thiophen-2-yl)-amide,
(Comp. D134) 2-(Cyclopropanecarbonyl-amino)-6-hydroxyimino-4,5,6,7-tetrahydro-benzo[b]thiophene-3-carboxylic acid amide,
(Comp. D135) 2-(3-Benzyl-ureido)-4,5,6,7-tetrahydro-benzo[b]thiophene-3-carboxylic acid amide,
(Comp. D136) 2-[3-(2-Chloro-5-trifluoromethyl-phenyl)-ureido]-4,5,6,7-tetrahydro-benzo[b]thiophene-3-carboxylic acid amide,
(Comp. D137) 2-[3-(3,5-Bis-trifluoromethyl-phenyl)-ureido]-4,5,6,7-tetrahydro-benzo[b]thiophene-3-carboxylic acid amide,
(Comp. D138) 2-[3-(4-Chloro-3-trifluoromethyl-phenyl)-ureido]-4,5,6,7-tetrahydro-benzo[b]thiophene-3-carboxylic acid amide,
(Comp. D139) 2-[3-(3,5-Dimethoxy-phenyl)-ureido]-4,5,6,7-tetrahydro-benzo[b]thiophene-3-carboxylic acid amide,
(Comp. D140) 2-[3-(4-lodo-phenyl)-ureido]-6-hydroxy-4,5,6,7-tetrahydro-benzo[b]thiophene-3-carboxylic acid amide,
(Comp. D141) 6-Hydroxy-2-[3-(4-methoxy-phenyl)-ureido]-4,5,6,7-tetrahydro-benzo[b]thiophene-3-carboxylic acid amide,
(Comp. D142) 2-[3-(4-Fluoro-phenyl)-ureido]-6-hydroxy-4,5,6,7-tetrahydro-benzo[b]thiophene-3-carboxylic acid amide,
(Comp. D143) 6-Hydroxy-2-(3-phenyl-ureido)-4,5,6,7-tetrahydro-benzo[b]thiophene-3-carboxylic acid amide,
(Comp. D144) 2-(Cyclopropanecarbonyl-amino)-6-methoxy-4,5,6,7-tetrahydro-benzo[b]thiophene-3-carboxylic acid amide,
(Comp. D145) 2-[3-(3-Bromo-phenyl)-ureido]-4,5,6,7-tetrahydro-benzo[b]thiophene-3-carboxylic acid amide,
(Comp. D146) 3-Carbamoyl-2-(cyclopropanecarbonyl-amino)-4,5,6,7-tetrahydro-benzo[b]thiophene-6-carboxylic acid ethyl ester,
(Comp. D147) 2-(3-Cyclopropyl-thioureido)-4,5,6,7-tetrahydro-benzo[b]thiophene-3-carboxylic acid amide,
(Comp. D148) 2-[3-(3-Chloro-phenyl)-ureido]-4,5,6,7-tetrahydro-benzo[b]thiophene-3-carboxylic acid amide,
(Comp. D149) 2-[3-(3-Trifluoromethyl-phenyl)-ureido]-4,5,6,7-tetrahydro-benzo[b]thiophene-3-carboxylic acid amide,
(Comp. D150) 2-(3-Pyridin-3-yl-thioureido)-4,5,6,7-tetrahydro-benzo[b]thiophene-3-carboxylic acid amide,
(Comp. D151) 5-Methyl-isoxazole-4-carboxylic acid (3-Carbamoyl-4,5,6,7-tetrahydro-benzo[b]thiophen-2-yl)-amide,
(Comp. D152) 2-({[(2-Furylmethyl)amino]carbonyl}amino)-4,7-dihydro-5H-spiro[1-benzothiophene-6,2′-[1,3]dioxolane]-3-carboxamide,
(Comp. D153) 2-[3-(2-Methoxy-5-nitro-phenyl)-ureido]-4,5,6,7-tetrahydro-benzo[b]thiophene-3-carboxylic acid amide,
(Comp. D154) 2-(3-Isopropyl-ureido)-6-methyl-4,5,6,7-tetrahydro-benzo[b]thiophene-3-carboxylic acid amide,
(Comp. D155) 2-(3-Cyclopropyl-ureido)-6-methyl-4,5,6,7-tetrahydro-benzo[b]thiophene-3-carboxylic acid amide,
(Comp. D156) 6-Methyl-2-(2-methyl-butyrylamino)-4,5,6,7-tetrahydro-benzo[b]thiophene-3-carboxylic acid amide,
(Comp. D157) 6-Hydroxy-2-[3-(4-methylsulfanyl-phenyl)-ureido]-4,5,6,7-tetrahydro-benzo[b]thiophene-3-carboxylic acid amide,
(Comp. D158) 2-(Cyclobutanecarbonyl-amino)-6-ethyl-4,5,6,7-tetrahydro-benzo[b]thiophene-3-carboxylic acid amide,
(Comp. D159) 2-(Cyclopropanecarbonyl-amino)-6-propyl-4,5,6,7-tetrahydro-benzo[b]thiophene-3-carboxylic acid amide,
(Comp. D160) 2-(Cyclopropanecarbonyl-amino)-6-methyl-4-oxo-4,5,6,7-tetrahydro-benzo[b]thiophene-3-carboxylic acid amide,
(Comp. D161) 2-(Cyclopropanecarbonyl-amino)-6-methyl-7-oxo-4,5,6,7-tetrahydro-benzo[b]thiophene-3-carboxylic acid amide,
(Comp. D162) 2-[3-(2-Methyl-3-nitro-phenyl)-ureido]-4,5,6,7-tetrahydro-benzo[b]thiophene-3-carboxylic acid amide,
(Comp. D163) 2-{3-[4-(2-Morpholin-4-yl-ethoxy)-naphthalen-1-yl]-ureido]-4,5,6,7-tetrahydro-benzo[b]thiophene-3-carboxylic acid amide,
(Comp. D164) 2-[3-(3-Chloro-4-fluoro-phenyl)-ureido]-4,5,6,7-tetrahydro-benzo[b]thiophene-3-carboxylic acid amide,
(Comp. D165) 2-[3-(4-lsopropyl-phenyl)-ureido]-4,5,6,7-tetrahydro-benzo[b]thiophene-3-carboxylic acid amide,
(Comp. D166) 2-[3-(3-Carbamoyl-4,5,6,7-tetrahydro-benzo[b]thiophen-2-yl)-ureido]-3-(4-chloro-phenyl)-propionic acid methyl ester,
(Comp. D167) 2-[3-(3-Nitro-phenyl)-ureido]-4,5,6,7-tetrahydro-benzo[b]thiophene-3-carboxylic acid amide,
(Comp. D168) 2-(3-Cyclopropyl-ureido)-6-hydroxy-4,5,6,7-tetrahydro-benzo[b]thiophene-3-carboxylic acid amide,
(Comp. D169) 6-Hydroxy-2-[3-(4-trifluoromethoxy-phenyl)-ureido]-4,5,6,7-tetrahydro-benzo[b]thiophene-3-carboxylic acid amide,
(Comp. D170) 2-[3-(4-Bromo-phenyl)-ureido]-6-hydroxy-4,5,6,7-tetrahydro-benzo[b]thiophene-3-carboxylic acid amide,
(Comp. D171) 2-[3-(4-Methoxy-phenyl)-ureido]-4,5,6,7-tetrahydro-benzo[b]thiophene-3-carboxylic acid amide,
(Comp. D172) 2-(Cyclopropanecarbonyl-amino)-7-oxo-4,5,6,7-tetrahydro-benzo[b]thiophene-3-carboxylic acid amide,
(Comp. D173) 6-Hydroxy-2-[3-(4-trifluoromethyl-phenyl)-ureido]-4,5,6,7-tetrahydro-benzo[b]thiophene-3-carboxylic acid amide,
(Comp. D174) 2-(3-Cyclohexyl-ureido)-6-hydroxy-4,5,6,7-tetrahydro-benzo[b]thiophene-3-carboxylic acid amide,
(Comp. D175) 2-({[(4-lodophenyl)amino]carbonyl}amino)-4,7-dihydro-5H-spiro[1-benzothiophene-6,2′-[1,3]dioxolane]-3-carboxamide,
(Comp. D176) 2-({[(4-Methoxyphenyl)amino]carbonyl}amino)-4,7-dihydro-5H-spiro[1-benzothiophene-6,2′-[1,3]dioxolane]-3-carboxamide,
(Comp. D177) Benzyl-3-(aminocarbonyl)-4,7-dihydro-5H-spiro[1-benzothiophene-6,2′-[1,3]dioxolan]-2-ylcarbamate,
(Comp. D178) 2-({[(4-Fluorophenyl)amino]carbonyl}amino)-4,7-dihydro-5H-spiro[1-benzothiophene-6,2′-[1,3]dioxolane]-3-carboxamide,
(Comp. D179) 2-[3-(3-Methoxy-phenyl)-ureido]-4,5,6,7-tetrahydro-benzo[b]thiophene-3-carboxylic acid amide,
(Comp. D180) 2-[3-(2-Nitro-phenyl)-ureido]-4,5,6,7-tetrahydro-benzo[b]thiophene-3-carboxylic acid amide,
(Comp. D181) 2-[3-(3,5-Dichloro-phenyl)-ureido]-4,5,6,7-tetrahydro-benzo[b]thiophene-3-carboxylic acid amide,
(Comp. D182) 2-[3-(3,4,5-Trimethoxy-phenyl)-ureido]-4,5,6,7-tetrahydro-benzo[b]thiophene-3-carboxylic acid amide,
(Comp. D183) 2-[3-(2,4-Difluoro-phenyl)-ureido]-4,5,6,7-tetrahydro-benzo[b]thiophene-3-carboxylic acid amide,
(Comp. D184) 2-[3-(4-Nitro-phenyl)-ureido]-4,5,6,7-tetrahydro-benzo[b]thiophene-3-carboxylic acid amide,
(Comp. D185) 2-(Cyclopropanecarbonyl-amino)-4,5,6,7-tetrahydro-benzo[b]thiophene-3-carboxylic acid methylamide,
(Comp. D186) 2-(Cyclopentanecarbonyl-amino)-6-methyl-4,5,6,7-tetrahydro-benzo[b]thiophene-3-carboxylic acid amide,
(Comp. D187) 2-(Cyclobutanecarbonyl-amino)-6-methyl-4,5,6,7-tetrahydro-benzo[b]thiophene-3-carboxylic acid amide,
(Comp. D188) 2-(3-Isopropyl-ureido)-4,5,6,7-tetrahydro-benzo[b]thiophene-3-carboxylic acid amide,
(Comp. D189) 6-Ethyl-2-[2-(4-nitro-phenyl)-acetylamino]-4,5,6,7-tetrahydro-benzo[b]thiophene-3-carboxylic acid amide,
(Comp. D190) 6-Methyl-2-[2-(4-nitro-phenyl)-acetylamino]-4,5,6,7-tetrahydro-benzo[b]thiophene-3-carboxylic acid amide,
(Comp. D191) 2-(Cyclopropanecarbonyl-amino)-4,5,6,7-tetrahydro-benzo[b]thiophene-3-carboxylic acid propylamide,
(Comp. D192) 2-[3-(2-Methoxy-benzyl)-ureido]-4,5,6,7-tetrahydro-benzo[b]thiophene-3-carboxylic acid amide,
(Comp. D193) 2-(Cyclopentanecarbonyl-amino)-6-phenyl-4,5,6,7-tetrahydro-benzo[b]thiophene-3-carboxylic acid amide,
(Comp. D194) 2-(Cyclopentanecarbonyl-amino)-6-ethyl-4,5,6,7-tetrahydro-benzo[b]thiophene-3-carboxylic acid amide,
(Comp. D195) (5-Amino-pentyl)-carbamic acid 3-carbamoyl-2-(cyclopropanecarbonyl-amino)-4,5,6,7-tetrahydro-benzo[b]thiophen-6-yl ester,
(Comp. D196) 2-(Cyclopropanecarbonyl-amino)-5,6-dihydro-4H-cyclopenta[b]thiophene-3-carboxylic acid amide,
(Comp. D197) 6-Hydroxy-2-(2-hydroxy-acetylamino)-4,5,6,7-tetrahydro-benzo[b]thiophene-3-carboxylic acid amide,
(Comp. D198) {5-[3-Carbamoyl-2-(cyclopropanecarbonyl-amino)-4,5,6,7-tetrahydro-benzo[b]thiophen-6-yloxycarbonylamino]-pentyl}-carbamic acid tert-butyl ester,
(Comp. D199) 2-[(Anilinocarbonyl)amino]-4,7-dihydro-5H-spiro[1-benzothiophene-6,2′-[1,3]dioxolane]-3-carboxamide,
(Comp. D200) 2-({[(4-Ethoxyphenyl)amino]carbonyl}amino)-4,7-dihydro-5H-spiro[1-benzothiophene-6,2′-[1,3]dioxolane]-3-carboxamide,
(Comp. D201) 2-(Cyclopropanecarbonyl-amino)-6-hydroxy-6-methyl-4,5,6,7-tetrahydro-benzo[b]thiophene-3-carboxylic acid amide,
(Comp. D202) 6-Hydroxy-2-[(2-methyl-cyclopropanecarbonyl)-amino]-4,5,6,7-tetrahydro-benzo[b]thiophene-3-carboxylic acid amide,
(Comp. D203) 6-Hydroxyimino-2-[3-(4-methoxy-phenyl)-ureido]-4,5,6,7-tetrahydro-benzo[b]thiophene-3-carboxylic acid amide,
(Comp. D204) 2-[(2-Methyl-cyclopropanecarbonyl)-amino]-6-oxo-4,5,6,7-tetrahydro-benzo[b]thiophene-3-carboxylic acid amide,
(Comp. D205) (3-Chloro-4-fluoro-phenyl)-carbamic acid 3-carbamoyl-2-(cyclopropanecarbonyl-amino)-4,5,6,7-tetrahydro-benzo[b]thiophen-6-yl ester,
(Comp. D206) 6-Methyl-2-[(2-methyl-cyclopropanecarbonyl)-amino]-4,5,6,7-tetrahydro-benzo[b]thiophene-3-carboxylic acid amide,
(Comp. D207) 2-[3-(4-Ethoxy-phenyl)-ureido]-6-hydroxy-4,5,6,7-tetrahydro-benzo[b]thiophene-3-carboxylic acid amide,
(Comp. D208) 2-[3-(3-Carbamoyl-4,5,6,7-tetrahydro-benzo[b]thiophen-2-yl)-ureido]-4-methylsulfanyl-butyric acid methyl ester,
(Comp. D209) 6-Hydroxy-2-{3-[4-(1-hydroxyethyl)-phenyl]-ureido}-4,5,6,7-tetrahydro-benz b]thiophene-3-carboxylic acid amide,
(Comp. D210) 2-(Cyclopropanecarbonyl-amino)-4,5,6,7,8,9-hexahydro-cycloocta[b]thiophene-3-carboxylic acid amide;
(Comp. D211) 6-Hydroxy-2-(3-isopropyl-ureido)-4,5,6,7-tetrahydro-benzo[b]thiophene-3-carboxylic acid amide,
(Comp. D212) 2-(3-Benzo[1,3]dioxol-5-yl-ureido)-6-hydroxy-4,5,6,7-tetrahydro-benzo[b]thiophene-3-carboxylic acid amide,
(Comp. D213) Phenyl-3-(aminocarbonyl)-4,7-dihydro-5H-spiro[1-benzothiophene-6,2′-[1,3]dioxolan]-2-ylcarbamate,
(Comp. D214) 2-(Cyclopropanecarbonylamino)-6-methoxyimino-4,5,6,7-tetrahydro-benzo[b]thiophene-3-carboxylic acid amide,
(Comp. D215) 6-Benzyloxyimino-2-(cyclopropanecarbonylamino)-4,5,6,7-tetrahydro-benzo[b]thiophene-3-carboxylic acid amide,
(Comp. D216) 2-(Cyclopropanecarbonyl-amino)-4,5,6,7-tetrahydro-benzo[b]thiophene-3-sulfonic acid,
(Comp. D217) 2-(3-exo-Bicyclo[2.2.1]hept-2-yl-methyl-ureido]-(6-oxo-ethylenylketal)-4,5,6,7-tetrahydro-benzo[b]thiophene-3-carboxylic acid amide,
(Comp. D218) 2-[3-(4-tert-Butyl-cyclohexyl)-ureido]-(6-oxo-ethylenylketal)-4,5,6,7-tetrahydro-benzo[b]thiophene-3-carboxylic acid amide,
(Comp. D219) 4-[3-(3-Carbamoyl-6-oxo-ethylenylketal-4,5,6,7-tetrahydro-benzo[b]thiophen-2-yl)-ureido]-piperidine-1-carboxylic acid ethyl ester,
(Comp. D220) 2-[3-(1-Benzyl-piperidin-4-yl)-ureido]-6-oxo-ethylenylketal-4,5,6,7-tetrahydro-benzo[b]thiophene-3-carboxylic acid amide,
(Comp. D221) 2-[3-(2-Hydroxy-ethyl)-ureido]-6-oxo-ethylenylketal-4,5,6,7-tetrahydro-benzo[b]thiophene-3-carboxylic acid amide,
(Comp. D222) 2-[3-(2,3-Dihydroxy-propyl)-ureido]-6-oxo-ethylenylketal-4,5,6,7-tetrahydro-benzo[b]thiophene-3-carboxylic acid amide,
(Comp. D223) 2-[3-(R(−)-1-Cyclohexyl-ethyl)-ureido]-6-oxo-ethylenylketal-4,5,6,7-tetrahydro-benzo[b]thiophene-3-carboxylic acid amide,
(Comp. D224) 6-Oxo-ethylenylketal-2-[3-(2-thiophen-2-yl-ethyl)-ureido]-4,5,6,7-tetrahydro-benzo[b]thiophene-3-carboxylic acid amide,
(Comp. D225) 6-Oxo-ethylenylketal-2-[3-(2-pyridin-2-yl-ethyl)-ureido]-4,5,6,7-tetrahydro-benzo[b]thiophene-3-carboxylic acid amide,
(Comp. D226) 2-[3-(3-Morpholin-4-yl-propyl)-ureido]-6-oxo-ethylenylketal-4,5,6,7-tetrahydro-benzo[b]thiophene-3-carboxylic acid amide,
(Comp. D227) 2-(3-Bicyclo[2.2.1]hept-2-yl-ureido)-6-oxo-ethylenylketal-4,5,6,7-tetrahydro-benzo[b]thiophene-3-carboxylic acid amide,
(Comp. D228) 6-Oxo-ethylenylketal-2-[3-(1R,2R,3R,5S-2,6,6-trimethyl-bicyclo[3.1.1 ]hept-3-yl)-ureido]-4,5,6,7-tetrahydro-benzo[b]thiophene-3-carboxylic acid amide,
(Comp. D229) 6-Oxo-ethylenylketal-2-[3-(1S,2S,3S,5R-2,6,6-trimethyl-bicyclo[3.1.1]hept-3-yl)-ureido]-4,5,6,7-tetrahydro-benzo[b]thiophene-3-carboxylic acid amide,
(Comp. D230) 2-[3-(2-Cyclohex-1-enyl-ethyl)-ureido]-6-oxo- ethylenylketal-4,5,6,7-tetrahydro-benzo[b]thiophene-3-carboxylic acid amide,
(Comp. D231) 2-[3-(3-Chloro-4-methyl-phenyl)-ureido]-(6-oxo-ethylenylketal)-4,5,6,7-tetrahydro-benzo[b]thiophene-3-carboxylic acid amide,
(Comp. D232) 2-[3-(3-Chloro-2-methyl-phenyl)-ureido]-(6-oxo-ethylenylketal)-4,5,6,7-tetrahydro-benzo[b]thiophene-3-carboxylic acid amide,
(Comp. D233) 2-(3-Benzo[1,3]dioxol-5-yl-ureido)-3-carbamoyl-4,5,6,7-tetrahydro-benzo[b]thiophene-6-carboxylic acid ethyl ester,
(Comp. D234) 3-Carbamoyl-2-[3-(4-methylsulfanyl-phenyl)-ureido]-4,5,6,7-tetrahydro-benzo[b]thiophene-6-carboxylic acid ethyl ester,
(Comp. D235) (3-carbamoyl-6,6-ethylenedioxy-4,5,6,7-tetrahydro-benzo[b]thiophen-2-yl)-carbamic acid phenyl ester,
(Comp. D236) 2-[3-(S(−)-1-Cyclohexyl-ethyl)-ureido]-6-oxo-ethylenylketal-4,5,6,7-tetrahydro-benzo[b]thiophene-3-carboxylic acid amide.
(Comp. D237) 2-{(3-chloro-4-fluorophenyl)- ureido]-4,7-dihydro-5H- spiro[1-benzothiophene-6,2′-[1,3]dioxolane]-3-carboxamide,
(Comp. D238) 4-[({[7′-(aminocarbonyl)spiro[1,3-dioxolane-2,3′-[9]thiabicyclo [4.3.1]deca[6(10),7]dien]-8′-yl]amino}carbonyl)amino]-1-hydroxypyridinium,
(Comp. D239) 2-{(3,4,5-trimethoxyphenyl)-ureido]-4,7-dihydro-5H-spiro[1-benzothiophene-6,2′- [1,3]dioxolane]-3-carboxamide,
(Comp. D240) (3-Carbamoyl-6-oxo-4,5,6,7-tetrahydrobenzo[b]thiophen-2-yl)-carbamic acid benzyl ester,
(Comp. D241) 2-{[(3-methylsulfanylphenyl)amino]carbonyl)amino}-4,7-dihydro-5H-spiro[1-benzothiophene-6,2′-[1,3]dioxolane]-3-carboxamide,
(Comp. D242) 2-[({[4-difluoromethoxyphenyl]amino}carbonyl)amino]-4,7-dihydro-5H-spiro[1-benzothiophene-6,2′-[1,3]dioxolane]-3-carboxamide,
(Comp. D243) 2-[({[3-Trifluoromethylsulfanylphenyl]amino}carbonyl)amino]-4,7-dihydro-5H spiro[1-benzothiophene-6,2′-[1,3]dioxolane]-3-carboxamide,
(Comp. D244) 6-Hydroxy-2-[3-(3-methylsulfanyl-phenyl)-ureido]-4,5,6,7-tetrahydro-benzo[b]thiophene-3-carboxylic acid amide,
(Comp. D245) 2-[3-(4-Difluoromethoxy-phenyl)-ureido]-6-hydroxy-4,5,6,7-tetrahydro-benzo[b]thiophene-3-carboxylic acid amide,
(Comp. D246) 2-[3-(2,4-difluoro-phenyl)-ureido]-(6-oxo-ethylenylketal)-4,5,6,7-tetrahydro-benzo[b]thiophene-3-carboxylic acid amide,
(Comp. D247) 2-[3-(2,4-Difluoro-phenyl)-ureido]-6-oxo-4,5,6,7-tetrahydro-benzo[b]thiophene-3-carboxylic acid amide,
(Comp. D248) 2-[3-(3-Chloro-4-methyl-phenyl)-ureido]-6-hydroxy-4,5,6,7-tetrahydro-benzo[b]thiophene-3-carboxylic acid amide,
(Comp. D249) 2-[3-(3-Chloro-2-methyl-phenyl)-ureido]-6-hydroxy-4,5,6,7-tetrahydro-benzo[b]thiophene-3-carboxylic acid amide,
(Comp. D250) 2-[3-(2-Methylsulfanyl-phenyl)-ureido]-(6-oxo-ethylenylketal)-4,5,6,7-tetrahydro-benzo[b]thiophene-3-carboxylic acid amide,
(Comp. D251) 2-[3-(2-Trifluoromethoxy-phenyl)-ureido]-(6-oxo-ethylenylketal)-4,5,6,7-tetrahydro-benzo[b]thiophene-3-carboxylic acid amide,
(Comp. D252) 2-[3-(2-Difluoromethoxy-phenyl)-ureido]-(6-oxo-ethylenylketal)-4,5,6,7-tetrahydro-benzo[b]thiophene-3-carboxylic acid amide,
(Comp. D253) 2-[3-(4-Trifluoromethylsulfanyl-phenyl)-ureido]-(6-oxo-ethylenylketal)-4,5,6,7-tetrahydro-benzo[b]thiophene-3-carboxylic acid amide,
(Comp. D254) 6-Hydroxy-2-[3-(2-methylsulfanyl-phenyl)-ureido]-4,5,6,7-tetrahydro-benzo[b]thiophene-3-carboxylic acid amide,
(Comp. D255) 6-Hydroxy-2-[3-(2-trifluoromethoxy-phenyl)-ureido]-4,5,6,7-tetrahydro-benzo[b]thiophene-3-carboxylic acid amide,
(Comp. D256) 2-[3-(2-Difluoromethoxy-phenyl)-ureido]-6-hydroxy-4,5,6,7-tetrahydro-benzo[b]thiophene-3-carboxylic acid amide,
(Comp. D257) 6-Hydroxy-2-[3-(4-trifluoromethylsulfanyl-phenyl)-ureido]-4,5,6,7-tetrahydro-benzo[b]thiophene-3-carboxylic acid amide.

8. Compound according to claim 1 for use as a pharmaceutically active agent.

9. Use of a compound according to claim 1 as an inhibitor for a protein kinase.

10. Use of a compound according to claim 1 for the manufacture of a pharmaceutical composition for prophylaxis and/or treatment of virally and/or bacterially induced diseases and/or infections.

11. Use according to claim 10, wherein the bacterially induced disease is one caused by a bacterium of the genus legionella.

12. Use according to claim 10, wherein the disease is legionnaires' disease, tuberculosis, leprosy or mycobacterially induced meningitis.

13. Use according to claim 10, wherein the bacterially induced disease or infection is caused by a mycobacterium.

14. Use according to claim 13, wherein the mycobacterium is Mycobacterium tuberculosis or Mycobacterium leprae.

15. Use according to claim 9, wherein the protein kinase is secreted from a cell to an environment of the cell.

16. Use according to claim 9, wherein the protein kinase is a mycobacterial kinase.

17. Use according to claim 16, wherein the protein kinase is from Mycobacterium tuberculosis or Mycobacterium leprae.

18. Use according to claim 17, wherein the protein kinase from Mycobacterium tuberculosis or Mycobacterium leprae is protein kinase G (PknG).

19. Use of at least one compound according to claim 1 for the preparation of a pharmaceutical composition for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke.

20. Use according to claim 19, wherein the infectious diseases, including opportunistic diseases, are virally induced infectious diseases, including opportunistic diseases.

21. Use according to claim 20, wherein the virally induced infectious diseases, including opportunistic diseases, are caused by retroviruses, human endogenous retroviruses, lentiviruses, oncoretroviruses, hepadnaviruses, herpesviruses, flaviviridae, and/or adenoviruses.

22. Use according to claim 21, wherein the lentivirus, oncoretrovirus, hepadnavirus or herpesivirus is selected from the group comprising: HIV-1, HIV-2, FIV, BIV, SIVs, SHIVs, CAEV, VMV or EIAV, preferably HIV-1 and HIV-2; HTLV-I, HTLV-II or BLV, preferably HTLV-I or HTLV-II; HBV, GSHV or WHV, preferably HBV; HSV I, HSV II, EBV, VZV, HCMV or HHV 8, preferably HCMV.

23. Use according to claim 19, wherein the infective disease including opportunistic infection is selected from the group comprising AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis.

24. Use according to claim 19, wherein the prion diseases is selected from the group comprising Scrapie, TME, CWD, BSE, CJD, vCJD, GSS, FFI, Kuru, and Alpers Syndrome.

25. Use according to claim 19, wherein the immunological disease and/or autoimmune disease is selected from the group comprising: asthma, diabetes, rheumatic diseases, AIDS, rejection of transplanted organs and tissues, rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper-IgE syndrome, Wiskott-Aldrich syndrome, ataxia-telangiectasia, immune mediated cancers, white cell defects, autoimmune diseases, systemic lupus erythematosus, rheumatoid arthritis (RA), multiple sclerosis (MS), immune-mediated or Type 1 Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, goodpastture syndrome, myasthenia gravis pseudoparalytica, ophtalmia sympatica, phakogene uveitis, chronical agressivce hepatitis, primary billiary cirrhosis, autoimunehemolytic anemy, Werlof disease.

26. Use according to claim 19, wherein the bipolar and/or clinical disorder is selected from the group comprising: adjustment disorders, anxiety disorders, delirium, dementia, amnestic and other cognitive disorders, disorders usually first diagnosed in infancy, childhood, or adolescence, dissociative disorders, eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder, panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

27. Use according to claim 26, wherein the anxiety disorders, delirium, dementia, amnestic and other cognitive disorders, disorders usually first diagnosed in infancy, childhood, or adolescence, dissociative disorders, eating disorders, mood disorders, schizophrenia and other psychotic disorders, sexual and gender identity disorders, sleep disorders, somatoform disorders, substance-related disorders are selected from the group comprising: acute stress disorder, agoraphobia without history of panic disorder, anxiety disorder due to general medical condition, generalized anxiety disorder, obsessive-compulsive disorder, panic disorder with agoraphobia, panic disorder without agoraphobia, posttraumatic stress disorder, specific phobia, social phobia, substance-induced anxiety disorder, delirium due to a general medical condition, substance intoxication delirium, substance withdrawal delirium, delirium due to multiple etiologies, Alzheimer's, Creutzfeldt-Jakob disease, head trauma, Huntington's disease, HIV disease, Parkinson's disease, Pick's disease, substance-induced persisting, vascular, dementia due to other general medical conditions, dementia due to multiple etiologies, amnestic disorder due to a general medical condition, substance-induced persisting amnestic disorder, mental retardation, learning disorders, mathematics disorder, reading disorder, disorder of written expression, learning disorder, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder, dissociative amnesia, depersonalization disorder, dissociative fugue, dissociative identity disorder, anorexia nervosa, bulimia nervosa, mood episodes, major depressive episode, hypomanic episode, manic episode, mixed episode, depressive disorders, dysthymic disorder, major depressive disorder, single episode, recurrent, bipolar disorders, bipolar I disorder, bipolar II disorder, cyclothymic disorder, mood disorder due to a general medical condition, substance-induced mood disorder, schizophreniform disorder, schizoaffective disorder, delusional disorder, brief psychotic disorder, shared psychotic disorder, psychotic disorder due to a general medical condition, delusions, hallucinations, substance-induced psychotic disorder, female sexual arousal disorder, orgasmic disorders, premature ejaculation, sexual pain disorders, dyspareunia, vaginismus, sexual dysfunction due to a general medical condition, female dyspareunia, female hypoactive sexual desire disorder, male erectile disorder, male hypoactive sexual desire disorder, male dyspareunia, other female sexual dysfunction, other male sexual dysfunction, substance-induced sexual dysfunction, sexual dysfunction, exhibitionism, fetishism, frotteurism, pedophilia, masochism, sadism, transvestic fetishism, voyeurism, paraphilia, gender identity disorder, dyssomnias, breathing-related sleep disorder, circadian rhythm sleep disorder, hypersomnia, hypersomnia related to another mental disorder, insomnia, insomnia related to another mental disorder, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, undifferentiated somatoform disorder, alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, sleep disorder.

28. Use according to claim 19, wherein the cardiovascular diseases are selected from the group consisting of:
adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

29. Use according to claim 19, wherein the proliferative disease is selected from the group comprising:
adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

30. Use according to claim 19, wherein said diabetes is selected from Type I diabetes or Type II diabetes.

31. Use according to claim 19, wherein said inflammation is mediated by the cytokines TNF-α, IL-1β, GM-CSF, IL-6 and/or IL-8.

32. Use according to claim 19 wherein the inflammatory disease is caused, induced, initiated and/or enhanced by bacteria, viruses, prions, parasites, fungi, and/or caused by irritative, traumatic, metabolic, allergic, autoimmune, or idiopathic reasons.

33. Use according to claim 32 wherein the viruses and bacteria are selected from the group comprising human immunodeficiency virus-I, herpes viruses, herpes simplex virus, herpes zoster virus, cytomegalovirus, mycoplasma pulmonis, ureaplasma urealyticum, mycoplasma pneumoniae, chlamydia pneumoniae, C. pneumoniae, Helicobacter pylori, and propriono-bacterium.

34. Use according to claim 19, wherein the inflammatory disease is selected from the group comprising inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx.

35. Use according to claim 34 wherein the inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx are selected from the group comprising:
abscessation, acanthameba, acanthamebiasis, acne vulgaris, actinomycosis, acute inflammatory dermatoses, acute laryngeal infections of adults, acute multifocal placoid pigmentary epitheliopathy, acute (thermal) injury, acute retinal necrosis, acute suppurative otitis media, algal disorders, allergic contact dermatitis, amyloidosis angioedema, ankylosing spondylitis, aspergillosis, atopic dermatitis, Aujeszky's disease, autoantibodies in vasculitis, babesiosis, bacterial disorders, bacterial laryngitis, bacterial meningitis, Behcet's disease, birdshot choroidopathy, blastomycosis, borna disease, brucellosis, bullous myringitis, bursitis, candidiasis, canine distemper encephalomyelitis, canine distemper encephalomyelitis in immature animals, canine ehrlichiosis, canine herpes virus encephalomyelitis, cholesteatoma, chronic (granulomatous) diseases, chronic inflammatory dermatoses, chronic relapsing encephalomyelitis, chronic suppurative otitis media, cicatricial pemphigoid, coccidiomycosis, coccidioidomycosis, common upper respiratory infection, contact ulcer and granuloma, Crohn's disease, cryptococcosis, cysticercosis, dermatomyositis, diphtheria, discoid lupus erythematosus, drug-induced vasculitis, drug or hypersensitivity reaction, encephalitozoonosis, eosinophilic meningoencephalitis, erythemal multiforme (EM minor), feline leukemia virus, feline immunodeficiency virus, feline infectious peritonitis, feline polioencephalomyelitis, feline spongiform encephalopathy, fibromyositis, Fuch's heterochromic cyclitis, gastroesophageal (laryngopharyngeal) reflux disease, giant cell arteritis, glanders, glaucomatocyclitic crisis, gonorrhea granular myringitis, granulomatous meningoencephalomyelitis, herpes simplex, histoplasmosis, idiopathic diseases, idiopathic inflammatory disorders, immune and idiopathic disorders, infections of the immunocompromised host, infectious canine hepatitis, inhalation laryngitis, interstitial nephritis, irritant contact dermatitis, juvenile rheumatoid arthritis, Kawasaki's disease, La Crosse virus encephalitis, laryngeal abscess, laryngotracheitis (croup), leishmaniasis, lens-induced uveitis, leprosy, leptospirosis, leukemia, lichen planus, lupus, lyme disease, lymphoma, meningitis, meningoencephalitis in greyhounds, miscellaneous meningitis/meningoencephalitis, microscopic polyangiitis, multifocal choroiditis, multifocal distemper encephalomyelitis in mature animals, multiple sclerosis, muscle tension dysphonias, mycotic (fungal) diseases, mycotic diseases of the CNS, necrotizing encephalitis, neosporosis, old dog encephalitis, onchocerciasis, parasitic encephalomyelitis, parasitic infections, pars planitis, parvovirus encephalitis, pediatric laryngitis, pollution and inhalant allergy, polymyositis, post-vaccinal canine distemper encephalitis, post-vaccinal rabies, prion protein induced diseases, protothecosis, protozoal encephalitis-encephalomyelitis, psoriasis, psoriatic arthritis, pug dog encephalitis, pyogranulomatous meningoencephalomyelitis, rabies, radiation injury, radiation laryngitis, radionecrosis, relapsing polychondritis, Reiters's syndrome, retinitis pigmentosa, retinoblastoma, rheumatoid arthritis, rickettsial disorders, rocky mountain spotted fever, salmon poisoning, sarcocystosis, sarcoidosis, schistosomiasis, scleroderma, scleroma, serpiginous choroiditis, shaker dog disease, Sjogren's syndrome, spasmodic croup, spirochetal (syphilis) diseases, spongiotic dermatitis, sporotrichosis, steroid responsive meningitis-arteritis, Stevens-Johnson syndrome (SJS, EM major), supraglottitis (epiglottitis), sympathetic ophthalmia, syngamus laryngeus, syphilis, systemic lupus erythematosus, systemic vasculitis in sarcoidosis, Takayasu's arteritis, tendinitis (tendonitis), thromboangiitis obliterans (Buerger's Disease), tick-borne encephalitis in dogs, toxic epidermal necrolysis (TEN), toxocariasis, toxoplasmosis, trauma, traumatic laryngitis, trichinosis, trypanosomiasis, tuberculosis, tularemia, ulcerative colitis, urticaria (hives), vasculitis, vasculitis and malignancy, vasculitis and rheumatoid arthritis, vasculitis in systemic lupus erythematosus, vasculitis in the idiopathic inflammatory myopathies, vasculitis of the central nervous system, vasculitis secondary to bacterial, fungal, and parasitic infection, viral disorders, viral laryngitis, vitiligo, vocal abuse, vocal-cord hemorrhage, Vogt Koyanagi Harada syndrome, Wegener's granulomatosis, and Whipple's disease.

36. Use according to claim 19, wherein the transplant rejection is selected from the group comprising heart transplant rejection, heart-lung transplant rejection, lung transplant rejection, liver transplant rejection, kidney transplant rejection, pancreas transplant rejection, spleen transplant rejection, skin transplant rejection, tissue transplant rejection, bone marrow transplant rejection, spinal marrow transplant rejection, hormone producing glands transplant rejection, gonads and gonadal gland transplant rejection, graft-versus-host-diseases and host-versus-graft-diseases.

37. Use according to claim 19, wherein the neurodegenerative diseases are selected from the group comprising:
Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

38. Pharmaceutical composition comprising at least one compound of the general formula (I) and/or pharmaceutically acceptable salts thereof together with pharmaceutically acceptable carrier, excipient and/or diluents.

39. Method for amidation of an carboxylic acid ester to give the corresponding primary carboxylic acid amide, comprising the step of reacting an carboxylic acid ester with an alkali metal amide in the presence of a polar aprotic solvent, which is essentially inert against the alkali metal amide, wherein the molar ratio of carboxylic acid ester to alkali metal amide lies in the range of 1:1 to 1:15.

40. The method according to claim 39, wherein a compound of the following general formula (Ia) 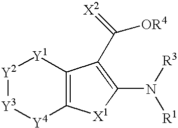
wherein
X² represents O or S, and
R¹, R³, R⁴, Y¹-Y⁴ and X¹ have the meaning as defined in claim 1,
is reacted with an alkali metal amide in the presence of a polar aprotic solvent to give the compound of the following general formula (Ib) 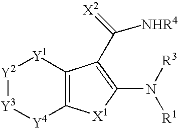
wherein
X² represents O or S, and
R¹, R³, R⁴, Y¹-Y⁴ and X¹ have the meaning as defined in claim 1.

41. The method according to claim 39, wherein the alkali metal amide is LiNH₂ or NaNH₂.

42. The method according to claim 39, wherein the carboxylic acid ester is a compound according to the following general formula (D): 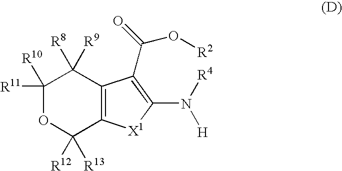
which is amidated to give the primary carboxylic acid amide according to formula (E), 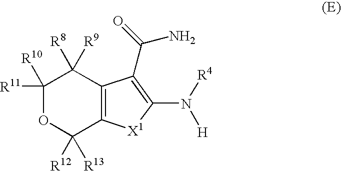
wherein in formulas (D) and (E)
X¹ is selected from S, O, or NR¹, and R¹ is selected from H, substituted or unsubstituted C₁-C₆-alkyl,
R² is linear or branched C₁-C₆ alkyl or aryl and preferably is methyl, ethyl, phenyl or benzyl,
R⁴ is selected from H, —C(═X²)R⁵ and —SO₂R⁵, wherein X² is O, S or NH and
R⁵ is selected from substituted or unsubstituted C₃-C₁₀-cycloalkyl, C₁-C₆-alkyl, aryl, heteroaryl, heterocycloalkyl, C₂-C₄-alkenyl, C₂-C₄-alkinyl, adamantyl, 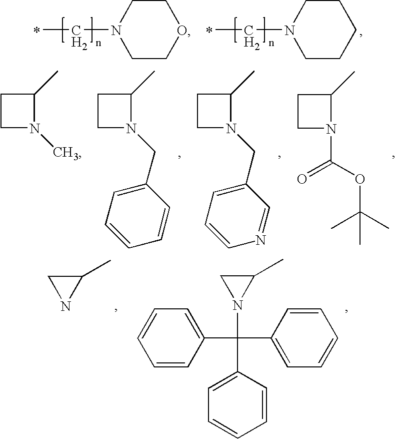
or —(CH₂)ₙ—NR⁶R⁷,
wherein R⁶ and R⁷ are independently selected from substituted or unsubstituted C₁-C₄-alkyl or C₂-C₄-alkenyl and wherein n=1 to 6,
or NR⁶R⁷,
wherein
R⁶ is selected from H, C₁-C₆-alkyl, and
R⁷ is selected from substituted or unsubstituted C₃-C₁₀-cycloalkyl, C₁-C₆-alkyl, aryl, heteroaryl, heterocycloalkyl, C₂-C₄-alkenyl, C₂-C₄-alkinyl, or adamantyl,
R⁸ is H and R⁹ is selected from H, substituted or unsubstituted C₁-C₆-alkyl
R¹⁰ is selected from H, substituted or unsubstituted C₁-C₆-alkyl, C₁-C₆-alkoxy, or OH
R₁₁ is selected from H and substituted or unsubstituted C₁-C₆-alkyl
R₁₂ is selected from H and substituted or unsubstituted C₁-C₆-alkyl, C₁-C₆-alkoxy, or OH, and
R¹³ is selected from H or substituted or unsubstituted C₁-C₆-alkyl,
and stereoisomeric and regioisomeric forms and pharmaceutically acceptable salts of these compounds.

43. The method according to claim 42, wherein in general formulas (D) and (E)
X¹ is S
R² is methyl or ethyl,
R⁴ is —C(═O)R⁵ and R⁵ is selected from methyl, ethyl, n-propyl, iso-propyl, n-butyl, sec.-butyl, iso-butyl, tert.-butyl, cyclopropyl, cyclobutyl, cyclopentyl, cyclohexyl, C₁-C₁₀-cycloalkyles substituted by at least one methyl or carboxyl group, phenyl, furanyl, thienyl, pyrrolyl, pyridyl, pyrrolidinyl, piperidinyl, tetrahydrofuranyl, ethenyl, cis-prop-1-enyl, trans-prop-1-enyl, cis-prop-2-enyl, trans-prop-2-enyl, but-1-enyl, cis-but-2-enyl, trans-but-2-enyl, but-3-enyl, prop-1-inyl, prop-2-inyl, but-1-inyl, but-2-inyl, but-3-inyl or adamantyl,
R⁸ is H and R⁹ is selected from H, substituted or unsubstituted C₁-C₆-alkyl,
R¹⁰ is selected from H, substituted or unsubstituted C₁-C₆-alkyl, C₁-C₆-alkoxy, or OH,
R₁₁ is selected from H and substituted or unsubstituted C₁-C₆-alkyl,
R₁₂ is selected from H and substituted or unsubstituted C₁-C₆-alkyl, C₁-C₆-alkoxy, or OH, and
R¹³ is selected from H or substituted or unsubstituted C₁-C₆-alkyl.
